SEQUENCE LISTING (209 sequences)

SEQ ID NO: 1 - DNA, 20 bp, Artificial Sequence
  Synthetic
gctacttcggttggactctg

SEQ ID NO: 2 - DNA, 19 bp, Artificial Sequence
  Synthetic
cctctcactcactctaggc

SEQ ID NO: 3 - DNA, 20 bp, Artificial Sequence
  Synthetic
gggcttgacaccacttgaac

SEQ ID NO: 4 - DNA, 22 bp, Artificial Sequence
  Synthetic
gagaatgaaatgaggctttgag

SEQ ID NO: 5 - DNA, 20 bp, Artificial Sequence
  Synthetic
ggccagcaatctggattaaa

SEQ ID NO: 6 - DNA, 20 bp, Artificial Sequence
  Synthetic
aatgctgctaacgccttcat

SEQ ID NO: 7 - DNA, 20 bp, Artificial Sequence
  Synthetic
tggtgcctgctactgtgtat

SEQ ID NO: 8 - DNA, 20 bp, Artificial Sequence
  Synthetic
tctcaggtcccacattcacc

SEQ ID NO: 9 - DNA, 97 bp, Artificial Sequence
  Synthetic
tgctgttgacagtgagcgcttggtgctgaaagattgtctatagtgaagccacagatgtatagacaatctttcagcaccaaatgcctactgcctcgga

SEQ ID NO: 10 - DNA, 97 bp, Artificial Sequence
  Synthetic
tgctgttgacagtgagcgacaggtccaggtttaggtctaatagtgaagccacagatgtattagacctaaacctggacctgctgcctactgcctcgga

SEQ ID NO: 11 - DNA, 97 bp, Artificial Sequence
  Synthetic
tgctgttgacagtgagcgccagagacaaagaagatcctaatagtgaagccacagatgtattaggatcttctttgtctctgttgcctactgcctcgga

SEQ ID NO: 12 - DNA, 97 bp, Artificial Sequence
  Synthetic
tgctgttgacagtgagcgacagaaagatgttgacagtgattagtgaagccacagatgtaatcactgtcaacatctttctggtgcctactgcctcgga

SEQ ID NO: 13 - DNA, 23 bp, Artificial Sequence
  Synthetic
aagctaaagctggagcagagagg

SEQ ID NO: 14 - DNA, 23 bp, Artificial Sequence
  Synthetic
gggaagaggccactatcaattcc

SEQ ID NO: 15 - DNA, 23 bp, Artificial Sequence
  Synthetic
cccaaggactggtctgaggtaac

SEQ ID NO: 16 - DNA, 23 bp, Artificial Sequence
  Synthetic
ttaagccatttgcttgtccttgc

SEQ ID NO: 17 - DNA, 20 bp, Artificial Sequence
  Synthetic
acatgaccttgaacccactc

SEQ ID NO: 18 - DNA, 20 bp, Artificial Sequence
  Synthetic
aatgtgagcgagtaacaacc

SEQ ID NO: 19 - DNA, 19 bp, Artificial Sequence
  Synthetic
catgtgactccgaccagga

SEQ ID NO: 20 - DNA, 19 bp, Artificial Sequence
  Synthetic
gggtctcggtcttcttgag

SEQ ID NO: 21 - DNA, 18 bp, Artificial Sequence
  Synthetic
aagacatctgctgcaagg

SEQ ID NO: 22 - DNA, 20 bp, Artificial Sequence
  Synthetic
cccacaacttctcattctca

SEQ ID NO: 23 - DNA, 20 bp, Artificial Sequence
  Synthetic
agatagtagcagacgaagcc

SEQ ID NO: 24 - DNA, 20 bp, Artificial Sequence
  Synthetic
aggctacggacacgtttttc

SEQ ID NO: 25 - DNA, 20 bp, Artificial Sequence
  Synthetic
gccctgtagtcctgggaatc

SEQ ID NO: 26 - DNA, 19 bp, Artificial Sequence
  Synthetic
ccagtgctacccggagaaa

SEQ ID NO: 27 - DNA, 21 bp, Artificial Sequence
  Synthetic
caggagtggctcagagtgacc

SEQ ID NO: 28 - DNA, 21 bp, Artificial Sequence
  Synthetic
tagctgtccaccattcaccat

SEQ ID NO: 29 - DNA, 21 bp, Artificial Sequence
  Synthetic
acgaataagttggtctgctct

SEQ ID NO: 30 - DNA, 20 bp, Artificial Sequence
  Synthetic
cccttgttgtagatgacgcc

SEQ ID NO: 31 - DNA, 20 bp, Artificial Sequence
  Synthetic
cccttgttgtagatgacgcc

SEQ ID NO: 32 - DNA, 20 bp, Artificial Sequence
  Synthetic
acttttggtcgttccttcgg

SEQ ID NO: 33 - DNA, 20 bp, Artificial Sequence
  Synthetic
gggggtactggttctgtgaa

SEQ ID NO: 34 - DNA, 20 bp, Artificial Sequence
  Synthetic
cagagcccctacacctaccc

SEQ ID NO: 35 - DNA, 20 bp, Artificial Sequence
  Synthetic
agctcccagctatctgtgac

SEQ ID NO: 36 - DNA, 22 bp, Artificial Sequence
  Synthetic
cccttgtgaactctgctcaaac

SEQ ID NO: 37 - DNA, 20 bp, Artificial Sequence
  Synthetic
atgctgggctcagtgaagat

SEQ ID NO: 38 - DNA, 20 bp, Artificial Sequence
  Synthetic
agagctgagtgggttcaagg

SEQ ID NO: 39 - DNA, 19 bp, Artificial Sequence
  Synthetic
cacggcgaggaggaaccta

SEQ ID NO: 40 - DNA, 21 bp, Artificial Sequence
  Synthetic
tcttgccattactgccacaca

SEQ ID NO: 41 - DNA, 20 bp, Artificial Sequence
  Synthetic
atgccccacctgtaacaaga

SEQ ID NO: 42 - DNA, 20 bp, Artificial Sequence
  Synthetic
ctcataggggcagacgtagg

SEQ ID NO: 43 - DNA, 20 bp, Artificial Sequence
  Synthetic
atgacacagctgcccttttc

SEQ ID NO: 44 - DNA, 20 bp, Artificial Sequence
  Synthetic
tcgcaaagaagtcaagttgc

SEQ ID NO: 45 - DNA, 21 bp, Artificial Sequence
  Synthetic
tgctaggcccaaaactgaaac

SEQ ID NO: 46 - DNA, 21 bp, Artificial Sequence
  Synthetic
cattcggtgtcttgttccaga

SEQ ID NO: 47 - DNA, 21 bp, Artificial Sequence
  Synthetic
ctgcggcagatggtctacttc

SEQ ID NO: 48 - DNA, 21 bp, Artificial Sequence
  Synthetic
atagtggagggtgctctgttg

SEQ ID NO: 49 - DNA, 22 bp, Artificial Sequence
  Synthetic
ggacaaatcggaagagtgatcg

SEQ ID NO: 50 - DNA, 20 bp, Artificial Sequence
  Synthetic
catccgtgtgctccgcttac

SEQ ID NO: 51 - DNA, 20 bp, Artificial Sequence
  Synthetic
cgtcaagggttatggatctc

SEQ ID NO: 52 - DNA, 20 bp, Artificial Sequence
  Synthetic
gggcgttatgaattgggatg

SEQ ID NO: 53 - DNA, 20 bp, Artificial Sequence
  Synthetic
tgacctcacctcaagcacac

SEQ ID NO: 54 - DNA, 20 bp, Artificial Sequence
  Synthetic
catcactctgcatttccagc

SEQ ID NO: 55 - DNA, 20 bp, Artificial Sequence
  Synthetic
cagagattccaaacgcttcc

SEQ ID NO: 56 - DNA, 20 bp, Artificial Sequence
  Synthetic
tggtactttccggtctccac

SEQ ID NO: 57 - DNA, 21 bp, Artificial Sequence
  Synthetic
cttcaagaccacctacttctg

SEQ ID NO: 58 - DNA, 20 bp, Artificial Sequence
  Synthetic
cagtaaatgtcgggcaaagg

SEQ ID NO: 59 - DNA, 21 bp, Artificial Sequence
  Synthetic
gcgttcatgaggactatgagg

SEQ ID NO: 60 - DNA, 20 bp, Artificial Sequence
  Synthetic
cggagcgagctgtcttagat

SEQ ID NO: 61 - DNA, 20 bp, Artificial Sequence
  Synthetic
accgttcatgcagggtagag

SEQ ID NO: 62 - DNA, 19 bp, Artificial Sequence
  Synthetic
aacatgacgctcatcggag

SEQ ID NO: 63 - DNA, 20 bp, Artificial Sequence
  Synthetic
ggggaaccagcttggcttac

SEQ ID NO: 64 - DNA, 23 bp, Artificial Sequence
  Synthetic
cttttgaaagcgacgattggatg

SEQ ID NO: 65 - DNA, 21 bp, Artificial Sequence
  Synthetic
ttgaacaacaaggagacaggg

SEQ ID NO: 66 - DNA, 20 bp, Artificial Sequence
  Synthetic
gccgtaagcctcagaaaatg

SEQ ID NO: 67 - DNA, 21 bp, Artificial Sequence
  Synthetic
gatctctgcaaccctaacccc

SEQ ID NO: 68 - DNA, 21 bp, Artificial Sequence
  Synthetic
tcctttcacacatgacgacag

SEQ ID NO: 69 - DNA, 20 bp, Artificial Sequence
  Synthetic
ccagagaaatcctccgattg

SEQ ID NO: 70 - DNA, 20 bp, Artificial Sequence
  Synthetic
gagcttagggagtccttggc

SEQ ID NO: 71 - DNA, 21 bp, Artificial Sequence
  Synthetic
cgatccgcaacatccgtatga

SEQ ID NO: 72 - DNA, 21 bp, Artificial Sequence
  Synthetic
tccgaacactcttccgtagga

SEQ ID NO: 73 - DNA, 19 bp, Artificial Sequence
  Synthetic
gagtccaggagcaatgagg

SEQ ID NO: 74 - DNA, 20 bp, Artificial Sequence
  Synthetic
ccatttcatcctccacagac

SEQ ID NO: 75 - DNA, 21 bp, Artificial Sequence
  Synthetic
ccggcatgagaaggactctac

SEQ ID NO: 76 - DNA, 21 bp, Artificial Sequence
  Synthetic
ttcttcaccggggatgagatg

SEQ ID NO: 77 - DNA, 18 bp, Artificial Sequence
  Synthetic
aggctgacaaggtgtggc

SEQ ID NO: 78 - DNA, 20 bp, Artificial Sequence
  Synthetic
cactggggcgacttgtagag

SEQ ID NO: 79 - DNA, 20 bp, Artificial Sequence
  Synthetic
tttcgattccgctatgtgtg

SEQ ID NO: 80 - DNA, 20 bp, Artificial Sequence
  Synthetic
gaacgataacctttgcaggc

SEQ ID NO: 81 - DNA, 19 bp, Artificial Sequence
  Synthetic
atggacgatatgagaggcg

SEQ ID NO: 82 - DNA, 18 bp, Artificial Sequence
  Synthetic
aattctctggcatccgac

SEQ ID NO: 83 - DNA, 19 bp, Artificial Sequence
  Synthetic
cgggcgcattgtgtatatc

SEQ ID NO: 84 - DNA, 20 bp, Artificial Sequence
  Synthetic
gttcctgggttcgattgtcc

SEQ ID NO: 85 - DNA, 20 bp, Artificial Sequence
  Synthetic
tcctccagacacactgatgc

SEQ ID NO: 86 - DNA, 20 bp, Artificial Sequence
  Synthetic
aaaggcaagcttcgtcaaca

SEQ ID NO: 87 - DNA, 20 bp, Artificial Sequence
  Synthetic
tggtttgaaacatcagccaa

SEQ ID NO: 88 - DNA, 20 bp, Artificial Sequence
  Synthetic
gcttcacaatgtccggttct

SEQ ID NO: 89 - DNA, 20 bp, Artificial Sequence
  Synthetic
gctgagcacttttcggaact

SEQ ID NO: 90 - DNA, 20 bp, Artificial Sequence
  Synthetic
ccctccttctctcccttctg

SEQ ID NO: 91 - DNA, 20 bp, Artificial Sequence
  Synthetic
gaccgctgacaaatagggtc

SEQ ID NO: 92 - DNA, 19 bp, Artificial Sequence
  Synthetic
gaaggaccgggaagatgaa

SEQ ID NO: 93 - DNA, 19 bp, Artificial Sequence
  Synthetic
ctttgcatagctgggggtt

SEQ ID NO: 94 - DNA, 20 bp, Artificial Sequence
  Synthetic
ccttccaggtggccattatt

SEQ ID NO: 95 - DNA, 20 bp, Artificial Sequence
  Synthetic
cactctaccatcgtcagcca

SEQ ID NO: 96 - DNA, 20 bp, Artificial Sequence
  Synthetic
atagcccaaggtaaaagccc

SEQ ID NO: 97 - DNA, 21 bp, Artificial Sequence
  Synthetic
aaagataaaggagcgaatccg

SEQ ID NO: 98 - DNA, 19 bp, Artificial Sequence
  Synthetic
gccgagcactcttaaacac

SEQ ID NO: 99 - DNA, 20 bp, Artificial Sequence
  Synthetic
ccagcaagaaaagaagccaa

SEQ ID NO: 100 - DNA, 20 bp, Artificial Sequence
  Synthetic
tgaccatgaggcaaaatcaa

SEQ ID NO: 101 - DNA, 23 bp, Artificial Sequence
  Synthetic
tggcaacaattctgcttcaaaac

SEQ ID NO: 102 - DNA, 21 bp, Artificial Sequence
  Synthetic
gaggtccctggataggcatgt

SEQ ID NO: 103 - DNA, 18 bp, Artificial Sequence
  Synthetic
ccaagctgaaggaccaag

SEQ ID NO: 104 - DNA, 19 bp, Artificial Sequence
  Synthetic
ggagatgagctgtgccgaa

SEQ ID NO: 105 - DNA, 19 bp, Artificial Sequence
  Synthetic
ggtgagccagatgtttgcc

SEQ ID NO: 106 - DNA, 21 bp, Artificial Sequence
  Synthetic
tccctcttcgatgaaccccat

SEQ ID NO: 107 - DNA, 18 bp, Artificial Sequence
  Synthetic
tgcacaaagagaaccccg

SEQ ID NO: 108 - DNA, 19 bp, Artificial Sequence
  Synthetic
cttccttctcctcctctgg

SEQ ID NO: 109 - DNA, 20 bp, Artificial Sequence
  Synthetic
gccggtgagtcagtctgttt

SEQ ID NO: 110 - DNA, 20 bp, Artificial Sequence
  Synthetic
gcaacgagagccaggactat

SEQ ID NO: 111 - DNA, 21 bp, Artificial Sequence
  Synthetic
ctttgaagctgttttgtctcc

SEQ ID NO: 112 - DNA, 18 bp, Artificial Sequence
  Synthetic
gttgatgctgtgaatgcg

SEQ ID NO: 113 - DNA, 20 bp, Artificial Sequence
  Synthetic
cccagtccgatgagaagaag

SEQ ID NO: 114 - DNA, 20 bp, Artificial Sequence
  Synthetic
gtttgcactcatggttcagc

SEQ ID NO: 115 - DNA, 97 bp, Artificial Sequence
  Synthetic
tgctgttgacagtgagcgcaagcagagagatcatgatcaatagtgaagccacagatgtattgatcatgatctctctgcttttgcctactgcctcgga

SEQ ID NO: 116 - DNA, 97 bp, Artificial Sequence
  Synthetic
tgctgttgacagtgagcgccaggaggaatttaataatacatagtgaagccacagatgtatgtattattaaattcctcctgatgcctactgcctcgga

SEQ ID NO: 117 - DNA, 97 bp, Artificial Sequence
  Synthetic
tgctgttgacagtgagcgccggaattgatttctctgtttatagtgaagccacagatgtataaacagagaaatcaattccgatgcctactgcctcgga

SEQ ID NO: 118 - DNA, 97 bp, Artificial Sequence
  Synthetic
tgctgttgacagtgagcgccatgactgtaactaatcactatagtgaagccacagatgtatagtgattagttacagtcatgttgcctactgcctcgga

SEQ ID NO: 119 - DNA, 97 bp, Artificial Sequence
  Synthetic
tgctgttgacagtgagcgacagcagaagcttaacaaacaatagtgaagccacagatgtattgtttgttaagcttctgctgctgcctactgcctcgga

SEQ ID NO: 120 - DNA, 97 bp, Artificial Sequence
  Synthetic
tgctgttgacagtgagcgccagagaggtgtttaagaagaatagtgaagccacagatgtattcttcttaaacacctctctgatgcctactgcctcgga

SEQ ID NO: 121 - DNA, 97 bp, Artificial Sequence
  Synthetic
tgctgttgacagtgagcgacaggacagagagattgtgacatagtgaagccacagatgtatgtcacaatctctctgtcctggtgcctactgcctcgga

SEQ ID NO: 122 - DNA, 97 bp, Artificial Sequence
  Synthetic
tgctgttgacagtgagcgacagcttggtgcttactcttaatagtgaagccacagatgtattaagagtaagcaccaagctggtgcctactgcctcgga

SEQ ID NO: 123 - DNA, 97 bp, Artificial Sequence
  Synthetic
tgctgttgacagtgagcgccagattgtttcttgtgaagtatagtgaagccacagatgtatacttcacaagaaacaatctgatgcctactgcctcgga

SEQ ID NO: 124 - DNA, 97 bp, Artificial Sequence
  Synthetic
tgctgttgacagtgagcgccagaaagtttacagaaccctatagtgaagccacagatgtatagggttctgtaaactttctgatgcctactgcctcgga

SEQ ID NO: 125 - DNA, 97 bp, Artificial Sequence
  Synthetic
tgctgttgacagtgagcgacagaagttagttgctatgagatagtgaagccacagatgtatctcatagcaactaacttctggtgcctactgcctcgga

SEQ ID NO: 126 - DNA, 97 bp, Artificial Sequence
  Synthetic
tgctgttgacagtgagcgccacagattaagacttcaaatatagtgaagccacagatgtatatttgaagtcttaatctgtgttgcctactgcctcgga

SEQ ID NO: 127 - DNA, 97 bp, Artificial Sequence
  Synthetic
tgctgttgacagtgagcgcccatgtcactgcttcaagatatagtgaagccacagatgtatatcttgaagcagtgacatggttgcctactgcctcgga

SEQ ID NO: 128 - DNA, 97 bp, Artificial Sequence
  Synthetic
tgctgttgacagtgagcgaaccagtcacctgtcttagcaatagtgaagccacagatgtattgctaagacaggtgactggtgtgcctactgcctcgga

SEQ ID NO: 129 - DNA, 97 bp, Artificial Sequence
  Synthetic
tgctgttgacagtgagcgcaactagaaatgtttcctttaatagtgaagccacagatgtattaaaggaaacatttctagttatgcctactgcctcgga

SEQ ID NO: 130 - DNA, 97 bp, Artificial Sequence
  Synthetic
tgctgttgacagtgagcgccaagacagaagacagtttgaatagtgaagccacagatgtattcaaactgtcttctgtcttgatgcctactgcctcgga

SEQ ID NO: 131 - DNA, 97 bp, Artificial Sequence
  Synthetic
tgctgttgacagtgagcgatcaggatttaagcacatacaatagtgaagccacagatgtattgtatgtgcttaaatcctgactgcctactgcctcgga

SEQ ID NO: 132 - DNA, 97 bp, Artificial Sequence
  Synthetic
tgctgttgacagtgagcgccagactgtacacccttatctatagtgaagccacagatgtatagataagggtgtacagtctgttgcctactgcctcgga

SEQ ID NO: 133 - DNA, 97 bp, Artificial Sequence
  Synthetic
tgctgttgacagtgagcgcccagaaactgttctagaagaatagtgaagccacagatgtattcttctagaacagtttctggatgcctactgcctcgga

SEQ ID NO: 134 - DNA, 97 bp, Artificial Sequence
  Synthetic
tgctgttgacagtgagcgccaactcctacatgaccttgaatagtgaagccacagatgtattcaaggtcatgtaggagttgatgcctactgcctcgga

SEQ ID NO: 135 - DNA, 97 bp, Artificial Sequence
  Synthetic
tgctgttgacagtgagcgacgaggtgtattctccagtgaatagtgaagccacagatgtattcactggagaatacacctcgctgcctactgcctcgga

SEQ ID NO: 136 - DNA, 97 bp, Artificial Sequence
  Synthetic
tgctgttgacagtgagcgccagtgagatcctagacaagtatagtgaagccacagatgtatacttgtctaggatctcactgttgcctactgcctcgga

SEQ ID NO: 137 - DNA, 97 bp, Artificial Sequence
  Synthetic
tgctgttgacagtgagcgatcagttattaccgttatattatagtgaagccacagatgtataatataacggtaataactgagtgcctactgcctcgga

SEQ ID NO: 138 - DNA, 97 bp, Artificial Sequence
  Synthetic
tgctgttgacagtgagcgacagctctttgagctcctccaatagtgaagccacagatgtattggaggagctcaaagagctggtgcctactgcctcgga

SEQ ID NO: 139 - DNA, 97 bp, Artificial Sequence
  Synthetic
tgctgttgacagtgagcgcaccagctgtggtaaactgaaatagtgaagccacagatgtatttcagtttaccacagctggtttgcctactgcctcgga

SEQ ID NO: 140 - DNA, 97 bp, Artificial Sequence
  Synthetic
tgctgttgacagtgagcgacagagacaacaggtatctaaatagtgaagccacagatgtatttagatacctgttgtctctgctgcctactgcctcgga

SEQ ID NO: 141 - DNA, 97 bp, Artificial Sequence
  Synthetic
tgctgttgacagtgagcgacatctgattgcagttatggaatagtgaagccacagatgtattccataactgcaatcagatgctgcctactgcctcgga

SEQ ID NO: 142 - DNA, 97 bp, Artificial Sequence
  Synthetic
tgctgttgacagtgagcgatgggacgaggttaccatcgaatagtgaagccacagatgtattcgatggtaacctcgtcccagtgcctactgcctcgga

SEQ ID NO: 143 - DNA, 97 bp, Artificial Sequence
  Synthetic
tgctgttgacagtgagcgccagtaaagttagtgtgatttatagtgaagccacagatgtataaatcacactaactttactgttgcctactgcctcgga

SEQ ID NO: 144 - DNA, 97 bp, Artificial Sequence
  Synthetic
tgctgttgacagtgagcgctcgggtgttgtctcactgatttagtgaagccacagatgtaaatcagtgagacaacacccgaatgcctactgcctcgga

SEQ ID NO: 145 - DNA, 97 bp, Artificial Sequence
  Synthetic
tgctgttgacagtgagcgccagtctgtttctgtgtcgtaatagtgaagccacagatgtattacgacacagaaacagactgttgcctactgcctcgga

SEQ ID NO: 146 - DNA, 97 bp, Artificial Sequence
  Synthetic
tgctgttgacagtgagcgccagtctggtgttcctgttcaatagtgaagccacagatgtattgaacaggaacaccagactgatgcctactgcctcgga

SEQ ID NO: 147 - DNA, 97 bp, Artificial Sequence
  Synthetic
tgctgttgacagtgagcgataggcgattgtcgagaagagatagtgaagccacagatgtatctcttctcgacaatcgcctactgcctactgcctcgga

SEQ ID NO: 148 - DNA, 97 bp, Artificial Sequence
  Synthetic
tgctgttgacagtgagcgaacgagatgttattacaacaaatagtgaagccacagatgtatttgttgtaataacatctcgtgtgcctactgcctcgga

SEQ ID NO: 149 - DNA, 97 bp, Artificial Sequence
  Synthetic
tgctgttgacagtgagcgccagcatagtggaagatagatatagtgaagccacagatgtatatctatcttccactatgctgttgcctactgcctcgga

SEQ ID NO: 150 - DNA, 97 bp, Artificial Sequence
  Synthetic
tgctgttgacagtgagcgatggcatcatgacaatgttgaatagtgaagccacagatgtattcaacattgtcatgatgccactgcctactgcctcgga

SEQ ID NO: 151 - DNA, 97 bp, Artificial Sequence
  Synthetic
tgctgttgacagtgagcgacaccacatacaggttactcaatagtgaagccacagatgtattgagtaacctgtatgtggtggtgcctactgcctcgga

SEQ ID NO: 152 - DNA, 97 bp, Artificial Sequence
  Synthetic
tgctgttgacagtgagcgcccaggactttctaaatgtttatagtgaagccacagatgtataaacatttagaaagtcctggttgcctactgcctcgga

SEQ ID NO: 153 - DNA, 97 bp, Artificial Sequence
  Synthetic
tgctgttgacagtgagcgatgggtaaacctcacaagtgtatagtgaagccacagatgtatacacttgtgaggtttacccactgcctactgcctcgga

SEQ ID NO: 154 - DNA, 97 bp, Artificial Sequence
  Synthetic
tgctgttgacagtgagcgcaccgcctatggaagattgtaatagtgaagccacagatgtattacaatcttccataggcggtatgcctactgcctcgga

SEQ ID NO: 155 - DNA, 97 bp, Artificial Sequence
  Synthetic
tgctgttgacagtgagcgctgggatgagaaagataatgattagtgaagccacagatgtaatcattatctttctcatcccaatgcctactgcctcgga

SEQ ID NO: 156 - DNA, 97 bp, Artificial Sequence
  Synthetic
tgctgttgacagtgagcgcccactccagacatcaaagatatagtgaagccacagatgtatatctttgatgtctggagtggatgcctactgcctcgga

SEQ ID NO: 157 - DNA, 97 bp, Artificial Sequence
  Synthetic
tgctgttgacagtgagcgccggagagaagagacagttatatagtgaagccacagatgtatataactgtctcttctctccgatgcctactgcctcgga

SEQ ID NO: 158 - DNA, 73 bp, Artificial Sequence
  Synthetic
caactgcttctaataatagtgaagccacagatgtattattagaagcagttgctctgctgcctactgcctcgga

SEQ ID NO: 159 - DNA, 97 bp, Artificial Sequence
  Synthetic
tgctgttgacagtgagcgatggaatgtatttgaacaacaatagtgaagccacagatgtattgttgttcaaatacattccactgcctactgcctcgga

SEQ ID NO: 160 - DNA, 97 bp, Artificial Sequence
  Synthetic
tgctgttgacagtgagcgcaaggctcagatgaaagtcacatagtgaagccacagatgtatgtgactttcatctgagccttttgcctactgcctcgga

SEQ ID NO: 161 - DNA, 97 bp, Artificial Sequence
  Synthetic
tgctgttgacagtgagcgaacggtacagagtcaatctccatagtgaagccacagatgtatggagattgactctgtaccgtgtgcctactgcctcgga

SEQ ID NO: 162 - DNA, 97 bp, Artificial Sequence
  Synthetic
tgctgttgacagtgagcgatcccaccacagtacaaagtcatagtgaagccacagatgtatgactttgtactgtggtgggagtgcctactgcctcgga

SEQ ID NO: 163 - DNA, 97 bp, Artificial Sequence
  Synthetic
tgctgttgacagtgagcgacagcctcactttccagagacatagtgaagccacagatgtatgtctctggaaagtgaggctggtgcctactgcctcgga

SEQ ID NO: 164 - DNA, 97 bp, Artificial Sequence
  Synthetic
tgctgttgacagtgagcgcaagagtggttctacaaagatatagtgaagccacagatgtatatctttgtagaaccactcttatgcctactgcctcgga

SEQ ID NO: 165 - DNA, 97 bp, Artificial Sequence
  Synthetic
tgctgttgacagtgagcgctccggtggaaacggagagtcatagtgaagccacagatgtatgactctccgtttccaccggaatgcctactgcctcgga

SEQ ID NO: 166 - DNA, 97 bp, Artificial Sequence
  Synthetic
tgctgttgacagtgagcgccggaagagtgttcggaatttatagtgaagccacagatgtataaattccgaacactcttccgttgcctactgcctcgga

SEQ ID NO: 167 - DNA, 97 bp, Artificial Sequence
  Synthetic
tgctgttgacagtgagcgccggcagcttaatgacagtcaatagtgaagccacagatgtattgactgtcattaagctgccgatgcctactgcctcgga

SEQ ID NO: 168 - DNA, 97 bp, Artificial Sequence
  Synthetic
tgctgttgacagtgagcgctaccttcaaatattagagcaatagtgaagccacagatgtattgctctaatatttgaaggtattgcctactgcctcgga

SEQ ID NO: 169 - DNA, 97 bp, Artificial Sequence
  Synthetic
tgctgttgacagtgagcgaaccaagcaggaagatgtagtatagtgaagccacagatgtatactacatcttcctgcttggtgtgcctactgcctcgga

SEQ ID NO: 170 - DNA, 97 bp, Artificial Sequence
  Synthetic
tgctgttgacagtgagcgaaggcgtcagattccaggttcatagtgaagccacagatgtatgaacctggaatctgacgcctgtgcctactgcctcgga

SEQ ID NO: 171 - DNA, 97 bp, Artificial Sequence
  Synthetic
tgctgttgacagtgagcgacaggttcatgtggacatttcttagtgaagccacagatgtaagaaatgtccacatgaacctggtgcctactgcctcgga

SEQ ID NO: 172 - DNA, 97 bp, Artificial Sequence
  Synthetic
tgctgttgacagtgagcgacgctgtggacctcaagctgtatagtgaagccacagatgtatacagcttgaggtccacagcgctgcctactgcctcgga

SEQ ID NO: 173 - DNA, 97 bp, Artificial Sequence
  Synthetic
tgctgttgacagtgagcgaccgctcagccttgtacatagatagtgaagccacagatgtatctatgtacaaggctgagcggctgcctactgcctcgga

SEQ ID NO: 174 - DNA, 97 bp, Artificial Sequence
  Synthetic
tgctgttgacagtgagcgatgccgtcatcttctatgttaatagtgaagccacagatgtattaacatagaagatgacggcagtgcctactgcctcgga

SEQ ID NO: 175 - DNA, 97 bp, Artificial Sequence
  Synthetic
tgctgttgacagtgagcgacaggcatatgatgataagtaatagtgaagccacagatgtattacttatcatcatatgcctgctgcctactgcctcgga

SEQ ID NO: 176 - DNA, 97 bp, Artificial Sequence
  Synthetic
tgctgttgacagtgagcgcagggaaggatgtgcttatgaatagtgaagccacagatgtattcataagcacatccttccctttgcctactgcctcgga

SEQ ID NO: 177 - DNA, 97 bp, Artificial Sequence
  Synthetic
tgctgttgacagtgagcgcgacctcctagagtctatagaatagtgaagccacagatgtattctatagactctaggaggtcatgcctactgcctcgga

SEQ ID NO: 178 - DNA, 97 bp, Artificial Sequence
  Synthetic
tgctgttgacagtgagcgatgcaagcaagtgtttaagaaatagtgaagccacagatgtatttcttaaacacttgcttgcagtgcctactgcctcgga

SEQ ID NO: 179 - DNA, 97 bp, Artificial Sequence
  Synthetic
tgctgttgacagtgagcgagaagatgctgagcaatgagaatagtgaagccacagatgtattctcattgctcagcatcttcctgcctactgcctcgga

SEQ ID NO: 180 - DNA, 97 bp, Artificial Sequence
  Synthetic
tgctgttgacagtgagcgacaacactgtacctcagttcattagtgaagccacagatgtaatgaactgaggtacagtgttggtgcctactgcctcgga

SEQ ID NO: 181 - DNA, 97 bp, Artificial Sequence
  Synthetic
tgctgttgacagtgagcgatctcataaagctcacatctaatagtgaagccacagatgtattagatgtgagctttatgagagtgcctactgcctcgga

SEQ ID NO: 182 - DNA, 97 bp, Artificial Sequence
  Synthetic
tgctgttgacagtgagcgcccgatacaagcggagagtttatagtgaagccacagatgtataaactctccgcttgtatcggatgcctactgcctcgga

SEQ ID NO: 183 - DNA, 97 bp, Artificial Sequence
  Synthetic
tgctgttgacagtgagcgccacaagtttctgtttctattttagtgaagccacagatgtaaaatagaaacagaaacttgtgatgcctactgcctcgga

SEQ ID NO: 184 - DNA, 97 bp, Artificial Sequence
  Synthetic
tgctgttgacagtgagcgacaggctcatcatttacttaattagtgaagccacagatgtaattaagtaaatgatgagcctgctgcctactgcctcgga

SEQ ID NO: 185 - DNA, 97 bp, Artificial Sequence
  Synthetic
tgctgttgacagtgagcgaagagatcacagttacagatgatagtgaagccacagatgtatcatctgtaactgtgatctctgtgcctactgcctcgga

SEQ ID NO: 186 - DNA, 97 bp, Artificial Sequence
  Synthetic
tgctgttgacagtgagcgacggctgagaagttgtcaccaatagtgaagccacagatgtattggtgacaacttctcagccggtgcctactgcctcgga

SEQ ID NO: 187 - DNA, 97 bp, Artificial Sequence
  Synthetic
tgctgttgacagtgagcgatacgaagactccattgtcctatagtgaagccacagatgtataggacaatggagtcttcgtagtgcctactgcctcgga

SEQ ID NO: 188 - DNA, 97 bp, Artificial Sequence
  Synthetic
tgctgttgacagtgagcgcccctgccgacaagaaagtgaatagtgaagccacagatgtattcactttcttgtcggcagggttgcctactgcctcgga

SEQ ID NO: 189 - DNA, 97 bp, Artificial Sequence
  Synthetic
tgctgttgacagtgagcgctagatggagagtagaaggagatagtgaagccacagatgtatctccttctactctccatctattgcctactgcctcgga

SEQ ID NO: 190 - DNA, 97 bp, Artificial Sequence
  Synthetic
tgctgttgacagtgagcgctcctgcgagactacaaggttatagtgaagccacagatgtataaccttgtagtctcgcaggattgcctactgcctcgga

SEQ ID NO: 191 - DNA, 97 bp, Artificial Sequence
  Synthetic
tgctgttgacagtgagcgccacagttcagtctaactacaatagtgaagccacagatgtattgtagttagactgaactgtgatgcctactgcctcgga

SEQ ID NO: 192 - DNA, 97 bp, Artificial Sequence
  Synthetic
tgctgttgacagtgagcgaacgcagttgactcgttccagatagtgaagccacagatgtatctggaacgagtcaactgcgtctgcctactgcctcgga

SEQ ID NO: 193 - DNA, 97 bp, Artificial Sequence
  Synthetic
tgctgttgacagtgagcgcacacgaggacctcagagacaatagtgaagccacagatgtattgtctctgaggtcctcgtgtttgcctactgcctcgga

SEQ ID NO: 194 - DNA, 97 bp, Artificial Sequence
  Synthetic
tgctgttgacagtgagcgcccggaacaatctgctgtagaatagtgaagccacagatgtattctacagcagattgttccggatgcctactgcctcgga

SEQ ID NO: 195 - DNA, 97 bp, Artificial Sequence
  Synthetic
tgctgttgacagtgagcgacaggatgttgaagaacatacatagtgaagccacagatgtatgtatgttcttcaacatcctggtgcctactgcctcgga

SEQ ID NO: 196 - DNA, 97 bp, Artificial Sequence
  Synthetic
tgctgttgacagtgagcgccacagagctgctactcaagaatagtgaagccacagatgtattcttgagtagcagctctgtgatgcctactgcctcgga

SEQ ID NO: 197 - DNA, 97 bp, Artificial Sequence
  Synthetic
tgctgttgacagtgagcgagaggaccagcatacatgtttatagtgaagccacagatgtataaacatgtatgctggtcctcgtgcctactgcctcgga

SEQ ID NO: 198 - DNA, 97 bp, Artificial Sequence
  Synthetic
tgctgttgacagtgagcgacccagcaaagtttgaccaaattagtgaagccacagatgtaatttggtcaaactttgctgggctgcctactgcctcgga

SEQ ID NO: 199 - DNA, 97 bp, Artificial Sequence
  Synthetic
tgctgttgacagtgagcgccacagtcatcactgtcagtaatagtgaagccacagatgtattactgacagtgatgactgtgttgcctactgcctcgga

SEQ ID NO: 200 - DNA, 97 bp, Artificial Sequence
  Synthetic
tgctgttgacagtgagcgcccgaatctactccgcactctatagtgaagccacagatgtatagagtgcggagtagattcggttgcctactgcctcgga

SEQ ID NO: 201 - DNA, 97 bp, Artificial Sequence
  Synthetic
tgctgttgacagtgagcgccagctgtatttactgcaacaatagtgaagccacagatgtattgttgcagtaaatacagctgttgcctactgcctcgga

SEQ ID NO: 202 - DNA, 97 bp, Artificial Sequence
  Synthetic
tgctgttgacagtgagcgacacagcagttagttcatgtattagtgaagccacagatgtaatacatgaactaactgctgtgctgcctactgcctcgga

SEQ ID NO: 203 - DNA, 7 bp, Artificial Sequence
  Synthetic
gggcggr

SEQ ID NO: 204 - DNA, 8 bp, Artificial Sequence
  Synthetic
gggaggrr

SEQ ID NO: 205 - DNA, 8 bp, Artificial Sequence
  Synthetic
scggaagy

SEQ ID NO: 206 - DNA, 6 bp, Artificial Sequence
  Synthetic
caggtg

SEQ ID NO: 207 - RNA, 10 nt, Artificial Sequence
  Synthetic
  misc_feature (6)..(6): n is a, c, g, or u
rcgcangcgy

SEQ ID NO: 208 - DNA, 6 bp, Artificial Sequence
  Synthetic
cagctg

SEQ ID NO: 209 - DNA, 9 bp, Artificial Sequence
  Synthetic
  misc_feature (6)..(6): n is a, c, g, t or u
gccatnttg

(19) United States
(12) Patent Application Publication
(10) Pub. No.: US 2019/0328791 A1
(43) Pub. Date: Oct. 31, 2019

(54) ENHANCED HETATOPOIETIC STEM CELL TRANSPLANTATION

(71) Applicant: ST. JUDE CHILDREN'S RESEARCH HOSPITAL, Memphis, TN (US)

(72) Inventors: Shannon Leigh McKinney-Freeman, Germantown, TN (US); Per Holmfedt, Uppsala (SE); Miguel Ganuza Fernandez, Memphis, TN (US)

(21) Appl. No.: 16/312,491

(22) PCT Filed: Jun. 22, 2017
(86) PCT No.: PCT/US17/38798

§ 371 Date: Dec. 21, 2018

Related U.S. Application Data
(60) Provisional application No. 62/353,393, filed on Jun. 22, 2016.

(51) Int. Cl. A61K 35/28; C12N 5/0789; C12N 15/113; A61K 35/51; C12N 5/073

(52) U.S. Cl. CPC A61K 35/28; C12N 5/0647; C12N 15/113; A61K 35/51; C12N 2310/531; C12N 2510/00; C12N 2310/14; C12N 2310/122; C12N 5/0605

(57) ABSTRACT

The present invention relates to methods of enhancing stem cell transplantation by treating pre-graft cells with silencing constructs for reducing expression of GASP (G-protein coupled receptor Associated Sorting Proteins) family genes, either permanently or transiently. In particular, methods of using a shRNA silencing construct for Gprasp1, Gprasp2 or Armcx1 (Gasp7) in pre-graft hematopoietic transplant cells are provided for improving the ability of these cells to replenish the hematopoietic system of host organisms. Further, the use of GASP gene silenced umbilical cord blood-derived cells is contemplated for transplantation into HLA mismatched (allogeneic) hosts.

### CROSS-REFERENCE TO RELATED APPLICATIONS

The present application claims the benefit of U.S. Provisional Patent Application No. 62/353,393, filed on Jun. 22, 2016, which is incorporated herein by reference.

This invention was made with government support under National Institute of Diabetes and Digestive and Kidney Diseases (NIDDK) Grant No. K01DK080846, NIDDK Grant No. R03DK093731 and National Human Genome Research Institute HG006130 awarded by the National Institutes of Health. The government has certain rights in the invention.

### FIELD OF THE INVENTION

The present invention relates to methods of enhancing stem cell transplantation by treating pre-graft cells with silencing constructs for reducing expression of GASP (G-protein coupled receptor Associated Sorting Proteins) family genes, either permanently or transiently. In particular, methods of using a shRNA silencing construct for Gprasp1, Gprasp2 or Armcx1 (Gasp7) in pre-graft hematopoietic transplant cells are provided for improving the ability of these cells to replenish the hematopoietic system of host organisms. Further, the use of GASP gene silenced umbilical cord blood-derived cells is contemplated for transplantation into HLA mismatched (allogeneic) hosts.

### BACKGROUND

Hematopoietic stem cells (HSC), in healthy mammals, maintain life-long hematopoiesis and have the capability to restore the entire blood system when transplanted into a host whose own hematopoietic system has been ablated by irradiation or chemotherapy. This capability is also used clinically to treat blood diseases and cancer. Thus, HSC transplantation represents a curative therapy for many hematologic diseases. It is also a life-saving therapy following high dose chemotherapy for many non-hematopoietic cancers.

Although most deaths post-transplant are due to disease relapse, much of this acute mortality is also due to infection and other complications that may be ameliorated by protocols that accelerate recovery of a functional hematopoietic system from transplanted cells and/or tissues. Such improvements might also lower long-term risks that plague survivors of cancer treatments, especially children, receiving transplants. Longer-term risks following HSC include secondary malignancies, adaptive immune dysfunction, growth failure, gonadal dysfunction, and thyroid dysfunction.

Therefore there is a need of methods for improving HSC engraftment in order to ameliorate post-transplant morbidity.

### SUMMARY OF THE INVENTION

The present invention relates to methods of enhancing stem cell transplantation by treating pre-graft cells with silencing constructs for reducing expression of GASP (G-protein coupled receptor Associated Sorting Proteins) family genes, either permanently or transiently. In particular, methods of using a shRNA silencing construct for Gprasp1, Gprasp2 or Armcx1 (Gasp7) in pre-graft hematopoietic transplant cells are provided for improving the ability of these cells to replenish the hematopoietic system of host organisms. Further, the use of GASP gene silenced umbilical cord blood-derived cells is contemplated for transplantation into HLA mismatched (allogeneic) hosts.

The invention provides a method for enhancing hematopoietic stem cell (HSC) engraftment, comprising, a) providing, i) a human hematopoietic stem cell (HSC) population, wherein said HSCs have a HLA haplotype and express a gene in the GASP (G-protein coupled receptor Associated Sorting Protein) gene family, and ii) a human patient having an HLA haplotype, b) treating said HSCs under conditions such that expression of said GASP gene in said HSC population is reduced, and c) transplanting said treated HSCs into said patient. In one embodiment, said treatment is shRNA-mediated knockdown of said GASP gene. In one embodiment, said knockdown is up to but not including a 100% reduction in gene expression. While the invention contemplates reduced expression it is not meant to limit the magnitude of the reduction, such that a reduction may be at least 10%, 20%, 30%, 40%, and preferably at least 50%, 60%, 70%, 80%, 90%, 95%, 98%, 99% up to 100%, but preferably not including a 100% reduction.

In one embodiment, said transplantation said GASP gene expression increases in treated HSCs. In one embodiment, said transplantation said GASP gene expression increases in progeny cells of said treated HSCs. In one embodiment, after said treatment said GASP gene is expressed in progeny cells of said treated HSCs. In one embodiment, after said treatment said GASP gene is not knocked down in progeny cells of said treated HSCs. In one embodiment, said GASP gene is selected from the group consisting of Gprasp2 and Armcx1. In one embodiment, said GASP gene is the Gprasp1 gene. In one embodiment, said GASP gene is a Basic Helix-Loop-Helix Domain Containing, Class B, 9. In one embodiment, said HSCs of step a) express two or more GASP genes. In one embodiment, said two GASP genes are Gprasp1 and Gprasp2. In one embodiment, said two GASP genes are Gprasp1 and Gprasp3. In one embodiment, said two GASP genes are Gprasp2 and Gprasp3. In one embodiment, said HSCs of step a) express three GASP genes, wherein said three GASP genes are Gprasp1, Gprasp2 and Basic Helix-Loop-Helix Domain Containing, Class B, 9. In one embodiment, said human hematopoietic stem population is obtained from a sample selected from the group consisting of bone marrow, mobilized peripheral blood and umbilical cord blood. In one embodiment, said human hematopoietic stem population is obtained from umbilical cord blood (UCB). In one embodiment, said HSC HLA haplotype is a mismatch (allogeneic) between the stem cell population of said umbilical cord blood (UCB) and said HLA haplotype of said patient.

The invention provides a method for enhancing hematopoietic stem cell (HSC) engraftment, comprising, a) providing, i) a human umbilical cord blood (UCB) stem cell population, wherein said UCBs have a HLA haplotype and express a gene in the GASP (G-protein coupled receptor Associated Sorting Protein) gene family, wherein said GASP gene is selected from the group consisting of Gprasp1, Gprasp2, Basic Helix-Loop-Helix Domain Containing, Class B, 9, and Armcx1, and ii) a human patient, wherein said patient has a major Human Leukocyte Antigen (HLA) haplotype, and b) treating said HSCs to reduce expression of said GASP gene, and c) transplanting said treated HSCs into said patient. In one embodiment, said HSC HLA haplotype is a mismatch (allogeneic) between said umbilical cord blood (UCB) stem cell population and said HLA haplotype of said patient. It is not meant to limit the amount of HLA mismatch between stems cells and a recipient of those stem cells. Indeed, for one example in humans, because there are up to 7400 and more alleles in MHC-HLA genes corresponding to more than 100 specific antigens (expressed antigenic proteins) for HLA-A, B, C and DR genes, commonly haplotyped for use in providing human cells for use in transplantation, a mismatch may be when the stem cells and the recipient do not share any one or more, up to six pairs, of major HLA antigens involved with tissue matching, i.e. transplantation, for example, a mismatch may be when any one or more of two pairs of A antigens, two pairs of B antigens, and two pairs of DR antigens are not shared; two pairs of A antigens, two pairs of B antigens, two pairs of C antigens, and two pairs of DRB 1 antigens are not shared; two pairs of A antigens, two pairs of B antigens, two pairs of C antigens, two pairs of DRB 1 antigens and two pairs of DQ are not shared, etc. A mismatch may also be considered any combination of HLA alleles between host and transplanted cells resulting in rejection, including but not limited to Graft vs. Host Disease (GVHD). As one example, a 100% allogeneic mismatch is highly likely to result in GVHD, while in contrast, a 100% match is unlikely to result in GVHD.

The invention provides a method for enhancing human hematopoietic stem cell (HSC) engraftment, comprising, a) providing, i) a human hematopoietic stem cell (HSC) population, wherein said HSCs express a gene in the GASP (G-protein coupled receptor Associated Sorting Protein) gene family, and ii) a human patient, b) treating said human HSCs under conditions such that expression of said GASP gene in said HSC population is transiently reduced under conditions of a time period and a magnitude sufficient for improving the engraftment potential of the HSCs, and c) transplanting said treated HSCs into said patient. In one embodiment, said time period is up to 24 hours. In one embodiment, said reduction of said GASP gene expression is of a magnitude between 80% up to but not including 100%. While the invention contemplates reduced expression it is not meant to limit the magnitude of the reduction, such that a reduction may be at least 10%, 20%, 30%, 40%, and preferably at least 50%, 60%, 70%, 80%, 90%, 95%, 98%, 99% up to 100% but preferably not including a 100% reduction. In one embodiment, said improving said engraftment potential is evidenced by an increase in number of progeny cells from said treated HSCs up to 16 weeks post-transplantation. In one embodiment, said treatment is shRNA-mediated transient knockdown of said GASP gene. In one embodiment, said GASP gene is selected from the group consisting of Gprasp2 and Armcx1 (Gprasp7). In one embodiment, said GASP gene is the Gprasp1 gene. In one embodiment, said GASP gene is the Basic Helix-Loop-Helix Domain Containing, Class B, 9. In one embodiment, said HSCs of step a) express at two or more GASP genes. In one embodiment, said two GASP genes are Gprasp1 and Gprasp2. In one embodiment, said HSCs of step a) express three GASP genes, wherein said three GASP genes are Gprasp1, Gprasp2 and Basic Helix-Loop-Helix Domain Containing, Class B, 9.

The invention provides a method of treating a hematopoietic stem cell (HSC) population, comprising, 1) providing a hematopoietic stem cell (HSC) population, wherein said HSCs express a gene in the GASP (G-protein coupled receptor Associated Sorting Protein) gene family, and 2) treating said HSCs ex vivo under conditions such that expression of said GASP gene in said HSC population is reduced. In one embodiment, said treatment is shRNA-mediated knockdown of said GASP gene. In one embodiment, said knockdown of said GASP gene is between 80% up to but not including 100% reduction in expression. While the invention contemplates reduced expression it is not meant to limit the magnitude of the reduction, such that a reduction may be at least 10%, 20%, 30%, 40%, and preferably at least 50%, 60%, 70%, 80%, 90%, 95%, 98%, 99% up to but not including a 100% reduction. In one embodiment, said GASP gene is selected from the group consisting of Gprasp2 and Gprasp7. In one embodiment, said GASP gene is the Gprasp1 gene. In one embodiment, said GASP gene is Gprasp3. In one embodiment, said HSCs of step a) express two or more GASP genes. In one embodiment, said two GASP genes are Gprasp1 and Gprasp2. In one embodiment, said two GASP genes are Gprasp1 and Gprasp3. In one embodiment, said two GASP genes are Gprasp2 and Gprasp3. In one embodiment, said HSCs of step a) express three GASP genes, wherein said three GASP genes are Gprasp1, Gprasp2 and Gprasp3. In one embodiment, said hematopoietic stem population is obtained from a sample selected from the group consisting of bone marrow, mobilized peripheral blood and umbilical cord blood. In one embodiment, said hematopoietic stem population is obtained from umbilical cord blood (UCB). In one embodiment, said hematopoietic stem population is obtained from a human subject. In one embodiment, said hematopoietic stem population is obtained from a non-human:non-rodent subject.

The invention provides a method of treating an umbilical cord blood (UCB) stem cell population, comprising, a) providing, an umbilical cord blood (UCB) stem cell population, wherein said UCBs express a gene in the GASP (G-protein coupled receptor Associated Sorting Protein) gene family, wherein said GASP gene is selected from the group consisting of Gprasp1, Gprasp2, Gprasp3, and Gprasp7, and b) treating said HSCs ex vivo to reduce expression of said GASP gene.

The invention provides a method of treating a hematopoietic stem cell (HSC) population, comprising, a) a hematopoietic stem cell (HSC) population, wherein said HSCs express a gene in the GASP (G-protein coupled receptor Associated Sorting Protein) gene family, and b) treating said HSCs ex vivo under conditions such that expression of said GASP gene in said HSC population is transiently reduced. In one embodiment, said treating is incubation of HSCs up to 24 hours. In one embodiment, said reduced expression of said GASP gene is a reduction between 80% up to but not including 100%. While the invention contemplates reduced expression it is not meant to limit the magnitude of the reduction, such that a reduction may be at least 10%, 20%, 30%, 40%, and preferably at least 50%, 60%, 70%, 80%, 90%, 95%, 98%, 99% up to but not including a 100% reduction. In one embodiment, said treatment is shRNA-mediated transient knockdown of said GASP gene. In one embodiment, said GASP gene is selected from the group consisting of Gprasp2 and Gprasp3. In one embodiment, said GASP gene is the Gprasp1 gene. In one embodiment, said GASP gene is the Gprasp3. In one embodiment, said HSCs of step a) express two or more GASP genes. In one embodiment, said GASP gene is two GASP genes, wherein said two GASP genes are Gprasp1 and Gprasp2. In one embodiment, said HSCs of step a) express three GASP genes, wherein said three GASP genes are Gprasp1, Gprasp2 and Basic Helix-Loop-Helix Domain Containing, Class B, 9.

The invention provides a method of treating a hematopoietic stem cell (HSC) population, comprising, a) providing, i) a hematopoietic stem cell (HSC) population, wherein said HSCs have a MHC haplotype and express a gene in the GASP (G-protein coupled receptor Associated Sorting Protein) gene family, and ii) a subject having a MHC haplotype, wherein said subject is a nonhuman:nonrodent animal, b) treating said HSCs under conditions such that expression of said GASP gene in said HSC population is reduced. In one embodiment, said treatment is shRNA-mediated knockdown of said GASP gene. In one embodiment, said magnitude is the reduction of said GASP gene expression between 80% up to but not including 100%. While the invention contemplates reduced expression it is not meant to limit the magnitude of the reduction, such that a reduction may be at least 10%, 20%, 30%, 40%, and preferably at least 50%, 60%, 70%, 80%, 90%, 95%, 98%, 99% up to but not including a 100% reduction. In one embodiment, said hematopoietic stem population is obtained from a sample selected from the group consisting of bone marrow, mobilized peripheral blood and umbilical cord blood. In one embodiment, said GASP gene is selected from the group consisting of Gprasp1, Gprasp2, Gprasp3 and Gprasp7. In one embodiment, said HSCs of step a) express two or more GASP genes wherein said two GASP genes are Gprasp1, Gprasp2 and Gprasp3. In one embodiment, said HSCs of step a) express three GASP genes, wherein said three GASP genes are Gprasp1, Gprasp2 and Gprasp3. In one embodiment, said nonhuman:nonrodent animal is selected from the group consisting of equines, bovines, canines, and felines. In one embodiment, said method further comprises step c) transplanting said treated HSCs into said subject. In one embodiment, said HSCs of step b) improves engraftment potential.

In one embodiment, said Gprasp3 gene is the human Basic Helix-Loop-Helix Domain Containing, Class B, 9.

The invention provides a method for enhancing HSC engraftment, comprising, a) providing, i) a human hematopoietic stem cell (HSC) population, wherein said HSCs have a HLA haplotype and express a gene in the GASP (G-protein coupled receptor Associated Sorting Proteins) gene family, and ii) a human patient having an HLA haplotype, and b) treating said HSCs under conditions such that expression of said GASP gene in said HSC population is reduced, and c) transplanting said treated HSCs into said patient. In one embodiment, said treatment is shRNA-mediated knockdown of said GASP gene. In one embodiment, said GASP gene is the Gprasp1 gene. In one embodiment, said GASP gene is the Gprasp2 gene. In one embodiment, said GASP gene is the Armcx1 gene. In one embodiment, said GASP gene is selected from the group consisting of Gprasp2 and Armcx1. In one embodiment, said a human hematopoietic stem population is obtained from a sample selected from the group consisting of bone marrow, mobilized peripheral blood and umbilical cord blood (UCB). In one embodiment, said treating further comprises treating said HSCs with a shRNA for a second GASP gene. In one embodiment, said human hematopoietic stem population is obtained from bone marrow. In one embodiment, said human hematopoietic stem population is obtained from mobilized peripheral blood. In one embodiment, said human hematopoietic stem population is obtained from umbilical cord blood (UCB). In one embodiment, said HSC HLA haplotype is a mismatch (allogeneic or semi-allogeneic) between said stem cell population of umbilical cord blood (UCB: umbilical cord blood HSCs) and said HLA haplotype of said patient.

The invention provides a method for enhancing HSC engraftment, comprising, a) providing, i) a human umbilical cord blood (UCB) stem cell population, wherein said UCBs have a HLA haplotype and express a gene in the GASP (G-protein coupled receptor Associated Sorting Proteins) gene family, wherein said GASP gene is selected from the group consisting of Gprasp1, Gprasp2 and Armcx1, and ii) a human patient, wherein said patient has a major Human Leukocyte Antigen (HLA) haplotype, and b) treating said HSCs to reduce expression of said GASP gene, and c) transplanting said treated HSCs into said patient. In one embodiment, said HSC HLA haplotype is a mismatch (allogeneic) between said umbilical cord blood (UCB) stem cell population and said HLA haplotype of said patient.

The invention provides a method for enhancing HSC engraftment, comprising, a) providing, i) a human hematopoietic stem cell (HSC) population, wherein said HSCs express a gene in the GASP (G-protein coupled receptor Associated Sorting Proteins) gene family, and ii) a human patient, b) treating said HSCs under conditions such that expression of said GASP gene in said HSC population is transiently reduced for a time period and magnitude sufficient to improve the engraftment potential of the HSCs, and c) transplanting said treated HSCs into said patient. Reduction need not, in this embodiment, be permanent. Indeed, it is preferred that GASP gene expression recovers or at least increases after it is transiently reduced.

The invention provides a method for enhancing HSC engraftment, comprising, a) providing, i) a human hematopoietic stem cell (HSC) population, wherein said HSCs express a gene in the GASP (G-protein coupled receptor Associated Sorting Protein) gene family, and ii) a human patient, b) treating said HSCs under conditions such that expression of said GASP gene in said HSC population is transiently reduced, and c) transplanting said treated HSCs into said patient. In one embodiment, said transiently reduced is under conditions sufficient for improving the engraftment of the HSCs. For example, the conditions involve a time period (hours to days) of lower expression, followed by increased expression.

### BRIEF DESCRIPTION OF THE DRAWINGS

FIG. 1A-E. Functional Screen For Regulators Of HSPC in vivo Repopulation. FIG. 1A) Screen schematic. 51 prioritized genes were assessed by qRT-PCR for expression in LSK cells. miR30-embedded shRNAs targeting each gene expressed in LSK cells were cloned into a lentiviral vector downstream of the MSCV promoter. Here, the PGK promoter drives mCherry. FIG. 1B) Heat map of qRT-PCR of GOI in LSK cells, Lineage− cells, and Lineage+ cells. Scale indicates gene expression relative to population expressing the highest level of each gene across each row (1=dark red). FIG. 1C) Bone marrow LSK cells transduced with shRNAs were assayed 3-4 days post-transduction for mCherry. Each circle is an independent transduction event. FIG. 1D) Bone marrow LSK cells transduced with shRNAs were examined 3-4 days post-transduction by qRT-PCR. Each circle is an independently screened shRNA. Circles in red denote shRNAs used in the screen. FIG. 1E) Transduction efficiency (% mCherry+) of LSK cells and HSC (i.e. LSK CD150+CD48−) at multiple MOI-4 day post-transduction.

FIG. 2A-G. Identification Of Genes Contributing To HSPC In vivo Repopulation. FIG. 2A) shRNAs were transduced into CD45.2⁺“Test” LSK cells that were then transplanted into CD45.1+/CD45.2⁺ mice with an equal number of CD45.1⁺ mock transduced “Competitor” LSK cells. Recipient PB was analyzed for >16 weeks for CD45.2+ cells. FIG. 2B) Transduction of Test LSK cells for each screen transplant. For each transplant, an aliquot of Test cells was assessed for % mCherry+ cells 4 days post-transduction. Each circle represents an independent transduction. Loss-of-function Hits FIG. 2C) and non-Hits FIG. 2D). % CD45.2 PB four and >16 weeks post-transplant of recipients of gene specific-shRNA treated Test cells normalized to that of recipients of control-shRNA treated Test cells. Each gene was interrogated with at least two independent shRNAs (labeled as a and b). FIG. 2E) % CD45.2 PB of mice transplanted with Grb10-shRNA or control-shRNA transduced Test cells. Knockdown of Grb10 had no effect on LSK cell repopulating activity. FIG. 2F) LSK cells transduced with control- or Grb10-shRNAs were examined 4 days post-transduction for % mCherry+ cells. FIG. 2G) 30 weeks post-transplant, CD45.2+ LSK cells were isolated from the bone marrow of individual mice transplanted with CD45.2+ LSK cells transduced with either control- or GrMO-shRNAs. These cells were examined by qRT-PCR for Grb10 transcript levels. For panels C-F, the average of five recipient mice is presented and error bars represent standard deviation. For C. a one sample t-test was performed testing the null hypothesis that the normalized measurements=1. P-values are two-sided. § denotes p<0.1, * denotes p<0.05, ** denotes p<0.005. *** denotes p<0.0001.P values calculated >16 weeks post-transplant are shown.

FIG. 3A-G. Validation of Loss-of-function Hits Identifies 15 Genes Contributing To Robust HSPC Repopulating Activity. FIG. 3A) For retesting Hits: mCherry⁺ CD45.2⁺“Test” HSPC (LSK cells] transduced with either control or gene-specific shRNAs were transplanted into CD45.1+/CD45.2⁺ mice with an equal number of CD45.1⁺ mock transduced and mock sorted “Competitor” HSPC. Recipient PB was analyzed for >16 weeks for CD45.2⁺ cells. FIG. 3B) Representative flow cytometry analysis of LSK cell and HSC (i.e. LSK CD150+CD48-) 40 hours post-transduction with control shRNA lentiviral vector. Samples were examined for the frequency of mCherry+ cells. FIG. 3C) Transduction efficiency (% mCherry+ cells) of Test LSK cells transduced with Smarca2- and Zfp251-shRNAs in primary screen. FIG. 3D) Knockdown efficacy of shRNAs targeting Smarca2, Zfp251, and Zbtb20 assessed by qRT-PCR 3-4 days post-transduction of LSK cells. FIG. 3E) Verified loss-of-function Hits. A one sample t-test was performed testing the null hypothesis that the normalized measurements=1. P-values are two-sided. § denotes p<0.1. * denotes p<0.05. ** denotes p<0.005. *** denotes p<0.0001. P values calculated >16 weeks post-transplant are shown. FIG. 3F) Functional screen non-Hits. In panels FIG. 3E) and FIG. 3F), each gene was interrogated with at least two independent shRNAs (labeled as a, b, or c) and % CD45.2 PB at four and >16 weeks post-transplant of recipients of gene specific-shRNA treated Test cells normalized to that of recipients of control-shRNA treated Test cells in shown. FIG. 3G) Distribution of T, B, and myeloid PB lineages in mCherry+CD45.2⁺ compartment of genes that scored as Hits after retesting >16 weeks post-transplant. In panels E-G, each bar is the average of at least four recipient mice and error bars=standard deviation. In G, asterisk denotes statistically significant difference in distribution of at least one lineage relative to control for both shRNAs tested (p<0.05). P values were calculated using the Exact Wilcoxon Mann-Whitney test. ND=not determined.

FIG. 4A-C. Functional Screen Identifies Gprasp2 And Armcx1 As Negative Regulators Of HSPC Repopulation. FIG. 4A) Gprasp2 or control-shRNAs were transduced into CD45.2⁺ LSK cells that were then transplanted into CD45.1+/CD45.2⁺ mice with an equal number of CD45.1⁺ mock transduced “Competitor” LSK cells. Recipient PB was analyzed for 20 weeks. % mCherry+CD45.2⁺ PB of recipients of Gprasp2-shRNA treated cells normalized to % mCherry+CD45.2⁺ PB of recipients of control-shRNA treated cells. Gprasp2 was tested in two independent experiments with three shRNAs (a, b, and c). Cumulative results shown for both experiments (n>5 at time points over a time period). FIG. 4B) Validation of Gain-of-function Hits (Gprasp2, Armcx1 and Leprel2). Gprasp2, Leprel2, Armcx1, or control-shRNAs were transduced into CD45.2⁺ HSPC. mCherry+ HSPC were resorted 40 hours post-transfection and transplanted either 1:1 or 1:4 with CD45.1⁺ mock transduced and mock sorted “Competitor” HSPC into CD45.1+/CD45.2⁺ mice. Data shown is % CD45.2⁺ recipient PB of gene specific-shRNA treated cells normalized to that of recipients of control-shRNA treated cells at >16 weeks post-transplant for 1:1 (i) or 1:4 (ii) transplants. Armcx1 was examined with three shRNAs (a, b, and c) in a single (i) and three (ii) independent experiments. Gprasp2 was interrogated with two shRNAs (b and d) in a single experiment (ii). Leprel2 was examined with two shRNAs (a and b) in a single experiment for both (i) and (ii). FIG. 4C) Distribution of T, B, and myeloid PB lineages in mCherry+CD45.2⁺ compartment of Gain-of-function Hits from >16 weeks post-transplant. In FIG. 4A and FIG. 4C, each value is the average of n≥5 mice, error bars=standard deviation. For panels, asterisks denote statistical significance. One asterisk=p<0.04, two asterisks=p<0.008. P values calculated via Exact Wilcoxon Mann-Whitney test, ns—not significant.

FIG. 5A-C. Functional Analysis Of Screen Hits. FIG. 5A) 500 mCherry+ LSK cells transduced with control or gene-specific shRNAs were assayed for CFU potential five days post-transduction. Values are the average of 2-3 independent experiments normalized to control ±standard error. FIG. 5B) Cell cycle status of the mCherry+ LSK cell compartment, the frequency of mCherry+ LSK cells, and apoptosis of mCherry+ LSK cells was analyzed five days post-transduction with control or gene-specific shRNAs. Values are the average of 2-3 independent experiments normalized to control standard error. For FIG. 5A) and FIG. 5B), a one sample t-test was performed testing the null hypothesis that the normalized measurements=1. P-values are two-sided. § denotes p<0.1, asterisk denotes p<0.05, and two asterisks indicate p<0.005. FIG. 5C) Heat map summarizing average % CD45.2⁺ (Test cell-derived) HSC, MPP, CMP, CLP, GMP, and MEP in recipients >16 weeks post-transplant. Values are normalized to control recipients (i.e. 1=yellow). Higher chimerism relative to control=darker green, lower chimerism relative to control=darker red. ND denotes “not determined”.

FIG. 6A-H. Foxa3 Is Dispensable For Native Hematopoiesis But Contributing To HSC Repopulating Potential. FIG. 6A) qRT-PCR of Foxa3 transcript. FIG. 6B) PB counts of Foxa3^{+/+}, Foxa3^{-/+,} and Foxa3^{-/-}, littermates. FIG. 6C) Absolute number of HSPC in one femur+one tibia+one pelvis of 6-10 week old Foxa3^{-/-} (n=5), Foxa3^{-/+} (n=6), and Foxa3^{+/+} (n=2) littermates. In FIG. 6B) and FIG. 6C), each circle represents an independent mouse. FIG. 6D) CFU activity of 150 Foxa3^{-/-} (n=5) or Foxa3^{+/+} (n=5) HSC. Error bars=standard deviation. P-value=6.2×10⁶. E) Schematic showing Foxa3^{-/-} or Foxa3^{+/+} HSC transplantation strategies. FIG. 6F) For 1° transplants, CD45.2⁺ Foxa3^{-/-} or Foxa3^{+/+} WBM was transplanted with CD45.1⁺ WBM into ablated CD45.1⁺/CD45.2⁺ recipients in a 1:1 ratio. % CD45.2⁺ recipient PB at 20 weeks post-transplant is shown (P-value=0.03). For 2° transplants, CD45.2⁺ WBM was isolated from 1° recipients 16 weeks post-transplant and transplanted into ablated CD45.1⁺/CD45.2⁺ mice. % CD45.2+ recipient PB is shown 16 weeks post-transplant for 2° transplant recipients (P-value=0.0001). Each circle is an independently transplanted mouse. FIG. 6G) The LSK, HSC, and MPP compartments of 1° recipients of CD45.2⁺ Foxa3^{-/-} (n=12) or Foxa3^{+/+} (n=11) cells were examined >16 weeks post-transplant for the absolute number of CD45.2⁺ cells (shown as number of cells/one femur+one tibia+one pelvis). Each circle is an independent mouse. P-values=0.02, 0.08, and 0.04, respectively. FIG. 6H) 15,000, 30,000, 50,000,100,000, or 200,000 CD45.2⁺ Foxa3^{-/-} or Foxa3^{+/+} WBM cells were transplanted with CD45.1⁺ WBM into CD45.1⁺/CD45.2⁺ recipients. Recipients were scored as repopulated if their CD45.2+PB chimerism was >1% in the T cell, B cell, and myeloid cell lineages 10-16 weeks post-transplant (data are the pooled results of two independently performed limiting dilution transplants). Each circle is an individual recipient (black circles label engrafted mice and red circles label non-engrafted mice). The number of mice engrafted/number of mice transplanted at each cell dose is shown. Significantly fewer repopulating HSC were detected in Foxa3^{-/-} WBM than Foxa3^{+/+} WBM (p=0.0046). Chi-square analysis revealed a fit to the limiting dilution (LD) model (see, Table 3). These analyses were performed using L-Calc.

FIG. 7A-B. Foxa3 Protects HSC From Cellular Stress. FIG. 7A) Genes predicted by IM-PET to be targets of FOXA3 binding motif+LT-HSC enhancers (Table 5) are significantly more perturbed in expression amongst genes differentially expressed between Foxa3^{-/-} and Foxa3^{+/+} HSC (Table 6). P-value=2.6×10⁻²⁹. FIG. 7B) CD45.2+ LSK CD150+CD48− cells were isolated from 1° recipients of Foxa3^{+/+} (n=6) and Foxa3^{-/-} (n=7) bone marrow and then stained with DCFDA to assess endogenous ROS levels (i) or treated with TBHP prior to DCFDA staining to induce elevated ROS (ii). Values represent the percentage of cells positive for DCFDA in Foxa3^{-/-} cells relative to Foxa3^{+/+} cells (i) or the relative fold change of DCFDA positive cells in Foxa3^{-/-} versus Foxa3^{+/+} CD45.2+ LSK CD150+CD48− following TBHP treatment (ii). For (i), P-value=0.001. P values calculated via Exact Wilcoxon Mann-Whitney test.

FIG. 8A-C. Representative Flow Cytometry Plots, Related To FIG. 5C, Presents Gating Flow Cytometry Gating Strategies For The Ex Vivo Analysis Of Cell Cycle, Cell Surface Phenotype, And Apoptosis. FIG. 8A) Representative gating strategy of mCherry+ LSK cells for cell cycle analysis five days post-transduction. FIG. 8B) Representative gating strategy for assessing frequency of LSK cells within the mCherry+ cell compartment five days post-transduction. FIG. 8C) Representative gating strategy of mCherry+ LSK cells for analysis of apoptotic cells five days post-transduction.

FIG. 9A-G. Gprasp2 and Armcx1 belong to the GASP gene family and are highly expressed in HSPC. FIG. 9A) Schematic of domains in Gprasp2, Armcx1 and Gprasp1, members of the G-protein coupled receptor Associated Sorting Protein (GASP) family. FIG. 9B) Representation of the predicted roles of Gprasp2 and Armcx1. FIG. 9C), FIG. 9D), FIG. 9E): qRT-PCR data showing enrichment of Gprasp2, Armcx1 and Gprasp1 expression in murine bone marrow (BM) HSPC compartments. FIG. 9F), FIG. 9G): (i) qRT-PCR shows higher expression of human GPRASP2 and ARMCX1 in BM HPSC relative to differentiated progenitors. This expression correlates with their predicted expression shown in the gene expression database, (i.e.) HemaExplorer.

FIG. 10. Gprasp1 And Gprasp2 shRNAs Demonstrate A Range Of Specificities Shown In A Comparative Chart. ShRNAs targeting murine Gprasp1 or Gprasp2 efficiently and specifically knock-down Gprasp1 and Gprasp2 gene expression, respectively, in murine hematopoietic stem cells and murine hematopoietic stem progenitor cells (HSPC).

FIGS. 11A-B. shRNA Induced Reduction Of Gprasp1 Or Gprasp2 Enhances The Repopulation Activity Of HSPC While Genetic Loss Of Gprasp1 Or Gprasp2 In HSC−/− Populations Does Not Enhance The Repopulation Activity Of HSPC. FIGS. 11A-B show a schematic diagram for an exemplary experimental method (left) and results in a chart (right). FIG. 11A CD45.2+ HSPC were transduced with control or Gprasp-shRNA, as shown, then transplanted with CD45.1 “Competitor” HSPCs into recipient mice. Recipient mouse blood was then analyzed for CD45.2+ cells. ShRNA knock-down of Gprasp1 or Gprasp2 enhances the blood repopulating activity of HSPC after 4 weeks and continues up to and after 16 weeks. Each dot in the chart on the right represents an independently transplanted mouse. FIG. 11B CD45.2+ Gprasp+/+ HSPCs or Gprasp−/− HSPCs were transplanted with CD45.1 HSPCs into irradiated CD45.1+/CD45.2+ recipient mice. Recipient mouse blood was then analyzed for CD45.2+ cells up to and over 16 weeks post-transplantation. Each dot in the chart on the right represents an independently transplanted mouse. Genetic loss of Gprasp1 or Gprasp2 gene translation into GPRASP1 or GPRASP2 protein, does not result in enhanced blood repopulating activity of HSPC.

FIGS. 12A-C. Gprasp1-shRNA Or Gprasp2-shRNA Do Not Enhance The Repopulating Activity Of Treated Gprasp1−/− HSPC Or Gprasp2−/− HSPC, Respectively: While shRNA Silencing Of A Second Gprasp Gene In Gprasp1−/− HSPC Or Gprasp2−/− HSPC Induces A Partial Gain Of Enhanced Repopulating Activity. FIG. 12A shows a schematic diagram for an exemplary experimental method, and FIGS. 12B-C show comparative charts of experimental results. FIGS. 12A-B In part, for testing off-target effects of Gprasp1-shRNA or Gprasp2-shRNA: CD45.2+ Gprasp1−/− HSPCs (ii) or Gprasp2−/− HSPCs (i) were transduced with either control shRNA or Gprasp1-shRNA (ii) or Gprasp2-shRNA (i) then transplanted along with CD45.1+ HSPCs into irradiated CD45.1+/CD45.2+ recipient mice (n=4)/group). Gprasp1−/− HSPCs and Gprasp2−/− HSPCs did not display enhanced repopulating activity when treated with Gprasp1-shRNA (ii) or Gprasp2-shRNA (i), respectively. Thus, Gprasp-shRNAs do not have off-target effects that causes enhanced repopulation. FIG. 12C CD45.2+ Gprasp1−/− HSPCs (ii) or Gprasp2−/− HSPCs (i) were transduced with either control shRNA or Gprasp1-shRNA (ii) or Gprasp2-shRNA (i) then transplanted along with CD45.1+ HSPCs into irradiated CD45.1+/CD45.2+ recipient mice. Recipient mouse blood was then analyzed for CD45.2+ cells up to and over 16 weeks post-transplantation. Loss of Gprasp1 expression in Gprasp2−/− HSPCs (i) and loss of Gprasp2 expression in Gprasp1−/− HSPCs (ii) enhanced blood repopulating activity of HSPC. Each dot in the charts represents an independently transplanted mouse.

FIGS. 13A-B. Bhlhb9 Is Upregulated In Murine Gprasp1−/− HSPCs And Gprasp2−/− HSPCs. FIG. 13A shows that Bhlhb9 is upregulated in Gprasp1−/− LT-HSCs (long-term HSC) and Gprasp2−/− LT-HSCs. Thus Bhlhb9 may functionally compensate for loss of Gprasp1 or Gprasp2 in HSC. FIG. 13B shows a schematic diagram for an exemplary experimental method (right) and a chart showing results (left) demonstrating that knock-down of Bhlhb9 in murine HSPC does not enhance their repopulating activity.

FIGS. 14A-B. GASP Family Members Gprasp1, Gprasp2 And Bhlhb9 Are Expressed By Human Hematopoietic Stem Cells (HSC) And Progenitor Cells (HSPC). FIG. 14A GPRASP1, GPRASP2 and BHLHB9 are structurally similar members of the GASP (G-protein coupled receptor Associated Sorting Proteins) protein family that FIG. 14B are expressed by human hematopoietic stem cells (HSC).

FIG. 15. Validation Of shRNAs That Efficiently Knock-Down Human GPRASP1 Or GPRASP2 RNA Expression In Human Cell Lines. Validation of shRNAs showing a robust knock-down of human Gprasp1 or Gprasp2 in human cell lines.

### DEFINITIONS

To facilitate an understanding of the present invention, a number of terms and phrases are defined below. The use of the article “a” or “an” is intended to include one or more. As used herein, terms defined in the singular are intended to include those terms defined in the plural and vice versa.

As used herein, the term “GASP” or “G-protein coupled receptor Associated Sorting Protein” or “GPCR-associated sorting protein” and “GPRASP” or “G protein-coupled receptor associated sorting protein” gene family” refers to a family of genes encoding at least 10 proteins that interact with G protein-coupled receptors (GPCRs).

As used herein, the term “construct” refers to an artificially constructed segment of nucleic acid, i.e. recombinant, wherein separate nucleic acid sequences are ligated together, for example attaching nucleic acid sequences by using the enzyme ligase. As one example, a shRNA GASP gene silencing vector may be a construct.

As used herein, the term “vector” is used in reference to a nucleic acid molecule that transfers DNA segment(s) into a cell. The term “vehicle” is sometimes used interchangeably with “vector.” A “vector” may be a plasmid, phage, transposon, cosmid, chromosome, virus, retrovirus, virion, particle, etc., which is capable of replication when associated with the proper control elements. Thus, the term includes cloning and expression vehicles, as well as viral and retroviral vectors.

As used herein, “express” in relation to a gene refers to a process by which genetic instructions in DNA are used to synthesize gene products, i.e. protein, via RNA, or numerous types of RNA that do not encode entire proteins, i.e. shRNA expressed by a DNA vector.

As used herein, the term “expression vector” or “expression construct” or “expression vector construct” refers to a virus or plasmid constructed for gene expression in cells, i.e. where a desired nucleic acid sequence or gene is inserted into the vector in operable combination. The vector is used to introduce a specific gene into a target cell, where the cell's mechanism for transcription produces an expressed RNA from the DNA of a desired nucleic acid sequence or gene inserted into the vector, where the gene may or may not be further translated into an expressed protein.

As used herein, the term “lentivirus vector” refers to a retroviral vector derived from the Lentiviridae family (e.g., human immunodeficiency virus, simian immunodeficiency virus, equine infectious anemia virus, bovine immunodeficiency virus (BIV), canine lentivirus, including but not limited to other lentiviral vectors capable of gene transfer in canine cells, e.g. Horn, et al., “Efficient lentiviral gene transfer to canine repopulating cells using an overnight transduction protocol.” BLOOD 103(10): 3710-3716 (2004), herein incorporated by reference), feline immunodeficiency virus (FIV), and caprine arthritis-encephalitis virus, etc.) that are capable of integrating into non-dividing cells (See, e.g., U.S. Pat. Nos. 5,994,136 and 6,013,516, both of which are incorporated herein by reference in their entirety).

As used herein, the term “gene silencing” refers to the ability of a cell to inhibit or prevent the expression of a certain desired gene, i.e. as their expression is reduced. Gene silencing can occur during either transcription or translation, such that if the desired gene encodes a protein then production of their encoded protein is reduced. Gene silencing is often considered the same as gene knockout, such that when a gene undergoes “knockdown” the expression of a target gene in an individual is selectively reduced, e.g. “shRNA-mediated knockdown” referring to the use of shRNA for gene silencing.

As used herein, the term “shRNA” or “short hairpin RNA” refers to a sequence of ribonucleotides comprising a single-stranded RNA polymer that makes a tight hairpin turn on itself to provide a “double-stranded” or duplexed region used to silence gene expression via RNA interference. A shRNA hairpin is cleaved into short interfering RNAs (siRNA) by cellular machinery resulting in siRNA hybridizing to and cleaving cellular RNAs (i.e. target) that match (are complementary to) the siRNA sequence.

As used herein, the term “RNA interference” or “RNAi” refers to the silencing or decreasing or reducing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the endogenous gene is either completely or partially inhibited. RNAi inhibits the gene by compromising the function of a target RNA, completely or partially.

As used herein, the term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand”; the strand homologous to the target RNA molecule is the “sense strand”, and is also complementary to the siRNA antisense strand. siRNAs may also contain additional sequences; non-limiting examples of such sequences include linking sequences, or loops, as well as stem and other folded structures. siRNAs appear to function as intermediaries in triggering RNA interference in vertebrates.

The terms “patient” and “subject” refers to any animal (e.g., a mammal), including, but not limited to, humans, rodents, and non-human:non-rodent such as non-human primates, equines (Equidae), bovines (Bovinae), canines (Canidae), felines (Felidae), etc. Typically, the terms “subject” and “patient” are used interchangeably herein in reference to a human, unless indicated otherwise herein. That said, a subject that is non-human and non-rodent (non-human:non-rodent) may find benefit from materials and methods described herein, when applied in immunological MHC context of the non-human:non-rodent subject. As one example, hematopoietic stem cell transplantation is contemplated for treating disease, including but not limited to immunological disorders in horses. See, for equine examples, Equine Clinical Immunology, Chapter 32. Hematopoietic Stem Cell Transplantation, Felippe, 2015. As one example, hematopoietic stem cell transplantation is contemplated for treating disease, including but not limited to lymphoma, malignant lymphoma, etc., in dogs. As one example, hematopoietic stem cell transplantation is contemplated for treating disease, including but not limited to mucopolysaccharidosis type I (MPS I) in felines.

As used herein, the term “control” refers to subjects, cells, vectors or samples, etc., which provide a basis for comparison for experimental subjects or samples. For instance, the use of control subjects or samples permits determinations to be made regarding the efficacy of experimental procedures. In some embodiments, the term “control” refers to a subject that which receives a mock treatment (e.g., vector without the target siRNA).

As used herein, the term “host” refers to an animal or cell comprising heterologous genes or heterologous cells, respectively. The term “host” also refers to a patient that is to be the recipient of a particular treatment, e.g. engraftment. Typically, the terms “host” and “patient” are used interchangeably herein in reference to a human subject.

As used herein, the term “host cell” refers to any eukaryotic cell or prokaryotic cell (e.g., bacterial cells such as E. coli, yeast cells, mammalian cells, etc.), whether located in vitro or in vivo comprising a heterologous gene, or fragments thereof. For one example, host cells may be located in a chimeric mammal.

As used herein, the term “heterologous” refers to a gene or cell that is derived from a different cell or different animal than the host.

As used herein, the term “transfection” or “transduction” refers to the introduction of foreign (or heterologous) DNA into a host cell, such as expression vectors or particles thereof, encoding shRNA of the present inventions. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduced refers to the past tense of transduction.

As used herein, the term “transient” refers to temporary, e.g. a short time period (hours to days). As opposed to “stable” referring to longer time periods (days to weeks). The term “transient” indicates the condition is not permanent.

As used herein, the term “reduce” or “decrease” or “lose” refers to a smaller, or lower, or lesser amount, as a comparative number, degree, or size, etc. For one example, a lower amount of expressed Gprasp RNA in a population of HSPCs after targeted Gprasp-shRNA treatment as compared to HSPCs treated with a control (nontargeted shRNA for that Gprasp gene), is a reduction, e.g. Gprasp2 RNA may be reduced after treatment with shRNA targeting Gprasp2, i.e. Gprasp2-shRNA as compared to the control.

As used herein, the term “increase” or “gain” refers to a larger, or higher, or greater amount, as a comparative number, degree, or size, etc. For example, an increase in an amount is a higher amount when compared to a control, such as when CD45.2 RNA is increased after certain Gprasp-shRNA treatments of CD45.2+ HSPCs over control shRNA treatments of CD45.2+ HSPCs.

As used herein, the term “magnitude” refers to a size, or length, or amount, or extent, as in extent in time. As one example, an amount of reduction may be referred to as the magnitude of reduction, for example,

CD45.2+ in expression of a GASP gene refers to an amount such that at least a 50% reduction of expression (relative to control expression of that particular GASP gene RNA) of at least one GASP gene is obtained, however it is not meant to limit the amount of reduction of at least one GASP gene's expression. Indeed, expression of a GASP gene may be reduced at least 10%, 20%, 30%, 40%, and preferably at least 50%, 60%, 70%, 80%, 90%, 95%, 98%, 99% up to but not including a 100% reduction.

As used herein, the term “potential” refers to having or showing a capability to become or develop into something in the future.

As used herein, the term “gene expression” refers to the process of converting genetic information encoded in a gene into RNA (e.g., mRNA, rRNA, tRNA, or snRNA) through “transcription” of a gene or a nucleic acid sequence, such as an shRNA sequence (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA, shRNA, or protein), while “down-regulation” or “repression” refers to regulation that decreases production. Molecules (e.g., transcription factors) that are involved in up-regulation or down-regulation are often called “activators” and “repressors,” respectively.

As used herein, the term “effective amount” refers to the amount of a composition (e.g., composition comprising a RNAi regulator inhibitor, i.e. shRNA) sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, test compound or other agent, or therapeutic treatment (e.g., compositions of the present invention) to a cell or subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the term “treating” refers to administering a compound or construct or cells to a cell or subject, including transducing a GASP shRNA into HSCs.

As used herein, the terms “co-administration” and “co-administering” refer to the administration of at least two agent(s) (e.g., a composition comprising at least two RNAi regulator inhibitor (e.g., siRNA), or and one or more other agents, e.g., a non-RNAi regulator siRNA) or therapies to a cell or subject. In some embodiments, the co-administration of two or more agents or therapies is concurrent. In other embodiments, a first agent/therapy is administered prior to a second agent/therapy. Formulations and/or routes of administration of the various agents or therapies used may vary. In some embodiments, when agents or therapies are co-administered, the respective agents or therapies are administered at lower dosages than when used for their administration alone. Thus, co-administration is especially desirable in embodiments when co-administration of two or more agents results in sensitization of a subject to beneficial effects of one of the agents via co-administration of the other agent. As used herein, the term “transplant” refers to tissue used in grafting, implanting, or transplanting, as well as the transfer of tissues from one part of the body to another, the return of cells to the original donor (autologous transplants) or the transfer of tissues or cells from one individual to another, or the introduction of biocompatible materials into or onto the body. The term “transplantation” refers to the grafting of tissues from one part of the body to another part, or to another individual.

As used herein, the term “engrafting” in reference to a stem cell refers to placing the stem cell (e.g. HSC) into an animal (e.g., by injection), wherein the stem cell persists in vivo. This can be readily measured, for HSCs, by the ability of the HSC to contribute to ongoing blood cell formation.

As used herein, the term “engraftment” refers to a capability of donor-derived cells to grow, divide and function. As one example, the capability of bone marrow stem cells and progenitor cells to establish donor-specific hematopoietic chimerism. “Engraftment” also refers to the growth and development of donor blood cells in a host.

As used herein, the term “stem cell” or “undifferentiated cell” refers to self-renewing cells that are capable of giving rise to phenotypically and genotypically identical daughters as well as at least one other final cell type (e.g., terminally differentiated cells). Stem cells include, but are not limited to, hematopoietic stem cells and progenitor cells derived therefrom (see U.S. Pat. No. 5,061,620, herein incorporated by reference); umbilical cord stem cells (e.g. derived from umbilical cord blood), placental stem cells (e.g. derived from placental tissues collected during or after birth); adult stem cells (e.g. derived from different parts of the body such as bone marrow, blood, and fat); neural crest stem cells; embryonic stem cells; mesenchymal stem cells; mesodermal stem cells; stromal stem cells, pulmonary epithelial stem cells, hepatic stem cells, induced pluripotent stem cells (iPSCs); and other stem cells.

As used herein, the term “stem cells” refers to cells that are pluripotent or multipotent and are capable of differentiating into one or more different cell types, including multipotent cells. In some embodiments, stem cells refer to cells that are capable of replicating “indefinitely” typically transplanted stem cells last for some portion of the remaining life span of the subject,

As use herein, the term “embryonic stem cells” refers to cells derived (originally obtained) from an embryo.

As used herein, the term “adult stem cells” means stem cells derived (originally obtained) from an organism after birth.

As used herein, the term “totipotent” refers to a cell capable of differentiating into any type of cell, such as a fertilized oocyte.

As used herein, the term “pluripotent” refers to a cell capable of differentiating into several cell types that are in turn capable of differentiating into specific cell types, for examples, iPSC, mESC, hESC, etc.

As used herein, the term “multipotent” refers to a cell capable of differentiating into at least two cell types, for example, adult stem cells.

As used herein, the term “hematopoietic stem cell” or “HSC” refers to multipotent stem cells that form blood and immune cell types, i.e. give rise to blood cells, through the process of haematopoiesis. Blood cells include both the myeloid and lymphoid lineages, i.e. Myeloid cells include monocytes, macrophages, neutrophils, basophils, eosinophils, erythrocytes, myeloid-dendritic cells, and megakaryocytes or platelets, etc., and lymphoid cells include T cells, B cells, natural killer cells, lymphoid-dendritic cells, etc. Hematopoietic stem cells are a population of heterogenous cells with long-term and short-term regeneration capacities, including progenitor cells (i.e. committed multipotent, oligopotent, and unipotent progenitor cells). HSCs are found in the bone marrow (e.g., in the pelvis, femur, and sternum). In general, a hematopoietic stem cell is a cell isolated from the blood, umbilical cord blood or bone marrow that can renew itself and has the capability to differentiate to a variety of specialized cells. HSC may move out of the bone marrow into circulating blood. A small number of HSCs can expand to generate a very large number of daughter HSCs. This phenomenon is used in “bone marrow transplantation”, when a small number of donor HSCs reconstitute the host's hematopoietic system.

As used herein, the term “heterogeneous” refers to mixture, such as a population of mixed cells that are diverse in character as opposed to “homogenous” referring to a population of the same kind, as when a sub population of cells having a same characteristic, for example, CD34 (CD: cluster of differentiation) expression, is isolated from a mixed population.

As used herein, the term “progenitor cell” refers to a cell that has the capability to differentiate into a specific type of cell, but is already more differentiated, i.e. specific, than a stem cell, and in some embodiments more differentiated than a pluripotent cell, and in some embodiments may be capable of differentiating into a specific cell type or cell lineage. Progenitor cells can divide a limited number of times as opposed to a stem cell (i.e. a progenitor cell has limited self-renewal, i.e. a more limited number of divisions that produce a progenitor cell as opposed to a stem cell that can divide numerous times for replicating the stem cell).

As used herein, the term “donor cells” refer to stem cells and progenitor cells. While stem cells and/or progenitor cells can be obtained (i.e. harvested) from bone marrow, it its not meant to limit the source of such cells for use in methods described herein. Thus, in one embodiment, stem cells and/or progenitor cells can be obtained (i.e. harvested) from bone marrow. As one example, bone marrow containing stem cells and progenitor cells, e.g. the pelvis, at the iliac crest, using a needle and syringe. The cells can be removed in a liquid (to perform a smear to look at the cell morphology) or they can be removed via a core biopsy. Donor cells may also be obtained from the circulating peripheral blood. Thus, in another embodiment, donor cells may be from white blood cell populations harvested from peripheral blood, e.g. isolated from peripheral blood white blood cell populations containing stem cells and progenitor cells.

As used herein, the term “isolated” when used in reference to a cell refers to a cell that is removed from its natural environment (e.g., bone marrow, blood, etc.) and that is separated (e.g., is at least about 25% free, 50% free, and most preferably about 90% free), from other cells with which it is naturally present.

As used herein, the term “expansion” of a stem cell indicates that there is an increase in the absolute number of stem cells (e.g., during the culturing of the cells). Analogously, a stem cell that has undergone such expansion has been “expanded.”

As used herein, the term “enhance” or improve” refers to an additional benefit, such as any one or more of a quality, a quantity, time period, outcome, etc.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, “mismatch” refers to tissues or cells that are genetically dissimilar and hence immunologically incompatible, although from individuals of the same species e.g. allogenic.

As used herein, “graft rejection” refers to when immune cells (T-lymphocytes) of the recipient (host) recognize specific HLA antigens on the donor's cells as foreign. The T-lymphocytes initiate a cellular immune response that result in graft rejection. Alternatively, T-lymphocytes present in the grafted tissue may recognize the host tissues as foreign and produce a cell-mediated immune response against the recipient. This is called “graft versus host disease” or “GVHD” and it can lead to life-threatening systemic damage in the recipient. Graft-versus-host disease may be acute or chronic. Human leukocyte antigen testing is performed to reduce the probability of both rejection and GVHD.

As used herein, the term “chimera” or “chimerism” is intended to encompass hosts comprising grafts such as, but not limited to, (a) a recipient (i.e. host) who may have cells exhibiting both donor and recipient surface histocompatibility antigens that are recognized as “self” by the recipient, co-existing in the recipient; (b) recipients who may have cells from three or multiple donors that are recognized as “self” by the chimeric recipient; and (c) combinations and permutations of the foregoing, without limitation.

As used herein, “mixed donor-recipient chimerism” is used to describe a state in which tissue or cells from a donor are able to live and function within a recipient host without graft rejection or the occurrence of GVHD. For example, in a semi-allogeneic transplantation, the donor and the recipient share at least one major histocompatibility complex (MHC) class I or class II locus, and the chimeric cells exhibit cell surface histocompatibility antigens of both the donor and the recipient (i.e., they are double positive). In a fully allogeneic transplantation, the donor and recipient do not share MHC locus molecules. In these chimeras, cells from the donor and cells from the recipient co-exist in the recipient, and these are both recognized as “self” and not rejected.

As used herein, the term “self” refers to any antigen-bearing endogenous material or foreign material that does not stimulate an attack on this material by the body's immune system. As used herein, “autologous” refers to self.

As used herein, “autologous” in reference to transplantation refers to a procedure in which cells are removed and later given back to the same person.

As used herein, the term “non-self” refers to any antigen-bearing foreign material (such as white blood cells and somatic cells) that enters the body and normally stimulates an attack on the foreign material by the body's immune system (as distinguished from self).

As used herein, “allogeneic” refers to non-self.

As used herein, “allogeneic” in reference to transplantation refers to a procedure in which cells are removed, e.g. sibling, relative or unrelated person, and later given to a different person, as in allograft, allogeneic transplant, or homograft.

As used herein, the term “niche” refers to a space that the cell occupies, for example, within the bone marrow.

As used herein, the term “preconditioning” in reference to a transplant recipient refers to creating a “space” needed for engraftment of the transplanted syngeneic or allogeneic cells. As one example, a niche is created by whole body irradiation, or other cytoablation procedures, and the like.

As used herein, “major histocompatibility complex” or “major histocompatibility locus” or “MHC” refers to certain proteins, i.e. molecules, located on the surface of the white blood cells and other cells and tissues in the body. MHC proteins are primarily grouped as Class I or II, depending upon their structure. MHC may also refer to a system of naming these molecules for each species, e.g. human leukocyte antigen (HLA), equine leucocyte antigen (ELA), bovine leucocyte antigen (BoLA), dog leucocyte antigen (DLA), feline leucocyte antigen (FLA), and the like. Each individual body uses some of these markers to recognize which cells belong in that body and which do not.

As used herein, “human leukocyte antigen” or “HLA” refers to the MHC molecules and system of naming these molecules in humans. HLA and MHC may be used interchangably. There are three major groups of HLA, i.e., HLA-A, HLA-B (i.e. Class I) and HLA-DR, HLA-DQ, and HLA-DP (i.e. Class II) and numerous minor groups. Class I molecules are expressed on the majority of cells in the body while Class II molecules are expressed mainly on white blood cells.

As used herein, “haplotype” refers to a specific set of MHC proteins of an individual, for one example in humans these are inherited as a “set” of the three HLA groups, A, B, and DR, each group having two molecules, one from the mother and one from the father. Further, each of the different HLA groups has subtypes identified with a numerical designation, for example, HLA-A1, HLA-A2, etc., such that a haplotype may be HLA-A1/HLA-A2, HLA-B1/HLA-B3, and HLA-DR3/HLA-DR4. Using specific antibodies for haplotyping, at least 26 HLA-A alleles, at least 59 HLA-B alleles, at least 10 HLA-C alleles, at least 26 HLA-D alleles, at least 22 HLA-DR alleles, at least 9 HLA-DQ alleles, and at least 6 HLA-DP alleles can be identified. Haplotypes may be different between animal species and certain subspecies.

Thus, a HLA haplotype or “HLA typing” or “histocompatibility testing” is used to match patients (hosts) and donors for tissue transplants, such as bone marrow or cord blood transplants.

As used herein, “match”, in reference to transplantation, refers to when two people share the same HLAs such that their tissues or cells are immunologically compatible with each other or in autologous stem cell transplantation. The probability that a transplant will be successful increases with the number of identical HLA antigens. Thus, the closer a match between a donor's and a patient's HLA markers increases successful transplant outcomes. Because some HLA types are more common than others, some patients may face a greater challenge in finding a matching donor. Some HLA types are found more often in certain racial and ethnic groups. Transplantation of umbilical-cord blood was successfully performed to treat individuals with blood-diseases where donors were newborn siblings being perfect HLA matches for the affected sibling.

“Histocompatibility testing” comprises three tests, HLA antigen typing (tissue typing), screening of the recipient for anti-HLA antibodies (antibody screen), and the lymphocyte crossmatch (compatibility test). HLA antigen typing may be performed by serological or DNA methods. The antibody screen is performed in order to detect antibodies in the recipient's serum that react with HLA antigens. The most commonly used method of HLA antibody screening is the microcytotoxicity test. If an antibody against an HLA antigen is present, it will bind to the cells. The higher the number of different HLA antibodies, the lower the probability of finding a compatible match. The third component of a histocompatibility study is the crossmatch test. In this test peripheral blood lymphocytes from the donor are separated into B and T lymphocyte populations. In the crossmatch, serum from the recipient is mixed with T-cells or B-cells from the donor. A positive finding indicates the presence of preformed antibodies in the recipient that are reactive against the donor tissues. An incompatible T-cell crossmatch contraindicates transplantation of a tissue from the T-cell donor.

As used herein, “ABO” refers to a system for classifying human blood on the basis of antigenic components of red blood cells and their corresponding antibodies for use in determining transplantation compatibility along with the MHC system. An ABO blood group is identified by the presence or absence of two different antigens, A and B, on the surface of the red blood cell. The four blood types in this grouping, A, B, AB, and O, are determined by and named for these antigens. Each ABO blood group also contains naturally occurring antibodies to the antigens it lacks. Group A has A antigens on the red cells, with anti-B antibodies in the plasma. Group B has B antigens on the red cells, and anti-A antibodies in the plasma. Group O has neither A nor B antigens, and both anti-A and anti-B in the plasma. AB has both A and B antigens on the red cells, and no anti-A or anti-B in the plasma.

The term “gene” refers to a nucleic acid (e.g., DNA or RNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one that is capable of directing a level of transcription of an operably linked nucleic acid sequence in the presence of a stimulus (e.g., heat shock, chemicals, etc.), which is different from the level of transcription of the operably linked nucleic acid sequence in the absence of the stimulus.

The term “recombinant DNA molecule” as used herein refers to a DNA molecule that is comprised of segments of DNA joined together by means of molecular biological techniques (e.g. using ligase for ligating a promoter to a DNA molecule into an expression plasmid).

The term “recombinant protein” or “recombinant polypeptide” as used herein refers to a protein molecule that is expressed from a recombinant DNA molecule.

As used herein, “amplification,” refers to the production of additional copies of a nucleic acid sequence. Amplification is generally carried out using polymerase chain reaction (PCR) technologies well known in the art. See, e.g., Dieffenbach C W & Dveksler G S, PCR Primer, a Laboratory Manual 1-5 (Cold Spring Harbor Press, Plainview, N.Y., 1995).

As used herein, “amplifying” refers to a PCR method wherein a target sequence i.e. amplicon, in a nucleic acid sample is copied.

As used herein, the term “PCR” or “polymerase chain reaction” refers to a general method for increasing the concentration of a target nucleic acid sequence within a mixture of DNA, performed by repeated cycles of three steps: denaturation, annealing, and extension. The DNA is denatured and then allowed to hybridize to primers. Following hybridization, the primers are extended with DNA polymerase so as to form complementary strands between the forward and reverse primers. The steps of denaturation, hybridization, and polymerase extension can be repeated as often as needed, in order to obtain relatively high concentrations of a segment of the desired target sequence. Exemplary techniques of the polymerase chain reaction as described in Saiki, et al., Nature 324:163 (1986); and Scharf et al., Science 233:1076-1078 (1986); Mullis et al. U.S. Pat. No. 4,683,195 and Mullis, U.S. Pat. No. 4,683,202, herein incorporated by reference. For PCR, two primers are used, the forward primer sequence and the reverse primer sequence which together define an amplicon sequence.

As used herein, the term “primer” refers to an oligonucleotide, whether as purified from a restriction digest or produced synthetically, which is capable of acting as a point of initiation of PCR synthesis when placed under conditions allowing synthesis of a primer extension product complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths and sequences of the primers will depend on several factors, including temperature of the reaction, source of polymerase, source of primer and the use of the method. Oligonucleotides may be synthesized by standard methods known in the art, e.g. by use of an automated DNA synthesizer (such as are commercially available from Biosearch, Applied Biosystems, etc.).

As used herein, “complementary” in reference to a DNA or RNA molecule refers to complementary base pairing, i.e. the manner in which the nitrogenous bases of the DNA or RNA molecules align with each other through hydrogen bonding. In other words, adenine (A) bonds with thymine (T) (or adenine bonds with uracil (U) in RNA), cytosine (C) bonds to guanine (G).

As used herein, “Quantitative PCR” or “qPCR” refers to a version of PCR method for both detecting the presence of a specific nucleic acid sequence and quantifying the number of copies present in a sample, at least relative to a control. “qRTPCR” may refer to “quantitative real-time PCR,” used interchangeably with “qPCR” as a technique for quantifying the amount of a specific DNA sequence in a sample. However, if the context so admits, the same abbreviation may refer to “quantitative reverse transcriptase PCR,” a method for determining the amount of messenger RNA present in a sample. Since the presence of a particular messenger RNA in a cell indicates that a specific gene is currently active (being expressed) in the cell, this quantitative technique finds use, for example, in gauging the level of expression of a gene.

The term “marker” refers to a fluorescent molecule or compound, such as expressed intercellular by an expression construct (vector), i.e. mCherry, or extracellular, identified using a fluorescent antibody attached to a fluorescent marker, i.e. Texas red, etc.

The term “fluorescent activated cell sorting” or “FACS”, as used herein, refers to a technique for counting, examining, and/or sorting cells suspended in a stream of fluid. It allows simultaneous multiparametric analysis of the physical and/or chemical characteristics of single cells flowing through an optical and/or electronic detection apparatus, and when desired used for sorting, e.g. isolating a subpopulation of cells having a certain level of granularity, as in enriched. Fluorescent chemicals found in the cell (i.e. mCherry) or attached to the cell (i.e. labeled antibody), may be detected and quantitated, and when desired used for sorting, i.e. isolating a subpopulation of cells, as in enriched.

The term “enriched” refers to increasing a characteristic or marker in the number of cells in a population, such as in a fractionated (or sorted) set, or subpopulation of cells as compared with the number of cells having that characteristic or marker in the unfractionated set, i.e. starting population of cells.

As used herein, the term “in vitro” refers to an artificial environment and to processes or reactions that occur within an artificial environment. In vitro environments can comprise, but are not limited to, test tubes and cell culture. “ex vivo” refers to that which takes place outside an organism, such as experimentation or measurements done in or on tissue from an organism in an external environment, ideally with minimal alteration of natural conditions. The term “in vivo” refers to a biological process occurring or made to occur within a living organism, such as within a living body.

### DESCRIPTION OF THE INVENTION

The present invention relates to methods of enhancing stem cell transplantation by treating pre-graft cells with silencing constructs for reducing expression of GASP (G-protein coupled receptor Associated Sorting Proteins) family genes, either permanently or transiently. In particular, methods of using a shRNA silencing construct for Gprasp1, Gprasp2 or Armcx1 (Gasp7) in pre-graft hematopoietic transplant cells are provided for improving the ability of these cells to replenish the hematopoietic system of host organisms. Further, the use of GASP gene silenced umbilical cord blood-derived cells is contemplated for transplantation into HLA mismatched (allogeneic) hosts.

Targeting GASP-family members for reduced expression in HSC is contemplated for enhancing the ability of these cells to replenish an ablated hematopoietic system in humans. shRNA-mediated knockdown of either Gprasp2 or Armcx1 in mouse HSC significantly enhances the ability of these cells to replenish the hematopoietic system of mice whose endogenous hematopoietic system has been ablated by irradiation. Methods of Hematopoietic stem cell (HSC) therapy using several genes in the GASP (G-protein coupled receptor Associated Sorting Proteins) gene family are contemplated. Examples of the genes are included but not limited to Gprasp2, Armcx1 (Gprasp7) and Gprasp1 as family members. These three genes are highly expressed by both mouse and human HSC. Further, the inventors contemplate that by targeting at least one GASP gene for reduced expression in HSCs, the efficiency of HSC transplantation would be improved. In addition to an improved outcome, the inventors further contemplated that by targeting at least one GASP gene for reduced expression in HSCs of umbilical cord blood (UCB) cells used for transplantation, these transplants would tolerate a greater degree of HLA mismatch between patient and donor than untreated UCBs and other HSC sources with fewer immunological complications, such as short-term graft rejection, graft vs. host disease, and longer term secondary immunological conditions triggered by engraftment. Thus, at least in part, by overcoming additional current limitations by providing additional donors and thus greater cell numbers available for transplantation due to an increase in donors. One major limitation in UCB transplantation is the small numbers of cells available for transplant from each donor, which leads to a longer delay time between injection of the cells and actual engraftment. Thus, not all patients can take advantage of UCB transplantation who might benefit (i.e. not enough cells to yield engraftment). The longer engraftment takes, the more prone the patient is to infection etc, which can lead to death. Therefore, decreasing the time to engraftment is beneficial to transplant patients. In particular, decreasing the time to engraftment is beneficial to transplant patients receiving UCBs. In order to hasten engraftment, i.e. for transplanted cells to protect the host from infections, the present invention contemplates UCB cells treated so as to silence a GASP gene. This should be a safer method and may be extended to more patients than when using untreated cells.

I. Hematopoietic Stem Cells (HSC).

Hematopoietic stem cells (HSC) can reconstitute the entire hematopoietic system following transplantation into hosts whose hematopoietic compartment has been ablated. This capability is used clinically as HSC transplantation (HSCT) to treat hematologic disease and represents the curative therapy for many disorders (Cavazzana et al., 2014; Cohen et al., 2014; Talano and Cairo, 2014).

Unfortunately, the application of HSCT can be limited by a paucity of HSC numbers, especially in cord blood transplantation (Zhong et al., 2010). As such, tremendous effort has been exerted to develop protocols that allow for the expansion of transplantable HSC ex vivo. Strategies range from identifying transcriptional regulators, developing supportive stroma, and identifying small molecules that promote expansion (Walasek et al., 2012). However, these approaches are limited by the tendency of HSC to differentiate in culture and have not yet been translated clinically.

One alternative for improving HSCT is to enhance HSC engraftment itself. Successful HSCT requires that donor HSC engage with the proper supporting niche, survive, proliferate, and differentiate into mature blood lineages. These processes are associated with numerous stresses including myelotoxic conditioning that alters the niche, ex vivo manipulation of HSC, and the requirement for supraphysiological hematopoietic expansion during engraftment and reconstitution.

Recent studies indicate that “stress hematopoiesis,” including that which occurs post-HSCT, is subject to distinct biological regulation compared to baseline hematopoiesis occurring in healthy individuals (Rossi et al., 2012). Further, the hematopoietic stem and progenitor cells (HSPC) that maintain hematopoiesis post-HSCT may differ from those that sustain native hematopoiesis (Busch et al., 2015; Sun et al., 2014). These differences indicate that factors that uniquely regulate the function of HSPC post-transplant might be useful for overcoming such limitations. For example, PGE2, shown to promote HSC engraftment by upregulating homing pathways and enhancing self-renewal, was recently tested in Phase 1 clinical trials where it enhanced the long-term engraftment of cord blood (Cutler et al., 2013; Hoggatt et al., 2009). This data indicates that enhancement of HSC engraftment would be able improve transplant outcomes. Thus, regulating or producing a stable repopulation of the hematopoietic compartment by HSPC is contemplated to improve HSCT.

Therefore, it is contemplated that the methods of the present invention can be used to produce red and white blood cells, such as lymphoid, myeloid and erythroid cells from hematopoietic stem cells. In one embodiment, the methods described herein would improve the efficiency of blood cell production. Blood cells include, but are not limited to the lymphoid lineage, comprising B-cells and T-cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers.

A. Regulators Of HSPC Repopulation.

Functional screens of murine and human HSC have focused on identifying genes that promote HSPC self-renewal and/or maintenance during ex vivo culture (Ali et al., 2009; Boitano et al., 2010; Deneault et al., 2009; Fares et al., 2014; Hope et al., 2010). In these studies, purified murine HSC or enriched human HSPC were transduced with the open reading frames of genes of interest (GOI), transduced with shRNAs targeting GOI, or treated with small molecule libraries. Cells were then maintained ex vivo for 5-17 days prior to downstream assays, which included transplantation into ablated mice for a rigorous functional assessment of HSC numbers; in vitro colony assays, or flow cytometry for retention of an HSPC cell surface phenotype. In each of these studies, extensive ex vivo culture prior to downstream analysis precluded a direct assessment of the effect of treatment on HSC engraftment, as this would be difficult to separate from effects on HSC expansion, differentiation during culture, or even non-cell autonomous effects on HSC maintenance, as was seen in one study (Deneault et al., 2009).

In contrast, our goal was to identify genes for enhancing the stable repopulation of an ablated hematopoietic system. To achieve this, we used the information obtained during the development of the present inventions in order to develop a system in which HSPC treated with shRNAs are subjected to minimal ex vivo culture prior to transplantation into cohorts of ablated mice, allowing us to directly assess any effect of the loss of gene expression on HSC engraftment and hematopoietic reconstitution.

Using a functional screen described herein, we identified 17 genes whose loss perturbs short and/or long term (i.e. stable) HSPC repopulation. Expression of 15 genes provided optimal repopulation while expression of two genes were inhibitors of stable HSPC engraftment, as their loss enhanced HSPC repopulation. Twelve (12) of these genes were not previously implicated in HSPC biology, including Foxa3 (formally known as hepatocyte nuclear factor 3γ or HNF-3γ). Foxa3 belongs to the Foxa sub-class of Fox (Forkhead Box) DNA-binding factors. FOXA proteins are transcriptional pioneer factors that establish competence for downstream transcriptional programs (Friedman and Kaestner, 2006). Foxa3 was studied for its role in endoderm and endoderm-derived tissue development (Friedman and Kaestner, 2006). However, a role for Foxa3 in several non-endodermal lineages was described (Behr et al., 2007; Ionescu et al., 2012; Xu et al., 2013), suggesting a broader role in tissue development and function. Here, we further demonstrate a novel role for Foxa genes in HSC biology via investigation of Foxa3^{-/-} mice.

B. Functional Screen For Novel Regulators of HSPC (Hematopoietic Stem and Progenitor Cell) Engraftment And Repopulation.

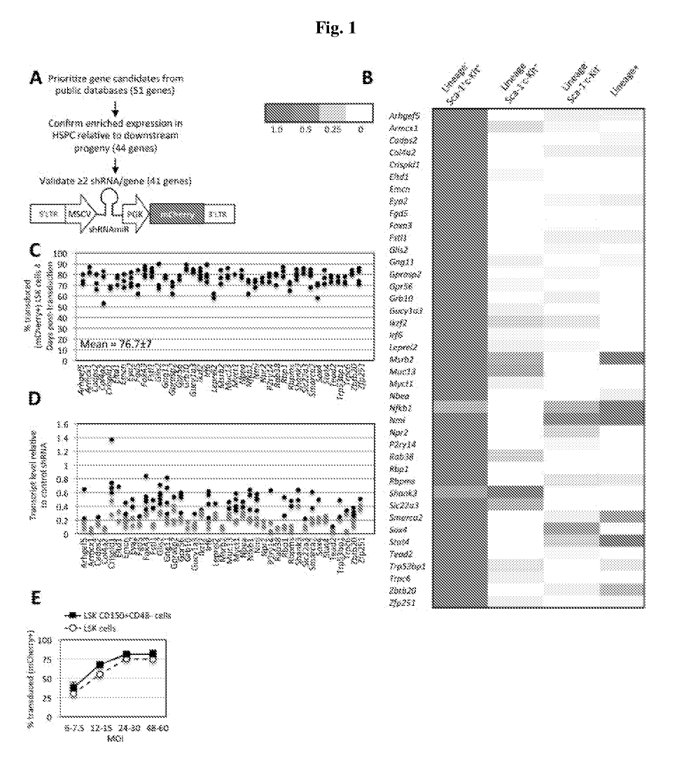

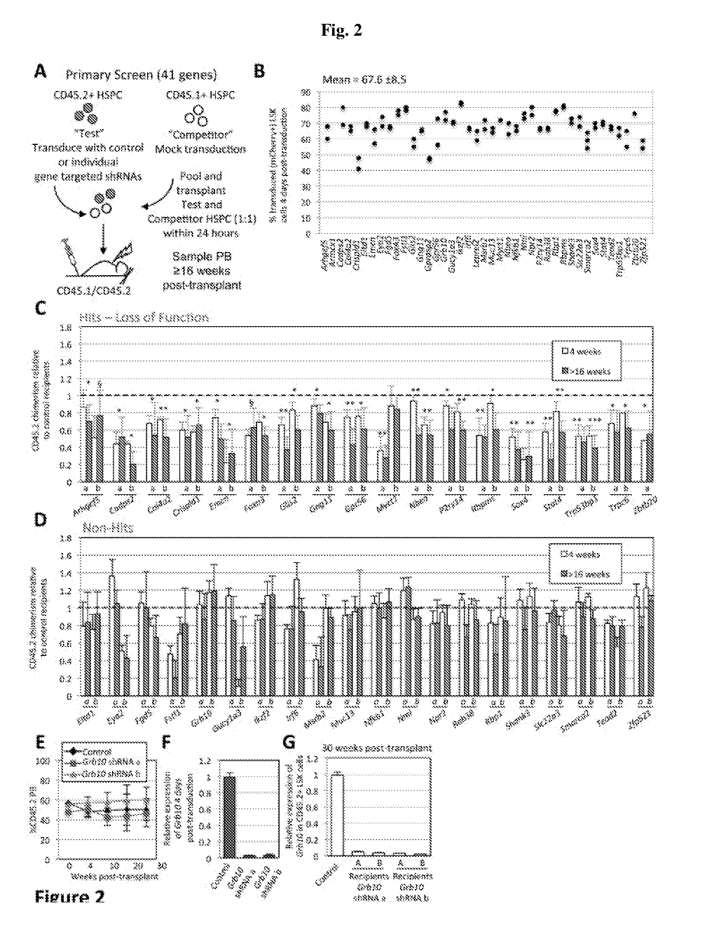

In order to discover novel regulators of HSPC (Hematopoietic Stem and Progenitor Cell) repopulation, we transplanted >1300 mice with shRNAs for one of 51 targeted prioritized gene candidates. Each shRNA was functionally validated to mediate robust gene knockdown in primary LSK cells (FIG. 1D). To ensure high resolution of Hits from non-Hits, we verified robust cell transduction for each experiment in our functional screen (FIG. 2B). Further, each putative Hit was validated by retesting, thereby minimizing the likelihood of false positives due to off-target effects or viral integration. These variables combined to yield a Hit rate of 41.5% (17/41 genes tested), illustrating the robustness of our approach and the fidelity of the publicly available resources from which our gene candidates were drawn (Chambers et al., 2007; Heng, et al., 2008; McKinney-Freeman, et al., 2012). Although homing contributes to HSPC engraftment, our screen was not technically designed to identify homing regulators.

1. Results Of Functional Screens.

shRNA-transduced mouse HSPC were transplanted into mice within a 24-hour time period of isolation and transduction in order to detect genes regulating repopulation. Thus, 17 new regulators of HSPC repopulation were identified for mouse HSCs, i.e. LSK cells in vivo repopulating activity: Arhgef5, Armcx1, Cadps2, Crispld1, Emcn, Foxa3, Fstl1, Glis2, Gprasp2, Gpr56, Myct1, Nbea, P2ry14, Smarca2, Sox4, Stat4, and Zfp251. Knockdown of each of these genes yielded a loss of function with the exception of Armcx1 and Gprasp2, whose loss surprisingly enhanced HSC repopulation instead. Thus, in one embodiment, ex vivo treatment of HSC with any one or more of Arhgef5, Cadps2, Crispld1, Emcn, Foxa3, Fstl1, Glis2, Gpr56, Myct1, Nbea, P2ry14, Smarca2, Sox4, Stat4, and Zfp251 protein or expression vector for increasing intracellular expression, in combination with treatment with an shRNA for a GASP gene, may also find use for promoting stable engraftment.

Twelve of these genes have not been implicated in HSPC biology, although five (e.g. P2ry14, Smarca2, Sox4, and Gpr56) have recently been shown to play a role in leukemia or HSC (Buscarlet, et al., 2014; Cho, et al., 2014; Solaimani Kartalaei, et al., 2015; Zhang et al., 2013). These studies confirm that our screen has identified genes relevant to HSC function.

In contrast, prior screens of mouse and human HSPC involved extensive culture time periods, (12-17 days) prior to transplant or followed the preservation of a stem cell phenotype or colony formation during culture (5 days to 10 weeks) (Ali et al., 2009; Boitano, et al, 2010; Deneault, et al., 2009; Hope et al., 2010), thus biasing their readout for genes involved in self-renewal or stem cell maintenance, two processes contributing to HSC function and culture but not necessarily contributing to stable engraftment. By minimizing LSK cell culture prior to transplant, we reasoned that our screen would identify genes specifically regulating self-renewal, which can also enhance HSPC repopulation and then allow us to identify genes relating to distinct cellular processes contributing to the long-term reconstitution (i.e. stable engraftment) of an ablated hematopoietic system that may not have been as readily discernable in these prior studies. Prior studies also focused on specific molecular processes (e.g. nuclear factors, polarity and asymmetric division, histone methylation).

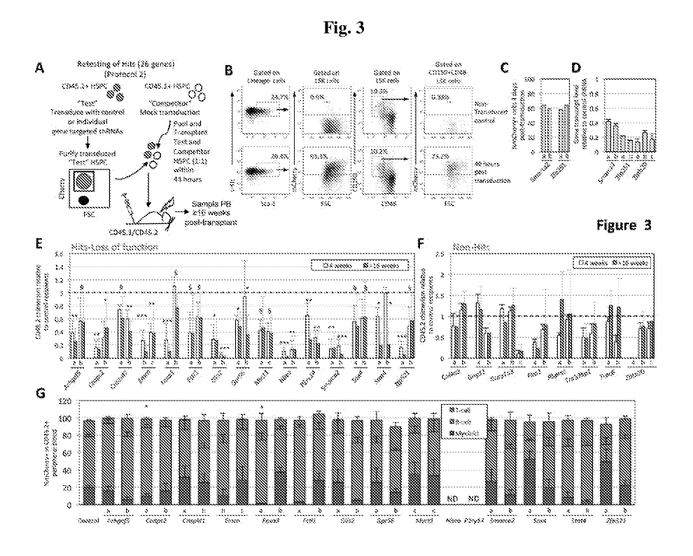

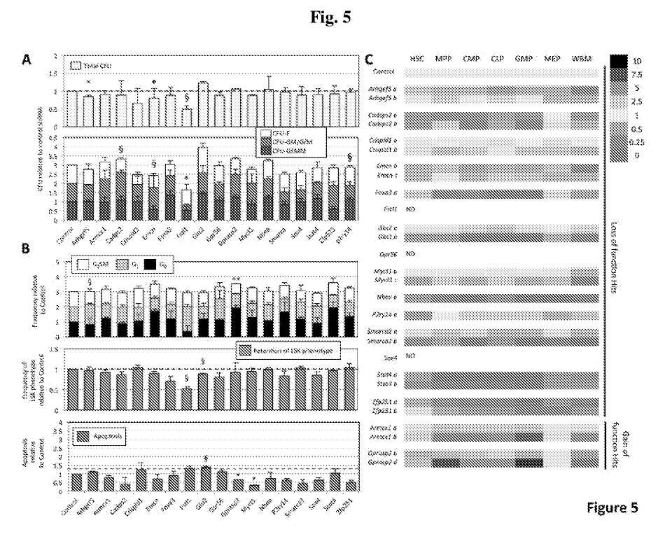

Our screen was unbiased in that our criteria were 1) confirmation by qRT-PCR of high expression in LSK cells and 2) identification of effective shRNAs. This approach discovered Hits involved in distinct cellular and molecular processes, some understudied in HSPC. For example, multiple likely regulators of vesicular trafficking and cell surface receptor turnover were identified as regulators of LSK cell repopulating activity (Nbea, Cadps2, Armcx1, and Gprasp2) (Abu-Helo and Simonin, 2010; Cisternas et al., 2003; Moser et al., 2010; Niesmann et al., 2011) (FIG. 3A-G). These genes may regulate stable HSPC/niche interactions or the transduction of survival signals during hematopoietic stress. Indeed, changes in CFU activity, cell cycle, and apoptosis in LSK cells maintained ex vivo after knockdown of Nbea, Cadps2, or Gprasp2 but not Armcx1 (FIG. 5A-C), suggest regulation of intrinsic pathways controlling differentiation, survival, and/or proliferation by these genes, i.e. Nbea, Cadps2, or Gprasp2.

Arhgef5, a Rho guanine nucleotide exchange factor, has been implicated in podosome formation (Kuroiwa et al., 2011). Podosomes, ring-like cell protrusions which mediates cell-extracellular matrix interactions, contribute to cell adhesion and migration. Knockdown of Arhgef5 in LSK cells maintained ex vivo resulted in an accumulation of cells in G1 as well as a loss of total CFU formation (FIG. 5A and FIG. 5B). Gpr56, previously implicated in neuronal migration, was recently shown to participate in HSC development and adhesion. Gpr56l-HSC also displays a repopulating defect, as seen in our study after gene knockdown (Rao et al., 2015; Saito et al., 2013; Singer et al., 2013; Solaimani Kartalaei et al., 2015). We also identified secreted molecules (Fstl1 and Crispld1). Fstl1 is a TGFp and BMP antagonist while Crispld1 is a likely protease targeting the extracellular matrix (Geng et al., 2011; Gibbs et al., 2008). Knockdown of Fstl1 in LSK cells led to fewer CFU and loss of the LSK cell surface phenotype, suggesting an intrinsic loss of HSPC potential (FIG. 5A-C). These genes suggest that to facilitate stable engraftment and in vivo repopulation HSPCs may autonomously condition their niche and culture by countering inhibitory signaling pathways (e.g. TGF(3) and remodeling the extracellular matrix (Arhgef5 and Crispld1).

Although Myct1 has never been implicated in HSPC function, it is a c-Myc target, which modulates HSC/niche interactions via N-cadherin (Wilson et al., 2004). There are currently no primary articles on Zfp251, a Krueppel-type C2H2 zinc finger gene family member and possible transcriptional repressor, given it contains a KRAB domain (Urrutia, 2003). Knockdown of this gene in LSK cells perturbed CFU formation, appeared to enhance survival ex vivo, and led to a dramatic loss of chimerism downstream of the HSC compartment in the bone marrow of transplanted mice, suggesting that Zfp251 regulates the differentiation and survival of HSPC (FIG. 5A-C). Although several of our Hits are known to be expressed by HSPC or have been implicated in leukemogenesis, here we reveal them as regulators of HSPC repopulation (Emcn, Glis2, Sox4, and Smarca2) (Buscarlet et al., 2014; Gruber et al, 2012; Ma et al, 2014; Masetti et al, 2013; Matsubara et al, 2005; Zhang et al, 2013).

Another Hit, the purinergic receptor, P2ry14, was very recently shown to be a regulator of stress hematopoiesis and HSC repopulation, further validating our screen (Cho et al, 2014). Globally, the results of our screen support a model in which active crosstalk between the bone marrow niche and HSPC contributes to stable hematopoietic repopulation following transplant. Thus, in one embodiment, exogenous (ex vivo) treatment of HSC with Fstl1 (Follistatin-Like 1) and Crispld1 (Cysteine-Rich Secretory Protein LCCL Domain Containing 1) protein or expression vector for increasing intracellular expression, in combination with treatment with an shRNA for a GASP gene, may also find use for promoting stable engraftment. It was recently reported that Fstl1, which is also expressed in cardiac epicardium, promotes the regeneration of cardiomyocytes both in vivo and ex vivo (Wei et al, 2015).

Mechanistically, the discovery of multiple genes regulating vesicular trafficking, cell surface receptor turnover, and secretion of extracellular matrix components indicates active crosstalk between HSC and the biological niche opened through irradiation. Thus indicating that transplanted HSCs may actively condition the niche to promote engraftment. We validated that Foxa3 contributes directly to HSC repopulating activity as Foxa3^{-/-} HSC fail to repopulate ablated hosts efficiently, implicating Foxa genes as positive regulators of HSPC. We further demonstrated that Foxa3 likely regulates the HSC response to hematologic stress. The results on these HSC genes discovered to affect HSC engraftment offers a window into the novel processes that regulate stable HSPC engraftment into an ablated host.

2. Gprasp2 And Armcx1 Genes.

During the development of the present inventions, the observed decreased expression in Gprasp2 or Armcx1 after targeting these genes with shRNAs in lentiviral vectors in mouse LSK cells, was interpreted as increasing the repopulating potential of CD45.2+ mouse cells (i.e. LSK cells) after observing increased chimerism in lethally irradiated mice transplanted with these cells. Thus, hematopoietic stem cell (HSC) transplantation for treating hematologic disease by improving HSC engraftment transplant morbidity might be ameliorated, i.e. Ganuza, et al., McKinney-Freeman. P1045: “Functional Screen Identifies Novel Regulators Of Hematopoietic Stem Cell In Vivo Repopulation.” Poster: 43^{rd} Annual Meeting of the International Society for Experimental Hematology (Canada, Montreal, QC) Aug. 21-24, 2014; and Fernandez, et al., McKinney-Freeman. “Functional screen identifies novel regulators of murine hematopoietic stem cell engraftment.” Abstract and Poster: 56^{th} Annual Meeting of the American Society of Hematology (San Francisco, Calif.). Dec. 6-9, 2014. Methods for overcoming the paucity of hematopoietic stem cells (HSC), which limits their application to treat disease, were proposed for enhancing HSC engraftment efficiency. In fact, a loss of function of Armcx1 and Gprasp2 enhanced repopulation of mouse LSK cells, in a presentation abstract by Shannon McKinney-Freeman, “Functional screen identifies novel regulators of murine hematopoietic stem cell engraftment.” Abstract ISSCR 2015 Annual Meeting (Stockholm, Sweden) Jun. 24-27, 2015. As published in the program, it was suggested that Gprasp2 or Armcx1 genes might regulate stable HSC engraftment into an ablated host. Schematics of methods and post-transplantation data (at 16 weeks) obtained after knocking down Armcx1 and Gprasp2 genes in LSK cells then transplanting into irradiated mice were shown in the corresponding presentation. shRNAs for Armcx1 and Gprasp2 showed variable results, with some shRNAs showing more consistent results than the other(s). Data was obtained from experiments in mice using knockdown cells co-transplanted with competitor CD45.1 LSK cells that do not contain a knockdown construct. Shannon McKinney-Freeman. “Functional screen identifies novel regulators of murine hematopoietic stem cell engraftment.” Oral Presentation (PowerPoint) ISSCR 2015 Annual Meeting (Stockholm, Sweden). Jun. 24-27, 2015.

Gprasp2 and Armcx1 were proposed as putative negative regulators of hematopoietic stem cell transplantation (HSCT) for mice and humans. Mouse recipients of either Gprasp2 or Armcx1 shRNA-treated CD45.2+(LSK) cells along with control LSK cells, displayed 3 fold enhanced CD45.2 chimerism in peripheral blood (PB) at 16 weeks post-transplant, relative to controls. Although loss of each gene did not favor a particular PB lineage, CD45.2+ chimerism was enhanced in bone marrow (BM) HSC and progenitor (HSPC) compartments in these recipients, correlating with their enhanced PB chimerism. Ferdous, et al., Shannon McKinney-Freeman. “The G Protein-Coupled Receptor Associated Sorting Proteins, Gprasp2 and Armcx1 Are Putative Negative Regulators of HSC Engraftment and Repopulation.” Blood: 126 (23): Dec. 3, 2015. Ferdous, et al., Shannon McKinney-Freeman. 2386 “The G Protein-Coupled Receptor Associated Sorting Proteins, Gprasp2 and Armcx1 Are Putative Negative Regulators of HSC Engraftment and Repopulation.” 57^{th} Annual Meeting of the American Society of Hematology (Orlando, Fla.). Dec. 5-8, 2015. Session: 504. Hematopoiesis: Cytokines, Signal Transduction, Apoptosis and Cell Cycle Regulation: Poster II. Published abstract: Dec. 6, 2015. Although Gprasp1 was not tested in our screen, qRT-PCR analysis reveals that it is also highly expressed by murine HSC relative to downstream progeny, suggesting that it too may play a role in HSC function. The associated poster describes methods for increasing the efficiency of HSC engraftment. In particular, mouse CD45.2+ lineage-Sca-1+c-Kit+(LSK) cells were treated with a shRNA for either Gprasp2 or Armcx1 linked to a m-Cherry fluorescent marker for reducing Gprasp2 and Armcx1 gene expression prior to transplantation. This publication mentioned that both murine Gprasp2 and Armcx1 and their human homologs, GPRASP2 and ARMCX1, are highly expressed in murine LSKCD150+CD48− and human Lin-CD34+CD38− HSPC, respectively. Knockdown of Gprasp2 and Armcx1 lead to significantly increased CD45.2+ chimerism in hematopoietic compartments of recipient BM. A related GASP family member is Gprasp1, is highly expressed in murine LSKCD150+CD48− cells. FIG. 95A-G. Ferdous, et al., Shannon McKinney-Freeman. 2386 “The G Protein-Coupled Receptor Associated Sorting Proteins, Gprasp2 and Armcx1 Are Putative Negative Regulators of HSC Engraftment and Repopulation.” 57^{th} Annual Meeting of the American Society of Hematology (Orlando, Fla.). Dec. 5-8, 2015. Session: 504. Hematopoiesis: Cytokines, Signal Transduction, Apoptosis and Cell Cycle Regulation: Poster II. Sunday, Dec. 6, 2015.

Gprasp2 and Armcx1 genes were mentioned in a publication that also discussed HSC transplantation and a drug is contemplated as a siRNA, although there was no mention of specifically using shRNA for knocking out Gprasp2 or Armcx1, nor mention of Gprasp1, in Onder, et al., US Patent Application Publication No. 20150223436 A1. “Hematopoietic stem cell specific reporter mouse and uses thereof.” Publication date Aug. 13, 2015. This patent application describes a method to screen for agents that affect the growth, proliferation, potency, expansion, or maintenance of human hematopoietic stem cells, including umbilical cord blood cells, and for promoting growth of stem cells in vitro or in vivo, including contemplated for use in animal transplantation. Genes with highly restricted expression, i.e. predominantly expressed, in hematopoietic stem cells in comparison to their downstream progenitor and effector progeny included Gprasp2 and Armcx1 as listed in Table 2. Three of the genes listed in Table 2 were chosen for knock-out studies in mouse cells, i.e. Clecla, Fgd5, and Sultlal, for transplantation into lethally irradiated adult congenic recipients. Screening methods and assays were also described and shown for identifying small molecules, including agents such as RNAi, shRNAi, and siRNA, that can maintain or expand HSCs using bone marrow cells in mice and humans.

ShRNAs for reducing expression of Gprasp2 and Armcx1 were used for treating mouse stem cells prior to transplantation where loss of expression for either Gprasp2 or Armcx1 in shRNA transduced mouse stem cells (CD45.2+ and LSK cells, a mixture of hematopoietic stem cells (HSC) and progenitor cells (HSPCs), enhanced HSC repopulation in lethally irradiated mice. Holmfeldt, et al., Shannon McKinney-Freeman. “Functional screen identifies regulators of murine hematopoietic stem cell repopulation.” J Exp Med., published February 2016. In other words, when HSPCs are treated with shRNA to lower expression of Gprasp2 or Armcx1, the treated HSPCs enhanced HSPC repopulation in mice. In particular, Table 2. “Summary of Genes Tested in Functional Screen” shows a list of genes tested along with shRNA sequences for reducing expression of the named mouse gene.

3. Comparative Gprasp Expression in shRNA Treated Murine Hematopoietic Stem Cells: Gprasp1 shRNA May Induce an Increase in Gprasp2 Expression.

Isolated and cultured murine hematopoietic stem cells and murine hematopoietic stem progenitor cells (HSPC) were treated with control shRNA, or Gprasp1-shRNAs A or B or Gprasp2-shRNAs A or B. Expression of Gprasp1-RNA (open bars-left) or Gprasp2 RNA (filled-in bars-right) was measured relative to expression when treated with control shRNA. Gprasp1-RNA was reduced with both A and B shRNA sequences while Gprasp2-RNA expression did not appear to be affected. Gprasp2-RNA was reduced with both A and B shRNA sequences. ShRNA knock-down was robust but not 100%. The percentage in reduction in expression of a targeted GASP gene expression appears to depend on the particular shRNA sequence used.

Although there did not appear to be an effect of Gprasp2-shRNA treatment on Gprasp1 expression, in at least one experiment the Gprasp1-shRNA B treatment was associated with a higher expression of Gprasp2. Based upon the results from HSC −/− experiments which indicated that compensatory mechanisms may be triggered by the genetic loss of a Gprasp gene, this result indicates that in some embodiments, more than one Gprasp gene targeted shRNA should be used for treating stem cells. Thus, in some embodiments, two or more Gprasp genes are targeted for reduction prior to transplantation, for enhancing transplantation potential.

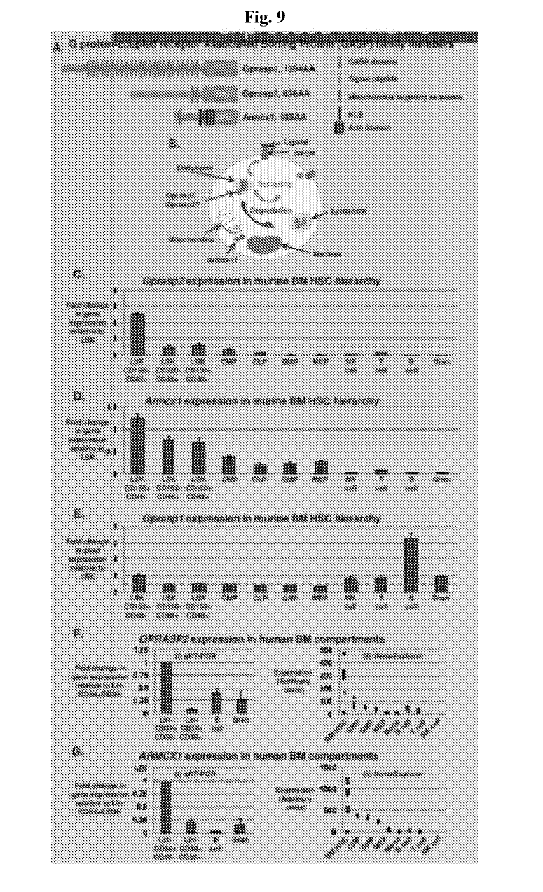

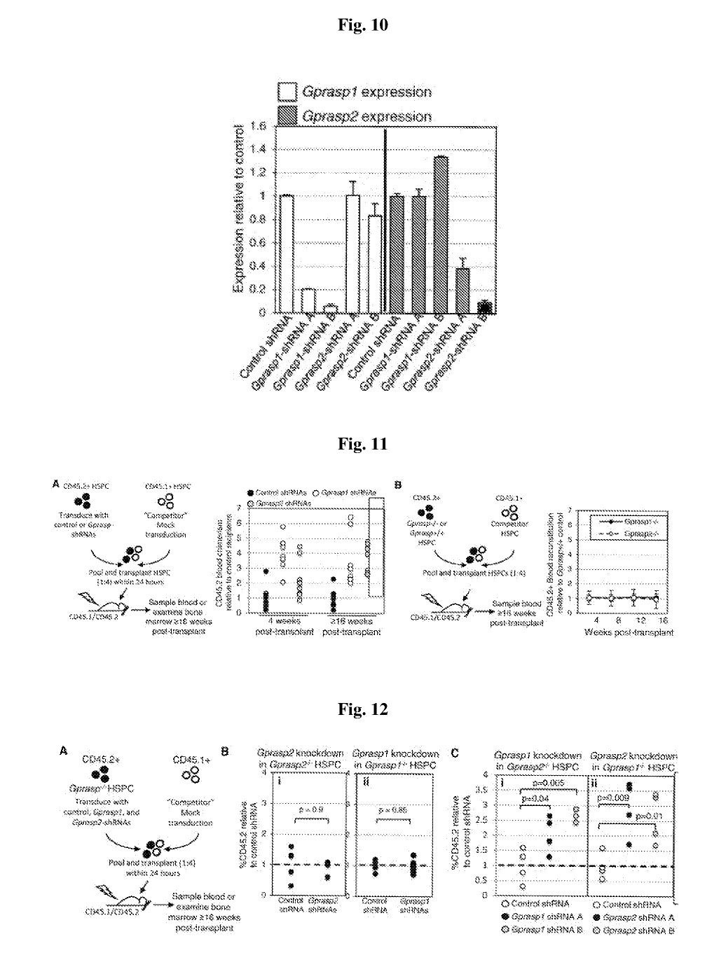

FIG. 10. Gprasp1 And Gprasp2 shRNAs Demonstrate A Range Of Specificities Shown In A Comparative Chart. ShRNAs targeting murine Gprasp1 or Gprasp2 efficiently and specifically knock-down Gprasp1 and Gprasp2 gene expression, respectively, in murine hematopoietic stem cells and murine hematopoietic stem progenitor cells (HSPC).

4. Repopulating Activity in Stem Cells does not Appear to be Altered by Genetically Knocking-Out Single Gprasp Genes as Shown in Gprasp1−/− and Gprasp2−/− Murine Hematopoetic Stem Cells.

Murine Stem Cells were genetically engineered to knock-out both alleles of Gprasp1 or both alleles of Gprasp2, providing Gprasp1−/− murine HSC populations or Gprasp2−/− HSC populations, respectively. However, unlike HSCs where Gprasp1 or Gprasp2 were silenced using respective Gprasp gene shRNA, neither of these −/− HSC populations demonstrated enhanced repopulating activity. Thus, in these experiments, shRNA treatment has no effect on the repopulating activity of the knock-out HSCs, indicating that the enhanced repopulating activity of HSC seen when wild-type HSC are treated with shRNAs is due to the specific knock-down of Gprasp1 or Gprasp2.

FIGS. 11A-B. shRNA Induced Reduction Of Gprasp1 Or Gprasp2 Enhances The Repopulation Activity Of HSPC While Genetic Loss Of Gprasp1 Or Gprasp2 In HSC−/− Populations Does Not Enhance The Repopulation Activity Of HSPC. FIGS. 11A-B show a schematic diagram for an exemplary experimental method (left) and results in a chart (right). FIG. 11A CD45.2+ HSPC were transduced with control or Gprasp-shRNA, as shown, then transplanted with CD45.1 “Competitor” HSPCs into recipient mice. Recipient mouse blood was then analyzed for CD45.2+ cells. ShRNA knock-down of Gprasp1 or Gprasp2 enhances the blood repopulating activity of HSPC after 4 weeks and continues up to and after 16 weeks. Each dot in the chart on the right represents an independently transplanted mouse. FIG. 11B CD45.2+ Gprasp+/+ HSPCs or Gprasp−/− HSPCs were transplanted with CD45.1 HSPCs into irradiated CD45.1+/CD45.2+ recipient mice. Recipient mouse blood was then analyzed for CD45.2+ cells up to and over 16 weeks post-transplantation. Each dot in the chart on the right represents an independently transplanted mouse. Genetic loss of Gprasp1 or Gprasp2 gene translation into GPRASP1 or GPRASP2 protein, does not result in enhanced blood repopulating activity of HSPC.

Further, when each of these populations was treated with a shRNA, via a silencing vector construct, there was no effect on the repopulating activity for either of these treated populations. In other words, Gprasp1−/− HSC populations treated with Gprasp1-shRNA and Gprasp2−/− HSC populations treated with shRNA for Gprasp2-shRNA, failed to show the repopulating activity than when HSCs were treated with Gprasp-shRNA alone.

FIGS. 12A-C. Gprasp1-shRNA Or Gprasp2-shRNA Do Not Enhance The Repopulating Activity Of Treated Gprasp1−/− HSPC Or Gprasp2−/− HSPC, Respectively: While shRNA Silencing Of A Second Gprasp Gene In Gprasp1−/− HSPC Or Gprasp2−/− HSPC Induces A Partial Gain Of Enhanced Repopulating Activity. FIG. 12A shows a schematic diagram for an exemplary experimental method, and FIGS. 12B-C show comparative charts of experimental results. FIGS. 12A-B In part, for testing off-target effects of Gprasp1-shRNA or Gprasp2-shRNA: CD45.2+ Gprasp1−/− HSPCs (ii) or Gprasp2−/− HSPCs (i) were transduced with either control shRNA or Gprasp1-shRNA (ii) or Gprasp2-shRNA (i) then transplanted along with CD45.1+ HSPCs into irradiated CD45.1+/CD45.2+ recipient mice (n=4)/group). Gprasp1−/− HSPCs and Gprasp2−/− HSPCs did not display enhanced repopulating activity when treated with Gprasp1-shRNA (ii) or Gprasp2-shRNA (i), respectively. Thus, Gprasp-shRNAs do not have off-target effects that causes enhanced repopulation.

5. Repopulating Activity Appears to be Altered by Double Gprasp-RNA Gene Silencing in Gprasp1−/− and Gprasp2−/− Murine Stem Cells.

Murine Gprasp1−/− HSC populations or Gprasp2−/− HSC populations were treated with shRNA for silencing a Gprasp gene that was not knocked-out. In other words, the Gprasp1−/− HSC populations were treated with Gprasp2-shRNA while the Gprasp2−/− HSC populations were treated with Gprasp1-shRNA. Surprisingly, unlike −/− HSC populations treated for silencing of the same Gprasp1 or Gprasp2 that was genetically knocked out, as in FIG. 12B, each of the −/− HSC populations treated with a silencing Gprasp-shRNA for one of the GASP genes that was not genetically knocked down, demonstrated enhanced repopulating activity. Further, the enhanced repopulating activity of wild-type HSCs treated with one Gprasp gene shRNA was greater than when a Gprasp−/− HSC population was treated with the Gprasp-shRNA that targeted one of the GASP genes that was not genetically knocked down. Therefore, the effect was not additive indicating the possibility of a compensatory effect of another expressed gene as part of the genetically altered HSC's attempt to overcome the loss of one or more Gprasp genes.

FIG. 12C CD45.2+ Gprasp1−/− HSPCs (ii) or Gprasp2−/− HSPCs (i) were transduced with either control shRNA or Gprasp1-shRNA (ii) or Gprasp2-shRNA (i) then transplanted along with CD45.1+ HSPCs into irradiated CD45.1+/CD45.2+ recipient mice. Recipient mouse blood was then analyzed for CD45.2+ cells up to and over 16 weeks post-transplantation. Loss of Gprasp1 expression in Gprasp2−/− HSPCs (i) and loss of Gprasp2 expression in Gprasp1−/− HSPCs (ii) enhanced blood-repopulating activity of transplanted HSPCs. Each dot in the charts represents an independently transplanted mouse.

It is contemplated that some subjects may have natural genetic alterations for reducing Gprasp endogenous expression. Thus, another contemplated use of Gprasp-shRNA is treating HSCs for knock down of compensatory Gprasp gene expression for enhancing repopulation activity of transplanted HSCs.

6. Identifying Compensatory Genes in Gprasp1−/− and Gprasp2−/− Murine Hematopoietic Stem Cells for Enhanced White Blood Cell Repopulating Activity.

Upregulated GASP genes were identified in Gprasp−/− murine Hematopoietic Stem Cells. Of these, GASP3, named Bhlhb9 (Basic Helix-Loop-Helix Domain Containing, Class B, 9 gene) in humans, was chosen for further study. GASP3 refers to a GASP family member that is structurally very similar to Gprasp1 and Gprasp2 and is upregulated in both Gprasp1−/− and Gprasp2−/− murine Hematopoietic Stem Cells. The inventor contemplated that upregulation of Bhlhb9 may compensate for loss of Gprasp1 and Gprasp2 in knock-out HSC. Thus in another embodiment, Bhlhb9-shRNA may be used alone, or in combination with one or more of Gprasp1-shRNA and Gprasp2-shRNA for transducing human HSCS in transplantation methods for enhancing white blood cell repopulation in patients.

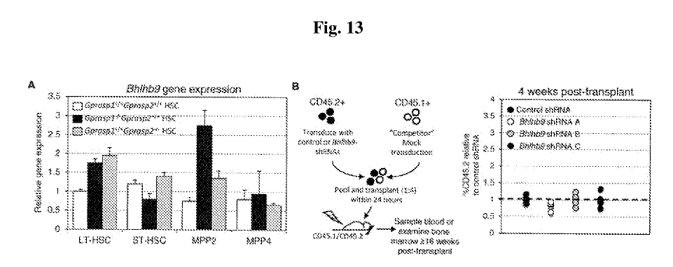

Gprasp3 (labeled Bhlhb9 when referring to the human ortholog of Gprasp3) expression was measured in wild-type (Gprasp1+/+ Gprasp2+/+) murine HSPCs in populations that were cultured long-term (LT-HSC), short-term (ST-HSC), and MPP2 and MPP4 populations, see, FIG. 13A. Silencing vectors for use in reducing expression of murine GASP3 (labeled Bhlhb9) in mouse stem cells were constructed and used for transducing CD45.2+ murine cells that were used for transplantation into mice, see, FIG. 13B. There was little repopulating activity of GASP3-shRNA treated CD45.2+ detected 4 weeks post-translation, see, FIG. 13C.

FIGS. 13A-B. Bhlhb9 Is Upregulated In Murine Gprasp1−/− HSPCs And Gprasp2−/− HSPCs. FIG. 13A shows that Bhlhb9 is upregulated in Gprasp1−/− LT-HSCs (long-term HSC) and Gprasp2−/− LT-HSCs. Thus Bhlhb9 may functionally compensate for loss of Gprasp1 or Gprasp2 in HSC. FIG. 13B shows a schematic diagram for an exemplary experimental method (right) and a chart showing results (left) demonstrating that knock-down of Bhlhb9 in murine HSPC does not enhance their repopulating activity.

7. Human Bhlhb9 Genes's Structural Components are Compared to Gprasp1 and Gprasp2 Genes.

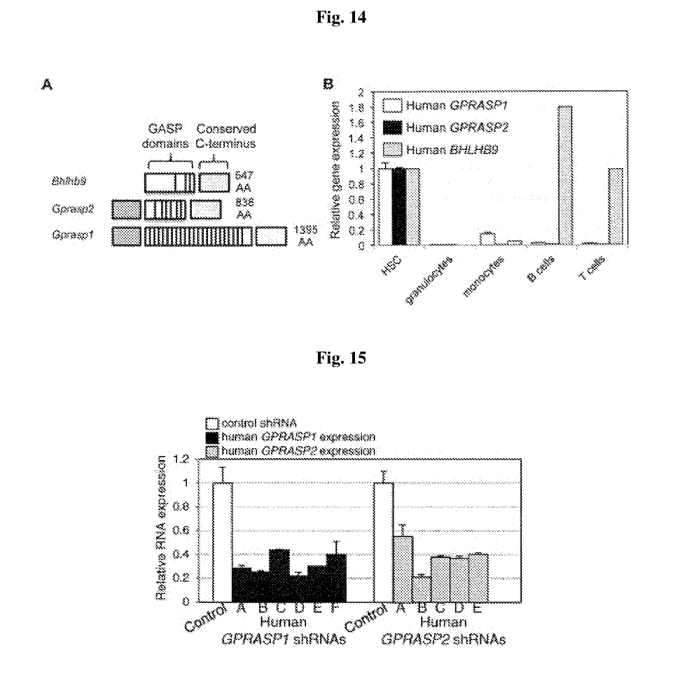

Structural similarities showing GASP domains and conserved C-terminus regions are found in Bhlhb9, Gprasp1 and Gprasp2, see FIG. 14A. Bhlhb9 information is shown at: www.ncbi.nlm.nih.gov/gene/80823, accessed 6-8-2017. Gprasp1, Gprasp2 and Bhlhb9 genes appear to be more similar in the 3′ region than in the 5′ regions. In contrast, Gprasp1 and Gprasp2 genes appear to have similar regions at the 5′ end that are not present in Bhlhb9.

Expression of human Bhlhb9, Gprasp1 and Gprasp2 were measured in white blood cells populations, including hematopoietic stem cells, granulocytes, monocytes, B cells and T cells. Measurements were made using qualitative measurements during the amplification of DNA using fluorescent dyes. Gene expression is detected through creation of complementary DNA (cDNA) transcripts from RNA, see exemplary primers in Table 10. Then qPCR is used to quantitatively measure the amplification of DNA using fluorescent dyes.

FIGS. 14A-B. GASP Family Members Gprasp1, Gprasp2 And Bhlhb9 Are Expressed By Human Hematopoietic Stem Cells (HSC) And Progenitor Cells (HSPC). FIG. 14A GPRASP1, GPRASP2 and BHLHB9 are structurally similar members of the GASP (G-protein coupled receptor Associated Sorting Proteins) protein family that FIG. 14B are expressed by human hematopoietic stem cells (HSC).

Bhlhb9, Gprasp1 and Gprasp2 were expressed in hematopoietic stem cells while Bhlhb9 was expressed, not Gprasp1 or Gprasp2, in B cells and T cells. A small amount of Gprasp1 expression, but not Gprasp2 or Bhlhb9, was measured in monocytes, while little expression of the three genes was measured in granulocytes.

TABLE 10
qRT-PCR primer sequences for human GRASP genes.

| Gene | | |
|---|---|---|
| symbol | Forward Primer | Reverse Primer |
| GPRASP1 | GCTACTTCGGTTGGACTCTG | CCTCTCACTCACTCTAGGC |
| GPRASP2 | GGGCTTGACACCACTTGAAC | GAGAATGAAATGAGGCTTTG |
| | | AG |
| BHLHB9 | GGCCAGCAATCTGGATTAAA | AATGCTGCTAACGCCTTCAT |
| (Gasp3) | | |
| ARMCX1 | TGGTGCCTGCTACTGTGTAT | TCTCAGGTCCCACATTCACC |
| (Gasp7) | | |

II. Exemplary GASP shRNA Silencing Constructs for Use with Treatment Methods of the Present Inventions.

In one embodiment, a human Gprasp shRNA is ligated into a retroviral expression vector. In one preferred embodiment, human Gprasp shRNA is ligated into a lentiviral expression vector for producing lentiviral particles for use in methods of transducing human HSCs. In other embodiments, mouse Gprasp shRNA is ligated into a retroviral expression vector. In one embodiment, mouse Gprasp shRNA is ligated into a lentiviral expression vector for producing lentiviral particles for use in methods of transducing mouse HSCs. Examples of mouse Gprasp shRNA sequences are provided herein. Examples of methods of making and using lentiviral vectors as constructs for transducing HSCs are provided herein.

Lentiviral expression vector constructs comprising predesigned shRNA inhibitory siRNA directed against mouse Gprasp1 and human Gprasp1; and against mouse Gprasp2 and human Gprasp2; and against mouse Armcx1 and human Armcx1, may be obtained commercially from several companies, including but not limited to Qiagen (27220 Turnberry Lane, Suite 200, Valencia, Calif. 91355: www.qiagen.com/us/), OriGene (9620 Medical Center Dr., Suite 200, Rockville, Md. 20850: www.origene.com) and Santa Cruz Biotechnology (10410 Finnell Street Dallas, Tex. 75220: www.scbt.com/). For at least one company, OriGene Technologies, Inc., (www.origene.com) predesigned shRNA inhibitory siRNA lentiviral particles for silencing Gprasp1, accessed 4-11-2016; Gprasp2 accessed 4-05-2016; and Armcx1 accessed 3-11-2016, have a guaranteed knockdown of >70%.

Another example of a shGASP-1 lentiviral vector for reducing expression of a human Gprasp1 shRNA in human cells that may find use in the present inventions includes a description in Kargl, et al., “The trafficking of GPR55 is regulated by the G protein-coupled receptor-associated sorting protein 1.” BMC Pharmacol. 10 (Suppl. 1): A1. Published online 2010. This reference describes knockdown of endogenous GASP-1 levels in Human Embryonic Kidney cells induced by infection with Lenti-shGASP-1 (shGASP-1).

Other examples of G Protein-Coupled Receptor Associated Sorting Protein shRNA are provided in gene cards for each protein, i.e. Gprasp1 (G Protein-Coupled Receptor Associated Sorting Protein 1) Gene Card. Copyright © 1996-2016, accessed 3-07-2016; Gprasp2 (G Protein-Coupled Receptor Associated Sorting Protein 2) Gene Card. Copyright © 1996-2016, accessed 3-07-2016; and ARMCX1 (Armadillo Repeat Containing, X-Linked 1) Gene Card. Copyright © 1996-2016, accessed 3-11-2016. These websites, respectively, show Gprasp1 (GASP-1) in addition to showing a thymus hematopoietic system and descriptions of shRNA; Gprasp2 (GASP-2), expression in hematopoietic stem cells-Hematopoietic Bone Marrow, and descriptions of shRNA; and ARMCX1 (GASP7), expression in Hematopoietic Stem Cells-Liver Bud, and descriptions of shRNA.

A. Human Gprasp1 and Gprasp2 shRNA Reduces Gprasp1 and Gprasp2 Expression in Human Hematopoetic Stem Cells, Respectively.

Silencing vectors for knocking down human Gprasp1 and Gprasp2 gene expression were constructed, including but were not limited to a promoter, a shRNA sequence and a lentiviral expression vector. Exemplary shRNA sequences are shown in Table 11. Exemplary FIG. 11 demonstrates knock down levels for each of the genes in human cell lines.

TABLE 11
Exemplary human shRNA sequences contemplated for use in HSC
transplantation.

| Gene | | | |
|---|---|---|---|
| Symbol | Gene Name | shRNA | shRNA sequences |
| Gprasp1 | G protein- | A | TGCTGTTGACAGTGAGCGCTTGGTGCTGAAAGAT |
| (GASP1) | coupled receptor | | TGTCTATAGTGAAGCCACAGATGTATAGACAATC |
| | associated | | TTTCAGCACCAAATGCCTACTGCCTCGGA |
| | sorting protein 1 | | |
| Gprasp1 | G protein- | B | TGCTGTTGACAGTGAGCGACAGGTCCAGGTTTAG |
| (GASP1) | coupled receptor | | GTCTAATAGTGAAGCCACAGATGTATTAGACCTA |
| | associated | | AACCTGGACCTGCTGCCTACTGCCTCGGA |
| | sorting protein 1 | | |
| Gprasp2 | G protein- | A | TGCTGTTGACAGTGAGCGCCAGAGACAAAGAAG |
| | coupled receptor | | ATCCTAATAGTGAAGCCACAGATGTATTAGGATC |
| | associated | | TTCTTTGTCTCTGTTGCCTACTGCCTCGGA |
| | sorting protein 2 | | |
| Gprasp2 | G protein- | B | TGCTGTTGACAGTGAGCGACAGAAAGATGTTGA |
| | coupled receptor | | CAGTGATTAGTGAAGCCACAGATGTAATCACTGT |
| | associated | | CAACATCTTTCTGGTGCCTACTGCCTCGGA |
| | sorting protein 2 | | |

Additional exemplary methods for enhancing stem cell transplantation includes reducing expression levels of Bhlhb9, alone or in combination with reducing expression of one or more additional GRASP genes.

An example for a Bhlhb9-shRNA may be obtained from Virigene Biosciences, See Table 12. As another example for reducing Bhlhb9 expression, BHLHB4 CRISPR/Cas9 KO Plasmid, sc-414328, Santa Cruz, Biotechnology, Inc. USA, may also be used for transducing human stem cells for use in transplantation.

TABLE 12
Exemplary Bhlhb9-shRNA Sequences for use in lenti-
viral silencing vectors.

| Description | shRNA Sequences |
|---|---|
| shRNA 1 for BHLHB9 | AAGCTAAAGCTGGAGCAGAGAGG |
| (NM_030639) | |
| shRNA 2 for BHLHB9 | GGGAAGAGGCCACTATCAATTCC |
| (NM_030639) | |
| shRNA 3 for BHLHB9 | CCCAAGGACTGGTCTGAGGTAAC |
| (NM_030639) | |
| shRNA 4 for BHLHB9 | TTAAGCCATTTGCTTGTCCTTGC |
| (NM_030639) | |

In some embodiments, human HSCs are transduced with at least one human GASP gene shRNA. In another embodiment, human HSCs are transduced with at least two human GASP gene shRNAs, including but not limited to Gprasp1, Gprasp2, Gprasp3 and Armcx1 (Gprasp7). Thus, in one contemplated embodiment, at least one GASP gene, such as Gprasp1 and Gprasp2, etc., are silenced (i.e. transiently knocked down) in human HSCs. In another contemplated embodiment, two or more GASP genes, such as Gprasp1 and Gprasp2; Gprasp1 and Gprasp3; Gprasp1, Gprasp2 and Gprasp3, etc., are silenced in human HSCs.

In some embodiments, mouse HSCs are transduced with at least one mouse GASP gene shRNA. In another embodiment, mouse HSCs are transduced with at least two mouse GASP gene shRNAs, including but not limited to Gprasp1, Gprasp2, Gprasp3 and Armcx1 (Gprasp7).

In some embodiments, equine (e.g. horse) HSCs are transduced with at least one GASP gene shRNA. In another embodiment, equine HSCs are transduced with at least two GASP gene shRNAs, including but not limited to Gprasp1, Gprasp2, Gprasp3 and Armcx1 (Gprasp7).

In some embodiments, canine (e.g. dog) HSCs are transduced with at least one GASP gene shRNA. In another embodiment, canine HSCs are transduced with at least two GASP gene shRNAs, including but not limited to Gprasp1, Gprasp2, Gprasp3 and Armcx1 (Gprasp7).

In some embodiments, feline (e.g. cat) HSCs are transduced with at least one GASP gene shRNA. In another embodiment, feline HSCs are transduced with at least two GASP gene shRNAs, including but not limited to Gprasp1, Gprasp2, Gprasp3 and Armcx1 (Gprasp7).

Reducing GASP gene expression is not limited to using shRNA, and may also be accomplished using CRISPR Knockout technology. Exemplary technology is commercially available, for example human GASP-1 CRISPR Knockout, sc-406921, human GASP-2 CRISPR Knockout, sc-418296, Santa Cruz, Biotechnology, Inc. USA.

Contemplated uses of Gprasp-shRNA treated HSCs include but are not limited to autologous hematopoietic stem cell transplantation (HSCT) and allogeneic HSCT, for treating patients with hematological cancer; acquired marrow failure; genetic hematological diseases; autoimmune diseases, etc.

III. Treatment Methods.

In one embodiment, a human Gprasp shRNA in a lentiviral expression vector for producing lentiviral particles

A. Experiments Related to the Development of the Present Inventions.

1. Exemplary Materials and Methods.

Mice.

C57BL/6J and C57BL/6.SJL-PtprcaPep3b/BoyJ mice were acquired from The Jackson Laboratory (Bar Harbor, Me.) and housed in a pathogen-free facility. All animal experiments were carried out according to procedures approved by the St. Jude Children's Research Hospital Institutional Animal Care and Use Committee. C57BL/6 Foxa3^{-/-} mice were a gift from the laboratory of Dr. Klaus Kaestner (University of Pennsylvania, Philadelphia, Pa.).

Genotyping.

Polymerase chain reactions (PCR) were performed using Go Taq DNA Polymerase (Promega, Madison Wis.) and performed as indicated by the manufacturer. PCR conditions: (95° C., 2′); ([95° C., 30″; 60° C., 30″; 72° C., 30″]×35); (72° C., 10′). Primers: Foxa3 F2 (5′ ACATGACCTTGAACCCACTC 3′), Foxa3 RI (5′ TAGTACGGGAAGAGGTCCAT 3′), Foxa3 LacZ3 (5′ AATGTGAGCGAGTAACAACC 3′). Wild type PCR: Foxa3 F2+ Foxa3 RI; Wild type band: 349 bp. KO PCR: Foxa3 F2+ Foxa2 LacZ3; Knock-out band: 648 bp.

qRT-PCR (q-RT-PCR).

Total RNA isolated from 70,000 LineageSca-1⁺c-Kit⁺ (LSK) cells (Qiagen RNeasy Micro Kit (Qiagen, Santa Clarita, Calif.) was reversed transcribed into cDNA (High Capacity cDNA Reverse Transcriptional Kit with RNase Inhibitor (Invitrogen, Carlsbad, Calif.). Quantitative real-time polymerase chain reaction (q-RT-PCR) was performed using Fast SYBR Green Master Mix (Applied Biosystems, Foster City, Calif.] on a ABI StepOnePlus thermal cycler (Applied Biosystems, Foster City, Calif.) according to manufacturers instructions. PCR program: 95° C. for 20″, (95° C. for 1″ and 60° C. for 20″)×40, (Melt curve) 95° C. for 15″, 60° C. for 15″, and 95° C. for 15″. Tbp expression levels were used to compensate differences in cDNA input. ΔΔCt method was applied to calculate changes in gene expression. Primers used at 0.4 M. Primer sequences are listed in Table 1.

TABLE 1
qRT-PCR primer sequences for gene candidates contemplated for use in HSC
transplantation.

| Gene symbol | Forward Primer | Reverse Primer |
|---|---|---|
| Arhgef5 | CATGTGACTCCGACCAGGA | GGGTCTCGGTCTTCTTGAG |
| Armcx1 | AAGACATCTGCTGCAAGG | CCCACAACTTCTCATTCTCA |
| Cadps2 | AGATAGTAGCAGACGAAGCC | AGGCTACGGACACGTTTTTC |
| Col4a2 | GCCCTGTAGTCCTGGGAATC | CCAGTGCTACCCGGAGAAA |
| Crispld1 | CAGGAGTGGCTCAGAGTGACC | TAGCTGTCCACCATTCACCAT |
| Eltd1 | ACGAATAAGTTGGTCTGCTCT | CCCTTGTTGTAGATGACGCC |
| Emcn | GTGAGGACGGCAAAGATGTT | ACTTTTGGTCGTTCCTTCGG |
| Eya2 | GGGGGTACTGGTTCTGTGAA | CAGAGCCCCTACACCTACCC |
| Fgd5 | AGCTCCCAGCTATCTGTGAC | CCCTTGTGAACTCTGCTCAAAC |
| Foxa3 | ATGCTGGGCTCAGTGAAGAT | AGAGCTGAGTGGGTTCAAGG |
| Fstl1 | CACGGCGAGGAGGAACCTA | TCTTGCCATTACTGCCACACA |
| Glis2 | ATGCCCCACCTGTAACAAGA | CTCATAGGGGCAGACGTAGG |
| Gngl1 | ATGACACAGCTGCCCTTTTC | TCGCAAAGAAGTCAAGTTGC |
| Gprasp2 | TGCTAGGCCCAAAACTGAAAC | CATTCGGTGTCTTGTTCCAGA |
| Gpr56 | CTGCGGCAGATGGTCTACTTC | ATAGTGGAGGGTGCTCTGTTG |
| Grb10 | GGACAAATCGGAAGAGTGATCG | CATCCGTGTGCTCCGCTTAC |
| Gucyla3 | CGTCAAGGGTTATGGATCTC | GGGCGTTATGAATTGGGATG |
| Ikzf2 | TGACCTCACCTCAAGCACAC | CATCACTCTGCATTTCCAGC |
| Irf6 | CAGAGATTCCAAACGCTTCC | TGGTACTTTCCGGTCTCCAC |
| Irf9 | CTTCAAGACCACCTACTTCTG | CAGTAAATGTCGGGCAAAGG |
| Leprel2 | GCGTTCATGAGGACTATGAGG | CGGAGCGAGCTGTCTTAGAT |
| Ltbp3 | ACCGTTCATGCAGGGTAGAG | AACATGACGCTCATCGGAG |
| Mansc1 | GGGGAACCAGCTTGGCTTAC | CTTTTGAAAGCGACGATTGGATG |
| Msrb2 | TTGAACAACAAGGAGACAGGG | GCCGTAAGCCTCAGAAAATG |
| Muc13 | GATCTCTGCAACCCTAACCCC | TCCTTTCACACATGACGACAG |
| Myct1 | CCAGAGAAATCCTCCGATTG | GAGCTTAGGGAGTCCTTGGC |
| Nbea | CGATCCGCAACATCCGTATGA | TCCGAACACTCTTCCGTAGGA |
| Nfia | GAGTCCAGGAGCAATGAGG | CCATTTCATCCTCCACAGAC |
| Nfic | CCGGCATGAGAAGGACTCTAC | TTCTTCACCGGGGATGAGATG |
| Nfix | AGGCTGACAAGGTGTGGC | CACTGGGGCGACTTGTAGAG |
| Nfkb1 | TTTCGATTCCGCTATGTGTG | GAACGATAACCTTTGCAGGC |
| Nmi | ATGGACGATATGAGAGGCG | AATTCTCTGGCATCCGAC |
| Npr2 | CGGGCGCATTGTGTATATC | GTTCCTGGGTTCGATTGTCC |
| P2ry14 | TCCTCCAGACACACTGATGC | AAAGGCAAGCTTCGTCAACA |
| Rab38 | TGGTTTGAAACATCAGCCAA | GCTTCACAATGTCCGGTTCT |
| Rbp1 | GCTGAGCACTTTTCGGAACT | CCCTCCTTCTCTCCCTTCTG |
| Rbpms | GACCGCTGACAAATAGGGTC | GAAGGACCGGGAAGATGAA |
| Shank3 | CTTTGCATAGCTGGGGGTT | CCTTCCAGGTGGCCATTATT |
| Slc22a3 | CACTCTACCATCGTCAGCCA | ATAGCCCAAGGTAAAAGCCC |
| Smarca2 | AAAGATAAAGGAGCGAATCCG | GCCGAGCACTCTTAAACAC |
| Sox4 | CCAGCAAGAAAAGAAGCCAA | TGACCATGAGGCAAAATCAA |
| Stat4 | TGGCAACAATTCTGCTTCAAAAC | GAGGTCCCTGGATAGGCATGT |
| Tead2 | CCAAGCTGAAGGACCAAG | GGAGATGAGCTGTGCCGAA |
| Trim47 | GGTGAGCCAGATGTTTGCC | TCCCTCTTCGATGAACCCCAT |
| Trp53bp1 | TGCACAAAGAGAACCCCG | CTTCCTTCTCCTCCTCTGG |
| Trpc6 | GCCGGTGAGTCAGTCTGTTT | GCAACGAGAGCCAGGACTAT |
| Zbtb20 | CTTTGAAGCTGTTTTGTCTCC | GTTGATGCTGTGAATGCG |
| Zfp521 | CCCAGTCCGATGAGAAGAAG | GTTTGCACTCATGGTTCAGC |

ShRNAs.

shRNAs were designed as described (Table 2A) (Fellmann et al., 2011; Holmfeldt et al., 2013). Gene knockdown efficiency in LSK cells was quantified by qRT-PCR and normalized to transduction frequency (Table 2A and 2B).

TABLE 2A
Summary of Genes Tested in Functional Screen.

| Gene | | | |
|---|---|---|---|
| Symbol | Gene Name | shRNA | shRNA sequences |
| Arhgef5 | Rho guanine | a | TGCTGTTGACAGTGAGCGCAAGCAGAGAGATCA |
| | nucleotide | | TGATCAATAGTGAAGCCACAGATGTATTGATCAT |
| | exchange factor | | GATCTCTCTGCTTTTGCCTACTGCCTCGGA |
| | (GEF) 5 | b | TGCTGTTGACAGTGAGCGCCAGGAGGAATTTAA |
| | | | TAATACATAGTGAAGCCACAGATGTATGTATTAT |
| | | | TAAATTCCTCCTGATGCCTACTGCCTCGGA |
| Armcx1 | armadillo repeat | a | TGCTGTTGACAGTGAGCGCCGGAATTGATTTC |
| | containing, X- | | TCTGTTTATAGTGAAGCCACAGATGTATAAAC |
| | linked 1 | | AGAGAAATCAATTCCGATGCCTACTGCCTCGG |
| | | | A |
| | | b | TGCTGTTGACAGTGAGCGCCATGACTGTAACT |
| | | | AATCACTATAGTGAAGCCACAGATGTATAGTG |
| | | | ATTAGTTACAGTCATGTTGCCTACTGCCTCGG |
| | | | A |
| Cadps2 | Ca2+-dependent | a | TGCTGTTGACAGTGAGCGACAGCAGAAGCTTAA |
| | activator protein | | CAAACAATAGTGAAGCCACAGATGTATTGTTTGT |
| | for secretion 2 | | TAAGCTTCTGCTGCTGCCTACTGCCTCGGA |
| | | b | TGCTGTTGACAGTGAGCGCCAGAGAGGTGTTTA |
| | | | AGAAGAATAGTGAAGCCACAGATGTATTCTTCTT |
| | | | AAACACCTCTCTGATGCCTACTGCCTCGGA |
| Col4a2 | collagen, type IV, | a | TGCTGTTGACAGTGAGCGACAGGACAGAGAGAT |
| | alpha 2 | | TGTGACATAGTGAAGCCACAGATGTATGTCACA |
| | | | ATCTCTCTGTCCTGGTGCCTACTGCCTCGGA |
| | | b | TGCTGTTGACAGTGAGCGACAGCTTGGTGCTTAC |
| | | | TCTTAATAGTGAAGCCACAGATGTATTAAGAGT |
| | | | AAGCACCAAGCTGGTGCCTACTGCCTCGGA |
| Crispld1 | cysteine-rich | a | TGCTGTTGACAGTGAGCGCCAGATTGTTTCTTGT |
| | secretory protein | | GAAGTATAGTGAAGCCACAGATGTATACTTCAC |
| | LCCL domain | | AAGAAACAATCTGATGCCTACTGCCTCGGA |
| | containing 1 | b | TGCTGTTGACAGTGAGCGCCAGAAAGTTTACAG |
| | | | AACCCTATAGTGAAGCCACAGATGTATAGGGTT |
| | | | CTGTAAACTTTCTGATGCCTACTGCCTCGGA |
| Eltd1 | EGF, latrophil in | a | TGCTGTTGACAGTGAGCGACAGAAGTTAGTTGCT |
| | seven | | ATGAGATAGTGAAGCCACAGATGTATCTCATAG |
| | transmembrane | | CAACTAACTTCTGGTGCCTACTGCCTCGGA |
| | domain | b | TGCTGTTGACAGTGAGCGCCACAGATTAAGACTT |
| | containing 1 | | CAAATATAGTGAAGCCACAGATGTATATTTGAA |
| | | | GTCTTAATCTGTGTTGCCTACTGCCTCGGA |
| Emcn | Endomucin | a | TGCTGTTGACAGTGAGCGCCCATGTCACTGCTTC |
| | | | AAGATATAGTGAAGCCACAGATGTATATCTTGA |
| | | | AGCAGTGACATGGTTGCCTACTGCCTCGGA |
| | | b | TGCTGTTGACAGTGAGCGAACCAGTCACCTGTCT |
| | | | TAGCAATAGTGAAGCCACAGATGTATTGCTAAG |
| | | | ACAGGTGACTGGTGTGCCTACTGCCTCGGA |
| | | c | TGCTGTTGACAGTGAGCGCAACTAGAAATGTTTC |
| | | | CTTTAATAGTGAAGCCACAGATGTATTAAAGGA |
| | | | AACATTTCTAGTTATGCCTACTGCCTCGGA |
| Eya2 | eyes absent 2 | a | TGCTGTTGACAGTGAGCGCCAAGACAGAAGACA |
| | homolog | | GTTTGAATAGTGAAGCCACAGATGTATTCAAACT |
| | | | GTCTTCTGTCTTGATGCCTACTGCCTCGGA |
| | | b | TGCTGTTGACAGTGAGCGATCAGGATTTAAGCA |
| | | | CATACAATAGTGAAGCCACAGATGTATTGTATGT |
| | | | GCTTAAATCCTGACTGCCTACTGCCTCGGA |
| Fgd5 | FYVE, RhoGEF | a | TGCTGTTGACAGTGAGCGCCAGACTGTACACCCT |
| | and PH domain | | TATCTATAGTGAAGCCACAGATGTATAGATAAG |
| | containing 5 | | GGTGTACAGTCTGTTGCCTACTGCCTCGGA |
| | | b | TGCTGTTGACAGTGAGCGCCCAGAAACTGTTCTA |
| | | | GAAGAATAGTGAAGCCACAGATGTATTCTTCTA |
| | | | GAACAGTTTCTGGATGCCTACTGCCTCGGA |
| Foxa3 | forkhead box A3 | a | TGCTGTTGACAGTGAGCGCCAACTCCTACATGAC |
| | | | CTTGAATAGTGAAGCCACAGATGTATTCAAGGT |
| | | | CATGTAGGAGTTGATGCCTACTGCCTCGGA |
| | | b | TGCTGTTGACAGTGAGCGACGAGGTGTATTCTCC |
| | | | AGTGAATAGTGAAGCCACAGATGTATTCACTGG |
| | | | AGAATACACCTCGCTGCCTACTGCCTCGGA |
| Fstl1 | follistatin-like 1 | a | TGCTGTTGACAGTGAGCGCCAGTGAGATCCTAG |
| | | | ACAAGTATAGTGAAGCCACAGATGTATACTTGT |
| | | | CTAGGATCTCACTGTTGCCTACTGCCTCGGA |
| | | b | TGCTGTTGACAGTGAGCGATCAGTTATTACCGTT |
| | | | ATATTATAGTGAAGCCACAGATGTATAATATAA |
| | | | CGGTAATAACTGAGTGCCTACTGCCTCGGA |
| Glis2 | GUS family zinc | a | TGCTGTTGACAGTGAGCGACAGCTCTTTGAGCTC |
| | finger 2 | | CTCCAATAGTGAAGCCACAGATGTATTGGAGGA |
| | | | GCTCAAAGAGCTGGTGCCTACTGCCTCGGA |
| | | b | TGCTGTTGACAGTGAGCGCACCAGCTGTGGTAA |
| | | | ACTGAAATAGTGAAGCCACAGATGTATTTCAGTT |
| | | | TACCACAGCTGGTTTGCCTACTGCCTCGGA |
| Gng11 | guanine | a | TGCTGTTGACAGTGAGCGACAGAGACAACAGGT |
| | nucleotide | | ATCTAAATAGTGAAGCCACAGATGTATTTAGAT |
| | binding protein | | ACCTGTTGTCTCTGCTGCCTACTGCCTCGGA |
| | (G protein), | b | TGCTGTTGACAGTGAGCGACATCTGATTGCAGTT |
| | gamma 11 | | ATGGAATAGTGAAGCCACAGATGTATTCCATAA |
| | | | CTGCAATCAGATGCTGCCTACTGCCTCGGA |
| Gprasp2 | G protein- | a | TGCTGTTGACAGTGAGCGATGGGACGAGGTT |
| | coupled receptor | | ACCATCGAATAGTGAAGCCACAGATGTATTCG |
| | associated | | ATGGTAACCTCGTCCCAGTGCCTACTGCCTCG |
| | sorting protein 2 | | GA |
| | | b | TGCTGTTGACAGTGAGCGCCAGTAAAGTTAGT |
| | | | GTGATTTATAGTGAAGCCACAGATGTATAAAT |
| | | | CACACTAACTTTACTGTTGCCTACTGCCTCGG |
| | | | A |
| | | c | TGCTGTTGACAGTGAGCGCTCGGGTGTTGTCT |
| | | | CACTGATTTAGTGAAGCCACAGATGTAAATCA |
| | | | GTGAGACAACACCCGAATGCCTACTGCCTCG |
| | | | GA |
| | | d | TGCTGTTGACAGTGAGCGCCAGTCTGTTTCTG |
| | | | TGTCGTAATAGTGAAGCCACAGATGTATTACG |
| | | | ACACAGAAACAGACTGTTGCCTACTGCCTCGG |
| | | | A |
| Gpr56 | G protein-coupled | a | TGCTGTTGACAGTGAGCGCCAGTCTGGTGTTCCT |
| | receptor 56 | | GTTCAATAGTGAAGCCACAGATGTATTGAACAG |
| | | | GAACACCAGACTGATGCCTACTGCCTCGGA |
| | | b | TGCTGTTGACAGTGAGCGATAGGCGATTGTCGA |
| | | | GAAGAGATAGTGAAGCCACAGATGTATCTCTTC |
| | | | TCGACAATCGCCTACTGCCTACTGCCTCGGA |
| Grb10 | growth factor | a | TGCTGTTGACAGTGAGCGAACGAGATGTTATTAC |
| | receptor bound | | AACAAATAGTGAAGCCACAGATGTATTTGTTGT |
| | protein 10 | | AATAACATCTCGTGTGCCTACTGCCTCGGA |
| | | b | TGCTGTTGACAGTGAGCGCCAGCATAGTGGAAG |
| | | | ATAGATATAGTGAAGCCACAGATGTATATCTATC |
| | | | TTCCACTATGCTGTTGCCTACTGCCTCGGA |
| Gucy1a3 | guanylate cyclase | a | TGCTGTTGACAGTGAGCGATGGCATCATGACAA |
| | 1, soluble, alpha 3 | | TGTTGAATAGTGAAGCCACAGATGTATTCAACAT |
| | | | TGTCATGATGCCACTGCCTACTGCCTCGGA |
| | | b | TGCTGTTGACAGTGAGCGACACCACATACAGGT |
| | | | TACTCAATAGTGAAGCCACAGATGTATTGAGTA |
| | | | ACCTGTATGTGGTGGTGCCTACTGCCTCGGA |
| | | c | TGCTGTTGACAGTGAGCGCCCAGGACTTTCTAAA |
| | | | TGTTTATAGTGAAGCCACAGATGTATAAACATTT |
| | | | AGAAAGTCCTGGTTGCCTACTGCCTCGGA |
| Ikzf2 | IKAROS family | a | TGCTGTTGACAGTGAGCGATGGGTAAACCTCAC |
| | zinc finger 2 | | AAGTGTATAGTGAAGCCACAGATGTATACACTT |
| | | | GTGAGGTTTACCCACTGCCTACTGCCTCGGA |
| | | b | TGCTGTTGACAGTGAGCGCACCGCCTATGGAAG |
| | | | ATTGTAATAGTGAAGCCACAGATGTATTACAATC |
| | | | TTCCATAGGCGGTATGCCTACTGCCTCGGA |
| Irf6 | interferon | a | TGCTGTTGACAGTGAGCGCTGGGATGAGAAAGA |
| | regulatory factor | | TAATGATTAGTGAAGCCACAGATGTAATCATTAT |
| | 6 | | CTTTCTCATCCCAATGCCTACTGCCTCGGA |
| | | b | TGCTGTTGACAGTGAGCGCCCACTCCAGACATCA |
| | | | AAGATATAGTGAAGCCACAGATGTATATCTTTG |
| | | | ATGTCTGGAGTGGATGCCTACTGCCTCGGA |
| Leprel2 | leprecan-like 2 | a | TGCTGTTGACAGTGAGCGCCGGAGAGAAGAGAC |
| | | | AGTTATATAGTGAAGCCACAGATGTATATAACT |
| | | | GTCTCTTCTCTCCGATGCCTACTGCCTCGGA |
| | | b | TGCTGTTGACAGTGAGCGACAGAGCAACTGCTT |
| | | | CTAATAATAGTGAAGCCACAGATGTATTATTAG |
| | | | AAGCAGTTGCTCTGCTGCCTACTGCCTCGGA |
| Msrb2 | methionine | a | TGCTGTTGACAGTGAGCGATGGAATGTATTTGAA |
| | sulfoxide | | CAACAATAGTGAAGCCACAGATGTATTGTTGTTC |
| | reductase B2 | | AAATACATTCCACTGCCTACTGCCTCGGA |
| | | b | TGCTGTTGACAGTGAGCGCAAGGCTCAGATGAA |
| | | | AGTCACATAGTGAAGCCACAGATGTATGTGACT |
| | | | TTCATCTGAGCCTTTTGCCTACTGCCTCGGA |
| Muc13 | mucin 13, | a | TGCTGTTGACAGTGAGCGAACGGTACAGAGTCA |
| | epithelial | | ATCTCCATAGTGAAGCCACAGATGTATGGAGAT |
| | transmembrane | | TGACTCTGTACCGTGTGCCTACTGCCTCGGA |
| | | b | TGCTGTTGACAGTGAGCGATCCCACCACAGTAC |
| | | | AAAGTCATAGTGAAGCCACAGATGTATGACTTT |
| | | | GTACTGTGGTGGGAGTGCCTACTGCCTCGGA |
| Myct1 | myc target 1 | a | TGCTGTTGACAGTGAGCGACAGCCTCACTTTCCA |
| | | | GAGACATAGTGAAGCCACAGATGTATGTCTCTG |
| | | | GAAAGTGAGGCTGGTGCCTACTGCCTCGGA |
| | | b | TGCTGTTGACAGTGAGCGCAAGAGTGGTTCTAC |
| | | | AAAGATATAGTGAAGCCACAGATGTATATCTTT |
| | | | GTAGAACCACTCTTATGCCTACTGCCTCGGA |
| | | c | TGCTGTTGACAGTGAGCGCTCCGGTGGAAACGG |
| | | | AGAGTCATAGTGAAGCCACAGATGTATGACTCT |
| | | | CCGTTTCCACCGGAATGCCTACTGCCTCGGA |
| Nbea | neurobeachin | a | TGCTGTTGACAGTGAGCGCCGGAAGAGTGTTCG |
| | | | GAATTTATAGTGAAGCCACAGATGTATAAATTCC |
| | | | GAACACTCTTCCGTTGCCTACTGCCTCGGA |
| | | b | TGCTGTTGACAGTGAGCGCCGGCAGCTTAATGA |
| | | | CAGTCAATAGTGAAGCCACAGATGTATTGACTG |
| | | | TCATTAAGCTGCCGATGCCTACTGCCTCGGA |
| Nfkb1 | nuclear factor of | a | TGCTGTTGACAGTGAGCGCTACCTTCAAATATTA |
| | kappa light | | GAGCAATAGTGAAGCCACAGATGTATTGCTCTA |
| | polypeptide gene | | ATATTTGAAGGTATTGCCTACTGCCTCGGA |
| | enhancer in B | b | TGCTGTTGACAGTGAGCGAACCAAGCAGGAAGA |
| | cells 1, p105 | | TGTAGTATAGTGAAGCCACAGATGTATACTACAT |
| | | | CTTCCTGCTTGGTGTGCCTACTGCCTCGGA |
| Nmi | N-myc (and | a | TGCTGTTGACAGTGAGCGAAGGCGTCAGATTCC |
| | STAT) interactor | | AGGTTCATAGTGAAGCCACAGATGTATGAACCT |
| | | | GGAATCTGACGCCTGTGCCTACTGCCTCGGA |
| | | b | TGCTGTTGACAGTGAGCGACAGGTTCATGTGGA |
| | | | CATTTCTTAGTGAAGCCACAGATGTAAGAAATGT |
| | | | CCACATGAACCTGGTGCCTACTGCCTCGGA |
| Npr2 | natriuretic peptide | a | TGCTGTTGACAGTGAGCGACGCTGTGGACCTCA |
| | receptor 2 | | AGCTGTATAGTGAAGCCACAGATGTATACAGCT |
| | | | TGAGGTCCACAGCGCTGCCTACTGCCTCGGA |
| | | b | TGCTGTTGACAGTGAGCGACCGCTCAGCCTTGTA |
| | | | CATAGATAGTGAAGCCACAGATGTATCTATGTA |
| | | | CAAGGCTGAGCGGCTGCCTACTGCCTCGGA |
| P2ry14 | purinergic | a | TGCTGTTGACAGTGAGCGATGCCGTCATCTTCTA |
| | receptor P2Y, G- | | TGTTAATAGTGAAGCCACAGATGTATTAACATA |
| | protein coupled, | | GAAGATGACGGCAGTGCCTACTGCCTCGGA |
| | 14 | b | TGCTGTTGACAGTGAGCGACAGGCATATGATGA |
| | | | TAAGTAATAGTGAAGCCACAGATGTATTACTTAT |
| | | | CATCATATGCCTGCTGCCTACTGCCTCGGA |
| Rab38 | RAB38, member | a | TGCTGTTGACAGTGAGCGCAGGGAAGGATGTGC |
| | RAS oncogene | | TTATGAATAGTGAAGCCACAGATGTATTCATAA |
| | family | | GCACATCCTTCCCTTTGCCTACTGCCTCGGA |
| | | b | TGCTGTTGACAGTGAGCGCGACCTCCTAGAGTCT |
| | | | ATAGAATAGTGAAGCCACAGATGTATTCTATAG |
| | | | ACTCTAGGAGGTCATGCCTACTGCCTCGGA |
| Rbp1 | retinol binding | a | TGCTGTTGACAGTGAGCGATGCAAGCAAGTGTTT |
| | protein 1, cellular | | AAGAAATAGTGAAGCCACAGATGTATTTCTTAA |
| | | | ACACTTGCTTGCAGTGCCTACTGCCTCGGA |
| | | b | TGCTGTTGACAGTGAGCGAGAAGATGCTGAGCA |
| | | | ATGAGAATAGTGAAGCCACAGATGTATTCTCATT |
| | | | GCTCAGCATCTTCCTGCCTACTGCCTCGGA |
| Rbpms | RNA binding | a | TGCTGTTGACAGTGAGCGACAACACTGTACCTCA |
| | protein gene with | | GTTCATTAGTGAAGCCACAGATGTAATGAACTG |
| | multiple splicing | | AGGTACAGTGTTGGTGCCTACTGCCTCGGA |
| | | b | TGCTGTTGACAGTGAGCGATCTCATAAAGCTCAC |
| | | | ATCTAATAGTGAAGCCACAGATGTATTAGATGT |
| | | | GAGCTTTATGAGAGTGCCTACTGCCTCGGA |
| Shank3 | SH3/ankyrin | a | TGCTGTTGACAGTGAGCGCCCGATACAAGCGGA |
| | domain gene 3 | | GAGTTTATAGTGAAGCCACAGATGTATAAACTCT |
| | | | CCGCTTGTATCGGATGCCTACTGCCTCGGA |
| | | b | TGCTGTTGACAGTGAGCGCCACAAGTTTCTGTTT |
| | | | CTATTTTAGTGAAGCCACAGATGTAAAATAGAA |
| | | | ACAGAAACTTGTGATGCCTACTGCCTCGGA |
| Slc22a3 | solute carrier | a | TGCTGTTGACAGTGAGCGACAGGCTCATCATTTA |
| | family 22 | | CTTAATTAGTGAAGCCACAGATGTAATTAAGTA |
| | (organic cation | | AATGATGAGCCTGCTGCCTACTGCCTCGGA |
| | transporter), | b | TGCTGTTGACAGTGAGCGAAGAGATCACAGTTA |
| | member 3 | | CAGATGATAGTGAAGCCACAGATGTATCATCTG |
| | | | TAACTGTGATCTCTGTGCCTACTGCCTCGGA |
| Smarca2 | SWI/SNF related, | a | TGCTGTTGACAGTGAGCGACGGCTGAGAAGTTG |
| | matrix associated, | | TCACCAATAGTGAAGCCACAGATGTATTGGTGA |
| | actin dependent | | CAACTTCTCAGCCGGTGCCTACTGCCTCGGA |
| | regulator of | b | TGCTGTTGACAGTGAGCGATACGAAGACTCCATT |
| | chromatin, | | GTCCTATAGTGAAGCCACAGATGTATAGGACAA |
| | subfamily a, | | TGGAGTCTTCGTAGTGCCTACTGCCTCGGA |
| | member 2 | | |
| Sox4 | SRY (sex | a | TGCTGTTGACAGTGAGCGCCCCTGCCGACAAGA |
| | determining | | AAGTGAATAGTGAAGCCACAGATGTATTCACTTT |
| | region Y)-box 4 | | CTTGTCGGCAGGGTTGCCTACTGCCTCGGA |
| | | b | TGCTGTTGACAGTGAGCGCTAGATGGAGAGTAG |
| | | | AAGGAGATAGTGAAGCCACAGATGTATCTCCTT |
| | | | CTACTCTCCATCTATTGCCTACTGCCTCGGA |
| Stat4 | signal transducer | a | TGCTGTTGACAGTGAGCGCTCCTGCGAGACTACA |
| | and activator of | | AGGTTATAGTGAAGCCACAGATGTATAACCTTGT |
| | transcription 4 | | AGTCTCGCAGGATTGCCTACTGCCTCGGA |
| | | b | TGCTGTTGACAGTGAGCGCCACAGTTCAGTCTAA |
| | | | CTACAATAGTGAAGCCACAGATGTATTGTAGTTA |
| | | | GACTGAACTGTGATGCCTACTGCCTCGGA |
| Tead2 | TEA domain | a | TGCTGTTGACAGTGAGCGAACGCAGTTGACTCGT |
| | family member 2 | | TCCAGATAGTGAAGCCACAGATGTATCTGGAAC |
| | | | GAGTCAACTGCGTCTGCCTACTGCCTCGGA |
| | | b | TGCTGTTGACAGTGAGCGCACACGAGGACCTCA |
| | | | GAGACAATAGTGAAGCCACAGATGTATTGTCTC |
| | | | TGAGGTCCTCGTGTTTGCCTACTGCCTCGGA |
| Trp53bp1 | transformation | a | TGCTGTTGACAGTGAGCGCCCGGAACAATCTGCT |
| | related protein 53 | | GTAGAATAGTGAAGCCACAGATGTATTCTACAG |
| | binding protein 1 | | CAGATTGTTCCGGATGCCTACTGCCTCGGA |
| | | b | TGCTGTTGACAGTGAGCGACAGGATGTTGAAGA |
| | | | ACATACATAGTGAAGCCACAGATGTATGTATGTT |
| | | | CTTCAACATCCTGGTGCCTACTGCCTCGGA |
| Trpc6 | transient receptor | a | TGCTGTTGACAGTGAGCGCCACAGAGCTGCTACT |
| | potential cation | | CAAGAATAGTGAAGCCACAGATGTATTCTTGAG |
| | channel, | | TAGCAGCTCTGTGATGCCTACTGCCTCGGA |
| | subfamily C, | b | TGCTGTTGACAGTGAGCGAGAGGACCAGCATAC |
| | member 6 | | ATGTTTATAGTGAAGCCACAGATGTATAAACAT |
| | | | GTATGCTGGTCCTCGTGCCTACTGCCTCGGA |
| Zbtb20 | zinc finger and | a | TGCTGTTGACAGTGAGCGACCCAGCAAAGTTTG |
| | BTB domain | | ACCAAATTAGTGAAGCCACAGATGTAATTTGGT |
| | containing 20 | | CAAACTTTGCTGGGCTGCCTACTGCCTCGGA |
| | | b | TGCTGTTGACAGTGAGCGCCACAGTCATCACTGT |
| | | | CAGTAATAGTGAAGCCACAGATGTATTACTGAC |
| | | | AGTGATGACTGTGTTGCCTACTGCCTCGGA |
| | | c | TGCTGTTGACAGTGAGCGCCCGAATCTACTCCGC |
| | | | ACTCTATAGTGAAGCCACAGATGTATAGAGTGC |
| | | | GGAGTAGATTCGGTTGCCTACTGCCTCGGA |
| Zfp521 | zinc finger | a | TGCTGTTGACAGTGAGCGCCAGCTGTATTTACTG |
| | protein 521 | | CAACAATAGTGAAGCCACAGATGTATTGTTGCA |
| | | | GTAAATACAGCTGTTGCCTACTGCCTCGGA |
| | | b | TGCTGTTGACAGTGAGCGACACAGCAGTTAGTTC |
| | | | ATGTATTAGTGAAGCCACAGATGTAATACATGA |
| | | | ACTAACTGCTGTGCTGCCTACTGCCTCGGA |

TABLE 2B
Summary of Genes Tested in Functional Screen.

| | | | | | Putative | |
|---|---|---|---|---|---|---|
| | | KD | Hit in | Hit in | Cellular | |
| Gene | | Efficiency in | Primary | Secondary | Function | |
| Symbol | shRNA | LSK cells | Screen | Screen | (if Hit) | References |
| Arhgef5 | a | 92.0356 | Yes | Yes | Podosome and | Kuroiwa et al., |
| | | | | | invadopodia | 2011 |
| | | | | | formation, cell | |
| | | | | | adhesion and | |
| | | | | | migration | |
| | b | 84.403 | — | — | — | — |
| Armcx1 | a | 97.074775 | Not tested | Yes | Unknown | Abu-Helo |
| | | | | | | and Simonin, |
| | | | | | | 2010 |
| | b | 96.943855 | — | — | — | — |
| Cadps2 | a | 84.885915 | Yes | Yes | Vesicle | Cisternas et |
| | | | | | trafficking and | al., 2003 |
| | | | | | exocytosis | |
| | b | 86.155605 | — | — | — | — |
| Col4a2 | a | 90.301155 | Yes | No | | |
| | b | 91.034466 | — | — | — | — |
| Crispld1 | a | 71.792755 | Yes | Yes | Putative | Gibbs et al., |
| | | | | | secreted | 2008 |
| | | | | | protease | |
| | b | 71.97974 | — | — | — | — |
| Eltd1 | a | 80.24435 | No | Not tested | | |
| | b | 91.386155 | — | — | — | — |
| Emcn | a | 70.207375 | Yes | Yes | Adhesion | Matsubara et |
| | | | | | | al., 2005 |
| | b | 83.065945 | — | — | — | — |
| | c | 89.721135 | — | — | — | — |
| Eya2 | a | 88.17265 | No | Not tested | | |
| | b | 76.4652 | — | — | — | — |
| Fgd5 | a | 84.85855 | No | Not tested | | |
| | b | 86.70709 | — | — | — | — |
| Foxa3 | a | 66.67249675 | Yes | Yes | Transcription | Friedman and |
| | | | | | factor | Kaestner, |
| | | | | | | 2006 |
| | b | 68.93917468 | — | — | — | — |
| Fstl1 | a | 88.80138 | Type 2 | Yes | Extracellular | Geng et al., |
| | | | non-Hit | | negative | 2011 |
| | | | | | regulator of | |
| | | | | | TGFb/BMP | |
| | | | | | signaling | |
| | b | 86.285331 | — | — | — | — |
| Glis2 | a | 70.129355 | Yes | Yes | Transcription | Gruber et al., |
| | | | | | factor | 2012 |
| | b | 67.723615 | — | — | — | — |
| Gng11 | a | 76.39386 | Yes | No | | |
| | b | 83.931255 | — | — | — | — |
| Gprasp2 | a | 79.80325 | Yes | Yes | Putative | Abu-Helo |
| | | | | | regulator of | and Simonin, |
| | | | | | G-protein | 2010; Moser |
| | | | | | coupled | et al., 2010 |
| | | | | | receptor cell | |
| | | | | | surface | |
| | | | | | turnover | |
| | b | 65.29157 | — | — | — | — |
| | c | 59.22959 | — | — | — | — |
| | d | 61.673965 | — | — | — | — |
| Gpr56 | a | 76.388505 | Yes | Yes | Migration | Singer et al., |
| | | | | | | 2013; |
| | | | | | | Solaimani- |
| | | | | | | Kartataei et |
| | | | | | | al., 2015 |
| | b | 81.10845 | — | — | — | — |
| Grb10 | a | 91.376335 | No | Not tested | | |
| | b | 92.15143 | — | — | — | — |
| Gucy1a3 | a | 90.6446495 | No | No | | |
| | b | 87.745385 | — | — | — | — |
| | c | 85.35236 | — | — | — | — |
| Ikzf2 | a | 73.050495 | No | Not tested | | |
| | b | 65.04349 | — | — | — | — |
| Irf6 | a | 84.69899 | No | Not tested | | |
| | b | 91.15006 | — | — | — | — |
| Leprel2 | a | 75.476437 | Yes | No | | |
| | b | 69.77763675 | — | — | — | — |
| Msrb2 | a | 89.5388025 | No | Not tested | | |
| | b | 92.680947 | — | — | — | — |
| Muc13 | a | 70.79097 | No | No | | |
| | b | 75.653045 | — | — | — | — |
| Myct1 | a | 80.31919 | Type 2 Hit | Yes | Transcription | Wilson et al., |
| | | | | | factor | 2004 |
| | b | 60.088565 | — | — | — | — |
| | c | 82.881685 | — | — | — | — |
| Nbea | a | 73.79806 | Yes | Yes | Vesicle | Niesmann et |
| | | | | | trafficking | al., 2011 |
| | b | 69.156605 | — | — | — | — |
| Nfkb1 | a | 76.948735 | No | Not tested | | |
| | b | 80.45947 | — | — | — | — |
| Nmi | a | 77.30752461 | No | Not tested | | |
| | b | 85.52914263 | — | — | — | — |
| Npr2 | a | 88.86343 | No | Not tested | | |
| | b | 80.93918 | — | — | — | — |
| P2ry14 | a | 96.671435 | Yes | Yes | Purinergic | Cho et al., |
| | | | | | receptor | 2014 |
| | b | 83.619455 | — | — | — | — |
| Rab38 | a | 88.49545 | No | Not tested | | |
| | b | 90.6497105 | — | — | — | — |
| Rbp1 | a | 91.147785 | No | No | | |
| | b | 87.745385 | — | — | — | — |
| Rbpms | a | 95.172672 | Yes | No | | |
| | b | 88.465241 | — | — | — | — |
| Shank3 | a | 59.678655 | No | Not tested | | |
| | b | 50.401845 | — | — | — | — |
| Slc22a3 | a | 92.053574 | No | Not tested | | |
| | b | 88.53625 | — | — | — | — |
| Smarca2 | a | 78.07302 | No | Yes | SWI2/SNF2- | Buscarlet et |
| | | | | | like ATPase, | al., 2014 |
| | | | | | member of | |
| | | | | | BAF | |
| | | | | | chromatin | |
| | | | | | remodeling | |
| | | | | | complex, also | |
| | | | | | known as Brm | |
| | b | 84.400475 | — | — | — | — |
| Sox4 | a | 82.19736 | Yes | Yes | Transcription | Zhang et al., |
| | | | | | factor | 2013; Ma et |
| | | | | | | al., 2014 |
| | b | 76.008135 | — | — | — | — |
| Stat4 | a | 75.518965 | Yes | Yes | Transcription | |
| | | | | | factor | |
| | b | 75.680085 | — | — | — | — |
| Tead2 | a | 88.966845 | No | Not tested | | |
| | b | 94.14039 | — | — | — | — |
| Trp53bp1 | a | 76.764715 | Yes | No | | |
| | b | 79.234965 | — | — | — | — |
| Trpc6 | a | 72.176435 | Yes | No | | |
| | b | 96.119432 | — | — | — | — |
| Zbtb20 | a | 86.649874 | Yes | No | | |
| | b | 73.51000667 | — | — | — | — |
| | c | 82.84994167 | — | — | — | — |
| Zfp521 | a | 58.359595 | No | Yes | Putative | None |
| | | | | | transcription | |
| | | | | | factor | |
| — | b | 64.5274 | — | — | — | — |

Lentiviral Production.

Vesicular stomatitis virus glycoprotein (VSV-G)-pseudotyped lentivirus was prepared as described via a four-plasmid system (Transfer vector-, Gag/Pol-, Rev/Tat-, and VSV-G envelope plasmid) by co-transfection of 293T cells using TransiT 293 (Mirus, Madison, Wis.) (Holmfeldt et al., 2013). Viral supernatant were collected 48 hours later, cleared, and stored at −80° C. Viral preparations were titered on 293T cells.

LSK Cell Culture and Transduction.

LSK (Lineage-Sca-1+c-Kit+) cells were isolated from 6-10 week old murine bone marrow and transduced with lentivirus as described (Holmfeldt et al., 2013). Briefly, non-tissue culture 96-well plates were coated with Retronectin (TaKaRA Bio USA, Madison, Wis.) according to the manufacturer's instructions. Lentiviral particles corresponding to a multiplicity of infection (MOI) of 25 were spin loaded onto the plates for 1 hour at 1000G and room temperature. Wells were washed with PBS followed by the addition of 15,000 freshly isolated LSK cells resuspended in 200 uL serum-free expansion medium (StemCell Technologies, Vancouver, British Columbia, Canada) with 10 ng/mL recombinant murine (RM) stem cell factor (SCF), 20 ng/mL RM thrombopoietin (Tpo), 20 ng/mL RM insulin-like growth factor 2 (IGF-2) (Peprotech, Rocky Hill, N.J.), 10 ng/mL recombinant human (RH) fibroblast growth factor 1 (FGF-1) (R&DSystems, Minneapolis, Minn.) and 5 μg/mL protamine sulfate (Sigma-Aldrich, St. Louis, Mo.). Cells were incubated overnight at 37° C. To collect cells for transplantation the next morning, media was slowly removed and cells were washed and resuspended in PBS+1.5% FCS.

To compare the transduction efficiency of LSK cells versus LSK CD150+CD48− cells, these cells were isolated in parallel, as previously described (Holmfeldt et al., 2013). 2500 cells were transduced on graded concentrations of indicated viruses, in retronectin coated 96-well plates, as described above. Transduction frequencies were analyzed four days post transduction using flow cytometry. To assess any non-specific effect of shRNAs on the viability of primitive hematopoietic cells, LSK cells transduced with lentivirus were cultured for two weeks in serum-free expansion medium (StemCell Technologies, Vancouver, British Columbia, Canada) with 10 ng/mL RM-SCF, 20 ng/mL RM thrombopoietin (Tpo), 20 ng/mL RM IGF-2 (Peprotech, Rocky Hill, N.J.), 10 ng/mL RH-FGF-1 (R&D Systems, Minneapolis, Minn.) and 10 mg/mL heparin (Sigma-Aldrich, St. Louis, Mo.). The persistence of mCherry+ cells was monitored using a BD LSRFortessa (BD Biosciences, San Diego, Calif.) and Flowjo version 9.4.11 (Tree Star, Ashland, Oreg.).

Bone Marrow Transplants.

Recipients were treated with 11 Gy of ionizing radiation in split doses of 5.5 Gy. For the functional screen, 5000 CD45.2⁺ Test LSK cells were injected 24 hours post transduction with 5000 mock transduced CD45.1⁺ Competitor LSK cells into recipients by tail vein. For retesting of Hits, 5000 CD45.2⁺ Test mCherry+/LSK cells were isolated by FACS 44 hours post transduction and injected with 5000 mock transduced and mock-sorted CD45.1⁺ Competitor LSK cells by tail vein. For 1:4 Test versus Competitor transplants, 2000 CD45.2⁺ Test mCherry+/LSK cells were isolated by FACS 44 hours post transduction and transplanted with 8000 mock transduced and mock-sorted CD45.1⁺ competitor LSK cells.

For investigating Foxa3, 4×10⁵ CD45.2⁺ Foxa3^{+/+} or Foxa3^{-/-} WBM cells were injected with 4×10⁵ CD45.1⁺ WBM cells into lethally irradiated CD45.1⁺/CD45.2⁺ recipients by tail vein. For secondary transplants, 4×10⁵ CD45.2⁺ WBM cells sorted from primary recipients of Foxa3^{+/+} or Foxa3^{-/-} WBM cells were transplanted with 4×10⁵ CD45.1⁺ WBM WT competitor cells into lethally irradiated CD45.1+/CD45.2⁺ recipients. For limiting dilution transplants, 15,000, 30,000, 50,000,100,000, or 200,000 CD45.2⁺ Foxa3^{+/+} or Foxa3^{-/-} WBM cells were injected with 2×10⁵ CD45.1⁺ WBM cells into lethally irradiated CD45.1+/CD45.2⁺ recipients by tail vein in two independent experiments. Engraftment was defined as >1% CD45.2 chimerism in the T cell, B cell, and myeloid lineages of recipient peripheral blood (PB) 10-16 weeks post-transplant. L-Calc (Stem Cell Technologies, Vancouver, Canada) was used to analyze the results of the limiting dilution transplants.

Antibodies for Whole Bone Marrow (WBM) and Peripheral Blood (PB) Analysis.

Antibodies used in this study for the analysis of Whole Bone Marrow and peripheral blood cell populations by flow cytometry are as previously described (Holmfeldt et al., 2013).

Analysis of Peripheral Blood.

Peripheral blood (PB) was collected from the retro-orbital plexus in heparinized capillary tubes and lysed in red blood cell lysis buffer (Sigma-Aldrich, St. Louis, Mo.). Cells were stained with the following antibodies: CD45.1-FITC, CD45.2-APC, (B220, Grl, Cdllb)-PerCPCy5.5, (B220, CD4, CD8)-PECy7 (BD Biosciences, San Diego, Calif.) followed by flow cytometry analysis using BD LSRFortessa (BD Biosciences, San Diego, Calif.) and data analysis using FlowJo version 9.4.11 (Tree Star, Ashland, Oreg.).

CFU Assays.

For analysis of CFU potential of LSK cells following knockdown of screen Hits, LSK cells were transduced overnight with control or gene-specific shRNAs and then cultured at 15,000 cells/well in non-tissue culture treated 96-well plates for 5-6 days in serum-free expansion medium (StemCell Technologies, Vancouver, British Columbia, Canada) with 10 ng/mL RM SCF, 20 ng/mL RM Tpo, 20 ng/mL RM IGF-2 (Peprotech, Rocky Hill, N.J.), 10 ng/mL RH FGF-1 (R&DSystems, Minneapolis, Minn.) and 10 ug/mL heparin (Sigma-Aldrich, St. Louis, Mo.). 500 mCherry+ LSK cells were then isolated by FACS and plated in M3434 methylcellulose (StemCell Technologies). For CFU analysis of Foxa3^{+/+} or Foxa3^{-/-} HSC, 150 HSC (LSK CD150+CD48-) were isolated by FACS from WBM and then plated in M3434. Colonies were analyzed 10 days after plating.

Cell Cycle Analysis of shRNA Transduced LSK Cells.

LSK cells were transduced overnight with control or gene-specific shRNAs and then cultured at 15,000 cells/well in non-tissue culture treated 96-well plates for 5-6 days in serum-free expansion medium (StemCell Technologies, Vancouver, British Columbia, Canada) with 10 ng/mL RM SCF, 20 ng/mL RM Tpo, 20 ng/mL RM IGF-2 (Peprotech, Rocky Hill, N.J.), 10 ng/mL RH FGF-1 (R&D Systems, Minneapolis, Minn.) and 10 ug/mL heparin (Sigma-Aldrich, St. Louis, Mo.). mCherry+ LSK cells were then collected by FACS and stained with the following antibodies: (B220, CD3, CD4, CD8, CD19, Gr-1, Ter119)-PerCP, Sca-1-PerCP-Cy5.5, c-Kit-APC-780. Cells were then fixed using the Cytofix/Cytoperm kit (BD Biosciences, San Diego, Calif.) followed by staining for Ki67-FITC (Clone SolA15)(eBioscience, San Diego, Calif.) and 4′,6-diamidino-2-phenylindole (DAPI). Cells were analyzed via a BD LSRFortessa (BD Biosciences, San Diego, Calif.) and FlowJo version 9.4.11 (Tree Star, Ashland, Oreg.).

Apoptosis Analysis of shRNA Transduced LSK Cells.

LSK cells were transduced overnight with control or gene-specific shRNAs and then cultured at 15,000 cells/well in non-tissue culture treated 96-well plates for 5-6 days in serum-free expansion medium (StemCell Technologies, Vancouver, British Columbia, Canada) with 10 ng/mL RM SCF, 20 ng/mL RM Tpo, 20 ng/mL RM IGF-2 (Peprotech, Rocky Hill, N.J.), 10 ng/mL RH FGF-1 (R&D Systems, Minneapolis, Minn.) and 10 ug/mL heparin (Sigma-Aldrich, St. Louis, Mo.). Cells were collected 5-6 days after plating and stained with the following antibodies: (B220, CD3, CD4, CD8, CD19, Gr-1, Ter119)-PerCP, Sca-1-PerCP-Cy5.5, c-Kit-APC-780. After staining for surface antigens, cells were labeled with Annexin V-FITC (BD Biosciences] and DAPI and then analyzed using a BD LSRFortessa (BD Biosciences, San Diego, Calif.) and FlowJo version 9.4.11 (Tree Star, Ashland, Oreg.).

Analysis of Total Blood Counts in Foxa3 Mice.

Peripheral blood was harvested from the retro-orbital plexus in heparinized capillary tubes and analyzed on a Forcyte instrument (Oxford Scientific, Oxford, Conn.).

Analysis of HSPC in Transplant Recipients and Foxa3 Mice.

Tibias, femurs, and pelvic bones were removed from mice and bone marrow isolated by crushing. Bone marrow was then lysed in red blood cell lysis buffer (Sigma-Aldrich, St. Louis, Mo.). Donor-derived HSC (LSK CD150+CD48), multipotent progenitors (MPP, LSK Flt3L⁺), common myeloid progenitors (CMP, Lineagec-Kit⁺Sca-1″FcR^{low}CD34⁺), common lymphoid progenitors (CLP, Lineagex-Kit^{Low}Sca-1^{Low}IL7R⁺), granulocyte-myeloid progenitors (GMP, Lineagex-Kit⁺Sca-1″FcR^{hi}s^{h}CD34⁺), and megakaryocyte-erythroid progenitors (MEP, Lineagec-Kit⁺Sca-1-FcR-CD34−) were visualized in transplant recipients by staining with the following antibodies: HSC ((B220, CD3, CD4, CD8, CD19, Gr-1, Ter119)-PerCP, Sca-1-PerCP-Cy5.5, c-Kit-APC-780, CD150-PE-Cy7, CD48-Alexa700, CD45.1-FITC, and CD45.2-v500); CMP/GMP/MEP (B220, CD3, CD4, CD8, CD19, Gr-1, Ter 19)-PerCP, Sca-1-PerCP-Cy5.5, c-Kit-APC-780, FcR II/III-Alexa700, CD34-FITC, CD45.1-APC, and CD45.2-v500); and CLP/MPP (B220, CD3, CD4, CD8, CD19, Gr-1, Ter119)-PerCP, Sca-1-PerCP-Cy5.5, c-Kit-APC-780, IL-7R-PE-Cy7, Flt3-APC, CD45.1-FITC, and CD45.2-v500).

HSPC were visualized in Foxa3^{-/} or Foxa3^{+/+-} mice as described above with the exclusion of CD45.1 and CD45.2. Cells were then analyzed using a BD LSRFortessa (BD Biosciences, San Diego, Calif.) and data analysis using FlowJo version 9.4.11 (Tree Star, Ashland, Oreg.). DAPI (Sigma-Aldrich) was used for dead cell exclusion.

Analysis of FOXA3 Binding Motifs in HSC Enhancers and Gene Targets.

Active and poised enhancers in LT-HSC, ST-HSC, MPP, and GMP were obtained from the enhancer compendium generated by Lara-Astiaso and colleagues (Lara-Astiaso et al., 2014). Poised enhancers refer to enhancers that, unlike active enhancers, do not drive gene expression in pluripotent cells, although they acquire such ability during differentiation. These enhancers were identified based on their histone modification signatures. For FOXA3 motif analysis, we downloaded the position weight matrix (PWM) of FOXA3 motif from the Cis-BP database (Weirauch et al., 2014). We used FIMO (a software tool for scanning DNA or protein sequences with motifs described as position-specific scoring matrices) to scan the enhancer sequences for the occurrence of FOXA3 binding motifs with a p-value threshold of 1×10⁵ (Grant et al., 2011). To predict the target genes of FOXA3 binding motif+ enhancers, we used the IM-PET software (He et al., 2014), which predicts enhancer-promoter interactions by integrating transcriptomic, epigenomic, and genomic sequence information. Histone modification and RNA-Seq data acquired by IM-PET were from (Cabezas-Wallscheid et al., 2014; Lara-Astiaso et al., 2014). The predicted targets of FOXA3 binding motif+ enhancers in LT-HSC were extracted for GSEA analysis.

Foxa3 Microarray.

Total RNA was isolated from 10,000 Foxa3^{+/+} or Foxa3^{-/-} HSC using the Qiagen RNeasy Micro Kit (Qiagen, Santa Clarita, Calif.). RNA was amplified by the NuGEN Ovation Pico WTA V2 system and labeled using the NuGEN Encore Biotin Module (NuGen, San Carlos, Calif.). Labeled targets were hybridized on the HT MG-430 PM plate array and processed utilizing the GeneTitan system (Affymetrix, Santa Clara, Calif.). Array data were quantile (i.e. cutpoints dividing the range of a probability distribution into contiguous intervals with equal probabilities, in other words a set of values of a variate that divide a frequency distribution into equal groups, each containing the same fraction of the total population) normalized and robust multi-array average summarized in Partek Genomics Suite 6.6 (Partek, St. Louis, Mo.). The complete dataset is deposited in the Gene Expression Omnibus (GSE63830.).

Analysis of Reactive Oxygen Species Content in Foxa3^{+/+} and Foxa3^{-/-} HSC. Foxa3^{+/+} or Foxa3^{-/-}

Whole Bone Marrow (WBM) was isolated, magnetically enriched for c-Kit⁺ cells, and then stained with Sca-1-PerCP-Cy5.5, c-Kit-APC-780, CD150-PE-Cy7, and CD48-Alexa700. Cells were then treated with vehicle or 500 μM tert-butyl Hydrogen Peroxide (TBHP). Three hours post-treatment, cells were stained with 5 μM 2′,7′-dichlorofluorescin diacetate (DCFDA) for 30 minutes on ice and then analyzed via a BD LSRFortessa (BD Biosciences, San Diego, Calif.) and FlowJo version 9.4.11 (Tree Star, Ashland, Oreg.). The peak excitation wavelength for oxidized DCF was 488 nm and emission was 525 nm.

Statistics.

Statistical significance for comparisons between two groups was assessed using two sample t-tests or Exact Wilcoxon Mann-Whitney tests, depending on the normality test based on the Shapiro-Wilk test. Measurements for each gene were normalized to their respective control and a one sample t-test was performed to assess if the mean of the normalized measurements is equal to one. These analyses were performed in SAS version 9.3. For limiting dilution analysis (LDA), parameters were estimated using a generalized linear model with a complementary log-log link. Chi-square (Pearson and Deviance) were used to assess the goodness-of-fit to the LDA model. Differences in the frequency of HSC between Foxa3^{+/+} and Foxa3^{-/-} mice were assessed by relying on the asymptotic normality of the maximum likelihood estimation. LDA were performed using L-Calc (Stem Cell Technologies, Vancouver, CA). Reported P-values are two-sided and considered statistically significant if <0.05, although P-values <0.1 are also noted in some instances as marginally significant.

2. HSPC Treated With shRNAs.

Identification of Candidate Genes in Functional Screens.

The following public databases of HSC gene expression were interrogated to prioritize 51 gene candidates for study: 1) Hematopoietic Fingerprints, 2) the Immunological Genome Project, and 3) StemSite (Chambers et al., 2007; Heng et al., 2008; McKinney-Freeman et al., 2012). Gene candidates were prioritized if their expression was enriched in adult HSC relative to downstream progeny or earlier stages of HSC ontogeny. qRT-PCR was used to interrogate the expression of each prioritized gene candidate in cells isolated from murine bone marrow (FIGS. 1A and 1B). We found that 44/51 GOI were expressed in lineage⁻ bone marrow hematopoietic cells, the majority of which were highly enriched for expression in Lineage⁻Sca-1⁺c-Kit⁺ (LSK) cells relative to downstream progeny (FIG. 1B).

To interrogate a role for GOI in HSC engraftment, we used shRNAs to disrupt their expression in LSK cells prior to transplantation into lethally irradiated mice. At least four miR-30 embedded shRNAs were designed to target each of the 44 GOI whose expression was validated in HSPC. shRNAs were cloned into a lentiviral vector downstream of an MSCV promoter and upstream of a PGK promoter driving the fluorescent reporter, mCherry (FIG. 1A). Each shRNA was transduced into LSK cells and tested for gene knockdown by qRT-PCR. Average transduction for these experiments was 76.7%±7 (FIG. 1C). At least two shRNA were identified that affected >75% transcript knockdown in LSK cells for 41/44 GOI (FIG. 1D, Table 2A and 2B). Thus, these genes were further screened.

We next conducted pilot studies to assess the feasibility of using highly purified HSC (LSK CD150⁺ CD48″ cells) in our screen. CD45.2⁺ HSC were transduced with control shRNAs and transplanted with an equal number of mock-transduced CD45.1⁺ HSC into CD45.1⁺/CD45.2⁺ recipients. These experiments showed high signal/noise incompatible with a robust screen. We determined that this high signal/noise resulted primarily from the technical difficulty of evenly distributing small cell numbers amongst mice in a cohort. Thus, we chose to utilize the more abundant LSK cell population for our screen. Although LSK cells are a mixture of HSPC, by following transplants >16 weeks we can still readily assess the effect of gene knockdown on stable HSC repopulation. Indeed, pilot studies in mice also revealed that HSC consistently transduced with a slightly higher frequency than LSK cells (FIG. 1E). Thus, HSC are robustly transduced in our system.

Functional Screen for Novel Regulators of HSC Engraftment.

CD45.2⁺“Test” LSK cells were transduced with individual shRNAs and then transplanted into ablated CD45.1⁺/CD45.2⁺ mice with an equal number of CD45.1⁺ mock transduced “Competitor” LSK cells (FIG. 2A). Cells were transplanted within 24-hours of their isolation and transduction; i.e. there was no extended ex vivo culture period as in previous functional screens of primary HSPC (Ali et al., 2009; Deneault et al., 2009; Hope et al., 2010). For each transplant, an aliquot of transduced cells was maintained in liquid culture and analyzed after 3-4 days for transduction efficiency. Average transduction for these experiments was 67.6%±8.5 (FIG. 2B). Recipient peripheral blood (PB) was analyzed for “Test” versus “Competitor” contribution for >16 weeks post-transplant. A total of 781 mice were transplanted.

a. Loss Of Function Hits.

Knockdown of 18 genes resulted in a loss of HSPC repopulating potential relative to control with two independent shRNAs in our initial screen (Arhgef5, Cadps2, Col4a2, Crispld1, Emcn, Foxa3, Glis2, Gng11, Gpr56, Myct1, Nbea, P2ry14, Rbpms, Sox4, Stat4, Trp53bpl, Trpc6, and Zbtb20) (FIG. 2C). Repopulation loss was apparent four weeks post-transplant and persisted for >16 weeks for GOI except Stat4 (FIG. 2C), where the loss of repopulation was most dramatic >16 weeks post-transplant. Knockdown of most of these genes did not affect the short-term (i.e. 14 days) maintenance of hematopoietic cells ex vivo. In contrast, knockdown of 20 GOI did not affect in vivo repopulating potential (FIG. 2D). To confirm stable gene knockdown in our system, mice transplanted with LSK cells transduced with Grb10-shRNAs, a non-Hit, were examined (FIG. 2E-G). Both Grb10-shRNAs effected >95% transcript loss in LSK cells (FIG. 2F). qRT-PCR analysis of CD45.2⁺ LSK cells isolated from mice transplanted 30 weeks prior with either control or Grb10-shRNA treated cells revealed persistent gene knockdown in these cells (FIG. 2G).

Knockdown of six GOI yielded a repopulating loss with ½ shRNAs tested (Eya2, Fstl1, Gucy1a3, Msrb2, Rbp1, and Myct1. FIG. 2C-D. In each case except Myct1 (discussed herein), this loss of repopulation was attributable to non-specific toxicity of the effecting shRNA (e.g. a third Gucy1a3-shRNA did not effect repopulation, FIG. 3F).

Confirmation of Loss of Function Screen Hits.

Eighteen (18) hits identified in our screen were retested to confirm their role as regulators of LSK cell in vivo repopulating activity. Here, to improve resolution, vector+ Test LSK cells (mCherry⁺ CD45.2⁺) were transplanted into ablated mice (FIG. 3A). Cells were sorted and transplanted 44 hours post-transduction along with an equal number of CD45.1+ mock transduced and mock-sorted “Competitor” LSK cells. A series of pilot studies revealed that a minimum of 40 hours was required post-transduction to visualize and isolate vector+ LSK cells by flow cytometry (FIG. 3B).

We also retested five genes that scored as non-Hits (Fstl1, Guc1a3, Rbp1, Smarca2, and Zfp521) (FIG. 2D). Smarca2 and Zfp251 were retested because transduction efficiency was low in our initial screen for these genes and/or their shRNAs did not yield a complete gene knockdown, resulting in a possible false negative (FIGS. 3C and D). Fstl1, Guc1la3 and Rbp1 were non-Hits whose two shRNAs yielded disparate outcomes in our initial screen, necessitating a more thorough analysis. A total of 527 mice were transplanted in these experiments.

Fifteen (15) “loss of function” Hits retested were confirmed for contributing to optimal HSPC repopulation (FIG. 3E). Repopulation loss was more dramatic in these experiments relative to our initial screen, likely due to greater resolution resulting from transplantation of vector+ cells. Three genes that initially scored as non-Hits were Hits when retested: Fstl1, Smarca2, and Zfp251. As mentioned, the transduction efficiencies for Smarca2 and Zfp251 were low in our initial screen (FIG. 3C), likely resulting in a false negative in those experiments. As both transduction and gene knockdown for Fstl1 were high in our initial screen (FIG. 2B), it appears that using transplantation of vector+ cells clearly shows a repopulating loss with both Fstl1 shRNAs. Alternatively, the prolonged culture in these experiments might exact additional stress on the cells, resulting in a loss of in vivo repopulation not apparent in our original screen. Six initial Hits did not effect repopulating potential when retested: Col4a2, Gng11, Rbpms, Trp53bpl, Trpc6, and Zbtb20 (FIG. 3F). As one Zbtb20-shRNA was tested in our initial screen, two additional Zbtb20-shRNAs were tested in our confirmation experiments (FIG. 3F).

The original Zbtb20-shRNA mediated a loss of repopulation, suggesting that this shRNA likely had off-target effects. Once again, Stat4 was a Hit that displayed a significant increase in repopulating loss between four and >16 weeks post-transplant (FIG. 3E), suggesting that Stat4 regulates the long-term repopulating potential of HSC, rather than their early engraftment. The distribution of T, B, or myeloid cells in the mCherry⁺ CD45.2⁺ compartment of recipients was significantly perturbed in recipients of Cadps2 and Foxa3-shRNA treated cells (FIG. 3G). Loss of Cadps2 resulted in a significant expansion of B cells and a concomitant loss of T cells, suggesting that lymphoid progenitor function might be perturbed. Loss of Foxa3 perturbed the myeloid compartment (FIG. 3G).

In sum, via our two-pronged screening approach, we rigorously identified 15 genes individually contributing to LSK cell in vivo repopulating activity: Arhgef5, Cadps2, Crispld1, Emcn, Foxa3, Fstl1, Glis2, Gpr56, Myct1, Nbea, P2ry14, Smarca2, Sox4, Stat4, and Zfp251 (FIG. 3E). These GOI regulate a diverse array of cellular processes, including epigenetics, adhesion and migration, vesicle trafficking and cell surface receptor turnover, and the extracellular matrix.

Interrogation of the Cellular Mechanism of Gene Loss on HSPC Repopulating Potential.

To illuminate the cellular mechanisms of gene knockdown on HSPC, LSK cells transduced with control or gene-specific shRNAs were assayed for colony forming unit (CFU) potential, cell cycle, and apoptosis (FIGS. 5A and B). We also examined CD45.2⁺chimerism in the bone marrow of recipients of gene-deficient CD45.2+ LSK cells >16 weeks post-transplant (FIG. 5C).

LSK cells lacking Nbea and Glis2 displayed an increase in CFU-GEMM potential (P=0.046 and 0.07, respectively] (FIG. 5A). This correlated with a loss of CD45.2+ chimerism downstream of HSC and/or MPPs in recipients of LSK cells deficient in these genes (FIG. 5C). These data suggest a block in differentiation at the HSC or MPP stage, resulting in an accumulation of CFU-GEMM. G//s2-deficient LSK cells also displayed elevated apoptosis ex vivo (P=0.08) (FIG. 5B), suggesting that this block in differentiation exists in concert with reduced progenitor survival downstream of HSC and MPPs.

Knockdown of Stat4, Zfp251, and Foxa3 also resulted in an enhanced loss of CD45.2+ chimerism downstream of HSC in transplanted mice (FIG. 5C). Knockdown of Zfp251 in LSK cells ex vivo resulted in a slight expansion of CFU-G/M/GM at the expense of CFU-GEMM (P=0.08) and about a 50% loss of apoptotic cells, although CFU-G/M/GM expansion and loss of apoptosis did not score as statistically significant here (FIGS. 5A and B). These data suggest that CFU-GEMM lacking Zfp251 differentiate rapidly to committed progenitors that display enhanced survival ex vivo, but fail to establish robust chimerism in vivo.

Arhgef5 and Emcn knockdown caused a significant loss in total CFU from LSK cells (P=0.027; P=0.035, respectively), while Fstl1 knockdown resulted in a dramatic, but marginally significant, loss in CFU (P=0.096) (FIG. 5A). This correlated with a loss of CD45.2+ chimerism across bone marrow compartments in recipients of LSK cells deficient in these genes except for Fstl1, for whom bone marrow chimerism was not determined (FIG. 5C). As Emcn-deficient LSK cells did not display significant perturbations in cell cycle or apoptosis ex vivo, loss of in vivo repopulating activity may result from perturbed niche interactions post-transplant effecting survival, differentiation, or proliferation.

However, Arhgef5-deficient LSK cells displayed about a 40% expansion of cells in G₁ ex vivo, relative to control (P=0.089), which was commiserate with a modest reduction of cells in both G₀ and G₂SM (P >0.05 for both). Thus, perturbations in cell cycle progression may contribute to the repopulating defect of Arhgef5-deficient LSK cells. In addition to a dramatic loss in total-CFU, Fstl1-deficient cells displayed a rapid loss of the LSK cell surface phenotype during culture (P=0.079), suggesting accelerated differentiation commiserate with a loss of stem and progenitor cell potential.

Knockdown of Cadps2 in CD45.2+ LSK cells also resulted in a loss of CD45.2+ chimerism across bone marrow compartments post-transplant (FIG. 5C). This correlated with perturbations in the frequency of select CFU: a marginally significant, albeit modest, loss of CFU-E (P=0.057) and a marginally significant increase in CFU-G/MIGM (P=0.06) was apparent after knockdown of this gene (FIG. 5A).

Foxa3^{-/-} HSC Displays Reduced In Vitro and In Vivo Hematopoietic Potential.

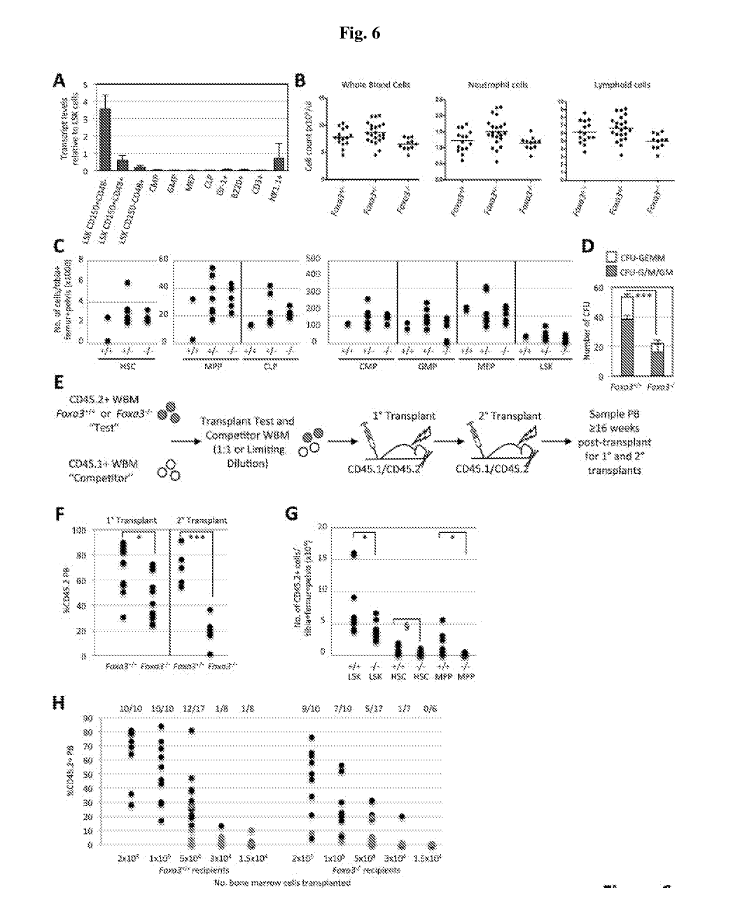

Our screen identified Foxa3 as a putative novel regulator of LSK cell in vivo repopulating activity (FIGS. 2C and 3E). As Foxa genes have not been implicated in hematopoiesis, we decided to explore Foxa3's putative role in HSC further by examining Foxa3^{-/-} mice. Although Foxa3 is selectively expressed by HSC in bone marrow (FIG. 6A), Foxa3^{-/-} mice display normal PB counts and bone marrow HSPC frequencies (FIG. 6B-C). Foxa3^{-/-} HSC generated fewer CFU than Foxa3^{+/+} HSC, suggesting a loss of functional HSC, which could result from fewer absolute numbers of functional HSC or a failure of HSC activation in culture (FIG. 6D). Surprisingly, Foxa3^{-/-} LSK cells showed no loss of CFU potential relative elative to Foxa3^{+/+} LSK cells. As LSK cells are a mix of HSC and progenitors, these data suggest that progenitors downstream of Foxa3^{+/+} HSC retain CFU potential.

CD45.2⁺ Foxa3^{-/-} or Foxa3^{+/+} WBM was transplanted with an equal amount of CD45.1⁺ WBM into ablated CD45.1⁺/CD45.2⁺ recipients (FIG. 6E-FJ). A significant loss in CD45.2⁺ PB reconstitution was apparent in Foxa3^{-/-} recipients relative to Foxa3^{+/+} recipients 20 weeks post-transplant (FIG. 6F). There was no obvious skewing in the reconstitution of specific PB lineages in Foxa3^{-/-} recipients. Although Foxa3^{-/-} cells contributed less than Foxa3^{+/+} cells to recipient LSK, HSC, and MPP compartments (FIG. 6G), Foxa3^{-/-} chimerism in downstream progenitor compartments was unperturbed. When CD45.2⁺ WBM from primary recipients was transplanted into secondary recipients, Foxa3^{-/-} WBM displayed an even more pronounced repopulating defect than in primary transplants (FIG. 6E-F), suggesting that Foxa3^{-/-} HSC do not self-renew efficiently. Foxa3^{-/-} WBM contained significantly fewer repopulating HSC, relative to control, when transplanted at limiting dilutions (FIG. 6H, Table 3, P=0.0046).

In sum, Foxa3^{-/-} HSC are defective in CFU potential, primary and secondary in vivo repopulation, and the ability to efficiently contribute to the most primitive HSPC WBM compartments (HSC and MPP). These data suggest that Foxa3^{-/-} bone marrow contains fewer repopulating cells than Foxa3^{+/+} marrow and that self-renewal may be compromised in Foxa3^{-/-} HSC.

b. Gain of Function Hits: Loss of Gprasp2 and Armcx1 Promotes HSPC Repopulation.

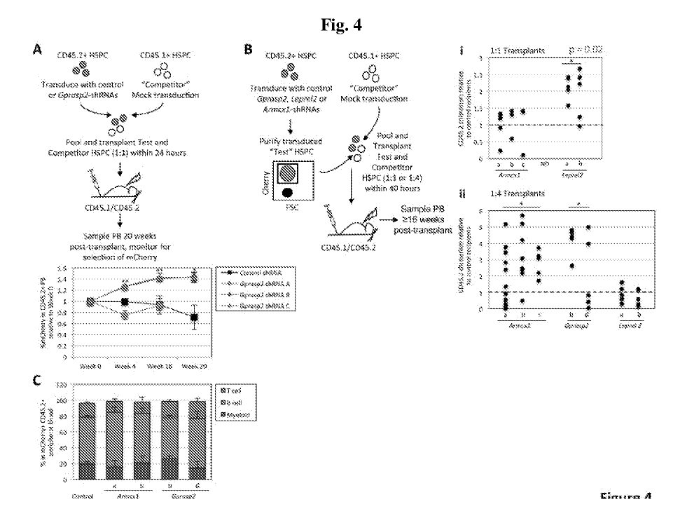

Mouse recipients of mouse Armcx1 and Gprasp2-shRNA treated mouse HSCs displayed enhanced CD45.2+ chimerism in HSPC compartments, correlating with enhanced PB chimerism (FIGS. 4B and 5C). In fact, loss of Gprasp2 appeared to favor LSK cell in vivo repopulating activity in this study. Here, mCherry⁺ CD45.2⁺ PB was selected for over a time period in 17/20 recipients of Gprasp2-shRNAs transduced LSK cells compared to 2/9 recipients of control cells (FIG. 4A). LSK cells treated with Gprasp2-shRNAs displayed significantly enhanced survival ex vivo and a two-fold expansion of cells in Go, commiserate with a loss of cells in G₂SM (P=0.002). Thus, enhanced survival and a slowing-growing phenotype may contribute to enhanced in vivo repopulation here, as has been seen in Runx1 mutants whose HSC also display a repopulating advantage (Cai et al., 2015).

Similarly, loss of Armcx1 also appeared to enhance HSPC repopulation (FIG. 4Bi). However, in contrast, knockdown of Armcx1 in LSK cells ex vivo had no significant effect on CFUs, cell cycle, or apoptosis (FIGS. 5A and B), suggesting that enhanced repopulation after knockdown of Armcx1 may result from specific in vivo interactions.

mCherry⁺ cells were transplanted in these experiments, so it was not possible to monitor for mCherry selection. However, although not statistically significant, 7/11 recipients of Armcx1-shRNA transduced Test HSPC showed moderately enhanced chimerism >16 weeks post-transplant relative to controls (FIG. 4Bi).

To rigorously assess whether loss of Armcx1 or Gprasp2 enhanced LSK cell in vivo repopulating activity. mCherry⁺ CD45.2⁺“Test” LSK cells (transduced with either gene-specific or control shRNAs) were transplanted 1:4 with CD45.1⁺ mock transduced and mock-sorted “Competitor” LSK cells, thus putting the Test cells at a significant repopulating disadvantage relative to Competitor.

Loss of Armcx1 and Gprasp2 enhanced the repopulating potential of Test LSK cells in the majority of transplanted mice (FIG. 4Bii). This result was true for multiple independent shRNAs tested for each gene. Loss of Armcx1 and Gprasp2 did not appear to perturb any specific hematopoietic PB lineages (FIG. 4C). Loss of the gene Leprel2 also appeared to enhance repopulation in both our initial screen and after retesting (P=0.02) (FIG. 4Bi). However, when Leprel2 was reexamined in a 1:4 Test versus Competitor transplant, enhanced repopulation was no longer apparent (FIG. 4Bii).

In sum, loss of Gprasp2 and Armcx1 enhanced LSK cell repopulating activity, suggesting that these genes may negatively impact HSPC engraftment. Surprisingly, Gprasp2 and Armcx1 belong to the same family of G-protein Coupled Receptor Associated Sorting Proteins (GASP) (Abu-Helo and Simonin, 2010), thus implicating genes in this gene family for negative regulation of HSPC repopulating potential.

TABLE 3
Foxa3^{-/-} And Foxa3^{+/+} WBM Predicted Repopulating Cell Frequency.

| | HSC Frequency | | | Fit to LD model | | | |
|---|---|---|---|---|---|---|---|
| | (95% Confidence | | KO vs. WT | Chi-square | | Chi-square | |
| | Interval) (×10⁻⁶) | | comparison | (Pearson) | | (Deviance) | |
| Foxa3^{+/+} | 21.2 | (14.2-31.2) | p = 0.0046 | 6.4 | (p = 0.17) | 8.46 | (p = 0.08) |
| Foxa3^{-/-} | 9 | (5.8-13.9) | — | 2.33 | (p = 0.67) | 3.19 | (p = 0.53) |

FOXA3 Binding Motifs are Enriched in LT-HSC Enhancers and Target Proliferative and Stress Pathways.

Because Foxa3 was implicated in regulating HSC function, then it was tested whether FOXA3 binding motif is significantly enriched in active and/or poised enhancers in long term HSC (LT-HSC) and progeny (Lara-Astiaso et al., 2014). We found the FOXA3 binding motif enriched in enhancers active in LT-HSC but poised in downstream populations (Table 4), suggesting that Foxa3 likely functions at the level of the LT-HSC, which agrees with our finding that Foxa3 is most highly expressed in HSC (FIG. 6A). These enhancers were not enriched for any other known transcription factor binding motifs, suggesting that Foxa3 either acts alone at these sites or cooperates with regulators whose motifs have not yet been defined.

TABLE 4
FOXA3 Binding Motif Enrichment In Enhancers Active
In LT-HSC And Poised In Other HSPC Compartments.
Enrichment of FOXA3 binding motif in HSPC enhancers

| Enhancer | | #FOXA3 binding motif + | |
|---|---|---|---|
| Type | #Enhancers | enhancers | p-value |
| Active in | 2783 | 594 | 0.00027 |
| LT-HSC, poised | | | |
| in others | | | |

We next used IM-PET (Integrated Method for Predicting Enhancer Targets) to identify the promoters likely targeted by these FOXA3 binding motif+ enhancers (Table 5) (He et al., 2014).

TABLE 5
Predicted Gene Targets of Foxa3 motif+ active LT-HSC enhancers.

| | | | | | Gene | | |
|---|---|---|---|---|---|---|---|
| Enhancer | | | Promoter | | | | Expression |
| Chr | Start | End | Chr | Position | ID | Name | (FPKM) |
| chr7 | 52079823 | 52081823 | chr7 | 52715254 | ENSMUST00000094434.4 | Ftl1 | 747.292 |
| chr19 | 4136521 | 4138521 | chr19 | 4154606 | ENSMUST00000061086.8 | Ptprcap | 559.718 |
| chr7 | 134624783 | 134626783 | chr7 | 133942768 | ENSMUST00000032934.5 | Aldoa | 486.681 |
| chr10 | 127517457 | 127519457 | chr10 | 128063562 | ENSMUST00000026420.5 | Rps26 | 414.077 |
| chr7 | 133271143 | 133273143 | chr7 | 133848330 | ENSMUST00000032949.7 | Coro1a | 389.515 |
| chr1 | 162790541 | 162792541 | chr1 | 163181350 | ENSMUST00000071718.5 | Prdx6 | 252.116 |
| chr14 | 55035149 | 55037149 | chr14 | 55045746 | ENSMUST00000010550.7 | Mrpl52 | 249.859 |
| chrX | 34634483 | 34636483 | chrX | 34625397 | ENSMUST00000115231.3 | Rpl39 | 223.308 |
| chr15 | 100461395 | 100463395 | chr15 | 100499933 | ENSMUST00000100198.2 | Bin2 | 213.072 |
| chr6 | 125428238 | 125430238 | chr6 | 125444809 | ENSMUST00000032492.8 | Cd9 | 169.745 |
| chr16 | 91358906 | 91360906 | chr16 | 91114844 | ENSMUSG00000081176.1 | H3f3a-ps2 | 169.34 |
| chr2 | 117626062 | 117628062 | chr2 | 118305447 | ENSMUST00000009693.8 | Srp14 | 167.374 |
| chr2 | 118314465 | 118316465 | chr2 | 118305447 | ENSMUST00000009693.8 | Srp14 | 167.374 |
| chr14 | 27735759 | 27737759 | chr14 | 27457661 | ENSMUST00000169243.1 | Arf4 | 163.419 |
| chr17 | 34345356 | 34347356 | chr17 | 34334666 | ENSMUST00000173441.1 | Psmb8 | 158.961 |
| chr4 | 137154503 | 137156503 | chr4 | 136913635 | ENSMUST00000051477.6 | Cdc42 | 156.819 |
| chr13 | 23655255 | 23657255 | chr13 | 23663265 | ENSMUST00000079251.5 | Hist1h2bg | 146.602 |
| chr7 | 35056737 | 35058737 | chr7 | 35015300 | ENSMUST00000038027.4 | Gpi1 | 140.204 |
| chr10 | 127517457 | 127519457 | chr10 | 128360580 | ENSMUST00000026405.3 | Bloc1s1 | 139.861 |
| chr9 | 65395253 | 65397253 | chr9 | 64585430 | ENSMUST00000169058.1 | Rab11a | 139.162 |
| chr11 | 52181889 | 52183889 | chr11 | 52187799 | ENSMUST00000109065.1 | Vdac1 | 138.309 |
| chr11 | 118285234 | 118287234 | chr11 | 118263406 | ENSMUST00000043722.3 | Lgals3bp | 129.102 |
| chr2 | 84208651 | 84210651 | chr2 | 84877391 | ENSMUST00000077798.6 | Ssrp1 | 128.321 |
| chr10 | 126563534 | 126565534 | chr10 | 126500660 | ENSMUST00000133115.1 | Cdk4 | 126.187 |
| chr1 | 95380382 | 95382382 | chr1 | 95375570 | ENSMUST00000172165.1 | 2-Sep | 125.251 |
| chr4 | 129494590 | 129496590 | chr4 | 129516395 | ENSMUST00000141754.1 | Ptp4a2 | 123.202 |
| chr4 | 129602909 | 129604909 | chr4 | 129516395 | ENSMUST00000141754.1 | Ptp4a2 | 123.202 |
| chr3 | 89686643 | 89688643 | chr3 | 89883587 | ENSMUST00000131354.1 | Tpm3 | 119.81 |
| chr12 | 86728378 | 86730378 | chr12 | 86815861 | ENSMUST00000134311.1 | Fos | 118.571 |
| chr13 | 101412223 | 101414223 | chr13 | 101421298 | ENSMUST00000084721.6 | Taf9 | 105.234 |
| chr10 | 127517457 | 127519457 | chr10 | 127496010 | ENSMUST00000052798.7 | Ptges3 | 95.4404 |
| chr15 | 36429054 | 36431054 | chr15 | 36532101 | ENSMUST00000156793.1 | Pabpc1 | 95.0908 |
| chr9 | 59326379 | 59328379 | chr9 | 59504383 | ENSMUST00000163694.1 | Pkm2 | 91.2134 |
| chr18 | 35093288 | 35095288 | chr18 | 35114011 | ENSMUST00000025217.8 | Hspa9 | 89.3101 |
| chr10 | 127517457 | 127519457 | chr10 | 127759515 | ENSMUST00000026446.2 | Cnpy2 | 86.5671 |
| chr10 | 127517457 | 127519457 | chr10 | 127525608 | ENSMUST00000126751.1 | Atp5b | 82.1594 |
| chr12 | 33853153 | 33855153 | chr12 | 33639049 | ENSMUST00000020885.6 | Sypl | 80.8468 |
| chr18 | 82699936 | 82701936 | chr18 | 82693439 | ENSMUST00000166332.1 | Rpl21-ps8 | 78.2873 |
| chr11 | 29687141 | 29689141 | chr11 | 29446671 | ENSMUST00000147782.1 | Rps27a | 77.6512 |
| chr5 | 122716393 | 122718393 | chr5 | 122845823 | ENSMUST00000031421.5 | Arpc3 | 76.4993 |
| chr19 | 29457752 | 29459752 | chr19 | 29436460 | ENSMUST00000143467.1 | 5033414D02Rik | 76.3381 |
| chr11 | 121339728 | 121341728 | chr11 | 120624108 | ENSMUSG00000089637.2 | Hmga1- | 75.286 |
| | | | | | | rs1 | |
| chr4 | 150427277 | 150429277 | chr4 | 150432062 | ENSMUST00000030797.3 | Vamp3 | 73.6629 |
| chr16 | 50069074 | 50071074 | chr16 | 49855821 | ENSMUST00000073477.7 | Cd47 | 73.1746 |
| chr8 | 26121545 | 26123545 | chr8 | 26127683 | ENSMUST00000033961.5 | Tm2d2 | 72.7007 |
| chr8 | 26154544 | 26156544 | chr8 | 26127683 | ENSMUST00000033961.5 | Tm2d2 | 72.7007 |
| chr10 | 126563534 | 126565534 | chr10 | 126507317 | ENSMUST00000060991.4 | Tspan31 | 71.1209 |
| chr4 | 102204209 | 102206209 | chr4 | 102242700 | ENSMUST00000097950.2 | Pde4b | 68.4863 |
| chr6 | 125428238 | 125430238 | chr6 | 124662354 | ENSMUST00000004375.9 | Phb2 | 66.5208 |
| chr10 | 127517457 | 127519457 | chr10 | 127523286 | ENSMUST00000124993.1 | Atp5b | 66.4817 |
| chr4 | 102204209 | 102206209 | chr4 | 101927608 | ENSMUST00000106908.2 | Pde4b | 66.2269 |
| chr5 | 117810007 | 117812007 | chr5 | 117083471 | ENSMUSG00000091578.1 | Gstm2- | 63.831 |
| | | | | | | ps1 | |
| chr10 | 127517457 | 127519457 | chr10 | 126959491 | ENSMUST00000026470.3 | Shmt2 | 63.7799 |
| chr13 | 23655255 | 23657255 | chr13 | 23663089 | ENSMUSG00000069272.4 | Hist1h2ae | 61.1146 |
| chr1 | 163003668 | 163005668 | chr1 | 162965315 | ENSMUST00000162163.1 | Gas5 | 61.0581 |
| chr2 | 155513435 | 155515435 | chr2 | 155555211 | ENSMUST00000040833.4 | Edem2 | 60.2875 |
| chr12 | 78295247 | 78297247 | chr12 | 77866525 | ENSMUST00000041262.7 | Churc1 | 60.2591 |
| chr11 | 100843840 | 100845840 | chr11 | 100832201 | ENSMUST00000060792.5 | Ptrf | 60.1656 |
| chr10 | 80080382 | 80082382 | chr10 | 80317954 | ENSMUST00000035775.8 | Lsm7 | 59.5431 |
| chr19 | 4136521 | 4138521 | chr19 | 4121575 | ENSMUST00000025767.7 | Aip | 59.215 |
| chr11 | 72769965 | 72771965 | chr11 | 72774671 | ENSMUST00000021142.7 | Atp2a3 | 57.7872 |
| chr17 | 34345356 | 34347356 | chr17 | 34341512 | ENSMUST00000127543.1 | Tap2 | 57.5822 |
| chr3 | 121993571 | 121995571 | chr3 | 121977332 | ENSMUST00000035776.8 | Dnttip2 | 56.8503 |
| chr1 | 146031714 | 146033714 | chr1 | 145851291 | ENSMUST00000127206.1 | Rgs2 | 56.4282 |
| chr7 | 52079823 | 52081823 | chr7 | 52083856 | ENSMUST00000171304.1 | Nup62 | 53.7819 |
| chr15 | 97556610 | 97558610 | chr15 | 97794710 | ENSMUST00000064200.6 | Tmem106c | 53.6799 |
| chr4 | 131753294 | 131755294 | chr4 | 131768170 | ENSMUST00000040654.7 | Ythdf2 | 53.5868 |
| chr14 | 78235494 | 78237494 | chr14 | 78274724 | ENSMUST00000022590.3 | Dnajc15 | 53.281 |
| chr7 | 133271143 | 133273143 | chr7 | 133839480 | ENSMUST00000052145.5 | Bola2 | 53.0679 |
| chr3 | 94856898 | 94858898 | chr3 | 94846536 | ENSMUST00000107237.1 | Psmd4 | 52.5335 |
| chr15 | 61995177 | 61997177 | chr15 | 61816946 | ENSMUST00000161976.1 | Myc | 51.4547 |
| chr1 | 163003668 | 163005668 | chr1 | 162965294 | ENSMUST00000161380.1 | Gas5 | 50.7121 |
| chr18 | 82699936 | 82701936 | chr18 | 82693413 | ENSMUST00000130005.1 | Rpl21-ps8 | 49.9488 |
| chr10 | 70800759 | 70802759 | chr10 | 70807702 | ENSMUST00000045887.8 | Cisd1 | 49.6574 |
| chr5 | 114282886 | 114284886 | chr5 | 114280442 | ENSMUST00000072194.4 | Selplg | 49.6225 |
| chr4 | 135414667 | 135416667 | chr4 | 135502363 | ENSMUST00000030432.7 | Hmgcl | 49.5445 |
| chr16 | 14160427 | 14162427 | chr16 | 14317468 | ENSMUST00000023357.7 | 0610037P05Rik | 47.9913 |
| chr11 | 75222724 | 75224724 | chr11 | 75231498 | ENSMUST00000168902.1 | Serpinf1 | 47.8969 |
| chr11 | 75378410 | 75380410 | chr11 | 75231498 | ENSMUST00000168902.1 | Serpinf1 | 47.8969 |
| chr8 | 87506801 | 87508801 | chr8 | 87493510 | ENSMUST00000005292.8 | Prdx2 | 47.0819 |
| chr9 | 107907557 | 107909557 | chr9 | 107673333 | ENSMUST00000035199.5 | Rbm5 | 46.9892 |
| chr17 | 26050333 | 26052333 | chr17 | 25929229 | ENSMUST00000072735.7 | Fam173a | 46.7898 |
| chr1 | 169268392 | 169270392 | chr1 | 169280122 | ENSMUST00000028004.9 | Aldh9a1 | 46.4586 |
| chr10 | 127517457 | 127519457 | chr10 | 127472402 | ENSMUST00000073868.7 | Naca | 46.1597 |
| chr8 | 87506801 | 87508801 | chr8 | 87604872 | ENSMUST00000148592.1 | BC056474 | 46.0604 |
| chr9 | 107907557 | 107909557 | chr9 | 107537673 | ENSMUST00000055704.6 | Gnai2 | 44.8509 |
| chr3 | 96077259 | 96079259 | chr3 | 96361883 | ENSMUST00000049093.7 | Txnip | 44.8287 |
| chr7 | 134657172 | 134659172 | chr7 | 135049251 | ENSMUST00000124533.1 | Bckdk | 44.8205 |
| chr4 | 135414667 | 135416667 | chr4 | 135429765 | ENSMUST00000030436.5 | Pnrc2 | 43.5124 |
| chr11 | 106299577 | 106301577 | chr11 | 106020723 | ENSMUST00000138094.1 | Limd2 | 42.8991 |
| chr12 | 86728378 | 86730378 | chr12 | 86715667 | ENSMUST00000040766.7 | Tmed10 | 42.494 |
| chr13 | 34345677 | 34347677 | chr13 | 34254833 | ENSMUST00000147632.1 | Psmg4 | 42.0083 |
| chr11 | 90217013 | 90219013 | chr11 | 90249521 | ENSMUST00000107881.1 | Hlf | 41.8407 |
| chr7 | 52079823 | 52081823 | chr7 | 52359192 | ENSMUST00000003512.7 | Fcgrt | 41.7388 |
| chr12 | 84935064 | 84937064 | chr12 | 84973184 | ENSMUST00000048155.8 | Rbm25 | 41.3059 |
| chr2 | 155513435 | 155515435 | chr2 | 155576853 | ENSMUST00000029140.5 | Procr | 41.2525 |
| chr2 | 153608621 | 153610621 | chr2 | 153567338 | ENSMUST00000123795.1 | Mapre1 | 40.9239 |
| chr10 | 80080382 | 80082382 | chr10 | 80065627 | ENSMUST00000079883.4 | Scamp4 | 40.8637 |
| chr10 | 93511891 | 93513891 | chr10 | 93661700 | ENSMUST00000020209.9 | Ndufa12 | 40.4148 |
| chr3 | 27228313 | 27230313 | chr3 | 27081887 | ENSMUST00000046515.8 | Nceh1 | 40.2301 |
| chr3 | 32326102 | 32328102 | chr3 | 32635912 | ENSMUST00000127477.1 | Ndufb5 | 39.9364 |
| chr5 | 134848944 | 134850944 | chr5 | 134755735 | ENSMUST00000172904.1 | Gtf2i | 39.6902 |
| chr2 | 35017577 | 35019577 | chr2 | 35056640 | ENSMUST00000028238.8 | Rab14 | 39.6781 |
| chr2 | 35046233 | 35048233 | chr2 | 35056640 | ENSMUST00000028238.8 | Rab14 | 39.6781 |
| chr1 | 36695504 | 36697504 | chr1 | 36748420 | ENSMUST00000168827.1 | Cox5b | 38.623 |
| chr9 | 21431017 | 21433017 | chr9 | 21142288 | ENSMUST00000034697.6 | Slc44a2 | 37.8794 |
| chr5 | 100463781 | 100465781 | chr5 | 100468241 | ENSMUST00000128187.1 | Hnrpdl | 37.1587 |
| chr13 | 34345677 | 34347677 | chr13 | 34254833 | ENSMUST00000124996.1 | Psmg4 | 37.0945 |
| chr18 | 35093288 | 35095288 | chr18 | 35278542 | ENSMUST00000042345.6 | Ctnna1 | 37.0445 |
| chr10 | 119620496 | 119622496 | chr10 | 119664126 | ENSMUST00000020444.8 | Llph | 35.6929 |
| chr7 | 111060108 | 111062108 | chr7 | 110962510 | ENSMUST00000098192.3 | Hbb-b2 | 35.5272 |
| chr17 | 26050333 | 26052333 | chr17 | 26012445 | ENSMUST00000026827.8 | 0610011F06Rik | 35.2006 |
| chr17 | 37129242 | 37131242 | chr17 | 37407165 | ENSMUST00000038580.6 | H2-M3 | 35.096 |
| chr4 | 154456986 | 154458986 | chr4 | 155135202 | ENSMUST00000030903.5 | Atad3a | 34.9598 |
| chr9 | 55999010 | 56001010 | chr9 | 56008596 | ENSMUST00000098723.2 | Tspan3 | 34.8509 |
| chr7 | 133271143 | 133273143 | chr7 | 134119863 | ENSMUST00000032920.3 | Cdipt | 34.7123 |
| chr10 | 127517457 | 127519457 | chr10 | 128258647 | ENSMUST00000116228.1 | Ormdl2 | 34.5156 |
| chr17 | 36271246 | 36273246 | chr17 | 36266370 | ENSMUST00000172968.1 | H2-T9 | 33.944 |
| chr11 | 100706701 | 100708701 | chr11 | 100800854 | ENSMUST00000127638.1 | Stat3 | 33.9368 |
| chr16 | 4719688 | 4721688 | chr16 | 4726363 | ENSMUST00000004172.8 | Hmox2 | 33.5265 |
| chr1 | 146031714 | 146033714 | chr1 | 145851265 | ENSMUST00000027606.3 | Rgs2 | 33.2077 |
| chr9 | 57496848 | 57498848 | chr9 | 57493460 | ENSMUST00000034863.6 | Csk | 32.9256 |
| chr15 | 36429054 | 36431054 | chr15 | 36426546 | ENSMUST00000057486.7 | Ankrd46 | 32.3146 |
| chr17 | 80875858 | 80877858 | chr17 | 80606629 | ENSMUSG00000024097.9 | Srsf7 | 32.2547 |
| chr9 | 57496848 | 57498848 | chr9 | 57613667 | ENSMUST00000065330.6 | Clk3 | 31.7278 |
| chr4 | 131753294 | 131755294 | chr4 | 131909601 | ENSMUST00000152943.1 | Snhg3 | 31.5411 |
| chr4 | 132127950 | 132129950 | chr4 | 131909601 | ENSMUST00000152943.1 | Snhg3 | 31.5411 |
| chr8 | 123238735 | 123240735 | chr8 | 122364479 | ENSMUST00000034285.6 | Cotl1 | 31.3453 |
| chr19 | 4136521 | 4138521 | chr19 | 4811634 | ENSMUST00000006625.6 | Rbm14 | 31.0976 |
| chr14 | 79713112 | 79715112 | chr14 | 79987498 | ENSMUST00000054908.8 | Sugt1 | 30.5844 |
| chr8 | 87506801 | 87508801 | chr8 | 87549177 | ENSMUST00000064314.8 | Asna1 | 30.5508 |
| chr7 | 134624783 | 134626783 | chr7 | 134613885 | ENSMUST00000106292.1 | Prr14 | 30.423 |
| chr7 | 134657172 | 134659172 | chr7 | 134613885 | ENSMUST00000106292.1 | Prr14 | 30.423 |
| chr11 | 72769965 | 72771965 | chr11 | 72812605 | ENSMUST00000021141.7 | P2rx1 | 30.0016 |
| chr8 | 87506801 | 87508801 | chr8 | 87604639 | ENSMUST00000079764.7 | BC056474 | 29.7689 |
| chr8 | 47708070 | 47710070 | chr8 | 47702803 | ENSMUST00000093517.5 | Casp3 | 29.5639 |
| chr9 | 63596837 | 63598837 | chr9 | 63450462 | ENSMUST00000041551.7 | Aagab | 29.3424 |
| chr9 | 114302873 | 114304873 | chr9 | 114310194 | ENSMUST00000063042.9 | Glb1 | 29.3269 |
| chr12 | 77464719 | 77466719 | chr12 | 78340052 | ENSMUST00000062804.7 | Fut8 | 29.299 |
| chr17 | 31978023 | 31980023 | chr17 | 31992737 | ENSMUST00000024839.4 | Sik1 | 29.2121 |
| chr9 | 58117801 | 58119801 | chr9 | 57758793 | ENSMUST00000163329.1 | Ubl7 | 29.1849 |
| chr10 | 88093529 | 88095529 | chr10 | 88193813 | ENSMUST00000116234.2 | Arl1 | 29.1034 |
| chr14 | 122272328 | 122274328 | chr14 | 122277828 | ENSMUST00000039803.5 | Ubac2 | 28.9842 |
| chr12 | 70617972 | 70619972 | chr12 | 70260282 | ENSMUST00000174924.1 | AC099934.1 | 28.9509 |
| chr12 | 70617972 | 70619972 | chr12 | 70462468 | ENSMUST00000175032.1 | AC157822.1 | 28.9509 |
| chr3 | 89686643 | 89688643 | chr3 | 89930457 | ENSMUSG00000064930.1 | SNORA41 | 28.6569 |
| chr9 | 96512363 | 96514363 | chr9 | 96653250 | ENSMUST00000152594.1 | Zbtb38 | 28.4454 |
| chr6 | 38485720 | 38487720 | chr6 | 38509215 | ENSMUST00000162530.1 | Luc7l2 | 27.7907 |
| chr18 | 69775855 | 69777855 | chr18 | 69753071 | ENSMUST00000114978.2 | Tcf4 | 27.6069 |
| chr10 | 96060592 | 96062592 | chr10 | 96079635 | ENSMUST00000038377.7 | Btg1 | 27.1549 |
| chr2 | 32725469 | 32727469 | chr2 | 32502218 | ENSMUST00000165273.1 | Eng | 27.0826 |
| chr9 | 45817658 | 45819658 | chr9 | 45792954 | ENSMUST00000172450.1 | Pafah1b2 | 27.0713 |
| chr4 | 114655661 | 114657661 | chr4 | 114659846 | ENSMUST00000030491.8 | Cmpk1 | 26.3761 |
| chr11 | 52181889 | 52183889 | chr11 | 51814264 | ENSMUST00000109086.1 | Ube2b | 26.1096 |
| chr13 | 98142062 | 98144062 | chr13 | 97440891 | ENSMUST00000022176.8 | Hmgcr | 25.9867 |
| chr11 | 75351840 | 75353840 | chr11 | 75300322 | ENSMUST00000102510.1 | Prpf8 | 25.9561 |
| chr11 | 84944311 | 84946311 | chr11 | 84729868 | ENSMUST00000103195.4 | Znhit3 | 25.8873 |
| chr14 | 78235494 | 78237494 | chr14 | 78304046 | ENSMUST00000022591.7 | Epsti1 | 25.7985 |
| chr6 | 146529552 | 146531552 | chr6 | 146526381 | ENSMUST00000067404.6 | Fgfr1op2 | 25.1325 |
| chr10 | 62472179 | 62474179 | chr10 | 61802925 | ENSMUST00000072357.6 | Hk1 | 24.9685 |
| chr15 | 59547763 | 59549763 | chr15 | 59479905 | ENSMUST00000067543.6 | Trib1 | 24.9402 |
| chr3 | 95950914 | 95952914 | chr3 | 95695928 | ENSMUST00000090476.3 | BC028528 | 24.8663 |
| chr10 | 93511891 | 93513891 | chr10 | 93241372 | ENSMUSG00000090610.1 | Gm3571 | 24.6327 |
| chr3 | 94856898 | 94858898 | chr3 | 94914995 | ENSMUST00000125548.1 | Vps72 | 24.4885 |
| chr2 | 155513435 | 155515435 | chr2 | 156005881 | ENSMUST00000137340.1 | Rbm39 | 24.4715 |
| chr2 | 155513435 | 155515435 | chr2 | 155518120 | ENSMUST00000041059.5 | Trpc4ap | 24.371 |
| chr6 | 5209639 | 5211639 | chr6 | 5206286 | ENSMUST00000031773.2 | Pon3 | 24.0857 |
| chr5 | 100463781 | 100465781 | chr5 | 100469013 | ENSMUST00000169390.1 | Enoph1 | 24.0495 |
| chr12 | 77365201 | 77367201 | chr12 | 77356219 | ENSMUST00000021443.5 | Mthfd1 | 23.6908 |
| chr12 | 77464719 | 77466719 | chr12 | 77356219 | ENSMUST00000021443.5 | Mthfd1 | 23.6908 |
| chr13 | 98142062 | 98144062 | chr13 | 97968312 | ENSMUST00000022169.7 | Hexb | 23.6374 |
| chr15 | 79745826 | 79747826 | chr15 | 79722838 | ENSMUST00000100424.4 | Apobec3 | 23.44 |
| chr16 | 11172524 | 11174524 | chr16 | 11176486 | ENSMUSG00000037965.8 | Zc3h7a | 23.1371 |
| chr7 | 99808096 | 99810096 | chr7 | 99709659 | ENSMUST00000032842.6 | Ccdc90b | 22.7159 |
| chr8 | 60069239 | 60071239 | chr8 | 60028572 | ENSMUSG00000077526.1 | SNORA65 | 22.7061 |
| chr19 | 4136521 | 4138521 | chr19 | 4125959 | ENSMUST00000140267.2 | Tmem134 | 22.4857 |
| chr5 | 117810007 | 117812007 | chr5 | 117839056 | ENSMUST00000086461.6 | Rfc5 | 22.4177 |
| chr1 | 163003668 | 163005668 | chr1 | 162965297 | ENSMUST00000159157.1 | Gas5 | 22.3981 |
| chr12 | 3423035 | 3425035 | chr12 | 3309969 | ENSMUST00000021001.8 | Rab10 | 22.3555 |
| chr16 | 91358906 | 91360906 | chr16 | 91373095 | ENSMUST00000117836.1 | Ifnar2 | 22.1961 |
| chr16 | 91378045 | 91380045 | chr16 | 91373095 | ENSMUST00000117836.1 | Ifnar2 | 22.1961 |
| chr10 | 127517457 | 127519457 | chr10 | 127962915 | ENSMUST00000026427.6 | Esyt1 | 22.1474 |
| chr9 | 14837735 | 14839735 | chr9 | 15110658 | ENSMUST00000164079.1 | Taf1d | 21.9577 |
| chr9 | 115532075 | 115534075 | chr9 | 114690947 | ENSMUST00000098322.3 | Cmtm7 | 21.9363 |
| chr8 | 87506801 | 87508801 | chr8 | 87364564 | ENSMUST00000128035.1 | Rad23a | 21.8956 |
| chr17 | 26050333 | 26052333 | chr17 | 26078993 | ENSMUST00000026823.8 | Pigq | 21.8666 |
| chr4 | 3583814 | 3585814 | chr4 | 3866061 | ENSMUST00000041122.4 | Chchd7 | 21.6624 |
| chr7 | 134657172 | 134659172 | chr7 | 134702900 | ENSMUST00000084563.4 | Srcap | 21.4888 |
| chr10 | 126563534 | 126565534 | chr10 | 126558216 | ENSMUST00000164259.1 | Os9 | 21.4731 |
| chr16 | 50395250 | 50397250 | chr16 | 50430025 | ENSMUST00000131695.1 | Bbx | 21.4308 |
| chr16 | 91694375 | 91696375 | chr16 | 91647197 | ENSMUST00000023684.7 | Gart | 21.417 |
| chr8 | 87506801 | 87508801 | chr8 | 87489940 | ENSMUST00000065049.8 | Rnaseh2a | 21.3564 |
| chr4 | 123422297 | 123424297 | chr4 | 123395445 | ENSMUST00000030401.7 | Ndufs5 | 21.2071 |
| chr15 | 78826239 | 78828239 | chr15 | 78818243 | ENSMUST00000041035.9 | Triobp | 21.0316 |
| chr3 | 102821347 | 102823347 | chr3 | 103664203 | ENSMUST00000029433.7 | Ptpn22 | 20.7144 |
| chr10 | 127517457 | 127519457 | chr10 | 127669147 | ENSMUST00000105243.2 | Timeless | 20.6351 |
| chr16 | 14160427 | 14162427 | chr16 | 13671951 | ENSMUST00000023365.6 | Bfar | 20.4973 |
| chr17 | 36271246 | 36273246 | chr17 | 36179445 | ENSMUST00000166442.1 | H2-T22 | 20.2821 |
| chr7 | 111252085 | 111254085 | chr7 | 110976610 | ENSMUST00000153218.1 | Hbb-b1 | 20.0287 |
| chr19 | 55348105 | 55350105 | chr19 | 55390522 | ENSMUST00000076891.5 | Zdhhc6 | 19.8872 |
| chr14 | 62047152 | 62049152 | chr14 | 62058784 | ENSMUST00000022496.7 | Kpna3 | 19.7937 |
| chr18 | 35093288 | 35095288 | chr18 | 35091657 | ENSMUST00000025218.6 | Etf1 | 19.755 |
| chr19 | 4136521 | 4138521 | chr19 | 4125989 | ENSMUST00000140405.2 | Tmem134 | 19.6813 |
| chr6 | 5209639 | 5211639 | chr6 | 5248455 | ENSMUST00000057792.8 | Pon2 | 19.2858 |
| chr13 | 101412223 | 101414223 | chr13 | 101421298 | ENSMUST00000022135.8 | Taf9 | 19.2063 |
| chr17 | 36271246 | 36273246 | chr17 | 35561795 | ENSMUSG00000073409.5 | H2-Q6 | 19.1799 |
| chr10 | 128039557 | 128041557 | chr10 | 127896292 | ENSMUST00000105235.2 | Smarcc2 | 19.0331 |
| chr9 | 21431017 | 21433017 | chr9 | 21397190 | ENSMUST00000034700.6 | Yipf2 | 18.7041 |
| chr14 | 73381754 | 73383754 | chr14 | 73637698 | ENSMUST00000044405.6 | Lpar6 | 18.6714 |
| chr14 | 73571234 | 73573234 | chr14 | 73637698 | ENSMUST00000044405.6 | Lpar6 | 18.6714 |
| chr14 | 35496063 | 35498063 | chr14 | 35487114 | ENSMUST00000048263.7 | Wapal | 18.53 |
| chr4 | 120700808 | 120702808 | chr4 | 120689852 | ENSMUST00000043200.7 | Smap2 | 18.4761 |
| chr19 | 32398488 | 32400488 | chr19 | 32423806 | ENSMUST00000142618.1 | Sgms1 | 18.4749 |
| chr9 | 107907557 | 107909557 | chr9 | 107904979 | ENSMUST00000167159.1 | Ip6k1 | 18.2205 |
| chr11 | 116261380 | 116263380 | chr11 | 116135531 | ENSMUST00000021133.9 | Srp68 | 18.0953 |
| chr4 | 102204209 | 102206209 | chr4 | 102242700 | ENSMUST00000171667.1 | Pde4b | 18.077 |
| chr4 | 102710870 | 102712870 | chr4 | 102986417 | ENSMUST00000035780.3 | Oma1 | 18.0525 |
| chr10 | 126563534 | 126565534 | chr10 | 126632783 | ENSMUST00000116229.1 | Dtx3 | 18.036 |
| chr10 | 57804381 | 57806381 | chr10 | 57786214 | ENSMUST00000020078.7 | Lims1 | 17.8597 |
| chr15 | 100461395 | 100463395 | chr15 | 100559807 | ENSMUST00000052069.5 | Galnt6 | 17.5707 |
| chr4 | 135414667 | 135416667 | chr4 | 135412017 | ENSMUST00000171299.1 | Srsf10 | 17.567 |
| chr16 | 91694375 | 91696375 | chr16 | 91717714 | ENSMUST00000124282.1 | Cryzl1 | 17.4287 |
| chr18 | 66635286 | 66637286 | chr18 | 67365074 | ENSMUSG00000073543.4 | Chmp1b | 17.3077 |
| chr9 | 14837735 | 14839735 | chr9 | 15110704 | ENSMUST00000171167.1 | Taf1d | 17.2543 |
| chr11 | 19995723 | 19997723 | chr11 | 20641592 | ENSMUST00000035350.5 | Aftph | 17.1661 |
| chr19 | 4136521 | 4138521 | chr19 | 4127572 | ENSMUST00000127555.1 | Tmem134 | 17.0733 |
| chr2 | 167772685 | 167774685 | chr2 | 167757827 | ENSMUST00000029053.7 | Ptpn1 | 17.0646 |
| chr16 | 91694375 | 91696375 | chr16 | 91729220 | ENSMUST00000073466.6 | Cryzl1 | 17.0518 |
| chr11 | 102891992 | 102893992 | chr11 | 103032421 | ENSMUST00000042286.5 | Fmnl1 | 16.8914 |
| chr17 | 80875858 | 80877858 | chr17 | 80689552 | ENSMUST00000061703.9 | Morn2 | 16.6389 |
| chr16 | 91825238 | 91827238 | chr16 | 91643391 | ENSMUST00000156713.1 | Gart | 16.6192 |
| chr14 | 73338176 | 73340176 | chr14 | 73561747 | ENSMUST00000164298.1 | Rcbtb2 | 16.5439 |
| chr14 | 73381754 | 73383754 | chr14 | 73561747 | ENSMUST00000164298.1 | Rcbtb2 | 16.5439 |
| chr14 | 73571234 | 73573234 | chr14 | 73561747 | ENSMUST00000164298.1 | Rcbtb2 | 16.5439 |
| chr14 | 73843970 | 73845970 | chr14 | 73561747 | ENSMUST00000164298.1 | Rcbtb2 | 16.5439 |
| chr11 | 115555939 | 115557939 | chr11 | 115397131 | ENSMUST00000121185.1 | Sumo2 | 16.503 |
| chr14 | 119328244 | 119330244 | chr14 | 119337154 | ENSMUST00000022734.7 | Dnajc3 | 16.4685 |
| chr7 | 135872166 | 135874166 | chr7 | 135884009 | ENSMUST00000057557.7 | Mcmbp | 16.4611 |
| chr1 | 163003668 | 163005668 | chr1 | 162966151 | ENSMUST00000160497.1 | Gas5 | 16.3081 |
| chr10 | 126563534 | 126565534 | chr10 | 126558216 | ENSMUST00000080975.4 | Os9 | 16.2354 |
| chr10 | 127517457 | 127519457 | chr10 | 126558216 | ENSMUST00000080975.4 | Os9 | 16.2354 |
| chr9 | 21423287 | 21425287 | chr9 | 21420708 | ENSMUST00000174008.1 | Smarca4 | 16.1805 |
| chr17 | 80283459 | 80285459 | chr17 | 80526811 | ENSMUST00000039205.4 | Galm | 16.1286 |
| chr11 | 116261380 | 116263380 | chr11 | 116295408 | ENSMUST00000057676.6 | Fam100b | 16.0857 |
| chr13 | 23655255 | 23657255 | chr13 | 23622919 | ENSMUST00000102972.2 | Hist1h4h | 16.0674 |
| chr18 | 38599182 | 38601182 | chr18 | 38498657 | ENSMUST00000063814.8 | Gnpda1 | 15.9541 |
| chr1 | 173435009 | 173437009 | chr1 | 173167659 | ENSMUST00000150108.1 | Ndufs2 | 15.8777 |
| chr17 | 34345356 | 34347356 | chr17 | 34340472 | ENSMUST00000025197.5 | Tap2 | 15.8249 |
| chr11 | 5267987 | 5269987 | chr11 | 4604337 | ENSMUST00000151559.1 | Uqcr10 | 15.7415 |
| chr9 | 69839142 | 69841142 | chr9 | 69837273 | ENSMUST00000034754.5 | Bnip2 | 15.6241 |
| chr5 | 135953210 | 135955210 | chr5 | 135485326 | ENSMUST00000154469.1 | Abhd11 | 15.3623 |
| chr15 | 97556610 | 97558610 | chr15 | 97536253 | ENSMUST00000023104.5 | Rpap3 | 15.3501 |
| chr3 | 89686643 | 89688643 | chr3 | 89802608 | ENSMUST00000079724.4 | Hax1 | 15.3465 |
| chr5 | 30219040 | 30221040 | chr5 | 30400062 | ENSMUST00000138520.1 | Tyms | 15.337 |
| chr10 | 127517457 | 127519457 | chr10 | 127524737 | ENSMUST00000144918.1 | Atp5b | 15.288 |
| chr16 | 91825238 | 91827238 | chr16 | 91854127 | ENSMUST00000099527.1 | Itsn1 | 15.2509 |
| chr3 | 133187535 | 133189535 | chr3 | 132973074 | ENSMUST00000029644.9 | Ppa2 | 15.2502 |
| chrX | 34634483 | 34636483 | chrX | 34650317 | ENSMUST00000076265.6 | Upf3b | 15.1495 |
| chr6 | 113579365 | 113581365 | chr6 | 113293978 | ENSMUST00000032409.8 | Camk1 | 15.1491 |
| chr13 | 64221561 | 64223561 | chr13 | 64414018 | ENSMUST00000099434.4 | 1110018J18Rik | 15.1292 |
| chr11 | 102076861 | 102078861 | chr11 | 102178601 | ENSMUST00000107119.2 | Ubtf | 15.0821 |
| chr7 | 117252131 | 117254131 | chr7 | 117161939 | ENSMUST00000084731.3 | Ipo7 | 15.0062 |
| chr11 | 115555939 | 115557939 | chr11 | 115560621 | ENSMUST00000106499.1 | Grb2 | 14.811 |
| chr9 | 58117801 | 58119801 | chr9 | 58081192 | ENSMUST00000148628.1 | Pml | 14.7887 |
| chr18 | 24380786 | 24382786 | chr18 | 24363845 | ENSMUST00000000430.7 | Galnt1 | 14.65 |
| chr6 | 98971588 | 98973588 | chr6 | 99113012 | ENSMUST00000113328.1 | Foxp1 | 14.6274 |
| chr6 | 31181816 | 31183816 | chr6 | 31418120 | ENSMUST00000141045.1 | Mkln1 | 14.571 |
| chr2 | 155513435 | 155515435 | chr2 | 155652172 | ENSMUST00000154841.1 | Eif6 | 14.5655 |
| chr4 | 135414667 | 135416667 | chr4 | 135412047 | ENSMUST00000129718.1 | Srsf10 | 14.4925 |
| chr9 | 62212191 | 62214191 | chr9 | 62221246 | ENSMUST00000145679.1 | Anp32a | 14.4165 |
| chr7 | 97195468 | 97197468 | chr7 | 96487297 | ENSMUST00000041968.3 | Tmem135 | 14.4008 |
| chr7 | 134624783 | 134626783 | chr7 | 134732212 | ENSMUST00000033088.6 | Rnf40 | 14.2992 |
| chr2 | 163246818 | 163248818 | chr2 | 163484135 | ENSMUST00000064703.6 | Pkig | 14.2773 |
| chr2 | 163543858 | 163545858 | chr2 | 163484135 | ENSMUST00000064703.6 | Pkig | 14.2773 |
| chr1 | 155447492 | 155449492 | chr1 | 155596556 | ENSMUST00000086209.3 | Rnasel | 14.2252 |
| chr8 | 26154544 | 26156544 | chr8 | 26212457 | ENSMUST00000128715.1 | Plekha2 | 14.1782 |
| chr17 | 71444810 | 71446810 | chr17 | 71533318 | ENSMUST00000129635.1 | Lpin2 | 14.167 |
| chr1 | 95380382 | 95382382 | chr1 | 95375385 | ENSMUST00000170883.1 | Hdlbp | 14.1403 |
| chr16 | 43950381 | 43952381 | chr16 | 44139418 | ENSMUST00000114666.2 | Atp6v1a | 14.0579 |
| chr5 | 130603974 | 130605974 | chr5 | 130328750 | ENSMUST00000137357.1 | Sumf2 | 14.0476 |
| chr18 | 35093288 | 35095288 | chr18 | 34811389 | ENSMUST00000133181.1 | Cdc23 | 13.9079 |
| chr1 | 163003668 | 163005668 | chr1 | 162965060 | ENSMUST00000159119.1 | Gas5 | 13.8853 |
| chr11 | 116261380 | 116263380 | chr11 | 116532974 | ENSMUST00000139934.1 | 1810032O08Rik | 13.8604 |
| chr5 | 114282886 | 114284886 | chr5 | 114280510 | ENSMUST00000100874.4 | Selplg | 13.8199 |
| chr17 | 24661305 | 24663305 | chr17 | 24873587 | ENSMUST00000115262.1 | Sepx1 | 13.8116 |
| chr4 | 135414667 | 135416667 | chr4 | 135412027 | ENSMUST00000154447.1 | Srsf10 | 13.7863 |
| chr17 | 24661305 | 24663305 | chr17 | 24656153 | ENSMUST00000088464.5 | Traf7 | 13.7801 |
| chr5 | 107438647 | 107440647 | chr5 | 107718648 | ENSMUST00000031224.8 | Tgfbr3 | 13.749 |
| chr5 | 114320348 | 114322348 | chr5 | 114222757 | ENSMUST00000026937.5 | Iscu | 13.5766 |
| chr11 | 75222724 | 75224724 | chr11 | 75281581 | ENSMUST00000108435.1 | Tlcd2 | 13.5726 |
| chr2 | 27337091 | 27339091 | chr2 | 27331206 | ENSMUST00000077737.6 | Brd3 | 13.5623 |
| chr3 | 94856898 | 94858898 | chr3 | 95085998 | ENSMUST00000015855.7 | Prune | 13.5195 |
| chr10 | 126563534 | 126565534 | chr10 | 126648678 | ENSMUST00000013970.7 | Pip4k2c | 13.4306 |
| chr12 | 56425473 | 56427473 | chr12 | 56403987 | ENSMUST00000021410.8 | Ppp2r3c | 13.387 |
| chr4 | 3583814 | 3585814 | chr4 | 3502022 | ENSMUST00000052712.5 | Tgs1 | 13.2671 |
| chr7 | 52079823 | 52081823 | chr7 | 51809318 | ENSMUST00000107911.1 | Nr1h2 | 13.232 |
| chr16 | 38405373 | 38407373 | chr16 | 38558811 | ENSMUST00000163884.1 | Tmem39a | 13.2239 |
| chr3 | 94856898 | 94858898 | chr3 | 95091631 | ENSMUST00000170282.1 | Fam63a | 13.103 |
| chr3 | 157739162 | 157741162 | chr3 | 157699664 | ENSMUST00000156597.1 | Lrrc40 | 13.0649 |
| chr7 | 109112980 | 109114980 | chr7 | 109118357 | ENSMUST00000170458.1 | Numa1 | 12.9959 |
| chr2 | 5850914 | 5852914 | chr2 | 5765987 | ENSMUST00000152519.1 | Cdc123 | 12.8947 |
| chr11 | 75378410 | 75380410 | chr11 | 75282213 | ENSMUST00000153236.1 | Tlcd2 | 12.8796 |
| chr16 | 8724299 | 8726299 | chr16 | 8738513 | ENSMUST00000160326.1 | Usp7 | 12.8767 |
| chr2 | 29525390 | 29527390 | chr2 | 29745315 | ENSMUST00000113756.1 | Odf2 | 12.8675 |
| chr11 | 88810671 | 88812671 | chr11 | 88816779 | ENSMUST00000000287.8 | Scpep1 | 12.8406 |
| chr11 | 100706701 | 100708701 | chr11 | 100712038 | ENSMUST00000004143.2 | Stat5b | 12.8082 |
| chr16 | 32533124 | 32535124 | chr16 | 32431010 | ENSMUST00000079791.4 | Pcyt1a | 12.5368 |
| chr9 | 63596837 | 63598837 | chr9 | 63605801 | ENSMUST00000034973.3 | Smad3 | 12.5153 |
| chr2 | 84208651 | 84210651 | chr2 | 83484592 | ENSMUST00000081591.6 | Zc3h15 | 12.5147 |
| chr3 | 51245689 | 51247689 | chr3 | 51212910 | ENSMUST00000038108.6 | Ndufc1 | 12.4887 |
| chr14 | 69867742 | 69869742 | chr14 | 69776911 | ENSMUSG00000091986.1 | Rps2-ps5 | 12.4827 |
| chr9 | 65395253 | 65397253 | chr9 | 65142067 | ENSMUST00000015501.4 | Clpx | 12.3567 |
| chr9 | 58117801 | 58119801 | chr9 | 58097593 | ENSMUST00000085673.4 | Pml | 12.3361 |
| chr1 | 9846060 | 9848060 | chr1 | 9838478 | ENSMUST00000097826.4 | Sgk3 | 12.3032 |
| chr16 | 91358906 | 91360906 | chr16 | 91729616 | ENSMUST00000056482.7 | Itsn1 | 12.2858 |
| chr16 | 91694375 | 91696375 | chr16 | 91729616 | ENSMUST00000056482.7 | Itsn1 | 12.2858 |
| chr1 | 153158856 | 153160856 | chr1 | 153191628 | ENSMUST00000111887.3 | Ivns1abp | 12.2584 |
| chr3 | 135227472 | 135229472 | chr3 | 135148575 | ENSMUST00000029814.9 | Manba | 12.1224 |
| chr1 | 36695504 | 36697504 | chr1 | 36748332 | ENSMUST00000081180.4 | Cox5b | 12.1213 |
| chr2 | 155513435 | 155515435 | chr2 | 155418450 | ENSMUST00000130881.1 | Gss | 11.7416 |
| chr2 | 154422388 | 154424388 | chr2 | 154336272 | ENSMUST00000137526.1 | Cbfa2t2 | 11.7194 |
| chr11 | 102891992 | 102893992 | chr11 | 102889660 | ENSMUST00000021314.7 | Nmt1 | 11.65 |
| chr9 | 58117801 | 58119801 | chr9 | 58100971 | ENSMUST00000034883.5 | Stoml1 | 11.6346 |
| chr16 | 11172524 | 11174524 | chr16 | 11176486 | ENSMUST00000037633.8 | Zc3h7a | 11.5969 |
| chr16 | 11172524 | 11174524 | chr16 | 11224591 | ENSMUST00000167025.1 | Gspt1 | 11.463 |
| chr2 | 84208651 | 84210651 | chr2 | 84215515 | ENSMUST00000099944.3 | Calcrl | 11.4231 |
| chr8 | 26121545 | 26123545 | chr8 | 26950929 | ENSMUST00000110609.1 | Ash2l | 11.326 |
| chr7 | 109112980 | 109114980 | chr7 | 109371542 | ENSMUST00000156529.1 | Pgap2 | 11.3151 |
| chr17 | 71347711 | 71349711 | chr17 | 71351894 | ENSMUST00000148960.1 | 2900073G15Rik | 11.28 |
| chr3 | 136369955 | 136371955 | chr3 | 136333088 | ENSMUST00000070198.7 | Ppp3ca | 11.2641 |
| chr2 | 90840821 | 90842821 | chr2 | 90588161 | ENSMUSG00000080873.2 | Rpl30-ps3 | 11.2587 |
| chr10 | 119604888 | 119606888 | chr10 | 119645836 | ENSMUST00000134797.1 | Tmbim4 | 11.1683 |
| chr10 | 119620496 | 119622496 | chr10 | 119645836 | ENSMUST00000134797.1 | Tmbim4 | 11.1683 |
| chr12 | 8526839 | 8528839 | chr12 | 8681030 | ENSMUST00000111122.2 | Pum2 | 11.1361 |
| chr11 | 29674081 | 29676081 | chr11 | 29030748 | ENSMUST00000020756.8 | Pnpt1 | 11.0687 |
| chr11 | 100843840 | 100845840 | chr11 | 100956714 | ENSMUST00000126386.1 | Psmc3ip | 11.0517 |
| chr10 | 80752437 | 80754437 | chr10 | 80730932 | ENSMUST00000099453.4 | Apba3 | 11.0436 |
| chr4 | 154609450 | 154611450 | chr4 | 155079244 | ENSMUST00000105595.1 | Ssu72 | 11.0135 |
| chr6 | 41174603 | 41176603 | chr6 | 40421467 | ENSMUST00000121360.1 | Ssbp1 | 10.9775 |
| chr18 | 6485002 | 6487002 | chr18 | 6490854 | ENSMUST00000050542.5 | Epc1 | 10.9537 |
| chr15 | 93081233 | 93083233 | chr15 | 93228781 | ENSMUST00000068457.7 | Pphln1 | 10.9426 |
| chr4 | 140690421 | 140692421 | chr4 | 140695690 | ENSMUST00000094549.4 | D4Ertd22e | 10.9122 |
| chr6 | 98971588 | 98973588 | chr6 | 99113012 | ENSMUST00000113324.1 | Foxp1 | 10.8039 |
| chr3 | 59038623 | 59040623 | chr3 | 58934546 | ENSMUST00000091112.4 | P2ry14 | 10.8012 |
| chr12 | 70558261 | 70560261 | chr12 | 70329177 | ENSMUST00000021359.5 | Pole2 | 10.7758 |
| chr4 | 135414667 | 135416667 | chr4 | 135411662 | ENSMUST00000126641.1 | Srsf10 | 10.7372 |
| chr6 | 146529552 | 146531552 | chr6 | 146526443 | ENSMUST00000058245.4 | Fgfr1op2 | 10.737 |
| chr12 | 56425473 | 56427473 | chr12 | 56403624 | ENSMUST00000021411.7 | 1110008L16Rik | 10.6165 |
| chr11 | 29674081 | 29676081 | chr11 | 29618563 | ENSMUST00000060992.5 | Rtn4 | 10.5334 |
| chr13 | 48661017 | 48663017 | chr13 | 49311137 | ENSMUST00000172021.1 | 1110007C09Rik | 10.4497 |
| chr6 | 124151275 | 124153275 | chr6 | 124443131 | ENSMUST00000049124.9 | C1rl | 10.307 |
| chr6 | 124151275 | 124153275 | chr6 | 124365085 | ENSMUST00000080557.5 | Pex5 | 10.2765 |
| chr12 | 32847765 | 32849765 | chr12 | 32893524 | ENSMUST00000053215.7 | Pik3cg | 10.2751 |
| chr3 | 95950914 | 95952914 | chr3 | 95861642 | ENSMUST00000140518.1 | Vps45 | 9.99186 |
| chr19 | 29457752 | 29459752 | chr19 | 29438952 | ENSMUST00000139860.1 | 5033414D02Rik | 9.9418 |
| chr19 | 29457752 | 29459752 | chr19 | 29435897 | ENSMUST00000155367.1 | 5033414D02Rik | 9.92093 |
| chr3 | 51245689 | 51247689 | chr3 | 51219938 | ENSMUST00000029303.7 | Naa15 | 9.83579 |
| chr7 | 133271143 | 133273143 | chr7 | 133943961 | ENSMUST00000106348.1 | Aldoa | 9.81297 |
| chr3 | 136369955 | 136371955 | chr3 | 136333797 | ENSMUST00000098590.3 | Ppp3ca | 9.79558 |
| chr11 | 52181889 | 52183889 | chr11 | 51912183 | ENSMUST00000020608.2 | Ppp2ca | 9.77632 |
| chr14 | 122100803 | 122102803 | chr14 | 122933561 | ENSMUST00000038374.6 | Pcca | 9.73802 |
| chr10 | 57804381 | 57806381 | chr10 | 57834236 | ENSMUST00000171062.1 | Lims1 | 9.71902 |
| chr14 | 27405756 | 27407756 | chr14 | 27457560 | ENSMUSG00000021877.4 | Arf4 | 9.71433 |
| chr14 | 27409444 | 27411444 | chr14 | 27457560 | ENSMUSG00000021877.4 | Arf4 | 9.71433 |
| chr6 | 135178173 | 135180173 | chr6 | 135147995 | ENSMUST00000111915.1 | 8430419L09Rik | 9.71222 |
| chr7 | 135872166 | 135874166 | chr7 | 135888384 | ENSMUST00000042942.8 | Sec23ip | 9.68704 |
| chr3 | 102821347 | 102823347 | chr3 | 102862152 | ENSMUST00000170829.1 | Nras | 9.66343 |
| chr11 | 78817188 | 78819188 | chr11 | 78798426 | ENSMUST00000108269.3 | Lgals9 | 9.66038 |
| chr13 | 101412223 | 101414223 | chr13 | 101421014 | ENSMUST00000022136.6 | Rad17 | 9.62121 |
| chr17 | 10500648 | 10502648 | chr17 | 10512226 | ENSMUST00000097414.3 | Qk | 9.54331 |
| chr2 | 155513435 | 155515435 | chr2 | 156108367 | ENSMUST00000147234.1 | Phf20 | 9.51937 |
| chr2 | 78901993 | 78903993 | chr2 | 79095657 | ENSMUST00000099972.4 | Itga4 | 9.50694 |
| chr2 | 78976919 | 78978919 | chr2 | 79095657 | ENSMUST00000099972.4 | Itga4 | 9.50694 |
| chr3 | 95950914 | 95952914 | chr3 | 95799645 | ENSMUST00000130043.1 | Plekho1 | 9.45962 |
| chr19 | 55348105 | 55350105 | chr19 | 55390491 | ENSMUST00000111682.2 | Zdhhc6 | 9.42245 |
| chr1 | 163003668 | 163005668 | chr1 | 163000898 | ENSMUST00000111620.3 | Cenpl | 9.38744 |
| chr14 | 52868009 | 52870009 | chr14 | 52857247 | ENSMUST00000089752.4 | Chd8 | 9.31758 |
| chr2 | 116804819 | 116806819 | chr2 | 117075475 | ENSMUST00000028825.4 | Fam98b | 9.28637 |
| chr1 | 36695504 | 36697504 | chr1 | 36604046 | ENSMUST00000001172.5 | Ankrd39 | 9.27306 |
| chr16 | 91378045 | 91380045 | chr16 | 91647751 | ENSMUST00000117633.1 | Son | 9.247 |
| chr4 | 131585487 | 131587487 | chr4 | 131563352 | ENSMUST00000054917.5 | Epb4.1 | 9.22769 |
| chr4 | 131753294 | 131755294 | chr4 | 131563352 | ENSMUST00000054917.5 | Epb4.1 | 9.22769 |
| chr2 | 78680699 | 78682699 | chr2 | 78709835 | ENSMUST00000121433.1 | Ube2e3 | 9.20667 |
| chr10 | 62472179 | 62474179 | chr10 | 62478916 | ENSMUST00000141616.1 | Hnrnph3 | 9.19269 |
| chr2 | 154422388 | 154424388 | chr2 | 154429444 | ENSMUST00000000896.4 | Pxmp4 | 9.13821 |
| chr11 | 106299577 | 106301577 | chr11 | 106576579 | ENSMUST00000103069.3 | Pecam1 | 9.12056 |
| chr1 | 93127676 | 93129676 | chr1 | 93147143 | ENSMUST00000171112.1 | Ube2f | 9.1014 |
| chr1 | 156464793 | 156466793 | chr1 | 157405259 | ENSMUST00000035560.3 | Acbd6 | 9.04867 |
| chr2 | 126505144 | 126507144 | chr2 | 126501238 | ENSMUST00000110424.2 | Gabpb1 | 9.03388 |
| chr7 | 135872166 | 135874166 | chr7 | 135605186 | ENSMUST00000106226.2 | Tial1 | 9.03011 |
| chr16 | 4719688 | 4721688 | chr16 | 4628871 | ENSMUST00000150028.1 | Coro7 | 9.02697 |
| chr14 | 69867742 | 69869742 | chr14 | 69955208 | ENSMUST00000064831.4 | Entpd4 | 9.01577 |
| chr8 | 129150266 | 129152266 | chr8 | 129117336 | ENSMUST00000054960.6 | Irf2bp2 | 9.00398 |
| chr17 | 36271246 | 36273246 | chr17 | 36116900 | ENSMUST00000166679.1 | Gnl1 | 8.96825 |
| chr17 | 26050333 | 26052333 | chr17 | 25981796 | ENSMUST00000043897.8 | Rhot2 | 8.92195 |
| chr10 | 126563534 | 126565534 | chr10 | 126338427 | ENSMUST00000168520.1 | Xrcc6bp1 | 8.87074 |
| chr15 | 95625250 | 95627250 | chr15 | 95621274 | ENSMUST00000071874.5 | Ano6 | 8.83965 |
| chr6 | 11963142 | 11965142 | chr6 | 11875881 | ENSMUST00000115511.2 | Phf14 | 8.80013 |
| chr10 | 62472179 | 62474179 | chr10 | 62486965 | ENSMUST00000140743.1 | Hnrnph3 | 8.74083 |
| chr2 | 34833570 | 34835570 | chr2 | 35111938 | ENSMUST00000113016.3 | Gsn | 8.71809 |
| chr19 | 4136521 | 4138521 | chr19 | 4151580 | ENSMUST00000148189.1 | Coro1b | 8.59624 |
| chr6 | 86781830 | 86783830 | chr6 | 86959883 | ENSMUST00000144776.1 | Nfu1 | 8.44171 |
| chr6 | 87024053 | 87026053 | chr6 | 86959883 | ENSMUST00000144776.1 | Nfu1 | 8.44171 |
| chr5 | 130679452 | 130681452 | chr5 | 130695614 | ENSMUST00000065329.6 | 0610007L01Rik | 8.41362 |
| chr7 | 134624783 | 134626783 | chr7 | 134628735 | ENSMUST00000048896.6 | Fbrs | 8.41005 |
| chr7 | 99808096 | 99810096 | chr7 | 99818443 | ENSMUST00000119954.1 | Pcf11 | 8.3824 |
| chr3 | 157739162 | 157741162 | chr3 | 157686386 | ENSMUST00000152274.2 | Srsf11 | 8.38224 |
| chr14 | 73571234 | 73573234 | chr14 | 73551292 | ENSMUST00000169479.1 | Rcbtb2 | 8.35216 |
| chr9 | 14837735 | 14839735 | chr9 | 15106000 | ENSMUST00000034414.7 | 4931406C07Rik | 8.35204 |
| chr2 | 126505144 | 126507144 | chr2 | 126501191 | ENSMUST00000089741.4 | Gabpb1 | 8.34456 |
| chr1 | 95380382 | 95382382 | chr1 | 95651415 | ENSMUST00000112905.2 | Thap4 | 8.27546 |
| chr7 | 80771375 | 80773375 | chr7 | 80686632 | ENSMUST00000169922.2 | Chd2 | 8.24347 |
| chr19 | 21647091 | 21649091 | chr19 | 21547162 | ENSMUST00000087600.3 | Gda | 8.22054 |
| chr17 | 31176554 | 31178554 | chr17 | 31433702 | ENSMUST00000114536.3 | Slc37a1 | 8.20171 |
| chr10 | 127517457 | 127519457 | chr10 | 127984830 | ENSMUST00000040572.3 | Zc3h10 | 8.18665 |
| chr3 | 121993571 | 121995571 | chr3 | 121518220 | ENSMUST00000029770.5 | Abcd3 | 8.18514 |
| chr3 | 94856898 | 94858898 | chr3 | 95659676 | ENSMUST00000161476.1 | Prpf3 | 8.18358 |
| chr19 | 4136521 | 4138521 | chr19 | 4365802 | ENSMUST00000047898.6 | Kdm2a | 8.17222 |
| chr10 | 80080382 | 80082382 | chr10 | 80097180 | ENSMUST00000019676.6 | Csnk1g2 | 8.1704 |
| chr7 | 142900654 | 142902654 | chr7 | 142908062 | ENSMUST00000033310.7 | Mki67 | 8.1613 |
| chr11 | 100706701 | 100708701 | chr11 | 100948591 | ENSMUST00000107302.1 | Mlx | 8.1507 |
| chr11 | 100843840 | 100845840 | chr11 | 100948591 | ENSMUST00000107302.1 | Mlx | 8.1507 |
| chr5 | 117810007 | 117812007 | chr5 | 117830860 | ENSMUST00000129369.1 | Rfc5 | 8.09233 |
| chr10 | 107636370 | 107638370 | chr10 | 107599456 | ENSMUST00000070663.5 | Ppp1r12a | 8.09189 |
| chr19 | 9113416 | 9115416 | chr19 | 9210126 | ENSMUST00000049948.5 | Asrgl1 | 8.04306 |
| chr13 | 43545784 | 43547784 | chr13 | 43574261 | ENSMUST00000171056.1 | Ranbp9 | 8.01401 |
| chr8 | 26121545 | 26123545 | chr8 | 26212666 | ENSMUST00000064883.6 | Plekha2 | 7.95934 |
| chr6 | 146529552 | 146531552 | chr6 | 146526381 | ENSMUST00000111663.2 | Fgfr1op2 | 7.90908 |
| chr11 | 100843840 | 100845840 | chr11 | 100480890 | ENSMUST00000155152.1 | Dnajc7 | 7.87912 |
| chr10 | 80080382 | 80082382 | chr10 | 80318020 | ENSMUST00000035597.8 | 3110056O03Rik | 7.85751 |
| chr11 | 72769965 | 72771965 | chr11 | 72734345 | ENSMUST00000125122.1 | Zzef1 | 7.82642 |
| chr10 | 19853457 | 19855457 | chr10 | 20067813 | ENSMUST00000020167.6 | Fam54a | 7.81228 |
| chr10 | 19908474 | 19910474 | chr10 | 20067813 | ENSMUST00000020167.6 | Fam54a | 7.81228 |
| chr10 | 80752437 | 80754437 | chr10 | 80841196 | ENSMUST00000020457.7 | Fzr1 | 7.7722 |
| chr12 | 77464719 | 77466719 | chr12 | 77487213 | ENSMUST00000070594.3 | Zbtb1 | 7.73777 |
| chr8 | 87506801 | 87508801 | chr8 | 87493666 | ENSMUST00000140561.1 | Rnaseh2a | 7.71619 |
| chr11 | 115555939 | 115557939 | chr11 | 115626747 | ENSMUST00000093912.4 | 2310067B10Rik | 7.70155 |
| chr10 | 39344769 | 39346769 | chr10 | 39231545 | ENSMUST00000136659.1 | Fyn | 7.70093 |
| chr6 | 146529552 | 146531552 | chr6 | 146526357 | ENSMUST00000032427.8 | 4933424B01Rik | 7.70082 |
| chr7 | 88146381 | 88148381 | chr7 | 87550317 | ENSMUST00000107362.3 | Furin | 7.64928 |
| chr5 | 20540637 | 20542637 | chr5 | 20561729 | ENSMUST00000030556.7 | Ptpn12 | 7.63344 |
| chr3 | 142519706 | 142521706 | chr3 | 142516848 | ENSMUST00000090108.4 | Pkn2 | 7.63148 |
| chr16 | 91358906 | 91360906 | chr16 | 91373028 | ENSMUST00000023693.7 | Ifnar2 | 7.63045 |
| chr16 | 91378045 | 91380045 | chr16 | 91373028 | ENSMUST00000023693.7 | Ifnar2 | 7.63045 |
| chr17 | 36271246 | 36273246 | chr17 | 35531071 | ENSMUSG00000055413.9 | H2-Q8 | 7.56616 |
| chr16 | 76335594 | 76337594 | chr16 | 76374072 | ENSMUST00000121927.1 | Nrip1 | 7.49674 |
| chr9 | 99460139 | 99462139 | chr9 | 99476218 | ENSMUST00000066650.5 | Dbr1 | 7.43805 |
| chr9 | 24978329 | 24980329 | chr9 | 25060169 | ENSMUST00000115272.1 | 7-Sep | 7.42588 |
| chr9 | 21423287 | 21425287 | chr9 | 21807479 | ENSMUST00000115331.2 | Prkcsh | 7.37175 |
| chr8 | 47708070 | 47710070 | chr8 | 47702554 | ENSMUST00000040468.8 | Ccdc111 | 7.35377 |
| chr10 | 39950579 | 39952579 | chr10 | 39978273 | ENSMUSG00000065870.1 | U3 | 7.32981 |
| chr4 | 132127950 | 132129950 | chr4 | 132925621 | ENSMUST00000030669.7 | Slc9a1 | 7.32626 |
| chr1 | 135588318 | 135590318 | chr1 | 135614506 | ENSMUST00000140810.1 | Atp2b4 | 7.27477 |
| chr11 | 104405333 | 104407333 | chr11 | 104411797 | ENSMUST00000093923.2 | Cdc27 | 7.26967 |
| chr11 | 75222724 | 75224724 | chr11 | 75492761 | ENSMUST00000017920.7 | Crk | 7.25923 |
| chr11 | 75351840 | 75353840 | chr11 | 75492761 | ENSMUST00000017920.7 | Crk | 7.25923 |
| chr3 | 14913354 | 14915354 | chr3 | 14533824 | ENSMUST00000091325.3 | Lrrcc1 | 7.23901 |
| chr4 | 108787213 | 108789213 | chr4 | 108874877 | ENSMUST00000030288.7 | Osbpl9 | 7.18877 |
| chr1 | 87737007 | 87739007 | chr1 | 87690016 | ENSMUST00000113360.1 | Cab39 | 7.1813 |
| chr14 | 47646852 | 47648852 | chr14 | 47380216 | ENSMUST00000067426.4 | Cdkn3 | 7.16601 |
| chr7 | 134657172 | 134659172 | chr7 | 134614533 | ENSMUST00000133817.1 | Prr14 | 7.15322 |
| chr9 | 45817658 | 45819658 | chr9 | 46091091 | ENSMUST00000074957.3 | Bud13 | 7.15123 |
| chr9 | 21423287 | 21425287 | chr9 | 21229376 | ENSMUST00000173397.1 | Dnm2 | 7.119 |
| chr6 | 86559277 | 86561277 | chr6 | 86619153 | ENSMUST00000001184.7 | Mxd1 | 7.109 |
| chr11 | 29687141 | 29689141 | chr11 | 29448109 | ENSMUSG00000020460.9 | Rps27a | 7.09806 |
| chr1 | 162790541 | 162792541 | chr1 | 162977273 | ENSMUST00000161748.1 | Dars2 | 7.0703 |
| chr2 | 5850914 | 5852914 | chr2 | 5872515 | ENSMUST00000060092.6 | Upf2 | 7.06658 |
| chr10 | 119620496 | 119622496 | chr10 | 119639186 | ENSMUST00000145665.1 | Irak3 | 7.06543 |
| chr9 | 65395253 | 65397253 | chr9 | 65477455 | ENSMUST00000169003.1 | Rbpms2 | 7.01537 |
| chr6 | 5209639 | 5211639 | chr6 | 5206235 | ENSMUST00000125686.1 | Pon3 | 7.00312 |
| chr2 | 91793939 | 91795939 | chr2 | 91790584 | ENSMUST00000111303.1 | Dgkz | 6.96447 |
| chr7 | 134657172 | 134659172 | chr7 | 134565364 | ENSMUST00000126756.1 | Zfp688 | 6.93207 |
| chr5 | 116072916 | 116074916 | chr5 | 116015263 | ENSMUST00000064454.7 | Gcn1l1 | 6.90903 |
| chr4 | 140690421 | 140692421 | chr4 | 140695681 | ENSMUST00000102487.3 | D4Ertd22e | 6.90075 |
| chr2 | 91793939 | 91795939 | chr2 | 91805744 | ENSMUST00000128152.1 | Dgkz | 6.89733 |
| chr11 | 100843840 | 100845840 | chr11 | 100873135 | ENSMUST00000092663.3 | Atp6v0a1 | 6.88308 |
| chr8 | 26121545 | 26123545 | chr8 | 26830536 | ENSMUST00000068916.8 | Ppapdc1b | 6.80839 |
| chr12 | 3770285 | 3772285 | chr12 | 3807030 | ENSMUST00000020991.8 | Dnmt3a | 6.80697 |
| chr11 | 121339728 | 121341728 | chr11 | 120657717 | ENSMUST00000106135.1 | Dus1l | 6.80295 |
| chr13 | 44841342 | 44843342 | chr13 | 44826640 | ENSMUST00000173246.1 | Jarid2 | 6.78179 |
| chr14 | 55035149 | 55037149 | chr14 | 55724984 | ENSMUST00000170285.1 | Ap1g2 | 6.76824 |
| chr4 | 108787213 | 108789213 | chr4 | 108760054 | ENSMUST00000159198.1 | Osbpl9 | 6.76807 |
| chr16 | 91825238 | 91827238 | chr16 | 91648125 | ENSMUST00000114036.2 | Son | 6.7441 |
| chr14 | 73043153 | 73045153 | chr14 | 73051883 | ENSMUST00000161550.1 | Fndc3a | 6.67312 |
| chr1 | 184341212 | 184343212 | chr1 | 184447655 | ENSMUST00000068505.7 | Capn2 | 6.65473 |
| chr9 | 70131770 | 70133770 | chr9 | 70351161 | ENSMUST00000113595.1 | Rnf111 | 6.64444 |
| chr12 | 4606585 | 4608585 | chr12 | 4881164 | ENSMUST00000045921.7 | Mfsd2b | 6.63573 |
| chr10 | 80080382 | 80082382 | chr10 | 80119239 | ENSMUST00000126980.1 | Btbd2 | 6.62603 |
| chr1 | 163003668 | 163005668 | chr1 | 163000738 | ENSMUST00000160591.1 | Dars2 | 6.62413 |
| chr7 | 134657172 | 134659172 | chr7 | 135021215 | ENSMUST00000050383.7 | Zfp646 | 6.61792 |
| chr2 | 32725469 | 32727469 | chr2 | 32818909 | ENSMUST00000134912.1 | Rpl12 | 6.52303 |
| chr10 | 19853457 | 19855457 | chr10 | 19868277 | ENSMUST00000116259.2 | Mtap7 | 6.49035 |
| chr12 | 33989005 | 33991005 | chr12 | 33987380 | ENSMUST00000090597.4 | Atxn7l1 | 6.46184 |
| chr12 | 86769069 | 86771069 | chr12 | 86815641 | ENSMUST00000140525.1 | Fos | 6.45913 |
| chr9 | 45817658 | 45819658 | chr9 | 45792867 | ENSMUST00000003215.4 | Pafah1b2 | 6.44814 |
| chr4 | 55835512 | 55837512 | chr4 | 55545347 | ENSMUST00000003116.6 | Klf4 | 6.44354 |
| chr8 | 114115301 | 114117301 | chr8 | 114061020 | ENSMUST00000173506.1 | Znrf1 | 6.44086 |
| chr16 | 91694375 | 91696375 | chr16 | 91699533 | ENSMUST00000144877.1 | Cryzl1 | 6.42932 |
| chr2 | 91793939 | 91795939 | chr2 | 91785414 | ENSMUST00000126473.1 | Dgkz | 6.38414 |
| chr11 | 45937156 | 45939156 | chr11 | 45768996 | ENSMUST00000011398.6 | Thg1l | 6.34099 |
| chr11 | 75222724 | 75224724 | chr11 | 75465012 | ENSMUST00000069057.6 | Myo1c | 6.32062 |
| chr11 | 75222724 | 75224724 | chr11 | 75281581 | ENSMUST00000043598.7 | Tlcd2 | 6.30207 |
| chr18 | 56879557 | 56881557 | chr18 | 56722097 | ENSMUST00000130163.1 | Phax | 6.27892 |
| chr9 | 45855653 | 45857653 | chr9 | 46081291 | ENSMUST00000114552.3 | Zfp259 | 6.26376 |
| chr9 | 45875364 | 45877364 | chr9 | 46081291 | ENSMUST00000114552.3 | Zfp259 | 6.26376 |
| chr2 | 31026010 | 31028010 | chr2 | 31101343 | ENSMUST00000000199.7 | Ncs1 | 6.26328 |
| chr7 | 74557778 | 74559778 | chr7 | 74517744 | ENSMUST00000156690.1 | Mef2a | 6.25363 |
| chr1 | 34890064 | 34892064 | chr1 | 34899910 | ENSMUST00000047534.5 | Fam168b | 6.25114 |
| chr11 | 118285234 | 118287234 | chr11 | 118280366 | ENSMUST00000106288.1 | Cant1 | 6.2405 |
| chr10 | 98656068 | 98658068 | chr10 | 98377786 | ENSMUST00000020107.7 | Atp2b1 | 6.23851 |
| chr17 | 71347711 | 71349711 | chr17 | 71351500 | ENSMUST00000129093.1 | 2900073G15Rik | 6.22336 |
| chr4 | 102710870 | 102712870 | chr4 | 102887408 | ENSMUST00000036195.6 | Slc35d1 | 6.20807 |
| chr11 | 30000686 | 30002686 | chr11 | 30098233 | ENSMUST00000039018.8 | Spnb2 | 6.20034 |
| chr5 | 122633877 | 122635877 | chr5 | 122608287 | ENSMUST00000102528.4 | Ppp1cc | 6.18906 |
| chr6 | 13550359 | 13552359 | chr6 | 13558100 | ENSMUST00000031554.2 | Tmem168 | 6.16111 |
| chr10 | 127517457 | 127519457 | chr10 | 127522802 | ENSMUST00000126040.1 | Atp5b | 6.12828 |
| chr16 | 91694375 | 91696375 | chr16 | 91647180 | ENSMUST00000120450.1 | Gart | 6.10021 |
| chr19 | 4136521 | 4138521 | chr19 | 4099998 | ENSMUST00000049658.7 | Pitpnm1 | 6.09677 |
| chr9 | 44376836 | 44378836 | chr9 | 44215797 | ENSMUSG00000009927.8 | Rps25 | 6.09549 |
| chr8 | 87506801 | 87508801 | chr8 | 87493486 | ENSMUST00000109734.1 | Prdx2 | 6.09138 |
| chr12 | 101776234 | 101778234 | chr12 | 101759032 | ENSMUST00000062957.6 | Ttc7b | 6.08534 |
| chr18 | 56879557 | 56881557 | chr18 | 56867467 | ENSMUST00000025486.8 | Lmnb1 | 6.0072 |
| chr19 | 55618261 | 55620261 | chr19 | 55390841 | ENSMUST00000095950.2 | Vti1a | 5.98345 |
| chr6 | 106934095 | 106936095 | chr6 | 106719135 | ENSMUST00000113249.1 | Trnt1 | 5.98287 |
| chr14 | 55035149 | 55037149 | chr14 | 55032856 | ENSMUST00000171812.1 | Slc7a7 | 5.95184 |
| chr2 | 27337091 | 27339091 | chr2 | 27319636 | ENSMUST00000164296.1 | Brd3 | 5.92184 |
| chr3 | 94856898 | 94858898 | chr3 | 95125598 | ENSMUST00000132761.1 | Lass2 | 5.89412 |
| chr4 | 135414667 | 135416667 | chr4 | 135411662 | ENSMUSG00000028676.10 | Srsf10 | 5.89234 |
| chr4 | 107753457 | 107755457 | chr4 | 107743968 | ENSMUST00000122878.1 | Scp2 | 5.86352 |
| chr4 | 114655661 | 114657661 | chr4 | 114729031 | ENSMUST00000030489.2 | Tal1 | 5.81873 |
| chr12 | 77464719 | 77466719 | chr12 | 77470547 | ENSMUST00000163120.1 | Zbtb25 | 5.79198 |
| chr4 | 154456986 | 154458986 | chr4 | 154338232 | ENSMUST00000030931.4 | Pank4 | 5.77221 |
| chr2 | 34833570 | 34835570 | chr2 | 34681755 | ENSMUST00000091020.3 | Fbxw2 | 5.74097 |
| chr14 | 79792441 | 79794441 | chr14 | 79790585 | ENSMUST00000022597.7 | Naa16 | 5.70185 |
| chr3 | 94856898 | 94858898 | chr3 | 94819160 | ENSMUST00000019482.1 | Zfp687 | 5.6951 |
| chr2 | 32725469 | 32727469 | chr2 | 32731634 | ENSMUST00000028135.8 | Fam129b | 5.66177 |
| chr11 | 75351840 | 75353840 | chr11 | 75401599 | ENSMUST00000143219.1 | Pitpna | 5.6381 |
| chr7 | 133271143 | 133273143 | chr7 | 133256407 | ENSMUST00000165608.1 | Xpo6 | 5.63739 |
| chr11 | 57766357 | 57768357 | chr11 | 57985178 | ENSMUST00000133038.1 | Mrpl22 | 5.63166 |
| chr16 | 58480474 | 58482474 | chr16 | 58508011 | ENSMUST00000137850.1 | St3gal6 | 5.61499 |
| chr12 | 4606585 | 4608585 | chr12 | 4599814 | ENSMUST00000062580.6 | Itsn2 | 5.59662 |
| chr4 | 154456986 | 154458986 | chr4 | 155186610 | ENSMUST00000139066.1 | Ccnl2 | 5.58912 |
| chr10 | 62472179 | 62474179 | chr10 | 62480832 | ENSMUST00000143689.1 | Hnrnph3 | 5.58006 |
| chr10 | 80752437 | 80754437 | chr10 | 80813225 | ENSMUST00000105323.1 | Hmg20b | 5.57087 |
| chr9 | 21423287 | 21425287 | chr9 | 21314631 | ENSMUST00000098951.3 | Tmed1 | 5.54661 |
| chr16 | 45388787 | 45390787 | chr16 | 45409166 | ENSMUST00000163230.1 | Cd200 | 5.53498 |
| chr4 | 108787213 | 108789213 | chr4 | 109149588 | ENSMUST00000064167.1 | Rnf11 | 5.52763 |
| chr10 | 24622664 | 24624664 | chr10 | 24589792 | ENSMUST00000020159.7 | Med23 | 5.50827 |
| chr10 | 24870510 | 24872510 | chr10 | 24589792 | ENSMUST00000020159.7 | Med23 | 5.50827 |
| chr11 | 59916200 | 59918200 | chr11 | 60034106 | ENSMUST00000020846.1 | Srebf1 | 5.49017 |
| chr9 | 40937169 | 40939169 | chr9 | 40966145 | ENSMUST00000044155.8 | Ubash3b | 5.45888 |
| chr9 | 40975876 | 40977876 | chr9 | 40966145 | ENSMUST00000044155.8 | Ubash3b | 5.45888 |
| chr6 | 106934095 | 106936095 | chr6 | 106750059 | ENSMUST00000013882.7 | Crbn | 5.43074 |
| chr15 | 79745826 | 79747826 | chr15 | 79763082 | ENSMUST00000109616.2 | Cbx7 | 5.42605 |
| chr10 | 62472179 | 62474179 | chr10 | 62486595 | ENSMUST00000119814.1 | Hnrnph3 | 5.42473 |
| chr14 | 79713112 | 79715112 | chr14 | 79701442 | ENSMUSG00000022018.6 | 1190002H23Rik | 5.37891 |
| chr14 | 79792441 | 79794441 | chr14 | 79701442 | ENSMUSG00000022018.6 | 1190002H23Rik | 5.37891 |
| chr14 | 27735759 | 27737759 | chr14 | 27489332 | ENSMUST00000052932.8 | Pde12 | 5.37715 |
| chr1 | 162790541 | 162792541 | chr1 | 162965297 | ENSMUST00000160429.1 | Gas5 | 5.35915 |
| chr2 | 61255296 | 61257296 | chr2 | 61431134 | ENSMUST00000112495.1 | Tank | 5.32413 |
| chr11 | 52181889 | 52183889 | chr11 | 51814264 | ENSMUSG00000020390.6 | Ube2b | 5.30511 |
| chr17 | 80283459 | 80285459 | chr17 | 80295368 | ENSMUST00000068282.5 | Atl2 | 5.27772 |
| chr15 | 57710337 | 57712337 | chr15 | 57908044 | ENSMUST00000110168.1 | Zhx1 | 5.2234 |
| chr8 | 108269176 | 108271176 | chr8 | 108225549 | ENSMUST00000093195.5 | Pard6a | 5.21853 |
| chr4 | 140690421 | 140692421 | chr4 | 140695642 | ENSMUST00000148204.1 | D4Ertd22e | 5.21805 |
| chr2 | 165793082 | 165795082 | chr2 | 165539069 | ENSMUST00000150638.1 | Eya2 | 5.19644 |
| chr2 | 153384805 | 153386805 | chr2 | 153171875 | ENSMUST00000036193.4 | Asxl1 | 5.18797 |
| chr6 | 113579365 | 113581365 | chr6 | 113573953 | ENSMUST00000035673.7 | Vhl | 5.18386 |
| chr10 | 80752437 | 80754437 | chr10 | 80951142 | ENSMUST00000151701.1 | Ncln | 5.18062 |
| chr15 | 59547763 | 59549763 | chr15 | 59480208 | ENSMUST00000118228.1 | Trib1 | 5.16592 |
| chr7 | 117252131 | 117254131 | chr7 | 117205216 | ENSMUST00000084727.2 | Zfp143 | 5.15558 |
| chr16 | 11172524 | 11174524 | chr16 | 11322985 | ENSMUST00000115814.2 | Snx29 | 5.15226 |
| chr19 | 16876042 | 16878042 | chr19 | 16855417 | ENSMUST00000163490.1 | Vps13a | 5.12253 |
| chr1 | 162790541 | 162792541 | chr1 | 163061642 | ENSMUST00000111611.1 | Klhl20 | 5.08124 |
| chr7 | 118106653 | 118108653 | chr7 | 118218899 | ENSMUST00000160552.1 | Eif4g2 | 5.06291 |
| chr13 | 98142062 | 98144062 | chr13 | 97907933 | ENSMUST00000161639.1 | Gfm2 | 5.05284 |
| chr2 | 31026010 | 31028010 | chr2 | 31007811 | ENSMUST00000133550.1 | D330023K18Rik | 5.04034 |
| chr16 | 91694375 | 91696375 | chr16 | 91465349 | ENSMUST00000149172.1 | A930006K02Rik | 5.03657 |
| chr4 | 132127950 | 132129950 | chr4 | 132119982 | ENSMUST00000070690.7 | Ptafr | 5.03473 |
| chr2 | 131937065 | 131939065 | chr2 | 131970844 | ENSMUST00000028815.8 | Slc23a2 | 5.03029 |
| chr12 | 88199859 | 88201859 | chr12 | 88225764 | ENSMUST00000038422.6 | 6430527G18Rik | 5.02061 |
| chr10 | 62472179 | 62474179 | chr10 | 62486642 | ENSMUST00000118898.1 | Hnrnph3 | 5.0013 |
| chr1 | 15839499 | 15841499 | chr1 | 16509321 | ENSMUST00000162007.1 | Stau2 | 4.97297 |
| chr18 | 84432112 | 84434112 | chr18 | 84255954 | ENSMUST00000060303.6 | Tshz1 | 4.95187 |
| chr16 | 45388787 | 45390787 | chr16 | 45409131 | ENSMUST00000172091.1 | Cd200 | 4.93362 |
| chr14 | 73338176 | 73340176 | chr14 | 73573691 | ENSMUST00000166875.1 | Rcbtb2 | 4.92406 |
| chr5 | 130679452 | 130681452 | chr5 | 130689036 | ENSMUST00000143865.1 | Rabgef1 | 4.92392 |
| chr11 | 5291490 | 5293490 | chr11 | 4428409 | ENSMUST00000123506.1 | Mtmr3 | 4.9001 |
| chr3 | 59038623 | 59040623 | chr3 | 58957447 | ENSMUSG00000074590.3 | F630111L10Rik | 4.89795 |
| chr14 | 79792441 | 79794441 | chr14 | 79786913 | ENSMUST00000163486.1 | Naa16 | 4.87997 |
| chr12 | 103985261 | 103987261 | chr12 | 103981970 | ENSMUST00000057416.6 | D230037D09Rik | 4.87884 |
| chr9 | 66113037 | 66115037 | chr9 | 66793898 | ENSMUST00000127896.1 | Rps27l | 4.86371 |
| chr6 | 31181816 | 31183816 | chr6 | 31170357 | ENSMUST00000151800.1 | 2210408F21Rik | 4.82381 |
| chr1 | 155256106 | 155258106 | chr1 | 155334790 | ENSMUST00000042141.5 | Dhx9 | 4.8236 |
| chr1 | 155447492 | 155449492 | chr1 | 155334790 | ENSMUST00000042141.5 | Dhx9 | 4.8236 |
| chr8 | 4347094 | 4349094 | chr8 | 4625840 | ENSMUST00000073201.5 | Zfp958 | 4.80962 |
| chr1 | 162790541 | 162792541 | chr1 | 162966826 | ENSMUST00000161623.1 | Gas5 | 4.80196 |
| chr14 | 21371163 | 21373163 | chr14 | 21365769 | ENSMUST00000161445.1 | Ppp3cb | 4.7746 |
| chr11 | 77257023 | 77259023 | chr11 | 77328623 | ENSMUST00000094004.4 | Abhd15 | 4.74702 |
| chr16 | 44725970 | 44727970 | chr16 | 44746472 | ENSMUST00000023348.4 | Gtpbp8 | 4.73754 |
| chr9 | 61905453 | 61907453 | chr9 | 62189882 | ENSMUST00000135395.1 | Anp32a | 4.7259 |
| chr9 | 62212191 | 62214191 | chr9 | 62189882 | ENSMUST00000135395.1 | Anp32a | 4.7259 |
| chr5 | 134745988 | 134747988 | chr5 | 135115218 | ENSMUSG00000040731.9 | Eif4h | 4.71855 |
| chr15 | 93081233 | 93083233 | chr15 | 93228765 | ENSMUST00000161409.1 | Zcrb1 | 4.6852 |
| chr11 | 75351840 | 75353840 | chr11 | 74986062 | ENSMUSG00000085609.1 | 1700016P03Rik | 4.67663 |
| chr3 | 32326102 | 32328102 | chr3 | 32335298 | ENSMUST00000108242.1 | Pik3ca | 4.66818 |
| chr9 | 21423287 | 21425287 | chr9 | 21420613 | ENSMUST00000034707.8 | Smarca4 | 4.6674 |
| chr9 | 21431017 | 21433017 | chr9 | 21420613 | ENSMUST00000034707.8 | Smarca4 | 4.6674 |
| chr10 | 92675904 | 92677904 | chr10 | 92773653 | ENSMUST00000105291.2 | Elk3 | 4.65331 |
| chr10 | 93511891 | 93513891 | chr10 | 92773653 | ENSMUST00000105291.2 | Elk3 | 4.65331 |
| chr2 | 165793082 | 165795082 | chr2 | 165818137 | ENSMUST00000099082.4 | Ncoa3 | 4.65232 |
| chr2 | 165845597 | 165847597 | chr2 | 165818137 | ENSMUST00000099082.4 | Ncoa3 | 4.65232 |
| chr7 | 87600110 | 87602110 | chr7 | 87377749 | ENSMUST00000163253.1 | D330012F22Rik | 4.64813 |
| chr2 | 90840821 | 90842821 | chr2 | 90744897 | ENSMUST00000111464.1 | Kbtbd4 | 4.64388 |
| chr9 | 69839142 | 69841142 | chr9 | 69860450 | ENSMUST00000140265.1 | Gtf2a2 | 4.63866 |
| chr8 | 96982139 | 96984139 | chr8 | 97086550 | ENSMUST00000156377.1 | Cpne2 | 4.63837 |
| chr15 | 36662475 | 36664475 | chr15 | 36722169 | ENSMUST00000126184.1 | Ywhaz | 4.63592 |
| chr13 | 34345677 | 34347677 | chr13 | 35085991 | ENSMUST00000171258.1 | Eci2 | 4.62627 |
| chr17 | 24661305 | 24663305 | chr17 | 24656153 | ENSMUST00000070777.6 | Traf7 | 4.62042 |
| chr15 | 73356433 | 73358433 | chr15 | 73342990 | ENSMUST00000043414.5 | Dennd3 | 4.61735 |
| chr17 | 71347711 | 71349711 | chr17 | 71325306 | ENSMUST00000137537.1 | Myl12b | 4.60745 |
| chr13 | 64221561 | 64223561 | chr13 | 64533861 | ENSMUST00000021939.6 | Cdk20 | 4.5981 |
| chr10 | 76368788 | 76370788 | chr10 | 76505245 | ENSMUST00000127249.1 | Slc19a1 | 4.59029 |
| chr11 | 16868043 | 16870043 | chr11 | 16952384 | ENSMUST00000020321.6 | Plek | 4.58556 |
| chr1 | 135588318 | 135590318 | chr1 | 135975697 | ENSMUSG00000020423.6 | Btg2 | 4.56703 |
| chr11 | 86221779 | 86223779 | chr11 | 86071052 | ENSMUST00000018212.6 | Ints2 | 4.54911 |
| chr5 | 134745988 | 134747988 | chr5 | 134719432 | ENSMUST00000173485.1 | Gtf2i | 4.51954 |
| chr18 | 69744124 | 69746124 | chr18 | 69505375 | ENSMUST00000078486.6 | Tcf4 | 4.49938 |
| chr18 | 69775855 | 69777855 | chr18 | 69505375 | ENSMUST00000078486.6 | Tcf4 | 4.49938 |
| chr19 | 4136521 | 4138521 | chr19 | 4000631 | ENSMUST00000122924.1 | Nudt8 | 4.4936 |
| chr7 | 88146381 | 88148381 | chr7 | 88182901 | ENSMUST00000125137.1 | Zfp592 | 4.48006 |
| chr12 | 86728378 | 86730378 | chr12 | 87423558 | ENSMUST00000077560.5 | 1700019E19Rik | 4.45907 |
| chr7 | 109112980 | 109114980 | chr7 | 109213672 | ENSMUST00000096639.5 | Rnf121 | 4.42931 |
| chr4 | 133391420 | 133393420 | chr4 | 133684704 | ENSMUSG00000028843.8 | Sh3bgrl3 | 4.42212 |
| chr12 | 33989005 | 33991005 | chr12 | 33999154 | ENSMUST00000144586.1 | Atxn7l1 | 4.4069 |
| chr5 | 135953210 | 135955210 | chr5 | 135870416 | ENSMUST00000111171.2 | Pom121 | 4.40312 |
| chr1 | 134926534 | 134928534 | chr1 | 134921943 | ENSMUST00000067398.6 | Mdm4 | 4.39784 |
| chr11 | 106299577 | 106301577 | chr11 | 106066749 | ENSMUST00000125383.1 | Ccdc47 | 4.39626 |
| chr2 | 34833570 | 34835570 | chr2 | 35192496 | ENSMUST00000028241.6 | Stom | 4.39427 |
| chr1 | 134926534 | 134928534 | chr1 | 135028324 | ENSMUST00000165011.1 | Ppp1r15b | 4.3908 |
| chr3 | 116302840 | 116304840 | chr3 | 116297926 | ENSMUST00000029571.8 | Sass6 | 4.38236 |
| chr11 | 116261380 | 116263380 | chr11 | 116533195 | ENSMUST00000134818.1 | 1810032O08Rik | 4.37337 |
| chr2 | 91793939 | 91795939 | chr2 | 91436227 | ENSMUSG00000077221.1 | Snord67 | 4.34883 |
| chr2 | 118361658 | 118363658 | chr2 | 119037378 | ENSMUST00000154185.1 | Zfyve19 | 4.34353 |
| chr9 | 57496848 | 57498848 | chr9 | 57006106 | ENSMUSG00000032299.9 | Commd4 | 4.33852 |
| chr1 | 36204991 | 36206991 | chr1 | 36301006 | ENSMUST00000174266.1 | Uggt1 | 4.33687 |
| chr2 | 6256774 | 6258774 | chr2 | 6243713 | ENSMUST00000114937.1 | Usp6nl | 4.31557 |
| chr9 | 66113037 | 66115037 | chr9 | 66198333 | ENSMUST00000042824.6 | Herc1 | 4.30501 |
| chr2 | 27337091 | 27339091 | chr2 | 27331174 | ENSMUST00000138693.1 | Brd3 | 4.30399 |
| chr4 | 129494590 | 129496590 | chr4 | 129317884 | ENSMUST00000142577.1 | Txlna | 4.29033 |
| chr15 | 100461395 | 100463395 | chr15 | 100559807 | ENSMUST00000159715.1 | Galnt6 | 4.2795 |
| chr11 | 102076861 | 102078861 | chr11 | 102268820 | ENSMUST00000153395.1 | Slc25a39 | 4.27864 |
| chr7 | 109112980 | 109114980 | chr7 | 109118357 | ENSMUST00000084852.5 | Numa1 | 4.27628 |
| chr11 | 102891992 | 102893992 | chr11 | 103128786 | ENSMUST00000021324.2 | Map3k14 | 4.27471 |
| chr16 | 91694375 | 91696375 | chr16 | 91675177 | ENSMUST00000151503.1 | Son | 4.27198 |
| chr13 | 63950593 | 63952593 | chr13 | 64230638 | ENSMUST00000099441.4 | Slc35d2 | 4.26419 |
| chr13 | 64221561 | 64223561 | chr13 | 64230638 | ENSMUST00000099441.4 | Slc35d2 | 4.26419 |
| chr11 | 75222724 | 75224724 | chr11 | 75460288 | ENSMUST00000149134.1 | Inpp5k | 4.25026 |
| chr3 | 14913354 | 14915354 | chr3 | 14641727 | ENSMUST00000029071.8 | Car13 | 4.23881 |
| chr9 | 114302873 | 114304873 | chr9 | 114310223 | ENSMUST00000111820.1 | Glb1 | 4.22831 |
| chr17 | 71438977 | 71440977 | chr17 | 71351505 | ENSMUST00000126529.1 | 2900073G15Rik | 4.21571 |
| chr6 | 146861041 | 146863041 | chr6 | 146591103 | ENSMUST00000134387.1 | Med21 | 4.20368 |
| chr9 | 107907557 | 107909557 | chr9 | 108166255 | ENSMUST00000080435.2 | Dag1 | 4.18905 |
| chr11 | 115555939 | 115557939 | chr11 | 115466181 | ENSMUST00000141556.1 | Mrps7 | 4.18699 |
| chr18 | 69312909 | 69314909 | chr18 | 69505857 | ENSMUST00000114980.1 | Tcf4 | 4.18041 |
| chr12 | 3770285 | 3772285 | chr12 | 3774525 | ENSMUST00000174414.1 | Dtnb | 4.17858 |
| chr14 | 79792441 | 79794441 | chr14 | 79797579 | ENSMUST00000022600.2 | Mtrf1 | 4.16868 |
| chr11 | 115555939 | 115557939 | chr11 | 115560848 | ENSMUST00000106497.1 | Grb2 | 4.16778 |
| chr6 | 113579365 | 113581365 | chr6 | 113588461 | ENSMUST00000059286.7 | Irak2 | 4.14335 |
| chr9 | 114302873 | 114304873 | chr9 | 114299793 | ENSMUST00000084881.4 | Crtap | 4.12363 |
| chr7 | 134624783 | 134626783 | chr7 | 134356193 | ENSMUST00000127710.1 | Mylpf | 4.11925 |
| chr2 | 73299341 | 73301341 | chr2 | 73150711 | ENSMUST00000112050.1 | Scrn3 | 4.11668 |
| chr9 | 75477514 | 75479514 | chr9 | 75473539 | ENSMUST00000034702.4 | Lysmd2 | 4.11486 |
| chr1 | 184341212 | 184343212 | chr1 | 184339303 | ENSMUST00000117245.1 | Trp53bp2 | 4.10629 |
| chr17 | 34345356 | 34347356 | chr17 | 34070870 | ENSMUST00000173284.1 | Rgl2 | 4.08925 |
| chr19 | 29457752 | 29459752 | chr19 | 29436460 | ENSMUST00000016639.5 | 5033414D02Rik | 4.0859 |
| chr1 | 9846060 | 9848060 | chr1 | 10028343 | ENSMUST00000117415.1 | Cspp1 | 4.08016 |
| chr2 | 35046233 | 35048233 | chr2 | 35056640 | ENSMUST00000113025.1 | Rab14 | 4.06534 |
| chr2 | 154422388 | 154424388 | chr2 | 154429424 | ENSMUST00000109703.2 | Pxmp4 | 4.05812 |
| chr2 | 118314465 | 118316465 | chr2 | 118227054 | ENSMUST00000110875.1 | Eif2ak4 | 4.05195 |
| chr5 | 122716393 | 122718393 | chr5 | 122804469 | ENSMUST00000154686.1 | Vps29 | 4.04352 |
| chr7 | 117252131 | 117254131 | chr7 | 117122443 | ENSMUST00000125703.1 | Tmem41b | 4.04074 |
| chr19 | 4136521 | 4138521 | chr19 | 4125975 | ENSMUST00000151401.2 | Tmem134 | 4.03169 |
| chr6 | 98971588 | 98973588 | chr6 | 98978186 | ENSMUST00000114905.2 | Foxp1 | 4.02055 |
| chr5 | 104226353 | 104228353 | chr5 | 104450938 | ENSMUST00000031251.9 | Hsd17b11 | 4.005 |
| chr14 | 63374765 | 63376765 | chr14 | 63379949 | ENSMUST00000053959.6 | Ints6 | 3.97313 |
| chr4 | 150427277 | 150429277 | chr4 | 150432055 | ENSMUST00000155446.1 | Vamp3 | 3.96486 |
| chr17 | 5800988 | 5802988 | chr17 | 6079786 | ENSMUST00000039487.3 | Gtf2h5 | 3.95932 |
| chr11 | 75222724 | 75224724 | chr11 | 75228697 | ENSMUST00000139403.1 | Serpinf1 | 3.94401 |
| chr14 | 62615075 | 62617075 | chr14 | 61928590 | ENSMUST00000055159.7 | Arl11 | 3.93908 |
| chr7 | 52079823 | 52081823 | chr7 | 52125158 | ENSMUST00000046575.9 | Ptov1 | 3.93202 |
| chr2 | 131937065 | 131939065 | chr2 | 132403969 | ENSMUST00000060955.5 | Gpcpd1 | 3.93146 |
| chr10 | 67904867 | 67906867 | chr10 | 67988515 | ENSMUST00000166919.1 | 1700040L02Rik | 3.925 |
| chr3 | 96077259 | 96079259 | chr3 | 96440976 | ENSMUST00000147821.1 | Pex11b | 3.91883 |
| chr1 | 163003668 | 163005668 | chr1 | 162967440 | ENSMUST00000163081.1 | Gas5 | 3.89314 |
| chr9 | 114302873 | 114304873 | chr9 | 114277421 | ENSMUST00000124664.1 | 4930520O04Rik | 3.88956 |
| chr7 | 52079823 | 52081823 | chr7 | 52104449 | ENSMUST00000145959.1 | Tbc1d17 | 3.87966 |
| chr6 | 125428238 | 125430238 | chr6 | 124706542 | ENSMUST00000088357.5 | Atn1 | 3.87835 |
| chr2 | 73332567 | 73334567 | chr2 | 73323820 | ENSMUST00000102680.1 | Wipf1 | 3.85976 |
| chr7 | 88146381 | 88148381 | chr7 | 88154318 | ENSMUST00000005761.8 | Zfp592 | 3.84735 |
| chr12 | 103985261 | 103987261 | chr12 | 103981841 | ENSMUST00000173760.1 | Moap1 | 3.83425 |
| chr10 | 80080382 | 80082382 | chr10 | 80024282 | ENSMUST00000051918.8 | Rexo1 | 3.82988 |
| chr17 | 36271246 | 36273246 | chr17 | 36258389 | ENSMUST00000074201.5 | H2-T10 | 3.82778 |
| chr7 | 118106653 | 118108653 | chr7 | 117911737 | ENSMUST00000155254.1 | Ampd3 | 3.8146 |
| chr2 | 27164892 | 27166892 | chr2 | 26446966 | ENSMUST00000173777.1 | Egfl7 | 3.79819 |
| chr18 | 56879557 | 56881557 | chr18 | 57135022 | ENSMUST00000025488.8 | C330018D20Rik | 3.79247 |
| chr1 | 13325378 | 13327378 | chr1 | 13362520 | ENSMUST00000081713.4 | Ncoa2 | 3.79214 |
| chr2 | 35046233 | 35048233 | chr2 | 34678701 | ENSMUST00000113078.1 | Fbxw2 | 3.76247 |
| chr10 | 62472179 | 62474179 | chr10 | 61790515 | ENSMUST00000139228.1 | Hk1 | 3.75628 |
| chr2 | 5850914 | 5852914 | chr2 | 5765897 | ENSMUST00000150876.1 | Cdc123 | 3.74913 |
| chr2 | 34833570 | 34835570 | chr2 | 34814132 | ENSMUST00000164457.1 | Traf1 | 3.746 |
| chr10 | 33848442 | 33850442 | chr10 | 33671065 | ENSMUST00000065640.3 | Zufsp | 3.74482 |
| chr17 | 50706806 | 50708806 | chr17 | 50329822 | ENSMUST00000156094.1 | Rftn1 | 3.72138 |
| chr6 | 41174603 | 41176603 | chr6 | 41071416 | ENSMUST00000103270.2 | Trbv13-2 | 3.71237 |
| chr7 | 133378789 | 133380789 | chr7 | 133344015 | ENSMUST00000168189.1 | Xpo6 | 3.70921 |
| chr19 | 4136521 | 4138521 | chr19 | 4125858 | ENSMUST00000117831.1 | Aip | 3.65198 |
| chr1 | 15839499 | 15841499 | chr1 | 16509322 | ENSMUST00000116646.1 | Stau2 | 3.64757 |
| chr1 | 172826679 | 172828679 | chr1 | 172797902 | ENSMUST00000027974.5 | Atf6 | 3.63664 |
| chr19 | 4136521 | 4138521 | chr19 | 4148619 | ENSMUST00000123874.1 | Coro1b | 3.63643 |
| chr9 | 59326379 | 59328379 | chr9 | 59334421 | ENSMUST00000171975.1 | Arih1 | 3.62887 |
| chr17 | 26050333 | 26052333 | chr17 | 25960594 | ENSMUST00000160349.1 | Wdr24 | 3.61248 |
| chr2 | 90840821 | 90842821 | chr2 | 91023975 | ENSMUST00000111372.1 | Madd | 3.60877 |
| chr17 | 37129242 | 37131242 | chr17 | 37140611 | ENSMUST00000174672.1 | Zfp57 | 3.58362 |
| chr17 | 36271246 | 36273246 | chr17 | 36248603 | ENSMUST00000172538.1 | C920025E04Rik | 3.58277 |
| chr1 | 15839499 | 15841499 | chr1 | 15795745 | ENSMUST00000093770.4 | Terf1 | 3.5802 |
| chr11 | 102076861 | 102078861 | chr11 | 102086069 | ENSMUST00000170762.1 | Hdac5 | 3.57897 |
| chr11 | 102891992 | 102893992 | chr11 | 102086069 | ENSMUST00000170762.1 | Hdac5 | 3.57897 |
| chr10 | 39950579 | 39952579 | chr10 | 40069114 | ENSMUST00000044672.4 | Cdk19 | 3.56723 |
| chr7 | 52079823 | 52081823 | chr7 | 52112774 | ENSMUST00000123015.1 | Pnkp | 3.55065 |
| chr14 | 27405756 | 27407756 | chr14 | 27399064 | ENSMUST00000037585.7 | Fam116a | 3.54065 |
| chr11 | 75351840 | 75353840 | chr11 | 75401599 | ENSMUSG00000017781.10 | Pitpna | 3.51837 |
| chr10 | 80080382 | 80082382 | chr10 | 79807985 | ENSMUST00000105353.2 | Adamtsl5 | 3.51015 |
| chr10 | 127517457 | 127519457 | chr10 | 127936420 | ENSMUST00000151955.1 | A430046D13Rik | 3.49404 |
| chr6 | 108783920 | 108785920 | chr6 | 108610623 | ENSMUSG00000030103.5 | Bhlhe40 | 3.48324 |
| chr7 | 52079823 | 52081823 | chr7 | 52119343 | ENSMUST00000128376.1 | Ptov1 | 3.47631 |
| chr7 | 134624783 | 134626783 | chr7 | 134722794 | ENSMUST00000133621.1 | Phkg2 | 3.47016 |
| chr11 | 102076861 | 102078861 | chr11 | 102268629 | ENSMUST00000149777.1 | Slc25a39 | 3.46816 |
| chr11 | 102076861 | 102078861 | chr11 | 102053339 | ENSMUST00000153178.1 | G6pc3 | 3.44142 |
| chr5 | 122716393 | 122718393 | chr5 | 122734374 | ENSMUST00000053426.8 | Pptc7 | 3.44017 |
| chr8 | 87506801 | 87508801 | chr8 | 87489860 | ENSMUST00000122931.1 | Rnaseh2a | 3.42691 |
| chr5 | 117810007 | 117812007 | chr5 | 117807313 | ENSMUST00000031309.9 | Wsb2 | 3.42239 |
| chr5 | 130679452 | 130681452 | chr5 | 130663031 | ENSMUST00000026390.7 | Rabgef1 | 3.40824 |
| chr7 | 133271143 | 133273143 | chr7 | 134035270 | ENSMUST00000154174.1 | Tmem219 | 3.40433 |
| chr19 | 4136521 | 4138521 | chr19 | 3897230 | ENSMUST00000162688.1 | Tcirg1 | 3.38399 |
| chr12 | 86728378 | 86730378 | chr12 | 86814840 | ENSMUSG00000021250.7 | Fos | 3.37888 |
| chr11 | 75222724 | 75224724 | chr11 | 75229232 | ENSMUST00000167281.1 | Serpinf1 | 3.35513 |
| chr2 | 91793939 | 91795939 | chr2 | 92061727 | ENSMUST00000159366.1 | Phf21a | 3.35378 |
| chr10 | 98704743 | 98706743 | chr10 | 98570793 | ENSMUSG00000090035.1 | Galnt4 | 3.35335 |
| chr17 | 80875858 | 80877858 | chr17 | 80879793 | ENSMUST00000068714.5 | Sos1 | 3.35033 |
| chr2 | 90840821 | 90842821 | chr2 | 90910386 | ENSMUST00000079976.3 | Slc39a13 | 3.32825 |
| chr12 | 3585919 | 3587919 | chr12 | 3426884 | ENSMUST00000111215.2 | Asxl2 | 3.31822 |
| chr7 | 52079823 | 52081823 | chr7 | 52071458 | ENSMUST00000057195.9 | Nup62 | 3.29152 |
| chr11 | 57766357 | 57768357 | chr11 | 57982041 | ENSMUST00000102711.2 | Gemin5 | 3.26764 |
| chr11 | 88810671 | 88812671 | chr11 | 88725900 | ENSMUST00000018572.4 | Akap1 | 3.26729 |
| chr7 | 135872166 | 135874166 | chr7 | 135604869 | ENSMUST00000033135.7 | Tial1 | 3.25621 |
| chr17 | 26862592 | 26864592 | chr17 | 26852595 | ENSMUST00000062519.7 | A930001N09Rik | 3.25433 |
| chr2 | 126505144 | 126507144 | chr2 | 127270216 | ENSMUST00000146437.1 | Fahd2a | 3.25376 |
| chr8 | 114115301 | 114117301 | chr8 | 114167343 | ENSMUST00000077791.6 | Zfp1 | 3.22743 |
| chr19 | 4136521 | 4138521 | chr19 | 3897831 | ENSMUST00000134698.1 | Tcirg1 | 3.22113 |
| chr7 | 134657172 | 134659172 | chr7 | 133920469 | ENSMUSG00000042675.9 | Ypel3 | 3.21826 |
| chr7 | 52079823 | 52081823 | chr7 | 52120879 | ENSMUST00000153085.1 | Ptov1 | 3.20626 |
| chr6 | 145052832 | 145054832 | chr6 | 145093994 | ENSMUST00000125029.1 | Lrmp | 3.19506 |
| chr2 | 153384805 | 153386805 | chr2 | 153270215 | ENSMUST00000109784.1 | 8430427H17Rik | 3.19224 |
| chr16 | 8724299 | 8726299 | chr16 | 8698923 | ENSMUST00000162929.1 | Usp7 | 3.18341 |
| chr8 | 108269176 | 108271176 | chr8 | 108231833 | ENSMUST00000013299.9 | E130303B06Rik | 3.16635 |
| chr1 | 134926534 | 134928534 | chr1 | 134921925 | ENSMUST00000112313.1 | Mdm4 | 3.145 |
| chr9 | 14837735 | 14839735 | chr9 | 14601597 | ENSMUST00000147676.1 | Mre11a | 3.12027 |
| chr16 | 4719688 | 4721688 | chr16 | 4684070 | ENSMUST00000060067.5 | Dnaja3 | 3.11059 |
| chr11 | 87047842 | 87049842 | chr11 | 86900276 | ENSMUST00000020801.7 | 1200011M11Rik | 3.10907 |
| chr11 | 75378410 | 75380410 | chr11 | 75401661 | ENSMUST00000102509.4 | Pitpna | 3.10861 |
| chr9 | 59326379 | 59328379 | chr9 | 59334425 | ENSMUSG00000025234.5 | Arih1 | 3.08415 |
| chr10 | 80752437 | 80754437 | chr10 | 80477670 | ENSMUST00000092285.3 | Gng7 | 3.06756 |
| chr17 | 34345356 | 34347356 | chr17 | 34335554 | ENSMUST00000172960.1 | Psmb8 | 3.04413 |
| chr10 | 75908344 | 75910344 | chr10 | 76015016 | ENSMUST00000162282.1 | Lss | 3.03484 |
| chr19 | 4136521 | 4138521 | chr19 | 4150543 | ENSMUST00000140419.1 | Coro1b | 3.03482 |
| chr17 | 36271246 | 36273246 | chr17 | 36024624 | ENSMUST00000172730.1 | Dhx16 | 3.01261 |
| chr7 | 134657172 | 134659172 | chr7 | 134565553 | ENSMUST00000148483.1 | Zfp688 | 2.97801 |
| chr18 | 35093288 | 35095288 | chr18 | 35091657 | ENSMUSG00000024360.6 | Etf1 | 2.97644 |
| chr2 | 165861063 | 165863063 | chr2 | 165594686 | ENSMUST00000128280.1 | Eya2 | 2.97266 |
| chr10 | 62472179 | 62474179 | chr10 | 62428724 | ENSMUST00000137378.1 | Dna2 | 2.96622 |
| chr9 | 44376836 | 44378836 | chr9 | 44575991 | ENSMUST00000154090.1 | Ift46 | 2.96434 |
| chr13 | 101412223 | 101414223 | chr13 | 101514612 | ENSMUST00000078573.4 | Mrps36 | 2.96101 |
| chr6 | 31181816 | 31183816 | chr6 | 31168433 | ENSMUST00000050386.4 | AB041803 | 2.95932 |
| chr14 | 21371163 | 21373163 | chr14 | 21365795 | ENSMUST00000159027.1 | Ppp3cb | 2.95146 |
| chr16 | 11172524 | 11174524 | chr16 | 10993164 | ENSMUST00000140170.1 | Litaf | 2.9482 |
| chr10 | 75908344 | 75910344 | chr10 | 75905531 | ENSMUST00000171940.1 | Pcnt | 2.91466 |
| chr9 | 75325049 | 75327049 | chr9 | 75257803 | ENSMUST00000172946.1 | Mapk6 | 2.9089 |
| chr2 | 34833570 | 34835570 | chr2 | 34629186 | ENSMUST00000155595.1 | Hspa5 | 2.90814 |
| chr15 | 78826239 | 78828239 | chr15 | 78813486 | ENSMUST00000109688.1 | Triobp | 2.89534 |
| chr13 | 101412223 | 101414223 | chr13 | 101250686 | ENSMUST00000124698.1 | Gtf2h2 | 2.88888 |
| chr12 | 86532623 | 86534623 | chr12 | 86560532 | ENSMUST00000128709.1 | Eif2b2 | 2.87768 |
| chr11 | 100843840 | 100845840 | chr11 | 100800854 | ENSMUSG00000004040.10 | Stat3 | 2.87322 |
| chr5 | 122716393 | 122718393 | chr5 | 122608365 | ENSMUST00000128309.1 | Ppp1cc | 2.8702 |
| chr8 | 96869929 | 96871929 | chr8 | 96910338 | ENSMUSG00000031770.9 | Herpud1 | 2.85987 |
| chr16 | 11172524 | 11174524 | chr16 | 11156169 | ENSMUST00000155340.1 | Zc3h7a | 2.85952 |
| chr2 | 153100512 | 153102512 | chr2 | 153067305 | ENSMUST00000123158.1 | Pofut1 | 2.83689 |
| chr9 | 114302873 | 114304873 | chr9 | 113840052 | ENSMUST00000084885.5 | Ubp1 | 2.82428 |
| chr11 | 100706701 | 100708701 | chr11 | 100948604 | ENSMUST00000149411.1 | Mlx | 2.82226 |
| chr16 | 50395250 | 50397250 | chr16 | 50432503 | ENSMUST00000138166.1 | Bbx | 2.81438 |
| chr19 | 25081144 | 25083144 | chr19 | 25074019 | ENSMUST00000025831.6 | Dock8 | 2.81088 |
| chr3 | 94856898 | 94858898 | chr3 | 94846465 | ENSMUST00000117355.1 | Psmd4 | 2.79949 |
| chr6 | 114836130 | 114838130 | chr6 | 114825125 | ENSMUST00000152710.1 | Vgl14 | 2.79523 |
| chr4 | 135414667 | 135416667 | chr4 | 135608621 | ENSMUST00000142440.1 | Rpl11 | 2.79404 |
| chr10 | 17494651 | 17496651 | chr10 | 17775746 | ENSMUST00000020001.7 | Reps1 | 2.78976 |
| chr11 | 49671870 | 49673870 | chr11 | 50047525 | ENSMUST00000122977.1 | Mgat4b | 2.77609 |
| chr11 | 110231568 | 110233568 | chr11 | 110260436 | ENSMUST00000020949.5 | Map2k6 | 2.77606 |
| chr12 | 77464719 | 77466719 | chr12 | 77471253 | ENSMUST00000042779.3 | Zbtb1 | 2.769 |
| chr2 | 31026010 | 31028010 | chr2 | 30752989 | ENSMUST00000126588.1 | Ptges | 2.76534 |
| chr6 | 38485720 | 38487720 | chr6 | 38501334 | ENSMUSG00000029823.9 | Luc7l2 | 2.76334 |
| chr9 | 45875364 | 45877364 | chr9 | 45931320 | ENSMUST00000122865.1 | Sik3 | 2.75767 |
| chr15 | 93081233 | 93083233 | chr15 | 93064794 | ENSMUSG00000065911.1 | 7SK | 2.7557 |
| chr10 | 75908344 | 75910344 | chr10 | 75260476 | ENSMUST00000139724.1 | Gstt1 | 2.75441 |
| chr11 | 72769965 | 72771965 | chr11 | 72774671 | ENSMUST00000108485.2 | Atp2a3 | 2.75155 |
| chr6 | 38485720 | 38487720 | chr6 | 38505723 | ENSMUST00000159936.1 | Luc7l2 | 2.74001 |
| chr11 | 86817754 | 86819754 | chr11 | 86621198 | ENSMUST00000018569.6 | Dhx40 | 2.7386 |
| chr17 | 24661305 | 24663305 | chr17 | 24686895 | ENSMUST00000035565.3 | Pkd1 | 2.73091 |
| chr15 | 37974875 | 37976875 | chr15 | 37988040 | ENSMUSG00000065852.1 | SNORA2 | 2.73007 |
| chr15 | 37995014 | 37997014 | chr15 | 37988040 | ENSMUSG00000065852.1 | SNORA2 | 2.73007 |
| chr5 | 114282886 | 114284886 | chr5 | 114443903 | ENSMUST00000159592.1 | Ssh1 | 2.72717 |
| chr5 | 122716393 | 122718393 | chr5 | 122621933 | ENSMUST00000151184.1 | Ppp1cc | 2.7116 |
| chr14 | 55035149 | 55037149 | chr14 | 55045746 | ENSMUSG00000010406.7 | Mrpl52 | 2.70478 |
| chr15 | 74726073 | 74728073 | chr15 | 74629963 | ENSMUST00000168815.1 | Ly6k | 2.6973 |
| chr16 | 32533124 | 32535124 | chr16 | 32148204 | ENSMUST00000150250.1 | Lrrc33 | 2.69686 |
| chr7 | 111060108 | 111062108 | chr7 | 111085896 | ENSMUSG00000073938.1 | Olfr632 | 2.67886 |
| chr2 | 29525390 | 29527390 | chr2 | 30210400 | ENSMUST00000140899.1 | Sh3glb2 | 2.67661 |
| chr2 | 6256774 | 6258774 | chr2 | 6513846 | ENSMUST00000168146.1 | Celf2 | 2.67248 |
| chr2 | 29525390 | 29527390 | chr2 | 30214785 | ENSMUST00000113620.3 | Sh3glb2 | 2.66653 |
| chr2 | 155513435 | 155515435 | chr2 | 155518068 | ENSMUST00000103140.4 | Trpc4ap | 2.65797 |
| chr11 | 86221779 | 86223779 | chr11 | 86171104 | ENSMUST00000043624.8 | Med13 | 2.64269 |
| chr9 | 45875364 | 45877364 | chr9 | 45714952 | ENSMUST00000161203.1 | Rnf214 | 2.64012 |
| chr8 | 26121545 | 26123545 | chr8 | 26127284 | ENSMUST00000084032.5 | Adam9 | 2.6364 |
| chr7 | 88146381 | 88148381 | chr7 | 88049962 | ENSMUST00000026817.4 | Nmb | 2.63118 |
| chr14 | 21371163 | 21373163 | chr14 | 21365662 | ENSMUST00000161989.1 | Ppp3cb | 2.62993 |
| chr7 | 109112980 | 109114980 | chr7 | 109045902 | ENSMUST00000137949.1 | 3200002M19Rik | 2.62841 |
| chr7 | 65983333 | 65985333 | chr7 | 65913572 | ENSMUST00000055764.6 | Atp10a | 2.62808 |
| chr10 | 41912365 | 41914365 | chr10 | 41208051 | ENSMUST00000122997.1 | Smpd2 | 2.59583 |
| chr18 | 32470773 | 32472773 | chr18 | 32227388 | ENSMUST00000025243.3 | Iws1 | 2.5939 |
| chr15 | 100461395 | 100463395 | chr15 | 100499933 | ENSMUSG00000075411.2 | Bin2 | 2.58022 |
| chr19 | 4136521 | 4138521 | chr19 | 4125960 | ENSMUST00000150627.2 | Tmem134 | 2.56416 |
| chr2 | 154422388 | 154424388 | chr2 | 154341698 | ENSMUST00000135647.1 | Cbfa2t2 | 2.55945 |
| chr4 | 137154503 | 137156503 | chr4 | 137174386 | ENSMUST00000105837.1 | Usp48 | 2.55412 |
| chr1 | 173435009 | 173437009 | chr1 | 173200505 | ENSMUST00000129985.1 | B4galt3 | 2.55007 |
| chr2 | 154422388 | 154424388 | chr2 | 154395628 | ENSMUST00000103145.4 | E2f1 | 2.54549 |
| chr2 | 91793939 | 91795939 | chr2 | 91775656 | ENSMUST00000128902.1 | Dgkz | 2.54474 |
| chr7 | 134624783 | 134626783 | chr7 | 134619015 | ENSMUST00000132124.1 | Prr14 | 2.54395 |
| chr14 | 73338176 | 73340176 | chr14 | 73725629 | ENSMUST00000022701.6 | Rb1 | 2.53809 |
| chr14 | 73571234 | 73573234 | chr14 | 73725629 | ENSMUST00000022701.6 | Rb1 | 2.53809 |
| chr14 | 73843970 | 73845970 | chr14 | 73725629 | ENSMUST00000022701.6 | Rb1 | 2.53809 |
| chr11 | 100706701 | 100708701 | chr11 | 100720665 | ENSMUST00000004145.7 | Stat5a | 2.53505 |
| chr6 | 108783920 | 108785920 | chr6 | 108778635 | ENSMUST00000169217.1 | Edem1 | 2.51779 |
| chr4 | 123422297 | 123424297 | chr4 | 123427588 | ENSMUST00000102636.3 | Akirin1 | 2.51503 |
| chr6 | 42340610 | 42342610 | chr6 | 42299827 | ENSMUSG00000029860.9 | Zyx | 2.51385 |
| chr5 | 134745988 | 134747988 | chr5 | 134575581 | ENSMUST00000016088.8 | Gatsl2 | 2.51351 |
| chr6 | 87024053 | 87026053 | chr6 | 87728130 | ENSMUSG00000030054.3 | Gp9 | 2.50747 |
| chr2 | 154422388 | 154424388 | chr2 | 154395456 | ENSMUST00000000894.5 | E2f1 | 2.50572 |
| chr11 | 72769965 | 72771965 | chr11 | 72774671 | ENSMUST00000163326.1 | Atp2a3 | 2.50446 |
| chr1 | 34890064 | 34892064 | chr1 | 34899895 | ENSMUST00000167518.1 | Fam168b | 2.5043 |
| chr2 | 118314465 | 118316465 | chr2 | 118214354 | ENSMUST00000005233.5 | Eif2ak4 | 2.50131 |
| chr4 | 120700808 | 120702808 | chr4 | 120887689 | ENSMUST00000056635.5 | Rlf | 2.48721 |
| chr9 | 75325049 | 75327049 | chr9 | 75079821 | ENSMUST00000036555.6 | Myo5c | 2.47681 |
| chr10 | 80080382 | 80082382 | chr10 | 79723757 | ENSMUST00000105363.1 | Gamt | 2.47141 |
| chr7 | 87193956 | 87195956 | chr7 | 87377381 | ENSMUST00000123279.1 | Cib1 | 2.471 |
| chr7 | 88146381 | 88148381 | chr7 | 87377381 | ENSMUST00000123279.1 | Cib1 | 2.471 |
| chr14 | 75321123 | 75323123 | chr14 | 75285062 | ENSMUST00000164780.1 | Lrch1 | 2.46179 |
| chr11 | 100843840 | 100845840 | chr11 | 100981207 | ENSMUST00000017946.5 | Fam134c | 2.45951 |
| chr11 | 100843840 | 100845840 | chr11 | 100943939 | ENSMUST00000107308.3 | Coasy | 2.45921 |
| chr3 | 133187535 | 133189535 | chr3 | 133207354 | ENSMUST00000098603.3 | Tet2 | 2.45776 |
| chr2 | 70623165 | 70625165 | chr2 | 70893838 | ENSMUST00000064141.5 | Dcaf17 | 2.45364 |
| chr3 | 96077259 | 96079259 | chr3 | 96072577 | ENSMUST00000098843.2 | Hist2h3b | 2.4516 |
| chr7 | 52079823 | 52081823 | chr7 | 52104260 | ENSMUST00000130081.1 | Tbc1d17 | 2.44526 |
| chr3 | 116302840 | 116304840 | chr3 | 116297778 | ENSMUST00000128687.1 | Ccdc76 | 2.44363 |
| chr3 | 95950914 | 95952914 | chr3 | 96002412 | ENSMUSG00000015943.4 | Bola1 | 2.42473 |
| chr8 | 47708070 | 47710070 | chr8 | 47698369 | ENSMUST00000125319.1 | Ccdc111 | 2.4194 |
| chr8 | 108269176 | 108271176 | chr8 | 108160468 | ENSMUST00000132679.1 | Ctcf | 2.41561 |
| chr6 | 124151275 | 124153275 | chr6 | 125021295 | ENSMUST00000171989.1 | Lpar5 | 2.40318 |
| chr10 | 119620496 | 119622496 | chr10 | 119638668 | ENSMUST00000145015.1 | Tmbim4 | 2.39625 |
| chr11 | 100843840 | 100845840 | chr11 | 100683842 | ENSMUST00000107358.2 | Stat5b | 2.3875 |
| chr10 | 80080382 | 80082382 | chr10 | 80065625 | ENSMUST00000038411.4 | Adat3 | 2.38357 |
| chr18 | 78140532 | 78142532 | chr18 | 78135239 | ENSMUST00000044622.4 | 5430411K18Rik | 2.37321 |
| chr14 | 47646852 | 47648852 | chr14 | 47917966 | ENSMUST00000043494.9 | Mapk1ip11 | 2.37272 |
| chr18 | 61131242 | 61133242 | chr18 | 60934164 | ENSMUSG00000024608.4 | Rps14 | 2.37047 |
| chr1 | 172826679 | 172828679 | chr1 | 173220571 | ENSMUST00000138974.1 | Ufc1 | 2.36835 |
| chr16 | 14160427 | 14162427 | chr16 | 13903075 | ENSMUST00000154150.1 | Pdxdc1 | 2.3548 |
| chr17 | 5800988 | 5802988 | chr17 | 5841346 | ENSMUST00000002436.8 | Snx9 | 2.35374 |
| chr17 | 71347711 | 71349711 | chr17 | 71368861 | ENSMUST00000024847.6 | Myom1 | 2.3458 |
| chr11 | 116261380 | 116263380 | chr11 | 116334557 | ENSMUST00000147858.1 | Prpsap1 | 2.34353 |
| chr7 | 96362890 | 96364890 | chr7 | 96552876 | ENSMUST00000058755.3 | Fzd4 | 2.33742 |
| chr16 | 91694375 | 91696375 | chr16 | 91672508 | ENSMUST00000167141.1 | Son | 2.33324 |
| chr4 | 137154503 | 137156503 | chr4 | 137150055 | ENSMUST00000105840.1 | Usp48 | 2.33075 |
| chr13 | 108645689 | 108647689 | chr13 | 109004598 | ENSMUST00000022207.8 | Elovl7 | 2.32573 |
| chr3 | 102821347 | 102823347 | chr3 | 102799718 | ENSMUST00000119450.1 | Sike1 | 2.31474 |
| chr9 | 59326379 | 59328379 | chr9 | 59598456 | ENSMUST00000051039.4 | Senp8 | 2.31358 |
| chr5 | 148252123 | 148254123 | chr5 | 148242156 | ENSMUSG00000029647.7 | Pan3 | 2.31172 |
| chr19 | 4136521 | 4138521 | chr19 | 4306030 | ENSMUST00000113837.2 | Adrbk1 | 2.31139 |
| chr16 | 45175490 | 45177490 | chr16 | 45158819 | ENSMUST00000023344.3 | Slc35a5 | 2.3045 |
| chr12 | 33023816 | 33025816 | chr12 | 33832628 | ENSMUST00000125192.1 | Atxn7l1 | 2.2931 |
| chr3 | 94856898 | 94858898 | chr3 | 95736724 | ENSMUST00000167876.1 | Anp32e | 2.28944 |
| chr7 | 52079823 | 52081823 | chr7 | 52347095 | ENSMUST00000141576.1 | Rcn3 | 2.28943 |
| chr11 | 49671870 | 49673870 | chr11 | 49526225 | ENSMUSG00000020362.7 | Cnot6 | 2.283 |
| chr2 | 78680699 | 78682699 | chr2 | 78708281 | ENSMUSG00000027011.8 | Ube2e3 | 2.2803 |
| chr7 | 133271143 | 133273143 | chr7 | 132588190 | ENSMUST00000033010.2 | Jmjd5 | 2.27465 |
| chr2 | 91793939 | 91795939 | chr2 | 91771937 | ENSMUST00000069423.6 | Mdk | 2.27399 |
| chr11 | 19995723 | 19997723 | chr11 | 20101612 | ENSMUST00000152728.1 | Rab1 | 2.27393 |
| chr14 | 21782499 | 21784499 | chr14 | 21834142 | ENSMUST00000090432.5 | Vcl | 2.26855 |
| chr4 | 135414667 | 135416667 | chr4 | 135528503 | ENSMUST00000145350.1 | Lypla2 | 2.26294 |
| chr10 | 75908344 | 75910344 | chr10 | 75905657 | ENSMUST00000001179.5 | Pcnt | 2.24931 |
| chr14 | 55035149 | 55037149 | chr14 | 55283514 | ENSMUST00000141993.1 | Acin1 | 2.24673 |
| chr3 | 94856898 | 94858898 | chr3 | 94815729 | ENSMUST00000146169.1 | Zfp687 | 2.22336 |
| chr19 | 4136521 | 4138521 | chr19 | 4125959 | ENSMUST00000139718.2 | Tmem134 | 2.22041 |
| chr5 | 130679452 | 130681452 | chr5 | 130729955 | ENSMUST00000125625.1 | Sbds | 2.21666 |
| chr6 | 125428238 | 125430238 | chr6 | 125596357 | ENSMUST00000141521.1 | Vwf | 2.2161 |
| chr4 | 131585487 | 131587487 | chr4 | 131631236 | ENSMUST00000146021.1 | Epb4.1 | 2.2147 |
| chr8 | 96982139 | 96984139 | chr8 | 96911453 | ENSMUST00000161085.1 | Herpud1 | 2.2124 |
| chr17 | 31978023 | 31980023 | chr17 | 32150348 | ENSMUST00000133308.1 | Hsf2bp | 2.20607 |
| chr15 | 93081233 | 93083233 | chr15 | 93228765 | ENSMUSG00000022635.3 | Zcrb1 | 2.20522 |
| chr11 | 49671870 | 49673870 | chr11 | 49667715 | ENSMUST00000102778.1 | Mapk9 | 2.19629 |
| chr11 | 59916200 | 59918200 | chr11 | 60591027 | ENSMUST00000056907.6 | Smcr8 | 2.19397 |
| chr9 | 99460139 | 99462139 | chr9 | 99476527 | ENSMUST00000138002.1 | Dbr1 | 2.19352 |
| chr3 | 100305904 | 100307904 | chr3 | 100293247 | ENSMUST00000061455.8 | Fam46c | 2.18813 |
| chr11 | 16868043 | 16870043 | chr11 | 16851121 | ENSMUST00000139493.1 | 2810442I21Rik | 2.1862 |
| chr13 | 12632904 | 12634904 | chr13 | 12548790 | ENSMUST00000155871.1 | Lgals8 | 2.18174 |
| chr11 | 86817754 | 86819754 | chr11 | 86807209 | ENSMUST00000018571.4 | Ypel2 | 2.16997 |
| chr3 | 95950914 | 95952914 | chr3 | 96042999 | ENSMUST00000117968.1 | Hist2h3c2 | 2.16531 |
| chr17 | 30396154 | 30398154 | chr17 | 30142181 | ENSMUST00000052403.8 | Zfand3 | 2.15948 |
| chr5 | 114320348 | 114322348 | chr5 | 114550342 | ENSMUST00000031588.7 | Usp30 | 2.15812 |
| chr3 | 94856898 | 94858898 | chr3 | 95111098 | ENSMUSG00000015702.7 | Anxa9 | 2.14391 |
| chr11 | 75351840 | 75353840 | chr11 | 75327042 | ENSMUSG00000038188.10 | Scarf1 | 2.14092 |
| chr11 | 75378410 | 75380410 | chr11 | 75327042 | ENSMUSG00000038188.10 | Scarf1 | 2.14092 |
| chr19 | 9113416 | 9115416 | chr19 | 8972604 | ENSMUST00000096247.3 | Ganab | 2.14018 |
| chr17 | 34345356 | 34347356 | chr17 | 34259262 | ENSMUSG00000024335.12 | Brd2 | 2.13239 |
| chr16 | 76335594 | 76337594 | chr16 | 76373294 | ENSMUST00000145649.1 | Nrip1 | 2.12739 |
| chr7 | 63212697 | 63214697 | chr7 | 63217615 | ENSMUST00000119041.1 | Nipa2 | 2.1264 |
| chr12 | 32847765 | 32849765 | chr12 | 32746161 | ENSMUST00000036497.9 | Prkar2b | 2.12564 |
| chr12 | 33023816 | 33025816 | chr12 | 32746161 | ENSMUST00000036497.9 | Prkar2b | 2.12564 |
| chr1 | 95380382 | 95382382 | chr1 | 95375541 | ENSMUST00000027495.8 | 2-Sep | 2.12485 |
| chr15 | 100461395 | 100463395 | chr15 | 100467296 | ENSMUST00000172334.1 | Smagp | 2.11851 |
| chr1 | 134926534 | 134928534 | chr1 | 134921925 | ENSMUST00000067429.3 | Mdm4 | 2.10349 |
| chr1 | 135588318 | 135590318 | chr1 | 136311955 | ENSMUSG00000026457.8 | Adipor1 | 2.10253 |
| chr7 | 74557778 | 74559778 | chr7 | 73852990 | ENSMUST00000065323.6 | Lins | 2.10237 |
| chr4 | 123422297 | 123424297 | chr4 | 123427588 | ENSMUSG00000023075.9 | Akirin1 | 2.09976 |
| chr5 | 104226353 | 104228353 | chr5 | 104183181 | ENSMUST00000054979.3 | Aff1 | 2.09663 |
| chr17 | 34345356 | 34347356 | chr17 | 34293790 | ENSMUST00000174765.1 | H2-DMb1 | 2.09327 |
| chr8 | 4347094 | 4349094 | chr8 | 4325100 | ENSMUSG00000040028.9 | Elavl1 | 2.0927 |
| chr9 | 116069641 | 116071641 | chr9 | 116084383 | ENSMUST00000061101.3 | Tgfbr2 | 2.08386 |
| chr11 | 75351840 | 75353840 | chr11 | 75323969 | ENSMUST00000156923.1 | Rilp | 2.07103 |
| chr6 | 145052832 | 145054832 | chr6 | 145070262 | ENSMUST00000149244.1 | Lrmp | 2.05202 |
| chr2 | 91793939 | 91795939 | chr2 | 92160586 | ENSMUST00000159727.1 | Phf21a | 2.03708 |
| chr11 | 118285234 | 118287234 | chr11 | 118280337 | ENSMUST00000092378.3 | Cant1 | 2.03632 |
| chr1 | 93127676 | 93129676 | chr1 | 93146914 | ENSMUST00000171165.1 | Ube2f | 2.0351 |
| chr1 | 163003668 | 163005668 | chr1 | 162964553 | ENSMUSG00000053332.7 | Gas5 | 2.02249 |
| chr1 | 173435009 | 173437009 | chr1 | 173044049 | ENSMUST00000129651.1 | 1700009P17Rik | 2.01342 |
| chr12 | 103985261 | 103987261 | chr12 | 103981870 | ENSMUST00000166916.1 | Moap1 | 2.0089 |
| chr17 | 37129242 | 37131242 | chr17 | 37182965 | ENSMUST00000173823.1 | Gabbr1 | 2.00662 |
| chr10 | 119620496 | 119622496 | chr10 | 119639707 | ENSMUST00000135794.1 | Tmbim4 | 2.00259 |
| chr1 | 173435009 | 173437009 | chr1 | 173347461 | ENSMUST00000159929.1 | Usf1 | 2.0022 |
| chr1 | 36695504 | 36697504 | chr1 | 36502116 | ENSMUST00000115011.1 | Lman2l | 2.0016 |
| chr1 | 95380382 | 95382382 | chr1 | 95375638 | ENSMUST00000168776.1 | 2-Sep | 1.99293 |
| chr16 | 91694375 | 91696375 | chr16 | 91804881 | ENSMUST00000156841.1 | Itsn1 | 1.99132 |
| chr15 | 37995014 | 37997014 | chr15 | 37891073 | ENSMUSG00000022292.9 | Rrm2b | 1.98799 |
| chr4 | 3583814 | 3585814 | chr4 | 3865529 | ENSMUST00000003369.3 | Plag1 | 1.98558 |
| chr5 | 135953210 | 135955210 | chr5 | 135850049 | ENSMUST00000124453.1 | Nsun5 | 1.98235 |
| chr16 | 23287655 | 23289655 | chr16 | 23108655 | ENSMUST00000135020.1 | Eif4a2 | 1.9745 |
| chr12 | 3423035 | 3425035 | chr12 | 3426912 | ENSMUST00000140046.2 | Asxl2 | 1.97141 |
| chr5 | 30219040 | 30221040 | chr5 | 30091730 | ENSMUST00000139126.1 | Dnajb6 | 1.96346 |
| chr2 | 90840821 | 90842821 | chr2 | 90898266 | ENSMUST00000145317.1 | Psmc3 | 1.95362 |
| chr16 | 49965080 | 49967080 | chr16 | 49699346 | ENSMUST00000046777.4 | Ift57 | 1.95238 |
| chr12 | 3585919 | 3587919 | chr12 | 3426644 | ENSMUST00000095903.1 | 1110002L01Rik | 1.94595 |
| chr19 | 4136521 | 4138521 | chr19 | 4111929 | ENSMUST00000127056.1 | Pitpnm1 | 1.9455 |
| chr7 | 91268003 | 91270003 | chr7 | 91032851 | ENSMUST00000094216.3 | Mesdc1 | 1.93238 |
| chr9 | 69839142 | 69841142 | chr9 | 69860372 | ENSMUST00000119905.1 | Gtf2a2 | 1.9254 |
| chr7 | 133271143 | 133273143 | chr7 | 134005200 | ENSMUST00000106342.1 | Ino80e | 1.92147 |
| chr10 | 119604888 | 119606888 | chr10 | 119645849 | ENSMUST00000141206.1 | Tmbim4 | 1.92113 |
| chr17 | 44328980 | 44330980 | chr17 | 44325521 | ENSMUST00000024755.5 | Clic5 | 1.91749 |
| chr8 | 111253696 | 111255696 | chr8 | 111238544 | ENSMUST00000043896.7 | Zfhx3 | 1.9128 |
| chr19 | 55939843 | 55941843 | chr19 | 55816958 | ENSMUST00000153888.1 | Tcf7l2 | 1.91165 |
| chr11 | 77257023 | 77259023 | chr11 | 77329233 | ENSMUST00000136101.1 | Trp53i13 | 1.91115 |
| chr17 | 71438977 | 71440977 | chr17 | 71711299 | ENSMUST00000147111.1 | Smchd1 | 1.89757 |
| chr11 | 88810671 | 88812671 | chr11 | 88860690 | ENSMUSG00000000275.9 | Trim25 | 1.8936 |
| chr1 | 162790541 | 162792541 | chr1 | 163000898 | ENSMUST00000143486.1 | Cenpl | 1.88456 |
| chr1 | 163003668 | 163005668 | chr1 | 163000898 | ENSMUST00000143486.1 | Cenpl | 1.88456 |
| chr10 | 62472179 | 62474179 | chr10 | 62486965 | ENSMUSG00000020069.9 | Hnrnph3 | 1.8834 |
| chr7 | 134657172 | 134659172 | chr7 | 134376769 | ENSMUST00000056232.6 | Zfp553 | 1.88073 |
| chr11 | 84944311 | 84946311 | chr11 | 84925295 | ENSMUST00000121801.1 | Rpl13-ps1 | 1.8764 |
| chr10 | 39950579 | 39952579 | chr10 | 39862062 | ENSMUST00000045307.5 | Slc16a10 | 1.86967 |
| chr14 | 52868009 | 52870009 | chr14 | 52833054 | ENSMUST00000140603.1 | Chd8 | 1.86654 |
| chr3 | 145874556 | 145876556 | chr3 | 146113434 | ENSMUST00000061937.6 | Ctbs | 1.85957 |
| chr2 | 131937065 | 131939065 | chr2 | 132111675 | ENSMUST00000089461.4 | Cds2 | 1.85443 |
| chr17 | 34345356 | 34347356 | chr17 | 34257328 | ENSMUST00000114241.3 | Brd2 | 1.84972 |
| chr11 | 102076861 | 102078861 | chr11 | 102060209 | ENSMUST00000140481.1 | Hdac5 | 1.83537 |
| chr10 | 128039557 | 128041557 | chr10 | 127962926 | ENSMUSG00000025366.6 | Esyt1 | 1.83308 |
| chr4 | 129494590 | 129496590 | chr4 | 129277843 | ENSMUST00000135055.1 | Eif3i | 1.81746 |
| chr4 | 133391420 | 133393420 | chr4 | 133524565 | ENSMUSG00000003038.9 | Hmgn2 | 1.81221 |
| chr5 | 30219040 | 30221040 | chr5 | 30090721 | ENSMUST00000149396.1 | Dnajb6 | 1.81021 |
| chr6 | 54702063 | 54704063 | chr6 | 54922606 | ENSMUST00000060655.8 | Nod1 | 1.80775 |
| chr15 | 79745826 | 79747826 | chr15 | 79377171 | ENSMUSG00000055065.6 | Ddx17 | 1.80233 |
| chr1 | 39768773 | 39770773 | chr1 | 39777842 | ENSMUST00000151913.1 | Rfx8 | 1.79489 |
| chr2 | 29525390 | 29527390 | chr2 | 30252941 | ENSMUST00000152303.1 | Dolpp1 | 1.7943 |
| chr16 | 44725970 | 44727970 | chr16 | 44746396 | ENSMUST00000161436.1 | Gtpbp8 | 1.78956 |
| chr11 | 72769965 | 72771965 | chr11 | 72861372 | ENSMUST00000144262.1 | 1200014J11Rik | 1.78863 |
| chr18 | 66605331 | 66607331 | chr18 | 66618258 | ENSMUST00000025399.7 | Pmaip1 | 1.78701 |
| chr18 | 66635286 | 66637286 | chr18 | 66618258 | ENSMUST00000025399.7 | Pmaip1 | 1.78701 |
| chrX | 34634483 | 34636483 | chrX | 34625397 | ENSMUSG00000079641.3 | Rpl39 | 1.78646 |
| chr8 | 87506801 | 87508801 | chr8 | 87432630 | ENSMUST00000134569.1 | Dnase2a | 1.77589 |
| chr13 | 12632904 | 12634904 | chr13 | 12658150 | ENSMUST00000071973.6 | Ero1lb | 1.77586 |
| chr7 | 87600110 | 87602110 | chr7 | 87550322 | ENSMUSG00000030530.9 | Furin | 1.77424 |
| chr2 | 165793082 | 165795082 | chr2 | 165818137 | ENSMUST00000088095.5 | Ncoa3 | 1.76901 |
| chr2 | 165845597 | 165847597 | chr2 | 165818137 | ENSMUST00000088095.5 | Ncoa3 | 1.76901 |
| chr2 | 165861063 | 165863063 | chr2 | 165818137 | ENSMUST00000088095.5 | Ncoa3 | 1.76901 |
| chr2 | 60701888 | 60703888 | chr2 | 60801249 | ENSMUST00000028347.6 | Rbms1 | 1.75939 |
| chr8 | 108269176 | 108271176 | chr8 | 107995322 | ENSMUST00000014990.5 | Tppp3 | 1.75718 |
| chr4 | 132127950 | 132129950 | chr4 | 132194979 | ENSMUST00000134868.1 | Eya3 | 1.75436 |
| chr2 | 126505144 | 126507144 | chr2 | 126501280 | ENSMUST00000103227.1 | Gabpb1 | 1.75238 |
| chr11 | 75351840 | 75353840 | chr11 | 74992289 | ENSMUST00000123489.1 | Ovca2 | 1.73447 |
| chr19 | 9113416 | 9115416 | chr19 | 8915025 | ENSMUST00000162071.1 | Bscl2 | 1.73326 |
| chr15 | 93081233 | 93083233 | chr15 | 93105592 | ENSMUST00000049484.6 | Gxylt1 | 1.72974 |
| chr12 | 80117826 | 80119826 | chr12 | 80398338 | ENSMUST00000171210.1 | Rad51l1 | 1.72592 |
| chr19 | 4136521 | 4138521 | chr19 | 4110800 | ENSMUST00000126620.1 | Pitpnm1 | 1.72199 |
| chr2 | 34833570 | 34835570 | chr2 | 35056640 | ENSMUSG00000026878.9 | Rab14 | 1.72129 |
| chr2 | 35017577 | 35019577 | chr2 | 35056640 | ENSMUSG00000026878.9 | Rab14 | 1.72129 |
| chr10 | 127517457 | 127519457 | chr10 | 127521490 | ENSMUST00000139295.1 | Atp5b | 1.7211 |
| chr9 | 107907557 | 107909557 | chr9 | 107981632 | ENSMUST00000162355.1 | Rnf123 | 1.72002 |
| chr19 | 9113416 | 9115416 | chr19 | 9090282 | ENSMUST00000170708.1 | Ahnak | 1.71716 |
| chr10 | 80080382 | 80082382 | chr10 | 79716060 | ENSMUST00000155336.1 | Ndufs7 | 1.70893 |
| chr1 | 93127676 | 93129676 | chr1 | 93146888 | ENSMUST00000059743.5 | Ube2f | 1.70347 |
| chr7 | 134624783 | 134626783 | chr7 | 134985484 | ENSMUST00000138399.1 | Stx4a | 1.70224 |
| chr2 | 118314465 | 118316465 | chr2 | 118702169 | ENSMUST00000154104.1 | Ivd | 1.69897 |
| chr6 | 31181816 | 31183816 | chr6 | 31168433 | ENSMUSG00000044471.4 | AB041803 | 1.69213 |
| chr7 | 117252131 | 117254131 | chr7 | 117204839 | ENSMUSG00000073867.2 | AA474408 | 1.68279 |
| chr9 | 45855653 | 45857653 | chr9 | 45820903 | ENSMUSG00000034135.7 | Sik3 | 1.68219 |
| chr8 | 96982139 | 96984139 | chr8 | 97374025 | ENSMUST00000160364.1 | Coq9 | 1.67489 |
| chr16 | 91378045 | 91380045 | chr16 | 91647751 | ENSMUSG00000022961.10 | Son | 1.67296 |
| chr10 | 126563534 | 126565534 | chr10 | 126558216 | ENSMUSG00000040462.6 | Os9 | 1.66107 |
| chr11 | 102076861 | 102078861 | chr11 | 102050983 | ENSMUST00000078975.7 | G6pc3 | 1.65994 |
| chr16 | 91694375 | 91696375 | chr16 | 91011553 | ENSMUST00000121759.1 | Synj1 | 1.65977 |
| chr4 | 140690421 | 140692421 | chr4 | 140695655 | ENSMUST00000128444.1 | D4Ertd22e | 1.65628 |
| chr11 | 94548275 | 94550275 | chr11 | 94515345 | ENSMUST00000125148.1 | Mrpl27 | 1.6542 |
| chrX | 34634483 | 34636483 | chrX | 34357180 | ENSMUST00000170210.1 | 2310010G23Rik | 1.65179 |
| chr6 | 13550359 | 13552359 | chr6 | 13558019 | ENSMUST00000146139.1 | Tmem168 | 1.64629 |
| chr10 | 119620496 | 119622496 | chr10 | 119638714 | ENSMUST00000156877.1 | Tmbim4 | 1.64145 |
| chr5 | 122633877 | 122635877 | chr5 | 122889383 | ENSMUST00000148266.1 | Anapc7 | 1.62935 |
| chr3 | 145874556 | 145876556 | chr3 | 145601006 | ENSMUST00000134575.1 | 2410004B18Rik | 1.62543 |
| chr7 | 133271143 | 133273143 | chr7 | 133248322 | ENSMUST00000166719.1 | Xpo6 | 1.61773 |
| chr2 | 35017577 | 35019577 | chr2 | 35039668 | ENSMUST00000142015.1 | Rab14 | 1.6156 |
| chr2 | 35046233 | 35048233 | chr2 | 35039668 | ENSMUST00000142015.1 | Rab14 | 1.6156 |
| chr4 | 154456986 | 154458986 | chr4 | 154441125 | ENSMUST00000103180.3 | Pex10 | 1.61435 |
| chr6 | 113579365 | 113581365 | chr6 | 113293898 | ENSMUST00000149497.1 | Camk1 | 1.60913 |
| chr16 | 8724299 | 8726299 | chr16 | 8830193 | ENSMUSG00000022507.5 | 1810013L24Rik | 1.60466 |
| chr17 | 37129242 | 37131242 | chr17 | 37182911 | ENSMUST00000025338.9 | Gabbr1 | 1.60235 |
| chr14 | 119328244 | 119330244 | chr14 | 119405939 | ENSMUST00000131424.1 | Uggt2 | 1.60223 |
| chr7 | 52079823 | 52081823 | chr7 | 51790461 | ENSMUST00000123787.1 | Pold1 | 1.59788 |
| chr19 | 4136521 | 4138521 | chr19 | 4125979 | ENSMUST00000148807.2 | Tmem134 | 1.59573 |
| chr13 | 28605724 | 28607724 | chr13 | 28612977 | ENSMUST00000134787.1 | 2610307P16Rik | 1.58622 |
| chr12 | 77464719 | 77466719 | chr12 | 77505163 | ENSMUSG00000059970.6 | Hspa2 | 1.58269 |
| chr7 | 52079823 | 52081823 | chr7 | 51723459 | ENSMUST00000136679.1 | Josd2 | 1.5779 |
| chr1 | 163003668 | 163005668 | chr1 | 163000787 | ENSMUST00000160759.1 | Dars2 | 1.57691 |
| chr14 | 73381754 | 73383754 | chr14 | 73109804 | ENSMUST00000162922.1 | Fndc3a | 1.56716 |
| chr7 | 134657172 | 134659172 | chr7 | 134717156 | ENSMUST00000138158.1 | Phkg2 | 1.55765 |
| chr4 | 154456986 | 154458986 | chr4 | 154460686 | ENSMUST00000030915.4 | Morn1 | 1.55734 |
| chr16 | 91694375 | 91696375 | chr16 | 91689010 | ENSMUST00000139324.1 | Donson | 1.5538 |
| chr16 | 76040422 | 76042422 | chr16 | 75767036 | ENSMUST00000114244.1 | Hspa13 | 1.55191 |
| chr16 | 44725970 | 44727970 | chr16 | 44746422 | ENSMUST00000162479.1 | Gtpbp8 | 1.54769 |
| chr10 | 128039557 | 128041557 | chr10 | 127669145 | ENSMUST00000105244.1 | Timeless | 1.54076 |
| chr10 | 19853457 | 19855457 | chr10 | 19868277 | ENSMUSG00000019996.9 | Mtap7 | 1.5402 |
| chr4 | 132127950 | 132129950 | chr4 | 132288443 | ENSMUST00000150104.1 | Xkr8 | 1.5398 |
| chr4 | 154609450 | 154611450 | chr4 | 154596701 | ENSMUSG00000029050.8 | Ski | 1.53819 |
| chr3 | 135227472 | 135229472 | chr3 | 135101261 | ENSMUSG00000078578.3 | Ube2d3 | 1.53816 |
| chr19 | 55618261 | 55620261 | chr19 | 55816300 | ENSMUST00000111657.3 | Tcf7l2 | 1.53674 |
| chr9 | 62212191 | 62214191 | chr9 | 62189100 | ENSMUST00000138226.1 | Anp32a | 1.53565 |
| chr10 | 128039557 | 128041557 | chr10 | 128026708 | ENSMUST00000082059.6 | Erbb3 | 1.53006 |
| chr18 | 25488310 | 25490310 | chr18 | 24812192 | ENSMUST00000068006.7 | Mocos | 1.52628 |
| chr11 | 72769965 | 72771965 | chr11 | 72774792 | ENSMUST00000149493.1 | Atp2a3 | 1.5248 |
| chr2 | 28978040 | 28980040 | chr2 | 29675595 | ENSMUST00000149379.1 | Urm1 | 1.52344 |
| chr2 | 29525390 | 29527390 | chr2 | 29675595 | ENSMUST00000149379.1 | Urm1 | 1.52344 |
| chr6 | 5209639 | 5211639 | chr6 | 4455697 | ENSMUST00000169615.1 | Col1a2 | 1.5188 |
| chr6 | 98971588 | 98973588 | chr6 | 98978260 | ENSMUST00000113321.1 | Foxp1 | 1.51758 |
| chr18 | 82699936 | 82701936 | chr18 | 82723908 | ENSMUST00000133193.1 | Mbp | 1.51724 |
| chr1 | 15839499 | 15841499 | chr1 | 16094743 | ENSMUSG00000043716.7 | Rpl7 | 1.51075 |
| chr11 | 57766357 | 57768357 | chr11 | 58133456 | ENSMUST00000155662.1 | Zfp672 | 1.51065 |
| chr5 | 23378406 | 23380406 | chr5 | 23356415 | ENSMUSG00000086802.1 | 2700038G22Rik | 1.4991 |
| chr17 | 26050333 | 26052333 | chr17 | 26068213 | ENSMUST00000139078.1 | Pigq | 1.49673 |
| chr15 | 78826239 | 78828239 | chr15 | 78813507 | ENSMUST00000130663.2 | Triobp | 1.49642 |
| chr5 | 122633877 | 122635877 | chr5 | 122821885 | ENSMUSG00000029464.4 | Gpn3 | 1.49508 |
| chr16 | 43950381 | 43952381 | chr16 | 43889913 | ENSMUST00000132859.1 | 2610015P09Rik | 1.49471 |
| chr1 | 108456848 | 108458848 | chr1 | 108438520 | ENSMUST00000144260.1 | D630008O14Rik | 1.48863 |
| chr1 | 108464140 | 108466140 | chr1 | 108438520 | ENSMUST00000144260.1 | D630008O14Rik | 1.48863 |
| chr10 | 80080382 | 80082382 | chr10 | 80783848 | ENSMUSG00000034889.7 | 2510012J08Rik | 1.48361 |
| chr7 | 25891065 | 25893065 | chr7 | 26131000 | ENSMUST00000153077.1 | MegfB | 1.47734 |
| chr4 | 131753294 | 131755294 | chr4 | 131768218 | ENSMUSG00000040025.10 | Ythdf2 | 1.47555 |
| chr11 | 59916200 | 59918200 | chr11 | 59646795 | ENSMUST00000136901.1 | Cops3 | 1.46765 |
| chr14 | 52868009 | 52870009 | chr14 | 52924758 | ENSMUST00000153539.2 | Mettl3 | 1.46737 |
| chr16 | 50395250 | 50397250 | chr16 | 50432453 | ENSMUST00000066037.6 | Bbx | 1.46258 |
| chr7 | 63212697 | 63214697 | chr7 | 63217846 | ENSMUST00000032635.7 | Nipa2 | 1.45847 |
| chr2 | 78976919 | 78978919 | chr2 | 79269111 | ENSMUST00000099974.3 | Cerkl | 1.45743 |
| chr7 | 99808096 | 99810096 | chr7 | 99818443 | ENSMUSG00000041328.9 | Pcf11 | 1.45664 |
| chr3 | 37502078 | 37504078 | chr3 | 37211476 | ENSMUST00000057975.7 | Bbs12 | 1.4563 |
| chr6 | 146529552 | 146531552 | chr6 | 146526464 | ENSMUST00000147862.1 | Fgfr1op2 | 1.45266 |
| chr10 | 80752437 | 80754437 | chr10 | 80720087 | ENSMUST00000150605.1 | Matk | 1.45208 |
| chr11 | 86221779 | 86223779 | chr11 | 86014695 | ENSMUST00000044423.3 | Brip1 | 1.44461 |
| chr3 | 95950914 | 95952914 | chr3 | 95238441 | ENSMUST00000149051.1 | Arnt | 1.44299 |
| chr2 | 90840821 | 90842821 | chr2 | 90838736 | ENSMUST00000111449.1 | Celf1 | 1.43701 |
| chr6 | 145052832 | 145054832 | chr6 | 145159695 | ENSMUST00000111724.1 | Lyrm5 | 1.42457 |
| chr15 | 78826239 | 78828239 | chr15 | 78939413 | ENSMUST00000040320.7 | Micall1 | 1.42397 |
| chr3 | 95950914 | 95952914 | chr3 | 95697892 | ENSMUSG00000015750.8 | Aph1a | 1.42394 |
| chr6 | 38485720 | 38487720 | chr6 | 38434093 | ENSMUST00000159925.1 | Ubn2 | 1.42203 |
| chr7 | 99808096 | 99810096 | chr7 | 99812047 | ENSMUST00000151177.1 | Pcf11 | 1.42022 |
| chr12 | 32847765 | 32849765 | chr12 | 33063569 | ENSMUSG00000090946.1 | 2010109K11Rik | 1.417 |
| chr19 | 9113416 | 9115416 | chr19 | 8831593 | ENSMUSG00000010097.6 | Nxf1 | 1.41635 |
| chr17 | 24661305 | 24663305 | chr17 | 24300697 | ENSMUST00000130520.1 | Amdhd2 | 1.41324 |
| chr6 | 149066954 | 149068954 | chr6 | 149050202 | ENSMUST00000111557.1 | Dennd5b | 1.40958 |
| chr15 | 34026620 | 34028620 | chr15 | 34012480 | ENSMUST00000163697.1 | Mtdh | 1.40787 |
| chr11 | 5267987 | 5269987 | chr11 | 5738019 | ENSMUST00000020767.3 | Polm | 1.40595 |
| chr12 | 86532623 | 86534623 | chr12 | 86337302 | ENSMUST00000021670.8 | Ylpm1 | 1.40329 |
| chr7 | 142900654 | 142902654 | chr7 | 142908062 | ENSMUSG00000031004.7 | Mki67 | 1.40051 |
| chr2 | 153100512 | 153102512 | chr2 | 153491538 | ENSMUST00000132132.1 | Dnmt3b | 1.40002 |
| chr7 | 52079823 | 52081823 | chr7 | 52317075 | ENSMUST00000133587.1 | Prrg2 | 1.39296 |
| chr5 | 134848944 | 134850944 | chr5 | 134932132 | ENSMUST00000111244.1 | Gtf2ird1 | 1.38594 |
| chr5 | 117810007 | 117812007 | chr5 | 117807314 | ENSMUST00000125522.1 | Wsb2 | 1.38568 |
| chr9 | 65395253 | 65397253 | chr9 | 65427847 | ENSMUSG00000050721.8 | Plekho2 | 1.38193 |
| chr3 | 94856898 | 94858898 | chr3 | 94641635 | ENSMUST00000042402.5 | Pogz | 1.37203 |
| chr10 | 80080382 | 80082382 | chr10 | 80217951 | ENSMUSG00000061589.7 | Dot11 | 1.36833 |
| chr12 | 93037330 | 93039330 | chr12 | 93017577 | ENSMUST00000170077.1 | Ston2 | 1.36566 |
| chr11 | 20923499 | 20925499 | chr11 | 21139284 | ENSMUST00000006221.7 | Vps54 | 1.35921 |
| chr11 | 57766357 | 57768357 | chr11 | 58453967 | ENSMUST00000075084.4 | Trim58 | 1.35554 |
| chr11 | 100843840 | 100845840 | chr11 | 100800718 | ENSMUST00000103114.1 | Stat3 | 1.35164 |
| chr9 | 45855653 | 45857653 | chr9 | 45714944 | ENSMUST00000162699.1 | Rnf214 | 1.35032 |
| chr7 | 134657172 | 134659172 | chr7 | 134592672 | ENSMUST00000053392.4 | Zfp689 | 1.34988 |
| chr4 | 132127950 | 132129950 | chr4 | 131885431 | ENSMUST00000146166.1 | Trnau1ap | 1.34702 |
| chr4 | 150427277 | 150429277 | chr4 | 150432072 | ENSMUSG00000028955.3 | Vamp3 | 1.34431 |
| chr8 | 87506801 | 87508801 | chr8 | 87417816 | ENSMUST00000003907.7 | Gcdh | 1.34055 |
| chr16 | 50395250 | 50397250 | chr16 | 50432502 | ENSMUST00000089404.3 | Bbx | 1.32952 |
| chr17 | 36271246 | 36273246 | chr17 | 35998283 | ENSMUST00000174873.1 | Nrm | 1.32674 |
| chr10 | 80080382 | 80082382 | chr10 | 80165438 | ENSMUSG00000055862.6 | Izumo4 | 1.3265 |
| chr1 | 74850633 | 74852633 | chr1 | 74735997 | ENSMUST00000155753.1 | Ttll4 | 1.32598 |
| chr4 | 107753457 | 107755457 | chr4 | 107838071 | ENSMUST00000130942.1 | Echdc2 | 1.32583 |
| chr12 | 77464719 | 77466719 | chr12 | 77469986 | ENSMUST00000070570.4 | Zbtb25 | 1.32496 |
| chr19 | 4136521 | 4138521 | chr19 | 4154606 | ENSMUSG00000045826.8 | Ptprcap | 1.32488 |
| chr8 | 26121545 | 26123545 | chr8 | 26127683 | ENSMUSG00000031556.5 | Tm2d2 | 1.32213 |
| chr16 | 11172524 | 11174524 | chr16 | 11176157 | ENSMUST00000142389.1 | Zc3h7a | 1.32163 |
| chr1 | 155256106 | 155258106 | chr1 | 155179916 | ENSMUST00000027752.8 | Lamc1 | 1.32085 |
| chr12 | 86532623 | 86534623 | chr12 | 86621256 | ENSMUST00000117138.2 | Acyp1 | 1.32006 |
| chr15 | 37995014 | 37997014 | chr15 | 37890646 | ENSMUST00000137636.1 | Rrm2b | 1.31444 |
| chr2 | 126505144 | 126507144 | chr2 | 126501222 | ENSMUST00000103226.3 | Gabpb1 | 1.30921 |
| chr4 | 137154503 | 137156503 | chr4 | 137150122 | ENSMUST00000055131.6 | Usp48 | 1.30813 |
| chr7 | 135872166 | 135874166 | chr7 | 135605166 | ENSMUST00000141079.1 | Tial1 | 1.29828 |
| chr4 | 132127950 | 132129950 | chr4 | 132288461 | ENSMUST00000045550.4 | Xkr8 | 1.29508 |
| chr4 | 59738804 | 59740804 | chr4 | 59639115 | ENSMUST00000052420.6 | E130308A19Rik | 1.28978 |
| chr4 | 132127950 | 132129950 | chr4 | 132194961 | ENSMUST00000081726.6 | Eya3 | 1.28514 |
| chr16 | 91378045 | 91380045 | chr16 | 91547392 | ENSMUST00000127644.1 | Ifngr2 | 1.28153 |
| chr7 | 109112980 | 109114980 | chr7 | 109358876 | ENSMUST00000120879.1 | Pgap2 | 1.28133 |
| chr3 | 96077259 | 96079259 | chr3 | 95976255 | ENSMUSG00000068856.3 | Sf3b4 | 1.27879 |
| chr17 | 34345356 | 34347356 | chr17 | 34280252 | ENSMUST00000173262.1 | H2-DMb2 | 1.27341 |
| chr10 | 80752437 | 80754437 | chr10 | 80720044 | ENSMUST00000105328.3 | Matk | 1.27157 |
| chr6 | 108517847 | 108519847 | chr6 | 108610793 | ENSMUST00000166346.1 | Bhlhe40 | 1.27118 |
| chr10 | 128039557 | 128041557 | chr10 | 127669136 | ENSMUST00000145710.1 | Timeless | 1.26473 |
| chr16 | 4719688 | 4721688 | chr16 | 3992984 | ENSMUST00000109180.2 | Slx4 | 1.26101 |
| chr13 | 112298206 | 112300206 | chr13 | 112280249 | ENSMUST00000116379.2 | Gpbp1 | 1.25991 |
| chr2 | 118361658 | 118363658 | chr2 | 118727189 | ENSMUST00000163517.1 | Bahd1 | 1.2596 |
| chr4 | 132127950 | 132129950 | chr4 | 132399038 | ENSMUST00000105919.1 | Ppp1r8 | 1.24863 |
| chr2 | 35046233 | 35048233 | chr2 | 34817292 | ENSMUST00000168557.1 | Traf1 | 1.24129 |
| chr10 | 19278153 | 19280153 | chr10 | 19654278 | ENSMUST00000095806.3 | Map3k5 | 1.23922 |
| chr7 | 134624783 | 134626783 | chr7 | 133944265 | ENSMUSG00000030695.7 | Aldoa | 1.23625 |
| chr7 | 52079823 | 52081823 | chr7 | 52108290 | ENSMUST00000142880.1 | Akt1s1 | 1.23616 |
| chr2 | 153384805 | 153386805 | chr2 | 153016135 | ENSMUST00000140988.1 | Tm9sf4 | 1.23558 |
| chr1 | 163003668 | 163005668 | chr1 | 163000789 | ENSMUST00000035430.3 | Dars2 | 1.2316 |
| chr11 | 118285234 | 118287234 | chr11 | 118338143 | ENSMUST00000135383.2 | Engase | 1.22475 |
| chr4 | 129494590 | 129496590 | chr4 | 129296324 | ENSMUST00000146378.1 | Iqcc | 1.2242 |
| chr11 | 16868043 | 16870043 | chr11 | 17092847 | ENSMUST00000154425.1 | Ppp3r1 | 1.22158 |
| chr13 | 63950593 | 63952593 | chr13 | 63971817 | ENSMUST00000109776.2 | 0610007P08Rik | 1.21772 |
| chr11 | 32596188 | 32598188 | chr11 | 32542724 | ENSMUST00000109366.1 | Fbxw11 | 1.21497 |
| chr7 | 87193956 | 87195956 | chr7 | 87469039 | ENSMUST00000134288.1 | Rccd1 | 1.21463 |
| chr16 | 50395250 | 50397250 | chr16 | 50432494 | ENSMUST00000023317.5 | Bbx | 1.21427 |
| chr10 | 33848442 | 33850442 | chr10 | 33671018 | ENSMUST00000048222.4 | Zufsp | 1.21357 |
| chr11 | 5267987 | 5269987 | chr11 | 5052260 | ENSMUST00000062821.6 | Emid1 | 1.21345 |
| chr11 | 5291490 | 5293490 | chr11 | 5052260 | ENSMUST00000062821.6 | Emid1 | 1.21345 |
| chr16 | 32533124 | 32535124 | chr16 | 32247313 | ENSMUST00000141820.1 | Wdr53 | 1.21336 |
| chr8 | 114115301 | 114117301 | chr8 | 114046007 | ENSMUST00000038193.7 | Wdr59 | 1.21222 |
| chr5 | 117810007 | 117812007 | chr5 | 117773655 | ENSMUST00000147182.1 | Vsig10 | 1.20073 |
| chr9 | 21431017 | 21433017 | chr9 | 21142288 | ENSMUSG00000057193.6 | Slc44a2 | 1.19888 |
| chr1 | 36204991 | 36206991 | chr1 | 36222656 | ENSMUST00000173999.1 | Uggt1 | 1.19637 |
| chr15 | 93081233 | 93083233 | chr15 | 93167366 | ENSMUST00000133736.1 | Yaf2 | 1.18672 |
| chr2 | 44900819 | 44902819 | chr2 | 44968799 | ENSMUST00000068415.4 | Zeb2 | 1.18483 |
| chr3 | 95950914 | 95952914 | chr3 | 96226656 | ENSMUSG00000065020.1 | U1 | 1.16987 |
| chr7 | 63212697 | 63214697 | chr7 | 63217901 | ENSMUST00000130189.1 | A230056P14Rik | 1.16935 |
| chr17 | 26050333 | 26052333 | chr17 | 25961607 | ENSMUST00000160275.1 | Wdr24 | 1.16796 |
| chr6 | 42340610 | 42342610 | chr6 | 42323267 | ENSMUSG00000029859.4 | Epha1 | 1.1679 |
| chr12 | 86532623 | 86534623 | chr12 | 86518246 | ENSMUST00000004913.6 | Pgf | 1.16684 |
| chr3 | 135227472 | 135229472 | chr3 | 135086889 | ENSMUSG00000028165.8 | Cisd2 | 1.16501 |
| chr11 | 78817188 | 78819188 | chr11 | 78349834 | ENSMUST00000108277.2 | Tnfaip1 | 1.16405 |
| chr10 | 127517457 | 127519457 | chr10 | 127684396 | ENSMUST00000105240.1 | Timeless | 1.1616 |
| chr1 | 74850633 | 74852633 | chr1 | 75209636 | ENSMUST00000156012.1 | Stk16 | 1.16081 |
| chr6 | 149066954 | 149068954 | chr6 | 149357506 | ENSMUST00000086829.4 | Bicd1 | 1.15866 |
| chrX | 13247059 | 13249059 | chrX | 12858096 | ENSMUSG00000000787.6 | Ddx3x | 1.15833 |
| chr10 | 80752437 | 80754437 | chr10 | 80841208 | ENSMUST00000138343.1 | Fzr1 | 1.15673 |
| chr18 | 82699936 | 82701936 | chr18 | 82644540 | ENSMUST00000114676.1 | Mbp | 1.15664 |
| chr11 | 52181889 | 52183889 | chr11 | 51814264 | ENSMUST00000147833.1 | Ube2b | 1.15447 |
| chr11 | 75351840 | 75353840 | chr11 | 75380340 | ENSMUST00000169547.1 | Slc43a2 | 1.15222 |
| chr3 | 89686643 | 89688643 | chr3 | 89883637 | ENSMUST00000119158.1 | Tpm3 | 1.15195 |
| chr16 | 11172524 | 11174524 | chr16 | 11134743 | ENSMUSG00000022498.10 | Txndc11 | 1.14892 |
| chr5 | 23378406 | 23380406 | chr5 | 23293537 | ENSMUST00000124680.1 | Rint1 | 1.14824 |
| chr2 | 153100512 | 153102512 | chr2 | 153146014 | ENSMUST00000099189.4 | Kif3b | 1.14565 |
| chr2 | 153384805 | 153386805 | chr2 | 153146014 | ENSMUST00000099189.4 | Kif3b | 1.14565 |
| chr9 | 115477117 | 115479117 | chr9 | 115219539 | ENSMUSG00000032437.9 | Stt3b | 1.14255 |
| chr19 | 4136521 | 4138521 | chr19 | 4269172 | ENSMUSG00000034616.9 | Ssh3 | 1.13731 |
| chr1 | 155447492 | 155449492 | chr1 | 155596556 | ENSMUSG00000066800.3 | Rnasel | 1.13613 |
| chr11 | 75222724 | 75224724 | chr11 | 75300279 | ENSMUSG00000020850.7 | Prpf8 | 1.13095 |
| chr12 | 80117826 | 80119826 | chr12 | 80108264 | ENSMUST00000056660.6 | Tmem229b | 1.12946 |
| chr2 | 118314465 | 118316465 | chr2 | 118305447 | ENSMUSG00000009549.8 | Srp14 | 1.12741 |
| chr10 | 19908474 | 19910474 | chr10 | 19847490 | ENSMUST00000142726.1 | Map3k5 | 1.12647 |
| chr6 | 38485720 | 38487720 | chr6 | 38483502 | ENSMUST00000147651.1 | 1110001J03Rik | 1.1259 |
| chr11 | 102891992 | 102893992 | chr11 | 102889618 | ENSMUST00000155490.1 | Dcakd | 1.12182 |
| chr6 | 145052832 | 145054832 | chr6 | 145064173 | ENSMUST00000135984.1 | Lrmp | 1.11887 |
| chr18 | 35093288 | 35095288 | chr18 | 35114011 | ENSMUSG00000024359.8 | Hspa9 | 1.11838 |
| chr4 | 137154503 | 137156503 | chr4 | 136913635 | ENSMUSG00000006699.10 | Cdc42 | 1.11585 |
| chr7 | 133271143 | 133273143 | chr7 | 134041795 | ENSMUST00000145307.1 | Tmem219 | 1.11549 |
| chr3 | 141935626 | 141937626 | chr3 | 142159864 | ENSMUST00000045254.7 | Gbp5 | 1.11005 |
| chr10 | 107636370 | 107638370 | chr10 | 107599249 | ENSMUSG00000019907.8 | Ppp1r12a | 1.10896 |
| chr3 | 94856898 | 94858898 | chr3 | 94846536 | ENSMUSG00000005625.9 | Psmd4 | 1.10805 |
| chr2 | 29525390 | 29527390 | chr2 | 29643150 | ENSMUST00000113803.1 | Trub2 | 1.10485 |
| chr3 | 116302840 | 116304840 | chr3 | 116297586 | ENSMUST00000134761.1 | Ccdc76 | 1.10445 |
| chr10 | 126563534 | 126565534 | chr10 | 126507317 | ENSMUSG00000006736.8 | Tspan31 | 1.1043 |
| chr2 | 90840821 | 90842821 | chr2 | 91023994 | ENSMUST00000135715.1 | Madd | 1.10125 |
| chr1 | 153532785 | 153534785 | chr1 | 153602504 | ENSMUST00000059498.5 | Edem3 | 1.09955 |
| chr4 | 107753457 | 107755457 | chr4 | 107842748 | ENSMUST00000133049.2 | Echdc2 | 1.098 |
| chr17 | 71438977 | 71440977 | chr17 | 71532896 | ENSMUST00000156570.1 | Lpin2 | 1.09536 |
| chr19 | 32398488 | 32400488 | chr19 | 32351442 | ENSMUST00000152340.1 | Sgms1 | 1.0948 |
| chr16 | 4719688 | 4721688 | chr16 | 4790292 | ENSMUSG00000004071.6 | 5730403B10Rik | 1.09408 |
| chr4 | 8637044 | 8639044 | chr4 | 8618512 | ENSMUST00000051558.3 | Chd7 | 1.09326 |
| chr2 | 28380033 | 28382033 | chr2 | 28404578 | ENSMUST00000140704.1 | Ralgds | 1.09323 |
| chr9 | 14837735 | 14839735 | chr9 | 14849922 | ENSMUST00000056755.7 | Panx1 | 1.0921 |
| chr4 | 132127950 | 132129950 | chr4 | 132102574 | ENSMUST00000156385.1 | Dnajc8 | 1.09182 |
| chr1 | 34890064 | 34892064 | chr1 | 34906821 | ENSMUST00000156687.1 | Plekhb2 | 1.08951 |
| chr16 | 76040422 | 76042422 | chr16 | 75767027 | ENSMUST00000137806.1 | Hspa13 | 1.08702 |
| chr3 | 100305904 | 100307904 | chr3 | 99947505 | ENSMUST00000129319.1 | Wdr3 | 1.08438 |
| chr3 | 59038623 | 59040623 | chr3 | 58329795 | ENSMUSG00000027808.7 | Serp1 | 1.08403 |
| chr10 | 39344769 | 39346769 | chr10 | 39365461 | ENSMUST00000139891.1 | E130307A14Rik | 1.06806 |
| chr13 | 52682169 | 52684169 | chr13 | 52678872 | ENSMUST00000150672.1 | Sykb | 1.06553 |
| chr16 | 32533124 | 32535124 | chr16 | 32165580 | ENSMUST00000143682.1 | Lrrc33 | 1.06473 |
| chr2 | 78976919 | 78978919 | chr2 | 79173839 | ENSMUST00000147402.1 | Cerkl | 1.0615 |
| chr2 | 90840821 | 90842821 | chr2 | 90894212 | ENSMUST00000002171.7 | Psmc3 | 1.06131 |
| chr7 | 134657172 | 134659172 | chr7 | 134655541 | ENSMUSG00000053877.6 | Srcap | 1.06113 |
| chr4 | 154609450 | 154611450 | chr4 | 154975525 | ENSMUST00000105608.2 | Slc35e2 | 1.05713 |
| chr17 | 10500648 | 10502648 | chr17 | 10512245 | ENSMUST00000042296.6 | Qk | 1.04833 |
| chr2 | 163246818 | 163248818 | chr2 | 163244880 | ENSMUST00000140454.1 | 3230401D17Rik | 1.04823 |
| chr9 | 58117801 | 58119801 | chr9 | 58100971 | ENSMUSG00000032333.5 | Stoml1 | 1.04587 |
| chr4 | 108787213 | 108789213 | chr4 | 108874877 | ENSMUSG00000028559.10 | Osbpl9 | 1.0346 |
| chr5 | 148252123 | 148254123 | chr5 | 148242156 | ENSMUST00000085571.5 | Pan3 | 1.03362 |
| chr9 | 63596837 | 63598837 | chr9 | 63591072 | ENSMUST00000137065.1 | Smad3 | 1.02868 |
| chr10 | 93987792 | 93989792 | chr10 | 94013617 | ENSMUST00000117460.1 | Tmcc3 | 1.02781 |
| chr3 | 96077259 | 96079259 | chr3 | 96050674 | ENSMUST00000091711.2 | Hist2h3c1 | 1.02685 |
| chr18 | 32470773 | 32472773 | chr18 | 32322743 | ENSMUSG00000024383.8 | Map3k2 | 1.02048 |
| chr17 | 37129242 | 37131242 | chr17 | 37082023 | ENSMUST00000174669.1 | Rnf39 | 1.01988 |
| chr4 | 131753294 | 131755294 | chr4 | 131768006 | ENSMUST00000085181.4 | Ythdf2 | 1.01962 |
| chr7 | 52079823 | 52081823 | chr7 | 52359192 | ENSMUSG00000003420.7 | Fcgrt | 1.01495 |
| chr14 | 21371163 | 21373163 | chr14 | 21365479 | ENSMUST00000142099.1 | 1810062O18Rik | 1.01423 |
| chr10 | 126563534 | 126565534 | chr10 | 126727849 | ENSMUST00000139091.1 | Ddit3 | 1.01409 |
| chr10 | 80080382 | 80082382 | chr10 | 80261371 | ENSMUSG00000035278.8 | Plekhj1 | 1.01275 |
| chr15 | 78826239 | 78828239 | chr15 | 78832612 | ENSMUST00000129922.1 | Triobp | 1.01086 |
| chr9 | 45817658 | 45819658 | chr9 | 45792954 | ENSMUSG00000003131.5 | Pafah1b2 | 1.01013 |
| chr11 | 75351840 | 75353840 | chr11 | 75345245 | ENSMUST00000143035.1 | Slc43a2 | 1.00608 |
| chr11 | 75789696 | 75791696 | chr11 | 76057153 | ENSMUST00000170017.1 | Glod4 | 1.0059 |
| chr11 | 20923499 | 20925499 | chr11 | 20641592 | ENSMUSG00000049659.7 | Aftph | 1.00553 |
| chr2 | 165793082 | 165795082 | chr2 | 165710374 | ENSMUST00000088113.4 | Zmynd8 | 1.00292 |
| chr19 | 29457752 | 29459752 | chr19 | 29485409 | ENSMUST00000112576.2 | Pdcd1lg2 | 1.00205 |

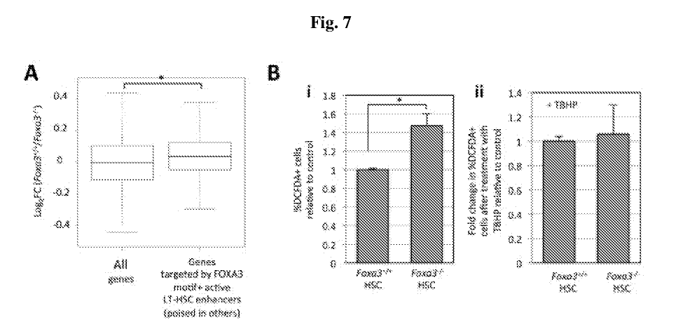

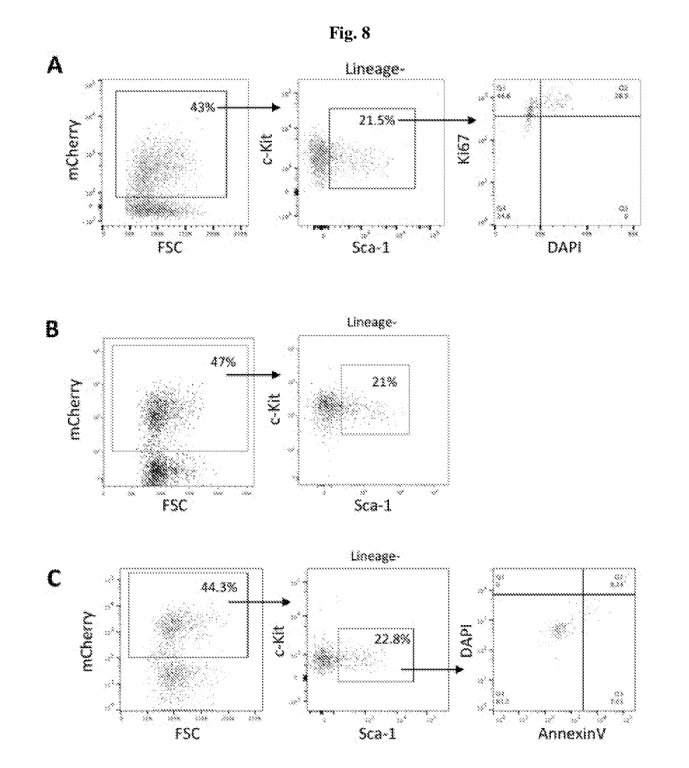

The resulting gene set was expressed higher than the rest of the genes in our microarray data (Foxa3^{+/+} HSC versus Foxa3^{-/-} HSC), confirming regulation of these genes by Foxa3 in LT-HSC (FIG. 7A, Table 6).

TABLE 6
Microarray results of genes significantly up and downregulated in Foxa3^{-/-}HSC versus Foxa3^{+/+} HSC.

| Genes significantly downregulated | | | | Genes significantly upregulated | | | |
|---|---|---|---|---|---|---|---|
| | Fold- | | | | Fold- | Log2 | |
| Gene symbol | change | Log2 ratio | p value | Gene symbol | change | ratio | p value |
| Mir421 | −2.46136 | −1.29946 | 0.039633 | Mt2 | 2.88633 | 1.52923 | 0.036518 |
| Trim43c | −2.16696 | −1.11567 | 0.003501 | Mir5103 | 2.53176 | 1.34014 | 0.038713 |
| Mir493 | −1.97426 | −0.981313 | 0.03655 | Calml4 | 2.41397 | 1.27141 | 0.040838 |
| Gm2178 | −1.89658 | −0.923401 | 0.004604 | Gm5833 | 2.37015 | 1.24498 | 0.023954 |
| D330045A20Rik | −1.81702 | −0.861574 | 0.038532 | Rps20 | 1.98209 | 0.987021 | 0.034299 |
| Olfr1299 | −1.70454 | −0.769384 | 0.012953 | Rab34 | 1.89606 | 0.923008 | 0.026085 |
| Pawr | −1.68746 | −0.754851 | 0.034265 | Gm6337 | 1.7533 | 0.810073 | 0.005988 |
| Rep15 | −1.66497 | −0.735498 | 0.016075 | Snora16a | 1.7409 | 0.799835 | 0.020319 |
| Epb4.1l3 | −1.64994 | −0.722418 | 0.041489 | Hoxb6 | 1.73767 | 0.797151 | 0.002114 |
| Zfp119b | −1.62802 | −0.70312 | 0.021007 | Ly6c2 | 1.73315 | 0.793397 | 0.024386 |
| Olfr894 | −1.59765 | −0.675948 | 0.011987 | Ager | 1.72548 | 0.786994 | 0.011279 |
| Gm9918 | −1.54117 | −0.624025 | 0.043055 | Aldh3a2 | 1.69043 | 0.757394 | 0.022301 |
| Cyp4f40 | −1.51866 | −0.602796 | 0.003764 | 8430403D17Rik | 1.68123 | 0.749517 | 0.021531 |
| Lce1d | −1.51679 | −0.601018 | 0.047403 | Zfp493 | 1.67409 | 0.743379 | 0.029382 |
| Hist1h2bk | −1.50609 | −0.590804 | 0.04991 | Gm6904 | 1.65892 | 0.730244 | 0.022431 |
| Nfkbil1 | −1.49937 | −0.584355 | 0.021864 | Gm5662 | 1.65825 | 0.729665 | 0.004467 |
| Vmn1r213 | −1.48036 | −0.565946 | 0.018624 | BC034090 | 1.63135 | 0.706067 | 0.036855 |
| Ptk6 | −1.47475 | −0.560467 | 0.025465 | Syne1 | 1.62784 | 0.702963 | 0.028766 |
| Olfr275 | −1.47287 | −0.55863 | 0.015135 | Neo1 | 1.60979 | 0.686873 | 0.04755 |
| Srsy | −1.46836 | −0.554209 | 0.009082 | Bdh2 | 1.59665 | 0.675052 | 0.031584 |
| Kcnv2 | −1.46372 | −0.549636 | 0.0065 | AA415398 | 1.5828 | 0.662481 | 0.000787 |
| 4930405D11Rik | −1.45556 | −0.541574 | 0.045067 | Nme2 | 1.56235 | 0.643713 | 0.04385 |
| Tfpi2 | −1.45268 | −0.538721 | 0.034273 | Snord66 | 1.52965 | 0.613205 | 0.04274 |
| Cntnap5b | −1.44684 | −0.532901 | 0.037478 | Procr | 1.52378 | 0.607656 | 0.005272 |
| Mir3106 | −1.44562 | −0.531693 | 0.025992 | Nedd4l | 1.52201 | 0.60598 | 0.006991 |
| Abpd | −1.43478 | −0.520829 | 0.009722 | 1700013G23Rik | 1.5193 | 0.603404 | 0.037097 |
| Olfr684 | −1.43177 | −0.517801 | 0.045926 | Wdr47 | 1.51687 | 0.601102 | 0.044863 |
| Vmn1r135 | −1.4257 | −0.511674 | 0.024838 | Tdg | 1.5123 | 0.59674 | 0.033727 |
| Vmn1r135 | −1.4257 | −0.511674 | 0.024838 | BC117090 | 1.51014 | 0.594685 | 0.022161 |
| 9530057J20Rik | −1.42209 | −0.508012 | 0.01928 | Gm19792 | 1.50583 | 0.590563 | 0.022273 |
| Rsph4a | −1.42147 | −0.507386 | 0.038352 | Tle1 | 1.50395 | 0.588753 | 0.042047 |
| Ano8 | −1.41689 | −0.50273 | 0.002349 | Loxl3 | 1.50075 | 0.58568 | 0.003134 |
| Apbb1 | −1.41657 | −0.502397 | 0.041116 | I730030J21Rik | 1.48301 | 0.56853 | 0.042971 |
| AB099516 | −1.41404 | −0.499826 | 0.038384 | Cd248 | 1.47919 | 0.564808 | 0.006957 |
| 6720489N17Rik | −1.41317 | −0.498937 | 0.019414 | Mmp14 | 1.47732 | 0.562981 | 0.016976 |
| D830029L11 | −1.41285 | −0.498609 | 0.007353 | Dio2 | 1.46905 | 0.554887 | 0.009785 |
| Ly96 | −1.40947 | −0.495155 | 0.007323 | Gm3173 | 1.46696 | 0.552826 | 0.005487 |
| 7530414M10Rik | −1.40932 | −0.495 | 0.026879 | Mrps23 | 1.46428 | 0.550187 | 0.010792 |
| Ica1l | −1.4014 | −0.486869 | 0.02793 | C330018D20Rik | 1.46266 | 0.548592 | 0.027631 |
| Olfr558 | −1.39654 | −0.481859 | 0.015271 | Unc13d | 1.45838 | 0.544364 | 0.045822 |
| Olfr1286 | −1.39616 | −0.481468 | 0.008866 | LOC100862515 | 1.45658 | 0.542587 | 0.001699 |
| Psg29 | −1.39535 | −0.480625 | 0.047619 | Gm3591 | 1.45085 | 0.536901 | 0.044933 |
| Gm9962 | −1.38785 | −0.472853 | 0.004537 | Urgcp | 1.44987 | 0.535921 | 0.014536 |
| Mycl1 | −1.3818 | −0.466552 | 0.030999 | Nupr1 | 1.44525 | 0.531316 | 0.028485 |
| Ldlrad1 | −1.38125 | −0.465978 | 0.023415 | Ift88 | 1.44478 | 0.530854 | 0.008731 |
| Bmp15 | −1.37856 | −0.463157 | 0.042601 | Tgfbi | 1.4433 | 0.529375 | 0.036521 |
| Gm757 | −1.37732 | −0.461869 | 0.046922 | Cpeb2 | 1.43641 | 0.522468 | 0.021809 |
| Cd300e | −1.37503 | −0.459459 | 0.008646 | 2310050C09Rik | 1.43623 | 0.522282 | 0.0051 |
| Cypt2 | −1.37407 | −0.458452 | 0.003764 | 9830147E19Rik | 1.43447 | 0.520523 | 0.041208 |
| Lactbl1 | −1.37363 | −0.457996 | 0.013665 | Gm20204 | 1.42823 | 0.51423 | 0.00068 |
| Srsy | −1.37023 | −0.454415 | 0.009917 | Ghr | 1.42303 | 0.508964 | 0.011107 |
| Srsy | −1.37023 | −0.454415 | 0.009917 | 6530439I21 | 1.42181 | 0.507725 | 0.003117 |
| Efcab4b | −1.36852 | −0.452619 | 0.041002 | Efna1 | 1.41737 | 0.50322 | 0.03351 |
| D730002M21Rik | −1.35971 | −0.443297 | 0.015297 | Sec22a | 1.4141 | 0.499888 | 0.025681 |
| D230022J07Rik | −1.35958 | −0.443162 | 0.011154 | Vmn1r17 | 1.41204 | 0.497777 | 0.034297 |
| Gm12695 | −1.35869 | −0.442215 | 0.00141 | 2310042E22Rik | 1.40911 | 0.494782 | 0.033865 |
| Zfp275 | −1.35695 | −0.440371 | 0.034702 | Creb3l2 | 1.40771 | 0.493351 | 0.026211 |
| Ssty2 | −1.35421 | −0.437453 | 0.033984 | Zfp28 | 1.40451 | 0.490068 | 0.007363 |
| Ssty2 | −1.35421 | −0.437453 | 0.033984 | Naip2 | 1.39987 | 0.485292 | 0.029072 |
| Srsy | −1.35392 | −0.437139 | 0.023016 | Nuak2 | 1.39569 | 0.480974 | 0.026107 |
| Olfr1361 | −1.35123 | −0.434278 | 0.027494 | Vmn1r60 | 1.39084 | 0.475956 | 0.001684 |
| Bmp5 | −1.34894 | −0.431826 | 0.046213 | Ccdc57 | 1.38431 | 0.469169 | 0.048919 |
| Gm19971 | −1.34627 | −0.428964 | 0.045975 | Grk5 | 1.3842 | 0.469054 | 0.015338 |
| LOC434003 | −1.34615 | −0.428836 | 0.043147 | Msl3 | 1.38405 | 0.468896 | 0.01891 |
| Gria2 | −1.34538 | −0.428016 | 0.004614 | Tbx1 | 1.38373 | 0.468561 | 0.048627 |
| E330021D16Rik | −1.3443 | −0.426856 | 0.046942 | Stk40 | 1.38038 | 0.465068 | 0.049476 |
| Olfr272 | −1.34424 | −0.426793 | 0.02498 | 6430527G18Rik | 1.37773 | 0.462293 | 0.041065 |
| Dact1 | −1.34152 | −0.423868 | 0.035965 | Ift81 | 1.37721 | 0.461745 | 0.008468 |
| BC037032 | −1.34128 | −0.423613 | 0.032792 | AW554918 | 1.3735 | 0.457853 | 0.047567 |
| Vmn1r143 | −1.3411 | −0.423415 | 0.001762 | Defa-ps12 | 1.37142 | 0.455673 | 0.026592 |
| Cx3cr1 | −1.3403 | −0.422558 | 0.03746 | Atpaf2 | 1.37054 | 0.454749 | 0.013061 |
| Olfr71 | −1.3401 | −0.422338 | 0.03527 | Zfp948 | 1.36992 | 0.45409 | 0.043317 |
| Vmn1r158 | −1.33893 | −0.421078 | 0.012153 | Vsig10 | 1.36724 | 0.45127 | 0.031475 |
| Vmn1r158 | −1.33893 | −0.421078 | 0.012153 | Nat9 | 1.36722 | 0.451247 | 0.002087 |
| Plek2 | −1.33845 | −0.420559 | 0.028283 | Cd72 | 1.36415 | 0.447999 | 0.046009 |
| Acmsd | −1.33428 | −0.416065 | 0.02265 | Cecr2 | 1.36266 | 0.44643 | 0.033856 |
| Blk | −1.33266 | −0.414308 | 0.048037 | 2010011I20Rik | 1.36254 | 0.446302 | 0.026422 |
| Ffar1 | −1.33139 | −0.412933 | 0.046195 | Tmem87b | 1.36246 | 0.44621 | 0.026602 |
| Cyp2c44 | −1.32984 | −0.41125 | 0.019243 | Gm6116 | 1.36165 | 0.445353 | 0.046873 |
| Lamp3 | −1.32875 | −0.410074 | 0.043558 | C1ql3 | 1.36164 | 0.44535 | 0.012315 |
| Mir370 | −1.32684 | −0.407999 | 0.045128 | Rundc1 | 1.35797 | 0.441447 | 0.016816 |
| Oscar | −1.32648 | −0.407603 | 0.016469 | Tmem186 | 1.35733 | 0.440774 | 0.005568 |
| Itga11 | −1.32642 | −0.407533 | 0.022178 | H2-Eb1 | 1.35651 | 0.439902 | 0.02902 |
| Vipr1 | −1.32584 | −0.406907 | 0.015221 | Kbtbd4 | 1.35215 | 0.435258 | 0.004938 |
| Rps15a | −1.32491 | −0.405898 | 0.044745 | Abcb8 | 1.34854 | 0.431402 | 0.006319 |
| Prelp | −1.3246 | −0.405558 | 0.019439 | Haghl | 1.3461 | 0.428782 | 0.016412 |
| Gm4776 | −1.32459 | −0.405546 | 0.012773 | Ppp2ca | 1.34224 | 0.424644 | 0.028678 |
| Serinc2 | −1.32288 | −0.403687 | 0.030237 | Alg12 | 1.34138 | 0.423722 | 0.004515 |
| Drd3 | −1.32106 | −0.401691 | 0.024939 | 1110051M20Rik | 1.34015 | 0.422399 | 0.032122 |
| Vmn1r174 | −1.32105 | −0.401687 | 0.042395 | Vmn1r3 | 1.339 | 0.421157 | 0.011189 |
| Srsy | −1.3205 | −0.401087 | 0.008648 | E330016L19Rik | 1.33817 | 0.420267 | 0.027437 |
| Gm16796 | −1.31859 | −0.399001 | 0.044475 | Zfp157 | 1.33562 | 0.417508 | 0.009547 |
| Sebox | −1.31824 | −0.398615 | 0.049752 | Il15ra | 1.33474 | 0.416556 | 0.03979 |
| Foxo4 | −1.31708 | −0.397341 | 0.020293 | Sqle | 1.33329 | 0.414991 | 0.043967 |
| Kansl2 | −1.31567 | −0.395798 | 0.04896 | Ppp1r10 | 1.33327 | 0.41497 | 0.007314 |
| Gm13288 | −1.31331 | −0.393211 | 0.039275 | Tmem181c-ps | 1.33184 | 0.413417 | 0.030232 |
| Slc22a21 | −1.31027 | −0.389861 | 0.027319 | Cyp2u1 | 1.33008 | 0.411513 | 0.025901 |
| Ear14 | −1.30956 | −0.389083 | 0.028557 | Olfr1390 | 1.33008 | 0.411509 | 0.040981 |
| Lrrc8e | −1.30769 | −0.387021 | 0.037264 | Gm10021 | 1.33006 | 0.41149 | 0.043245 |
| Glt8d2 | −1.30705 | −0.386318 | 0.047777 | Lysmd1 | 1.3296 | 0.410995 | 0.033894 |
| Mir382 | −1.30669 | −0.385915 | 0.001361 | AU040320 | 1.32945 | 0.410831 | 0.003736 |
| Cys1 | −1.30563 | −0.384741 | 0.013745 | Enpp5 | 1.32781 | 0.409044 | 0.01754 |
| Gm4251 | −1.30559 | −0.384699 | 0.017299 | Trim3 | 1.32648 | 0.407598 | 0.003626 |
| Rgs5 | −1.30441 | −0.383397 | 0.039955 | 2010002N04Rik | 1.32631 | 0.407416 | 0.024639 |
| Acvrl1 | −1.30292 | −0.381751 | 0.002318 | Mir1950 | 1.32541 | 0.406439 | 0.022323 |
| F2rl1 | −1.30192 | −0.380645 | 0.010249 | Fbxo4 | 1.32511 | 0.406108 | 0.020527 |
| Iqub | −1.30084 | −0.379443 | 0.027888 | Gzme | 1.32246 | 0.403219 | 0.03798 |
| Vwa3b | −1.30015 | −0.378677 | 0.024942 | Hs6st1 | 1.32209 | 0.402825 | 0.023608 |
| Gm20382 | −1.29958 | −0.378042 | 0.000943 | BC067074 | 1.31927 | 0.399744 | 0.01556 |
| Sftpb | −1.2984 | −0.376732 | 0.008054 | Capn9 | 1.31876 | 0.399182 | 0.018654 |
| Dnaja3 | −1.29797 | −0.376261 | 0.026933 | Rpap2 | 1.31856 | 0.398959 | 0.032932 |
| Tspan18 | −1.29789 | −0.376168 | 0.032261 | Dmc1 | 1.31847 | 0.398865 | 0.04732 |
| Rbp2 | −1.29726 | −0.375473 | 0.030506 | Grem2 | 1.31789 | 0.398233 | 0.036522 |
| Gm2287 | −1.29725 | −0.375453 | 0.038632 | Vav2 | 1.31777 | 0.398095 | 0.035934 |
| Mir378 | −1.29515 | −0.373114 | 0.036728 | Sfxn5 | 1.31717 | 0.397439 | 0.034285 |
| Sparc | −1.29424 | −0.372103 | 0.037645 | Pfkm | 1.31714 | 0.397413 | 0.023477 |
| C430042M11Rik | −1.29358 | −0.371365 | 0.008355 | C130026I21Rik | 1.31441 | 0.394411 | 0.03258 |
| Gm3238 | −1.2935 | −0.371276 | 0.020869 | Sox13 | 1.31324 | 0.393127 | 0.046586 |
| Bhmt | −1.29245 | −0.370105 | 0.019702 | Csgalnact2 | 1.31262 | 0.392451 | 0.01421 |
| LOC100505026 | −1.2923 | −0.36994 | 0.03228 | Rsad1 | 1.30895 | 0.388405 | 0.027267 |
| Mtap6 | −1.29138 | −0.368919 | 0.038132 | Ndrg1 | 1.30894 | 0.3884 | 0.037842 |
| Ust | −1.28912 | −0.366384 | 0.035764 | Olfr490 | 1.30563 | 0.384742 | 0.04126 |
| Tulp1 | −1.28605 | −0.362945 | 0.048363 | Gbp8 | 1.30509 | 0.384147 | 0.031806 |
| Zfp110 | −1.28434 | −0.361029 | 0.019743 | Foxo6 | 1.30494 | 0.383989 | 0.003663 |
| Krt10 | −1.2842 | −0.360869 | 0.044232 | Plekhg6 | 1.30438 | 0.383366 | 0.048792 |
| Vmn1r93 | −1.28219 | −0.35861 | 0.014672 | Psen1 | 1.30412 | 0.383079 | 0.007715 |
| Vmn1r93 | −1.28219 | −0.35861 | 0.014672 | Rcbtb2 | 1.30381 | 0.38273 | 0.038905 |
| Cypt12 | −1.28093 | −0.357191 | 0.01899 | Stxbp3a | 1.30278 | 0.38159 | 0.040643 |
| Zcchc16 | −1.27847 | −0.354423 | 0.006371 | Gm19910 | 1.30163 | 0.380318 | 0.03026 |
| Cd14 | −1.27676 | −0.352491 | 0.03403 | Sdc2 | 1.3013 | 0.379953 | 0.031516 |
| Akap3 | −1.27517 | −0.35069 | 0.001782 | Ccpg1 | 1.29881 | 0.377188 | 0.019661 |
| Cdc45 | −1.27513 | −0.350649 | 0.031378 | Jrkl | 1.29694 | 0.375116 | 0.049785 |
| Gm9340 | −1.27508 | −0.350586 | 0.004405 | N6amt2 | 1.29631 | 0.374413 | 0.044931 |
| Crct1 | −1.27485 | −0.350329 | 0.004735 | Atp6v0a2 | 1.29596 | 0.37402 | 0.039216 |
| Ssxb9 | −1.27305 | −0.348294 | 0.049075 | Gm19569 | 1.29565 | 0.37368 | 0.026121 |
| A930009A15Rik | −1.27298 | −0.348204 | 0.019904 | Arfip1 | 1.29544 | 0.373445 | 0.046852 |
| Rad51c | −1.27247 | −0.34763 | 0.040523 | Orc1 | 1.29489 | 0.37283 | 0.034528 |
| Mettl7b | −1.27197 | −0.347064 | 0.008241 | Alox5 | 1.29459 | 0.372494 | 0.029277 |
| Vmn1r107 | −1.27187 | −0.346948 | 0.007001 | Ddx49 | 1.29443 | 0.372318 | 0.020901 |
| Vmn1r107 | −1.27187 | −0.346948 | 0.007001 | Pdzk1ip1 | 1.29388 | 0.371706 | 0.016291 |
| Col5a2 | −1.26952 | −0.34428 | 0.028173 | Itga2b | 1.28988 | 0.367236 | 0.027861 |
| LOC100862215 | −1.26906 | −0.343759 | 0.011683 | Shf | 1.28986 | 0.367214 | 0.035858 |
| Vmn1r126 | −1.26842 | −0.343038 | 0.016051 | Vmn2r90 | 1.28833 | 0.3655 | 0.039589 |
| Lamb1 | −1.26723 | −0.341682 | 0.039036 | Ccdc13 | 1.28787 | 0.364984 | 0.026871 |
| Gm19984 | −1.26695 | −0.341363 | 0.014201 | Olfr707 | 1.28762 | 0.364704 | 0.005583 |
| A930011G23Rik | −1.26466 | −0.338747 | 0.040835 | 2310008H04Rik | 1.28691 | 0.363907 | 0.001892 |
| Oc90 | −1.26412 | −0.338139 | 0.046793 | Lgi4 | 1.28483 | 0.361578 | 0.044618 |
| Stoml3 | −1.26168 | −0.33535 | 0.016545 | Ccdc115 | 1.2845 | 0.361207 | 0.014686 |
| Etv2 | −1.26035 | −0.333827 | 0.032693 | Nek4 | 1.28434 | 0.361032 | 0.02228 |
| Trim59 | −1.25983 | −0.333224 | 0.022804 | Nav2 | 1.28309 | 0.359623 | 0.004624 |
| Gm5606 | −1.25935 | −0.332682 | 0.034961 | Mrpl41 | 1.28299 | 0.359506 | 0.03766 |
| 4930513D17Rik | −1.25901 | −0.332292 | 0.01246 | Tceanc | 1.28225 | 0.358681 | 0.023591 |
| Kctd8 | −1.25881 | −0.332066 | 0.035586 | Sec23b | 1.28213 | 0.358545 | 0.042424 |
| Rab9b | −1.25707 | −0.330068 | 0.018111 | Lrpap1 | 1.28081 | 0.357061 | 0.046338 |
| 5330411J11Rik | −1.25557 | −0.328341 | 0.01927 | Krtap12-1 | 1.27877 | 0.354757 | 0.010884 |
| Mir3962 | −1.2552 | −0.32792 | 0.023414 | Gm3187 | 1.27761 | 0.353443 | 0.027363 |
| Mcf2 | −1.25491 | −0.327578 | 0.014087 | Vmn2r1 | 1.27644 | 0.352122 | 0.004155 |
| BC046251 | −1.25449 | −0.327106 | 0.044489 | Clcn6 | 1.2764 | 0.352081 | 0.020631 |
| Pcsk5 | −1.25425 | −0.326828 | 0.039558 | Krt35 | 1.2763 | 0.351962 | 0.026767 |
| Ak5 | −1.25382 | −0.326335 | 0.024482 | Pon2 | 1.27627 | 0.351929 | 0.038543 |
| Sec1 | −1.25382 | −0.326325 | 0.036434 | Ensa | 1.27414 | 0.349526 | 0.011783 |
| Gm3227 | −1.25362 | −0.326095 | 0.037101 | Csrp1 | 1.27197 | 0.347069 | 0.039556 |
| Taar5 | −1.25174 | −0.323937 | 0.041408 | Itm2c | 1.2716 | 0.34664 | 0.002487 |
| Ppp1r3c | −1.25158 | −0.323746 | 0.006226 | Gp1ba | 1.27087 | 0.345813 | 0.023916 |
| Prosapip1 | −1.25107 | −0.323162 | 0.019054 | Ccdc134 | 1.26766 | 0.342165 | 0.011857 |
| Apoc1 | −1.25106 | −0.32315 | 0.027501 | Gp1bb | 1.26723 | 0.341675 | 0.03046 |
| Mum1 | −1.25056 | −0.322572 | 0.045699 | Gss | 1.26699 | 0.341406 | 0.00065 |
| Txlng | −1.25045 | −0.322448 | 0.01749 | Nradd | 1.26657 | 0.340924 | 0.035929 |
| Gm19396 | −1.25038 | −0.322364 | 0.032719 | Myo1f | 1.26571 | 0.339948 | 0.025168 |
| — | — | — | — | Tubb4a | 1.26535 | 0.339537 | 0.017563 |
| — | — | — | — | Ift57 | 1.26434 | 0.338381 | 0.019749 |
| — | — | — | — | Tfec | 1.26376 | 0.337718 | 0.013378 |
| — | — | — | — | 4930563D23Rik | 1.2609 | 0.334451 | 0.029223 |
| — | — | — | — | Fyb | 1.26065 | 0.334164 | 0.041566 |
| — | — | — | — | Gm16576 | 1.25968 | 0.33306 | 0.015253 |
| — | — | — | — | Fam151b | 1.25879 | 0.332042 | 0.020006 |
| — | — | — | — | Metrn1 | 1.25873 | 0.331974 | 0.037403 |
| — | — | — | — | Csrp2 | 1.25835 | 0.331533 | 0.024785 |
| — | — | — | — | Klhdc5 | 1.25803 | 0.331172 | 0.005083 |
| — | — | — | — | Rpl27 | 1.25473 | 0.327379 | 0.021739 |
| — | — | — | — | E430025E21Rik | 1.25395 | 0.326475 | 0.025502 |
| — | — | — | — | Slco6c1 | 1.25339 | 0.32584 | 0.035209 |
| — | — | — | — | 1520402A15Rik | 1.25236 | 0.324647 | 0.035245 |
| — | — | — | — | Trp53bp2 | 1.25227 | 0.324544 | 0.010169 |
| — | — | — | — | Pcdhb21 | 1.25218 | 0.324439 | 0.019117 |
| — | — | — | — | Pde4a | 1.25076 | 0.322808 | 0.048793 |
| — | — | — | — | Tmem189 | 1.25013 | 0.322084 | 0.045644 |
| — | — | — | — | | | | |

Foxa3^{-/-} LSK CD150+CD48− versus Foxa3^{+/+} LSK CD150+CD48− cells
Listed are fold changes >1.25
p value threshold <0.05

Gene Ontology (GO) enrichment analysis (Ashburner et al., 2000) of this gene set yielded terms including cell cycle (“mitotic cell cycle” and “DNA replication”), metabolism (“nucleic acid biosynthesis” and “peptidyl-asparagine modification”), and stress (“ER overload response”, “response to ER stress”, “ER-nuclear signaling pathway”) as putative regulated processes (Table 7).

TABLE 7
Gene Ontology (GO) Analysis.

| GO ID | Description | #Genes | Q-value |
|---|---|---|---|
| GO:0034654 | Nucleobase, nucleoside, | 200 | 1.01 × 10⁻¹¹ |
| | nucleotide, and nucleic acid | | |
| | biosynthetic process | | |
| GO:0000278 | Mitotic cell cycle | 53 | 7.45 × 10−7 |
| GO:0008380 | RNA splicing | 31 | 5.5 × 10−6 |
| GO:0006260 | DNA replication | 25 | 0.000515 |
| GO:0018196 | Peptidyl-asparagine modification | 12 | 0.000817 |
| GO:0042692 | Muscle cell differentiation | 24 | 0.000832 |
| GO:0006984 | ER-nuclear signaling pathway | 12 | 0.00161 |
| GO:0002260 | Lymphocyte homeostasis | 8 | 0.00204 |
| GO:0007610 | Behavior | 50 | 0.00239 |
| GO:0006983 | ER overload response | 4 | 0.00283 |
| GO:0034976 | Response to ER stress | 12 | 0.0044 |

Ingenuity Pathway analysis yielded multiple pathways that matched our gene set because of a common signature that included: Myc, Fos, Stat5a, PIK3CA, Nras, Grb2, PIK3CG, SOS1, and Stat3 (Table 8). These are molecules commonly found downstream of growth and cytokine receptors that interface with survival, cell cycle, and metabolic signaling.

“Unfolded Protein Response” and “Endoplasmic Reticulum Stress Pathways” also matched to our dataset. Top Predicted Regulators included Myc, TP53, and TGFβ (Table 8).

TABLE 8
Ingenuity Pathway Analysis Results.

| Top Canonical | | | Genes assigned to pathway that are |
|---|---|---|---|
| Pathways | P value | Ratio | present in interrogated gene set |
| Prolactin Signaling | 0.000125893 | 0.15 | MYC, FYN, FOS, STAT5A, PIK3CA, NRAS, |
| | | | GRB2, PIK3CG, SOS1, STAT3, STAT5B |
| IL-2 Signaling | 0.001023293 | 0.151 | FOS, STAT5A, PIK3CA, NRAS, GRB2, |
| | | | PIK3CG, SOS1, STAT5B |
| Myc Mediated Apoptosis | 0.000416869 | 0.155 | MYC, PIK3CA, NRAS, CASP3, GRB2, |
| Signaling | | | PIK3CG, SOS1, YWHAZ, MAPK9 |
| Chronic Myeloid | 3.38844E−06 | 0.161 | STAT5A, PIK3CA, NRAS, GRB2, SMAD3, |
| Leukemia Signaling | | | CDK4, CRK, HDAC5, MYC, TGFBR2, RB1, |
| | | | PIK3CG, SOS1, E2F1, STAT5B |
| GM-CSF Signaling | 0.000144544 | 0.161 | PIK3CA, NRAS, PPP3CB, GRB2, PIK3CG, |
| | | | PPP3R1, SOS1, STAT3, STAT5B, PPP3CA |
| Antigen Presentation | 0.003019952 | 0.162 | HLA-G, HLA-A, HLA-DMB, |
| Pathway | | | PSMB8, TAP2, HLA-E |
| Role of p14/p19ARF in | 0.005888437 | 0.167 | RB1, PIK3CA, PIK3CG, E2F1, UBTF |
| Tumor Suppression | | | |
| Acute Myeloid Leukemia | 8.91251E−06 | 0.169 | MYC, MAP2K6, STAT5A, TCF4, PIK3CA, |
| Signaling | | | NRAS, GRB2, PIK3CG, SOS1, STAT3, PML, |
| | | | STAT5B, TCF7L2 |
| IL-3 Signaling | 1.94984E−05 | 0.169 | FOS, STAT5A, PIK3CA, NRAS, PPP3CB, |
| | | | GRB2, PIK3CG, PPP3R1, SOS1, STAT3, |
| | | | STAT5B, PPP3CA |
| Cell Cycle Regulation by | 0.002238721 | 0.171 | RB1, PPP2CA, E2F1, BTG2, CDK4, BTG1 |
| BTG Family Proteins | | | |
| ErbB2-ErbB3 Signaling | 6.91831E−05 | 0.175 | MYC, STAT5A, PIK3CA, NRAS, GRB2, |
| | | | PIK3CG, SOS1, ERBB3, STAT3, STAT5B |
| Oncostatin M Signaling | 0.001905461 | 0.176 | STAT5A, NRAS, GRB2, SOS1, STAT3, |
| | | | STAT5B |
| Thrombopoietin | 5.01187E−05 | 0.182 | MYC, FOS, STAT5A, PIK3CA, NRAS, GRB2, |
| Signaling | | | PIK3CG, SOS1, STAT3, STAT5B |
| Unfolded protein | 6.76083E−06 | 0.2 | DDIT3, SREBF1, EDEM1, ERO1LB, HSPA9, |
| response | | | DNAJC3, ATF6, OS9, MAP3K5, HSPA5, |
| | | | HSPA2 |
| Endoplasmic Reticulum | 0.000120226 | 0.286 | CASP3, DDIT3, DNAJC3, ATF6, MAP3K5, |
| Stress Pathway | | | HSPA5 |
| Calcium Transport I | 0.004265795 | 0.333 | ATP2B1, ATP2A3, ATP2B4 |
| UDP-N-acetyl-D- | 0.004265795 | 0.333 | HK1, GNPDA1, GPI |
| galactosamine | | | |
| Biosynthesis II | | | |
| N-acetylglucosamine | 0.008912509 | 0.5 | GNPDA1, AMDHD2 |
| Degradation II | | | |
| N-acetylglucosamine | 0.004570882 | 0.667 | GNPDA1, AMDHD2 |
| Degradation I | | | |

| Upstream Regulator | Molecule Type | P value | Regulator targets in interrogated data set |
|---|---|---|---|
| camptothecin | chemical | 2.05E−08 | ALDOA, BFAR, BTG2, CASP3, CD200, CD47, |
| | reagent | | CDIPT, CDK4, CHCHD7, CSNK1G2, DENND5B, |
| | | | DGKZ, E2F1, FNDC3A, FOS, FUT8, |
| | | | GAMT, GNAI2, GNG7, GPI, HIST1H3B, |
| | | | HLA-A, HLA-E, HLA-G, |
| | | | ITSN1, KIF3B, LITAF, MOAP1, MYC, |
| | | | NUMA1, P2RX1, PECAM1, PHF21A, PIK3CA, |
| | | | PIK3CG, PKN2, PPP3CA, PRKAR2B, PRUNE, |
| | | | PTGES, PTP4A2, PTPN1, RB1, RBMS1, |
| | | | RRM2B, STAT5B, TIAL1, TNFAIP1, TRAF1, |
| | | | TRIB1, TSPAN3 |
| HNF4A | transcription | 4.18E−08 | ACBD6, ACIN1, ADIPOR1, AHNAK, AIP, |
| | regulator | | ANXA9, APH1A, ARL1, ATP10A, BLOC1S1, |
| | | | BOLA1, BRIP1, BTG1, C11orf54, CCDC47, |
| | | | CCDC90B, CDC123, CDC23, CDIPT, CHMP1B, |
| | | | CLPX, COASY, CRYZL1, CSK, DAG1, DNAJA3, |
| | | | ECI2, EDEM3, ESYT1, FBXW2, FURIN, |
| | | | FUT8, GALM, GSN, GSPT1, GSS, GTF2I, HLA-G, |
| | | | HMOX2, HSPA5, IVNS1ABP, KBTBD4, |
| | | | KIF3B, KLHL20, LIMS1, LMAN2L, LRRC40, |
| | | | LUC7L2, LYPLA2, MED23, MOCOS, MRPL22, |
| | | | MRPL27, MRPS7, MTHFD1, MTRF1, |
| | | | MYC, NCOA3, NDUFB5, NRAS, NUP62, ORMDL2, |
| | | | P2RY14, PCYT1A, PEX11B, PHB2, PKN2, |
| | | | PNKP, PPP1R15B, PPP2R3C, PRR14, PRRG2, |
| | | | PTGES3, RAB10, RAB11A, RABGEF1, |
| | | | RAD17, RBM39, RFC5, RNF40, RPAP3, |
| | | | RPL12, RPS25, RPS27A, SCP2, SEC23IP, |
| | | | SF3B4, SKI, SLC35A5, SLC35D1, SRP68, SRSF11, |
| | | | SSBP1, SSU72, STAU2, STOM, STOML1, |
| | | | SUGT1, TBC1D17, TCF7L2, TCIRG1, TM9SF4, |
| | | | TNFAIP1, TRPC4AP, TRUB2, TXNIP, |
| | | | TYMS, UBE2B, UBE2D3, UBL7, UBP1, UPF3B, |
| | | | USF1, USP30, VDAC1, VPS29, YPEL3, |
| | | | ZC3H10, ZDHHC6, ZFYVE19 |
| ELAVL1 | other | 2.52E−06 | ELAVL1, FOS, GSS, HSPA2, MYC, NXF1, |
| | | | RFC5, RPS14, SLC7A7, SRSF7, STAT3, TAF9, |
| | | | TRIOBP |
| MYC | transcription | 3.57E−06 | ADIPOR1, AKAP1, ALDOA, ATAD3A, BRD2, |
| | regulator | | CAPN2, CASP3, CD47, CD9, Cdc42, CDK4, |
| | | | COL1A2, CTBS, CTNNA1, DDIT3, DDX3X, |
| | | | E2F1, ELAVL1, FCGRT, FOS, FUT8, GAMT, |
| | | | GART, GPI, HLA-A, HLA-E, |
| | | | HMGA1, Hmgn2 (includes |
| | | | others), HMOX2, HSPA9, IPO7, JARID2, |
| | | | KLF4, LIMS1, LRMP, MBP, MKI67, MRE11A, |
| | | | MTHFD1, MYC, MYLPF, MYO1C, PECAM1, |
| | | | PHB2, PHF20, PHF21A, PML, POLD1, Ppp1cc, |
| | | | PPP2CA, PRDX2, PSMB8, RAB10, RB1, |
| | | | RPL7, RRM2B, SCPEP1, SHMT2, SUMO2, |
| | | | TAF1D, TGFBR2, TXNIP, TYMS, VAMP3, VHL |
| TP53 | transcription | 8.21E−06 | ALDH9A1, ASXL1, BHLHE40, BTG1, BTG2, |
| | regulator | | CASP3, CD47, Cdc42, CDK4, CDKN3, CISD1, |
| | | | COL1A2, Cox5b/LOC102638382, CSK, |
| | | | DDIT3, DDX3X, E2F1, ENG, FOS, FYN, GART, |
| | | | GDA, GLB1, GPI, GRB2, GSN, HDAC5, |
| | | | HDLBP, HMGCR, Hmgn2 (includes |
| | | | others), IPO7, KLF4, LSS, MAP2K6, MDM4, |
| | | | MICALL1, MKI67, MOCOS, MTDH, MYC, |
| | | | MYO1C, OMA1, PAFAH1B2, PCCA, PDE4B, |
| | | | PECAM1, PLEKHB2, Pmaip1, PML, POLD1, |
| | | | POLE2, PPP2CA, PPP3CA, PRDX2, |
| | | | PRDX6, PRPSAP1, PTPN1, PTPN12, RAD17, |
| | | | RAD23A, RB1, RPS25, RPS27L, RRM2B, SCP2, |
| | | | SCPEP1, SLC19A1, SON, SREBF1, SSH1, |
| | | | TANK, TAP2, TCF7L2, TGFBR2, TP53BP2, |
| | | | TYMS, UBE2B, VCL, WSB2, ZYX |
| miR-124-3p (and other | mature | 8.63E−06 | ALDH9A1, CDK4, DNM2, ECI2, ELK3, |
| miRNAs w/seed | microrna | | FAM129B, GSN, GTPBP8, LAMC1, LITAF, |
| AAGGCAC) | | | LMNB1, MAPK1IP1L, NAA15, PGF, PTPN12, |
| | | | RBMS1, SENP8, SERP1, STAT3, STOM, |
| | | | SYPL1, USP48, VAMP3 |
| EIF2AK3 | kinase | 1.01E−05 | ATF6, BTG2, DDIT3, DNAJC3, ERO1LB, |
| | | | HERPUD1, HSPA5, HSPA9, KLF4, MYC, PON2, |
| | | | RNASEL, SHMT2, TXNIP |
| FLT1 | kinase | 1.07E−05 | CAPN2, DDX3X, FOS, HK1, LIMS1, |
| | | | SMARCA4, TCF4, TGFBR2, UBE2B, VDAC1, |
| | | | VWF, YWHAZ |
| TGFB1 | growth factor | 2.14E−05 | AHNAK, ARF4, BHLHE40, BRIP1, BTG1, |
| | | | CAB39, CASP3, CDK4, CDKN3, CELF2, |
| | | | COL1A2, COTL1, CTCF, DNAJB6, DNMT3A, |
| | | | DNMT3B, E2F1, EIF4H, ELK3, ENG, FOS, |
| | | | FTL, FURIN, FUT8, FYN, GABBR1, GALM, |
| | | | GNAI2, GNG7, GNL1, GSN, HEXB, HMGA1, |
| | | | HMOX2, HSF2BP, HSPA5, IRAK2, IRAK3, |
| | | | ITGA4, KLF4, KPNA3, LAMC1, LIMS1, LITAF, |
| | | | MAPK6, MKI67, MRE11A, MYC, MYL12A, |
| | | | MYLPF, MYO1C, NCOA3, NIPA2, NUP62, |
| | | | P2RY14, PECAM1, PITPNM1, PKIG, PML, |
| | | | POLD1, POLE2, PPP2CA, PSMC3, PTAFR, |
| | | | PTGES, RAB1A, RB1, RBMS1, RFC5, RNF111, |
| | | | SERP1, SKI, SLC23A2, SLC35A5, SMAD3, |
| | | | SRCAP, SSRP1, STAT3, STAT5A, STAT5B, |
| | | | STK16, TGFBR2, TGFBR3, TPM3, TRAF1, |
| | | | TXNIP, TYMS, VCL, VWF, ZEB2, ZYX |
| ERBB2 | kinase | 2.20E−05 | AHNAK, ATP6V1A, BHLHE40, BNIP2, BRIP1, |
| | | | BTG2, CD47, CD9, CDKN3, DAG1, DDIT3, |
| | | | DNAJB6, EIF6, ELK3, EPSTI1, ERBB3, FOS, |
| | | | FZD4, Hbb-b1, Hbb-b2, |
| | | | HSD17B11, KLF4, LITAF, MKI67, MYC, |
| | | | NR1H2, POLD1, POLE2, PRDX2, PSMC3, |
| | | | PTGES, PTPN1, PTRF, QKI, SERP1, SMAD3, |
| | | | ST3GAL6, STAT3, TAP2, TRAF1, TYMS, |
| | | | UQCR10, VCL, VWF, WSB2 |

GSEA analysis also returned categories indicative of perturbed stress, signaling, and metabolic pathways (e.g. “apoptosis by doxyrubicin”, “up in CML”, “biopolymer metabolic process”, Table 9).

TABLE 9
Gene Set Enrichment Analysis (GSEA) Results.

| | # Genes | | | | | |
|---|---|---|---|---|---|---|
| | in Gene | | # Genes in | | | FDR |
| Gene Set Name | Set (K) | Description | Overlap (k) | k/K | p-value | q-value |
| PILON_KLF1_TARGETS_DN | 1972 | Genes down-regulated | 181 | 0.0918 | 2.22E−75 | 2.21E−71 |
| | | in erythroid progenitor | | | | |
| | | cells from fet al livers | | | | |
| | | of E13.5 embryos with | | | | |
| | | KLF1 | | | | |
| | | [GeneID = 10661] | | | | |
| | | knockout compared to | | | | |
| | | those from the wild | | | | |
| | | type embryos. | | | | |
| GGGCGGR_V$SP1_Q6 | 2940 | Genes with promoter | 213 | 0.0724 | 7.31E−71 | 3.64E−67 |
| | | regions [−2 kb, 2 kb] | | | | |
| | | around transcription | | | | |
| | | start site containing the | | | | |
| | | motif GGGCGGR | | | | |
| | | which matches | | | | |
| | | annotation for SP1: | | | | |
| | | Sp1 transcription | | | | |
| | | factor | | | | |
| GRAESSMANN_APOPTOSIS_BY_DOXORUBICIN_DN | 1781 | Genes down-regulated | 157 | 0.0882 | 1.98E−62 | 6.56E−59 |
| | | in ME-A cells (breast | | | | |
| | | cancer) undergoing | | | | |
| | | apoptosis in response | | | | |
| | | to doxorubicin | | | | |
| | | [PubChem = 31703]. | | | | |
| DIAZ_CHRONIC_MEYLOGENOUS_LEUKEMIA_UP | 1382 | Genes up-regulated in | 139 | 0.1006 | 4.97E−62 | 1.24E−58 |
| | | CD34+ [GeneID = 947] | | | | |
| | | cells isolated from | | | | |
| | | bone marrow of CML | | | | |
| | | (chronic myelogenous | | | | |
| | | leukemia) patients, | | | | |
| | | compared to those | | | | |
| | | from normal donors. | | | | |
| MARSON_BOUND_BY_FOXP3_UNSTIMULATED | 1229 | Genes with promoters | 122 | 0.0993 | 1.28E−53 | 2.55E−50 |
| | | bound by FOXP3 | | | | |
| | | [GeneID = 50943] in | | | | |
| | | unstimulated | | | | |
| | | hybridoma cells. | | | | |
| CYTOPLASM | 2131 | Genes annotated by | 157 | 0.0737 | 5.25E−52 | 8.72E−49 |
| | | the GO term | | | | |
| | | GO: 0005737. Cntents | | | | |
| | | of a cell excluding the | | | | |
| | | plasma membrane and | | | | |
| | | nucleus, but including | | | | |
| | | other subcellular | | | | |
| | | structures. | | | | |
| BIOPOLYMER_METABOLIC_PROCESS | 1684 | Genes annotated by | 132 | 0.0784 | 2.23E−46 | 3.18E−43 |
| | | the GO term | | | | |
| | | GO: 0043283. The | | | | |
| | | chemical reactions and | | | | |
| | | pathways involving | | | | |
| | | biopolymers, long, | | | | |
| | | repeating chains of | | | | |
| | | monomers found in | | | | |
| | | nature e.g. | | | | |
| | | polysaccharides and | | | | |
| | | proteins. | | | | |
| PUJANA_BRCA1_PCC_NETWORK | 1652 | Genes constituting the | 122 | 0.0738 | 3.70E−40 | 4.61E−37 |
| | | BRCA1-PCC network | | | | |
| | | of transcripts whose | | | | |
| | | expression positively | | | | |
| | | correlated (Pearson | | | | |
| | | correlation coefficient, | | | | |
| | | PCC >= 0.4) with that | | | | |
| | | of BRCA1 | | | | |
| | | [GeneID = 672] across | | | | |
| | | a compendium of | | | | |
| | | normal tissues. | | | | |
| MARSON_BOUND_BY_FOXP3_STIMULATED | 1022 | Genes with promoters | 95 | 0.093 | 2.98E−39 | 3.31E−36 |
| | | bound by FOXP3 | | | | |
| | | [GeneID = 50943] in | | | | |
| | | hybridoma cells | | | | |
| | | stimulated by PMA | | | | |
| | | [PubChem = 4792] and | | | | |
| | | ionomycin | | | | |
| | | [PubChem = 3733]. | | | | |
| NUCLEUS | 1430 | Genes annotated by | 112 | 0.0783 | 3.47E−39 | 3.46E−36 |
| | | the GO term | | | | |
| | | GO: 0005634. A | | | | |
| | | membrane-bounded | | | | |
| | | organelle of eukaryotic | | | | |
| | | cells in which | | | | |
| | | chromosomes are | | | | |
| | | housed and replicated. | | | | |
| | | In most cells, the | | | | |
| | | nucleus contains the | | | | |
| | | cell's chromosomes | | | | |
| | | except the organellar | | | | |
| | | chromosomes, and is | | | | |
| | | the site of RNA | | | | |
| | | synthesis and | | | | |
| | | processing. In some | | | | |
| | | species, or in | | | | |
| | | specialized cell types, | | | | |
| | | RNA metabolism or | | | | |
| | | DNA replication may | | | | |
| | | be absent. | | | | |
| GGGAGGRR_V$MAZ_Q6 | 2274 | Genes with promoter | 142 | 0.0624 | 1.25E−38 | 1.13E−35 |
| | | regions [−2 kb, 2 kb] | | | | |
| | | around transcription | | | | |
| | | start site containing the | | | | |
| | | motif GGGAGGRR | | | | |
| | | which matches | | | | |
| | | annotation for MAZ: | | | | |
| | | MYC-associated zinc | | | | |
| | | finger protein (purine- | | | | |
| | | binding transcription | | | | |
| | | factor) | | | | |
| DACOSTA_UV_RESPONSE_VIA_ERCC3_DN | 855 | Genes down-regulated | 86 | 0.1006 | 3.77E−38 | 3.14E−35 |
| | | in fibroblasts | | | | |
| | | expressing mutant | | | | |
| | | forms of ERCC3 | | | | |
| | | [GeneID = 2071] after | | | | |
| | | UV irradiation. | | | | |
| NUCLEOBASENUCLEOSIDENUCLEOTIDE_AND_- | 1244 | Genes annotated by | 103 | 0.0828 | 4.18E−38 | 3.20E−35 |
| NUCLEIC_ACID_METABOLIC_PROCESS | | the GO term | | | | |
| | | GO: 0006139. The | | | | |
| | | chemical reactions and | | | | |
| | | pathways involving | | | | |
| | | nucleobases, | | | | |
| | | nucleosides, | | | | |
| | | nucleotides and | | | | |
| | | nucleic acids. | | | | |
| DANG_BOUND_BY_MYC | 1103 | Genes whose | 93 | 0.0843 | 4.95E−35 | 3.52E−32 |
| | | promoters are bound | | | | |
| | | by MYC | | | | |
| | | [GeneID = 4609], | | | | |
| | | according to MYC | | | | |
| | | Target Gene Database. | | | | |
| INTRACELLULAR_ORGANELLE_PART | 1192 | Genes annotated by | 96 | 0.0805 | 1.53E−34 | 1.02E−31 |
| | | the GO term | | | | |
| | | GO: 0044446. A | | | | |
| | | constituent part of an | | | | |
| | | intracellular organelle, | | | | |
| | | an organized structure | | | | |
| | | of distinctive | | | | |
| | | morphology and | | | | |
| | | function, occurring | | | | |
| | | within the cell. | | | | |
| | | Includes constituent | | | | |
| | | parts of the nucleus, | | | | |
| | | mitochondria, plastids, | | | | |
| | | vacuoles, vesicles, | | | | |
| | | ribosomes and the | | | | |
| | | cytoskeleton but | | | | |
| | | excludes the plasma | | | | |
| | | membrane. | | | | |
| ORGANELLE_PART | 1197 | Genes annotated by | 96 | 0.0802 | 2.14E−34 | 1.33E−31 |
| | | the GO term | | | | |
| | | GO: 0044422. Any | | | | |
| | | constituent part of an | | | | |
| | | organelle, an | | | | |
| | | organized structure of | | | | |
| | | distinctive morphology | | | | |
| | | and function. Includes | | | | |
| | | constituent parts of the | | | | |
| | | nucleus, mitochondria, | | | | |
| | | plastids, vacuoles, | | | | |
| | | vesicles, ribosomes | | | | |
| | | and the cytoskeleton, | | | | |
| | | but excludes the | | | | |
| | | plasma membrane. | | | | |
| CYTOPLASMIC_PART | 1383 | Genes annotated by | 102 | 0.0738 | 1.82E−33 | 1.07E−30 |
| | | the GO term | | | | |
| | | GO: 0044444. Any | | | | |
| | | constituent part of the | | | | |
| | | cytoplasm, the | | | | |
| | | contents of a cell | | | | |
| | | excluding the plasma | | | | |
| | | membrane and | | | | |
| | | nucleus, but including | | | | |
| | | other subcellular | | | | |
| | | structures. | | | | |
| BLALOCK_ALZHEIMERS_DISEASE_DN | 1237 | Genes down-regulated | 96 | 0.0776 | 2.98E−33 | 1.65E−30 |
| | | in brain from patients | | | | |
| | | with Alzheimer's | | | | |
| | | disease. | | | | |
| PROTEIN_METABOLIC_PROCESS | 1231 | Genes annotated by | 95 | 0.0772 | 1.02E−32 | 5.37E−30 |
| | | the GO term | | | | |
| | | GO: 0019538. The | | | | |
| | | chemical reactions and | | | | |
| | | pathways involving a | | | | |
| | | specific protein, rather | | | | |
| | | than of proteins in | | | | |
| | | general. Includes | | | | |
| | | protein modification. | | | | |
| SCGGAAGY_V$ELK1_02 | 1199 | Genes with promoter | 91 | 0.0759 | 8.33E−31 | 4.15E−28 |
| | | regions [−2 kb, 2 kb] | | | | |
| | | around transcription | | | | |
| | | start site containing the | | | | |
| | | motif SCGGAAGY | | | | |
| | | which matches | | | | |
| | | annotation for ELK1: | | | | |
| | | ELK1, member of ETS | | | | |
| | | oncogene family | | | | |
| BENPORATH_MYC_MAX_TARGETS | 775 | Set ‘Myc targets2’: | 73 | 0.0942 | 1.25E−30 | 5.92E−28 |
| | | targets of c-Myc | | | | |
| | | [GeneID = 4609] and | | | | |
| | | Max [GeneID = 4149] | | | | |
| | | identified by ChIP on | | | | |
| | | chip in a Burkitt's | | | | |
| | | lymphoma cell line; | | | | |
| | | overlap set. | | | | |
| CAGGTG_V$E12_Q6 | 2485 | Genes with promoter | 135 | 0.0543 | 1.75E−30 | 7.93E−28 |
| | | regions [−2 kb, 2 kb] | | | | |
| | | around transcription | | | | |
| | | start site containing the | | | | |
| | | motif CAGGTG which | | | | |
| | | matches annotation for | | | | |
| | | TCF3: transcription | | | | |
| | | factor 3 (E2A | | | | |
| | | immunoglobulin | | | | |
| | | enhancer binding | | | | |
| | | factors E12/E47) | | | | |
| NUYTTEN_EZH2_TARGETS_UP | 1037 | Genes up-regulated in | 82 | 0.0791 | 5.16E−29 | 2.24E−26 |
| | | PC3 cells (prostate | | | | |
| | | cancer) after | | | | |
| | | knockdown of EZH2 | | | | |
| | | [GeneID = 2146] by | | | | |
| | | RNAi. | | | | |
| NUYTTEN_NIPP1_TARGETS_DN | 848 | Genes down-regulated | 74 | 0.0873 | 6.42E−29 | 2.67E−26 |
| | | in PC3 cells (prostate | | | | |
| | | cancer) after | | | | |
| | | knockdown of NIPP1 | | | | |
| | | [GeneID = 5511] by | | | | |
| | | RNAi. | | | | |
| WAKABAYASHI_ADIPOGENESIS_PPARG_RXRA_- | 882 | Genes with promoters | 75 | 0.085 | 1.40E−28 | 5.57E−26 |
| BOUND_8D | | bound by both PPARG | | | | |
| | | and RXRA | | | | |
| | | [GeneID = 5468, 6256] | | | | |
| | | at 8 day time point of | | | | |
| | | adipocyte | | | | |
| | | differentiation of 3T3- | | | | |
| | | L1 cells | | | | |
| | | (preadipocyte). | | | | |
| RNA_METABOLIC_PROCESS | 841 | Genes annotated by | 73 | 0.0868 | 2.15E−28 | 8.24E−26 |
| | | the GO term | | | | |
| | | GO: 0016070. The | | | | |
| | | chemical reactions and | | | | |
| | | pathways involving | | | | |
| | | RNA, ribonucleic acid, | | | | |
| | | one of the two main | | | | |
| | | type of nucleic acid, | | | | |
| | | consisting of a long, | | | | |
| | | unbranched | | | | |
| | | macromolecule formed | | | | |
| | | from ribonucleotides | | | | |
| | | joined in 3′,5′- | | | | |
| | | phosphodiester | | | | |
| | | linkage. | | | | |
| MILI_PSEUDOPODIA_HAPTOTAXIS_DN | 668 | Transcripts depleted | 65 | 0.0973 | 3.61E−28 | 1.33E−25 |
| | | from pseudopodia of | | | | |
| | | NIH/3T3 cells | | | | |
| | | (fibroblast) in response | | | | |
| | | to haptotactic | | | | |
| | | migratory stimulus by | | | | |
| | | fibronectin, FN1 | | | | |
| | | [GeneID = 2335]. | | | | |
| CTTTGT_V$LEF1_Q2 | 1972 | Genes with promoter | 114 | 0.0578 | 5.92E−28 | 2.11E−25 |
| | | regions [−2 kb, 2 kb] | | | | |
| | | around transcription | | | | |
| | | start site containing the | | | | |
| | | motif CTTTGT which | | | | |
| | | matches annotation for | | | | |
| | | LEF1: lymphoid | | | | |
| | | enhancer-binding | | | | |
| | | factor 1 | | | | |
| BLALOCK_ALZHEIMERS_DISEASE_UP | 1691 | Genes up-regulated in | 104 | 0.0615 | 1.28E−27 | 4.38E−25 |
| | | brain from patients | | | | |
| | | with Alzheimer's | | | | |
| | | disease. | | | | |
| BUYTAERT_PHOTODYNAMIC_THERAPY_- | 811 | Genes up-regulated in | 69 | 0.0851 | 2.26E−26 | 7.51E−24 |
| STRESS_UP | | T24 (bladder cancer) | | | | |
| | | cells in response to the | | | | |
| | | photodynamic therapy | | | | |
| | | (PDT) stress. | | | | |
| LOPEZ_MBD_TARGETS | 957 | Genes up-regulated in | 75 | 0.0784 | 2.37E−26 | 7.62E−24 |
| | | HeLa cells (cervical | | | | |
| | | cancer) after | | | | |
| | | simultaneus | | | | |
| | | knockdown of three | | | | |
| | | MBD (methyl-CpG | | | | |
| | | binding domain) | | | | |
| | | proteins MeCP2, | | | | |
| | | MBD1 and MBD2 | | | | |
| | | [GeneID = 4204; 4152; 8932] | | | | |
| | | by RNAi. | | | | |
| RCGCANGCGY_V$NRF1_Q6 | 918 | Genes with promoter | 73 | 0.0795 | 4.71E−26 | 1.47E−23 |
| | | regions [−2 kb, 2 kb] | | | | |
| | | around transcription | | | | |
| | | start site containing the | | | | |
| | | motif | | | | |
| | | RCGCANGCGY | | | | |
| | | which matches | | | | |
| | | annotation for NRF1: | | | | |
| | | nuclear respiratory | | | | |
| | | factor 1 | | | | |
| FLECHNER_BIOPSY_KIDNEY_TRANSPLANT_OK_- | 555 | Genes up-regulated in | 57 | 0.1027 | 5.25E−26 | 1.58E−23 |
| VS_DONOR_UP | | kidney biopsies from | | | | |
| | | patients with well | | | | |
| | | functioning kidneys | | | | |
| | | more than 1-year post | | | | |
| | | transplant compared to | | | | |
| | | the biopsies from | | | | |
| | | normal living kidney | | | | |
| | | donors. | | | | |
| CAGCTG_V$AP4_Q5 | 1524 | Genes with promoter | 95 | 0.0623 | 1.12E−25 | 3.27E−23 |
| | | regions [−2 kb, 2 kb] | | | | |
| | | around transcription | | | | |
| | | start site containing the | | | | |
| | | motif CAGCTG which | | | | |
| | | matches annotation for | | | | |
| | | REPIN1: replication | | | | |
| | | initiator 1 | | | | |
| DACOSTA_UV_RESPONSE_VIA_ERCC3_- | 483 | Common down- | 53 | 0.1097 | 1.26E−25 | 3.59E−23 |
| COMMON_DN | | regulated transcripts in | | | | |
| | | fibroblasts expressing | | | | |
| | | either XP/CS or TDD | | | | |
| | | mutant forms of | | | | |
| | | ERCC3 | | | | |
| | | [GeneID = 2071], after | | | | |
| | | UVC irradiation. | | | | |
| PUJANA_ATM_PCC_NETWORK | 1442 | Genes constituting the | 92 | 0.0638 | 1.35E−25 | 3.74E−23 |
| | | ATM-PCC network of | | | | |
| | | transcripts whose | | | | |
| | | expression positively | | | | |
| | | correlated (Pearson | | | | |
| | | correlation coefficient, | | | | |
| | | PCC >= 0.4) with that | | | | |
| | | of ATM | | | | |
| | | [GeneID = 472] across | | | | |
| | | a compendium of | | | | |
| | | normal tissues. | | | | |
| CELLULAR_MACROMOLECULE_METABOLIC_- | 1131 | Genes annotated by | 80 | 0.0707 | 3.71E−25 | 9.99E−23 |
| PROCESS | | the GO term | | | | |
| | | GO: 0044260. The | | | | |
| | | chemical reactions and | | | | |
| | | pathways involving | | | | |
| | | macromolecules, large | | | | |
| | | molecules including | | | | |
| | | proteins, nucleic acids | | | | |
| | | and carbohydrates, as | | | | |
| | | carried out by | | | | |
| | | individual cells. | | | | |
| CUI_TCF21_TARGETS_2_DN | 830 | Significantly down- | 68 | 0.0819 | 4.61E−25 | 1.21E−22 |
| | | regulated genes in | | | | |
| | | kidney glomeruli | | | | |
| | | isolated from TCF21 | | | | |
| | | [Gene ID = 6943] | | | | |
| | | knockout mice. | | | | |
| KINSEY_TARGETS_OF_EWSR1_FLII_FUSION_UP | 1278 | Genes up-regulated in | 85 | 0.0665 | 6.92E−25 | 1.77E−22 |
| | | TC71 and EWS502 | | | | |
| | | cells (Ewing's | | | | |
| | | sarcoma) by EWSR1- | | | | |
| | | FLI1 | | | | |
| | | [GeneID = 2130; 2314] | | | | |
| | | as inferred from RNAi | | | | |
| | | knockdown of this | | | | |
| | | fusion protein. | | | | |
| REGULATION_OF_GENE_EXPRESSION | 673 | Genes annotated by | 61 | 0.0906 | 7.39E−25 | 1.84E−22 |
| | | the GO term | | | | |
| | | GO: 0010468. Any | | | | |
| | | process that modulates | | | | |
| | | the frequency, rate or | | | | |
| | | extent of gene | | | | |
| | | expression. Gene | | | | |
| | | expression is the | | | | |
| | | process in which a | | | | |
| | | gene's coding | | | | |
| | | sequence is converted | | | | |
| | | into a mature gene | | | | |
| | | product or products | | | | |
| | | (proteins or RNA). | | | | |
| | | This includes the | | | | |
| | | production of an RNA | | | | |
| | | transcript as well as | | | | |
| | | any processing to | | | | |
| | | produce a mature RNA | | | | |
| | | product or an mRNA | | | | |
| | | (for protein-coding | | | | |
| | | genes) and the | | | | |
| | | translation of that | | | | |
| | | mRNA into protein. | | | | |
| | | Some protein | | | | |
| | | processing events may | | | | |
| | | be included when they | | | | |
| | | are required to form an | | | | |
| | | active form of a | | | | |
| | | product from an | | | | |
| | | inactive precursor | | | | |
| | | form. | | | | |
| CELLULAR_PROTEIN_METABOLIC_PROCESS | 1117 | Genes annotated by | 79 | 0.0707 | 7.55E−25 | 1.84E−22 |
| | | the GO term | | | | |
| | | GO: 0044267. The | | | | |
| | | chemical reactions and | | | | |
| | | pathways involving a | | | | |
| | | specific protein, rather | | | | |
| | | than of proteins in | | | | |
| | | general, occurring at | | | | |
| | | the level of an | | | | |
| | | individual cell. | | | | |
| | | Includes protein | | | | |
| | | modification. | | | | |
| BENPORATH_NANOG_TARGETS | 988 | Set ‘Nanog targets’: | 74 | 0.0749 | 8.17E−25 | 1.94E−22 |
| | | genes upregulated and | | | | |
| | | identified by ChIP on | | | | |
| | | chip as Nanog | | | | |
| | | [GeneID = 79923] | | | | |
| | | transcription factor | | | | |
| | | targets in human | | | | |
| | | embryonic stem cells. | | | | |
| GRAESSMANN_RESPONSE_TO_MC_AND_- | 770 | Genes down-regulated | 65 | 0.0844 | 1.04E−24 | 2.41E−22 |
| DOXORUBICIN_DN | | in ME-A cells (breast | | | | |
| | | cancer, sensitive to | | | | |
| | | apoptotic stimuli) | | | | |
| | | exposed to | | | | |
| | | doxorubicin | | | | |
| | | [PubChem = 31703] in | | | | |
| | | the presence of | | | | |
| | | medium concentrate | | | | |
| | | (MC) from ME-C cells | | | | |
| | | (breast cancer, | | | | |
| | | resistant to apoptotic | | | | |
| | | stimuli). | | | | |
| MORF_GNB1 | 303 | Neighborhood of | 42 | 0.1386 | 1.63E−24 | 3.69E−22 |
| | | GNB1 | | | | |
| GOBERT_OLIGODENDROCYTE_- | 1080 | Genes down-regulated | 77 | 0.0713 | 1.87E−24 | 4.15E−22 |
| DIFFERENTIATION_DN | | during differentiation | | | | |
| | | of Oli-Neu cells | | | | |
| | | (oligodendroglial | | | | |
| | | precursor) in response | | | | |
| | | to PD174265 | | | | |
| | | [PubChemID = 4709]. | | | | |
| ZHENG_BOUND_BY_FOXP3 | 491 | Genes whose | 52 | 0.1059 | 1.92E−24 | 4.17E−22 |
| | | promoters are bound | | | | |
| | | by FOXP3 | | | | |
| | | [GeneID = 50943] | | | | |
| | | based an a ChIP-chip | | | | |
| | | analysis. | | | | |
| KRIGE_RESPONSE_TO_TOSEDOSTAT_6HR_UP | 953 | Genes up-regulated in | 72 | 0.0756 | 2.18E−24 | 4.63E−22 |
| | | HL-60 cells (acute | | | | |
| | | promyelocytic | | | | |
| | | leukemia, APL) after | | | | |
| | | treatment with the | | | | |
| | | aminopeptidase | | | | |
| | | inhibitor tosedostat | | | | |
| | | (CHR-2797) | | | | |
| | | [PubChem = 15547703] | | | | |
| | | for 6 h. | | | | |
| REGULATION_OF_METABOLIC_PROCESS | 799 | Genes annotated by | 65 | 0.0814 | 7.85E−24 | 1.63E−21 |
| | | the GO term | | | | |
| | | GO: 0019222. Any | | | | |
| | | process that modulates | | | | |
| | | the frequency, rate or | | | | |
| | | extent of the chemical | | | | |
| | | reactions and pathways | | | | |
| | | within a cell or an | | | | |
| | | organism. | | | | |
| GCCATNTTG_V$YY1_Q6 | 427 | Genes with promoter | 48 | 0.1124 | 8.95E−24 | 1.82E−21 |
| | | regions [−2 kb, 2 kb] | | | | |
| | | around transcription | | | | |
| | | start site containing the | | | | |
| | | motif GCCATNTTG | | | | |
| | | which matches | | | | |
| | | annotation for YY1: | | | | |
| | | YY1 transcription | | | | |
| | | factor | | | | |
| KRIGE_RESPONSE_TO_TOSEDOSTAT_24HR_UP | 783 | Genes up-regulated in | 64 | 0.0817 | 1.37E−23 | 2.73E−21 |
| | | HL-60 cells (acute | | | | |
| | | promyelocytic | | | | |
| | | leukemia, APL) after | | | | |
| | | treatment with the | | | | |
| | | aminopeptidase | | | | |
| | | inhibitor tosedostat | | | | |
| | | (CHR-2797) | | | | |
| | | [PubChem = 15547703] | | | | |
| | | for 24 h. | | | | |

Cumulatively, these analyses implicate Foxa3 in the regulation of HSC metabolic and proliferative stress. To explore this further, CD45.2+ HSC (i.e. LSK CD150+CD48− cells) were isolated from recipients of CD45.2+ Foxa3^{+/+} or Foxa3^{-/-} WBM >8 months post-transplant and examined by staining with DCFDA for reactive oxygen species (ROS). Foxa3^{-/-} HSC displayed a 50% increase in ROS relative to Foxa3^{+/+} HSC (p=0.006, FIG. 7B). Despite the increase in basal ROS levels, Foxa3^{-/-} HSC were able to recover from induced ROS similar to control HSC (FIG. 7B). These data confirm bioinformatics predictions that Foxa3^{-/-} HSC are subject to elevated metabolic stress.

In sum, Foxa3 is dispensable to the hematopoietic compartment during homeostasis (FIGS. 6B-C), yet contributes to optimal HSC function post-transplant (FIG. 6F). Indeed, the Foxa3^{-/-} repopulating phenotype is most dramatic when greater pressure to repopulate is placed on individual cells (e.g. in limiting dilution transplants and serial transplantation) (FIG. 6H) and Foxa3^{-/-} HSC display a significant increase in ROS, which is known to compromise HSC self-renewal, maintenance, and repopulating potential (Ito et al., 2006; Jang and Sharkis, 2007; Taniguchi Ishikawa et al., 2012; Tothova et al., 2007).

Thus, we identified Foxa3 as a novel regulator of HSPC repopulation (FIGS. 2C and 3E). Foxa genes have not been implicated in HSPC biology. We found that Foxa3 is highly expressed by HSC (FIG. 6A) and although Foxa3^{-/-} mice display normal hematopoiesis (FIG. 6B-C), Foxa3^{-/-} HSC are deficient in CFUs and primary and secondary in vivo repopulation (FIG. 6D-F). Other genes are also known to be dispensable for homeostasis but contribute to HSC function under pathophysiological conditions, such as hematopoietic stress (e.g. p21, β-catenin, FoxOs, Gadd45a, and Gab2) (Chen et al, 2014; Cheng et al, 2000; Zhang et al, 2007; Zhao et al, 2007).

Indeed, P2ry14, also identified here, is not required for steady-state hematopoiesis but contributes to HSC function following stress and injury (Cho et al, 2014). Thus, mechanisms that preserve the hematopoietic compartment during stress (e.g. post-transplant) are often not required for homeostasis and Foxa3 appears to be a newly discovered regulator of these processes. Indeed, genes targeted by active LT-HSC enhancers containing FOXA3 binding motifs were enriched for pathways controlling cell cycle, metabolism, and stress and Foxa3^{-/-} HSC display a significant increase in ROS content (FIG. 7B, Tables 4 and 7-9). Increased ROS levels are known to compromise HSC self-renewal, quiescence, and repopulating potential (Ito et al, 2006; Jang and Sharkis, 2007; Taniguchi Ishikawa et al, 2012; Tothova et al, 2007). However, Foxa3^{-/-} HSC's failure to efficiently repopulate ablated mice was most pronounced when limiting cell numbers were transplanted and after serial transplantation (FIG. 6H). These are both scenarios in which the pressure on individual repopulating cells to expand and differentiate is extreme. In contrast, during homeostasis, when the pressure on individual cells to maintain steady state hematopoiesis is low, Foxa3 is dispensable. Thus, in the absence of Foxa3, HSPC fail to respond efficiently to hematologic stress.

3. Advantages of Using Methods of the Present Inventions.

Currently, there are several limitations for successful hematopoietic stem cell engraftment. These include but are not limited to: donor availability, i.e. finding HLA matches for reducing graft rejections and GVHD; small numbers of cells, in particular for transplants using umbilical cord derived blood cells, transplant cells or tissues spending an extended time in culture prior to transplantation, etc. Transplants with small cell numbers result in a delay in stable engraftment. Extended time in cell culture has multiple deleterious effects on cells with respect to transplantation activity, including increased risk for opportunistic bacteria and yeast infections in the cells and/or tissues intended for transplantation, increasing cell death of certain cell types, and differentiation of cells intended for transplant. Each of which results in a loss of engraftment potential. Thus, one advantage of using methods of the present inventions is to enhance the repopulating activity of the HSC prior to transplant, such that the need for extended cell culture is minimized. Additional characteristics such as successful niche lodgment and retention, survival under stress, activation, and differentiation may also contribute to stable engraftment.

Further, because using UCB cells results in delayed engraftment, some physicians are remiss to using UCB for transplants. A limitation of using UCB cells includes but is not limited to a failure of engraftment due to too few cells. Too few cells, as when using bone marrow transplants, leaves the patient susceptible to infection while waiting for engraftment. Therefore, it is contemplated that by increasing the efficiency of engraftment, i.e. by using methods of the present inventions for silencing at least one GASP gene, even when transplanting small cell numbers engraftment might be achieved in a reasonable time frame and thus UCB cells might provide transplants to a wider range of patients. UCB is particularly valuable as a cell source because there tend to be fewer immunological side-effects (i.e. will tolerate a greater HLA mismatch than HSC isolated from mPB or bone marrow). Thus, it is an attractive option for patients who lack a perfectly matched donor. In some embodiments, UCBs treated for silencing at least one GASP gene are contemplated to provide cells having faster time periods to engraftment. In some embodiments, the use of UCB treated cells of the present inventions may provide UCB cells capable of engraftment in patients with a greater mismatch of HLA haplotypes.

HSPC in vivo repopulating activity is complex, requiring the orchestration of many molecular and cellular processes. This is evident by the disparate putative functions of the molecules with positive or negative regulation identified in our screen. Manipulating the regulation of stable HSPC engraftment is contemplated as a strategy for improving the efficiency of HSCT.

B. Human Patients.

The following are exemplary materials and methods for use with the inventions described herein in particular for human patients. In one preferred embodiment, methods for pre-treatment of hematopoietic stem and progenitor cells with shRNA for a GASP gene family member prior to transplant to enhance their ability to stably engraft and reconstitute an ablated hematopoietic system are provided herein.

### Exemplary Human Cell Populations.

Sources of human cell populations contemplated for use in human transplantation include, but are not limited to, bone marrow cells, umbilical cord blood-derived cells, mobilized peripheral blood cells (mPB), etc. Exemplary bone marrow cells are obtained from bone marrow (e.g. collected via syringe from the pelvic bone). Umbilical cord blood HSCs may be obtained from umbilical cord blood (e.g. collected via syringe from newborn umbilical cords and then frozen for storage until needed). Blood banking facilities may also be sources of cells for transplant (e.g. from blood or umbilical cord blood banking). Cord blood cells from siblings is contemplated for use as host cells for transplantation. Mobilized peripheral blood may be collected via apheresis from donors pre-treated for 4-6 days with GM-CSF (Granulocyte-macrophage colony-stimulating factor). In one embodiment, these populations are not enriched for specific populations prior to transplantation. In other embodiments, populations for use in transplantation may be enriched for specific cell populations. For example, apheresis involves removal of whole blood from a patient or donor with an instrument that is designed as a centrifuge for separating components of whole blood. The components which are separated and withdrawn include: Plasma (plasmapheresis); Platelets (plateletpheresis); and Leukocytes (leukapheresis).

As used herein, treatment includes non-enriched populations (total cells from each of these sources), since this is the more common current therapy, in addition to treating enriched population of CD34+ cells prior to transplant. One example of obtaining CD34+ enriched populations includes staining hematopoetic cells with fluorescently labeled anti-CD34 antibodies and then collecting this population via fluorescence activated cell sorting using a flow cytometer. In other examples, CD34+ enriched populations may be obtaining by using a combination of monoclonal antibodies (negative selections) using the Stem Sep method or with positive selection based on collecting cells having surface CD34 antigens using the Mini Macs system, panning, bead separation, etc.

### Exemplary Procedure for Human Transformation.

Methods of using shRNAs targeting GASP family members for delivery to human hematopoietic stem and progenitor cells (i.e. human CD34+ cells) are briefly, as follows. Human CD34+(CD: cluster of differentiation) cells will be isolated from a human cell population by flow cytometry and cultured in tissue culture medium such as X-vivo-10 (Lonza Group Ltd., Basel, Switzerland) in the presence of recombinant human cytokines such as SCF (Stem cell factor), TPO (thrombopoietin) and FLT3 (receptor-type tyrosine-protein kinase FLT3) for 24-48 hours. These cells will then be transduced with lentiviral vectors or integration defective lentiviral vectors carrying the appropriate shRNAs in tissue culture plates or flasks that are coated with retronectin. shRNAs may also be introduced into cells via electroporation. Lentiviral vectors will be used at a multiplicity of infection (MOI) of 25-150.

### Exemplary Procedure for Human Implantation.

Patients will be conditioned for transplant according to the standard recommendation of care for their disease and indication for transplant. Bone marrow, mPB, or umbilical cord blood will then be infused into patients intravenously.

### Examples of how Engraftment Will be Evaluated as a Success.

Patients are considered engrafted when their absolute neutrophil count (ANC) exceeds 500 cells/μL of peripheral blood. This typically occurs between 14-35 days and >35 days post infusion of cells for bone marrow/mPB and umbilical cord blood, respectively, and depending on the disease indication and conditioning of patient prior to transplant. Any acceleration of engraftment will be considered a success, especially for umbilical cord blood, where delayed engraftment is a particular problem in adult transplant recipients. Also, enhanced hematopoietic chimerism of the transplanted cells will also be considered a success, especially for umbilical cord blood transplantation where hematopoietic chimerism can be poor.

In one contemplative embodiment, autologous human hematopoietic stem cells may be used in methods described herein for medical treatments requiring bone marrow transplantation. In another contemplated embodiment, human hematopoietic stem cells considered having a matching HLA haplotype may be used as described herein for bone marrow transplantation.

Treatment of umbilical cord blood-derived cells (HSCs) with shRNA for reducing expression of a GASP gene is unexpected in part because although there was no mention of lowering expression of Gprasp1 or Gprasp2, Lanza, et al., U.S. Pat. No. 8,796,021. “Blastomere culture to produce mammalian embryonic stem cells.” Publication date Aug. 5, 2014, lists Gprasp1 and Gprasp2 as factors for adding to cell cultures of blastomeres for producing blastomere-derived human (h) ESCs in order to produce hematopoietic precursors for therapeutic use, including transplantation.

### REFERENCES, EACH REFERENCE IS HEREIN INCORPORATED IN ITS ENTIRETY

Abu-Helo and Simonin. 2010. Identification and biological significance of G protein-coupled receptor associated sorting proteins (GASPs). Pharmacology & therapeutics 126:244-250.
Ali, et al., 2009. Forward RNAi screens in primary human hematopoietic stem/progenitor cells. Blood 113:3690-3695.
Ashburner, et al., 2000. Gene ontology: tool for the unification of biology. The Gene Ontology Consortium. Nat Genet 25:25-29.
Behr, et al., 2007. Impaired male fertility and atrophy of seminiferous tubules caused by haploinsufficiency for Foxa3. Dev Biol 306:636-645.
Boitano, et al., 2010. Aryl hydrocarbon receptor antagonists promote the expansion of human hematopoietic stem cells. Science 329:1345-1348.
Buscarlet, et al., 2014. Essential role of BRG, the ATPase subunit of BAF chromatin remodeling complexes, in leukemia maintenance. Blood 123:1720-1728.
Busch, et al., 2015. Fundamental properties of unperturbed haematopoiesis from stem cells in vivo. Nature 518:542-6.
Cabezas-Wallscheid, et al., 2014. Identification of regulatory networks in HSCs and their immediate progeny via integrated proteome, transcriptome, and DNA Methylome analysis. Cell Stem Cell 15:507-522.
Cai, et al., 2015. Runx1 Deficiency Decreases Ribosome Biogenesis and Confers Stress Resistance to Hematopoietic Stem and Progenitor Cells. Cell Stem Cell 17:165-177.
Cavazzana, et al., 2014. Stem cell transplantation for primary immunodeficiencies: the European experience. Current opinion in allergy and clinical immunology 14:516-520.
Chambers, et al., 2007. Hematopoietic fingerprints: an expression database of stem cells and their progeny. Cell Stem Cell 1:578-591.
Chen, et al., 2014. Gadd45a regulates hematopoietic stem cell stress responses in mice. Blood 123:851-862.
Cheng, et al., 2000. Hematopoietic stem cell quiescence maintained by p21cip1/waf1. Science 287:1804-1808.
Cho, et al., 2014. Purinergic P2Y(1J(4) receptor modulates stress-induced hematopoietic stem/progenitor cell senescence./Clin Invest 124:3159-3171.
Cisternas, et al., 2003. Cloning and characterization of human CADPS and CADPS2, new members of the Ca2+-dependent activator for secretion protein family. Genomics 81:279-291.
Cohen, et al., 2014. Role of allogeneic stem cell transplantation in mantle cell lymphoma. European journal of hematology 94: 290-7.
Cutler, et al., 2013. Prostaglandin-modulated umbilical cord blood hematopoietic stem cell transplantation. Blood 122:3074-3081.
Deneault, et al., 2009. A functional screen to identify novel effectors of hematopoietic stem cell activity. Cell 137:369-379.
Fares, et al., 2014. Cord blood expansion. Pyrimidoindole derivatives are agonists of human hematopoietic stem cell self-renewal. Science 345:1509-1512.
Fellmann, et al., 2011. Functional identification of optimized RNAi triggers using a massively parallel sensor assay. Molecular cell 41:733-746.
Friedman and Kaestner. 2006. The Foxa family of transcription factors in development and metabolism. Cellular and molecular life sciences: CMLS 63:2317-2328.
Geng, et al., 2011. Follistatin-like 1 (Fstl) is a bone morphogenetic protein (BMP) 4 signaling antagonist in controlling mouse lung development. Proc Natl Acad Sci USA 108:7058-7063.
Gibbs, et al., 2008. The CAP superfamily: cysteine-rich secretory proteins, antigen 5, and pathogenesis-related 1 proteins-roles in reproduction, cancer, and immune defense. Endocrine reviews 29:865-897.
Grant, et al., 2011. FIMO: scanning for occurrences of a given motif. Bioinformatics 27:1017-1018.
Gruber, et al., 2012. An Inv(16)(p13.3q24.3)-encoded CBFA2T3-GLIS2 fusion protein defines an aggressive subtype of pediatric acute megakaryoblastic leukemia. Cancer Cell 22:683-697.
He, et al., 2014. Global view of enhancer-promoter interactome in human cells. Proc Natl Acad Sci USA 111:E2191-2199.
Heng, et al., Immunological Genome Project. 2008. The Immunological Genome Project: networks of gene expression in immune cells. Nat Immunol 9:1091-1094.
Hoggatt, et al., 2009. Prostaglandin E2 enhances hematopoietic stem cell homing, survival, and proliferation. Blood 113:5444-5455.
Holmfeldt, et al., 2013. Nfix is a novel regulator of murine hematopoietic stem and progenitor cell survival. Blood 122:2987-2996.
Hope, et al., 2010. An RNAi screen identifies Msi2 and Proxl as having opposite roles in the regulation of hematopoietic stem cell activity. Cell Stem Cell 7:101-113.
Ionescu, et al., 2012. FoxA family members are crucial regulators of the hypertrophic chondrocyte differentiation program. Dev Cell 22:927-939.
Ito, et al., 2006. Reactive oxygen species act through p38 MAPK to limit the lifespan of hematopoietic stem cells. Nat Med 12:446-451.
Jang and Sharkis. 2007. A low level of reactive oxygen species selects for primitive hematopoietic stem cells that may reside in the low-oxygenic niche. Blood 110:3056-3063.
Kuroiwa, et al., 2011. The guanine nucleotide exchange factor Arhgef5 plays crucial roles in Src-induced podosome formation. Journal of Cell Science 124:1726-1738.
Lara-Astiaso, et al., 2014. Immunogenetics. Chromatin state dynamics during blood formation. Science 345:943-949.
Ma, et al., 2014. The Sox4/Tcf711 axis promotes progression of BCR-ABL-positive acute lymphoblastic leukemia. Haematologica 99:1591-1598.
Masetti, et al., 2013. CBFA2T3-GLIS2 fusion transcript is a novel common feature in pediatric, cytogenetically normal AML, not restricted to FAB M7 subtype. Blood 121:3469-3472.
Matsubara, et al., 2005. Endomucin, a CD34-like sialomucin, marks hematopoietic stem cells throughout development. The Journal of experimental medicine 202:1483-1492.
McKinney-Freeman, et al., 2012. The transcriptional landscape of hematopoietic stem cell ontogeny. Cell Stem Cell 11:701-714.
Mose, et al., 2010. G protein-coupled receptor-associated sorting protein 1 regulates the postendocytic sorting of seven-transmembrane-spanning G protein-coupled receptors. Pharmacology 86:22-29.
Niesmann, et al., 2011. Dendritic spine formation and synaptic function require neurobeachin. Nature communications 2:557.
Rao, et al., 2015. High-level Gpr56 expression is dispensable for the maintenance and function of hematopoietic stem and progenitor cells in mice. Stem Cell Research 14:307-322.
Rossi, et al., 2012. The sixth sense: hematopoietic stem cells detect danger through purinergic signaling. Blood 120:2365-2375.
Saito, et al., 2013. Maintenance of the hematopoietic stem cell pool in bone marrow niches by EVIl-regulated GPR56. Leukemia 27:1637-1649.
Singer, et al., 2013. GPR56 and the developing cerebral cortex: cells, matrix, and neuronal migration. Molecular neurobiology 47:186-196.
Solaimani, et al., 2015. Whole-transcriptome analysis of endothelial to hematopoietic stem cell transition reveals a requirement for Gpr56 in HSC generation. The Journal of experimental medicine 212:93-106.
Sun, et al., 2014. Clonal dynamics of native haematopoiesis. Nature 514:322-327.
Talano and Cairo. 2014. Hematopoietic stem cell transplantation for sickle cell disease: state of the science. European journal of haematology 94:391-9.
Taniguchi, et al., 2012. Connexin-43 prevents hematopoietic stem cell senescence through transfer of reactive oxygen species to bone marrow stromal cells. Proc Natl Acad Sci USA 109:9071-9076.
Tothova, et al., 2007. FoxOs are critical mediators of hematopoietic stem cell resistance to physiologic oxidative stress. Cell 128:325-339.
Urrutia, 2003. KRAB-containing zinc-finger repressor proteins. Genome biology 4:231.
Walasek, et al., 2012. Hematopoietic stem cell expansion: challenges and opportunities. Annals of the New York Academy of Sciences 1266:138-150.
Wei, et al., 2015. Epicardial FSTL1 reconstitution regenerates the adult mammalian heart. Nature 525:479-485.
Weirauch, et al., 2014. Determination and inference of eukaryotic transcription factor sequence specificity. Cell 158:1431-1443.
Wilson, et al., 2004. c-Myc controls the balance between hematopoietic stem cell self-renewal and differentiation. Genes Dev 18:2747-2763.
Xu, et al., 2013. The winged helix transcription factor Foxa3 regulates adipocyte differentiation and depot-selective fat tissue expansion. Mol Cell Biol 33:3392-3399.
Zhang, et al., 2013. Sox4 is a key oncogenic target in C/EBPalpha mutant acute myeloid leukemia. Cancer Cell 24:575-588.
Zhang, et al., 2007. Abnormal hematopoiesis in Gab2 mutant mice. Blood 110:116-124.
Zhao, C, J. Blum, et al., 2007. Loss of beta-catenin impairs the renewal of normal and CML stem cells in vivo. Cancer Cell 12:528-541.
Zhong, et al., 2010. Umbilical cord blood stem cells: what to expect. Annals of the New York Academy of Sciences 1205:17-22.

### EXPERIMENTAL

The following examples serve to illustrate certain embodiments and aspects of the present invention and are not to be construed as limiting the scope thereof.

The following abbreviations are used herein: CFU (Colony Forming Unit), DCFDA (2′,7′-dichlorofluorescin diacetate), FACS (Fluorescence Activated Cell Sorting), GSEA (Gene Set Enrichment Analysis), GO (Gene Ontology), GOI (Gene of Interest), HSC (Hematopoietic Stem Cell), HSCT (Hematopoietic Stem Cell Transplantation), HSPC (Hematopoietic Stem and Progenitor Cell), KO (Knock Out), IM-PET (Integrated Method for Predicting Enhancer Targets), LDA (Limiting Dilution Analysis), LSK (Lineage″Sca-1⁺c-Kit⁺), MSCV (Murine Stem Cell Virus), PB (Peripheral Blood), PWM (Position Weight Matrix), RH FGF-1 (Recombinant Human Fibroblast Growth Factor-1), RM IGF2 (Recombinant Murine Insulin-like Growth Factor 2), RM SCF (Recombinant Murine Stem Cell Factor), ROS (Reactive Oxygen Species), TBHP (tert-Butyl hydroperoxide), VSV-G (Vesicular Stomatitis Virus Glycoprotein), WBM (Whole Bone Marrow), WT (Wild Type), PGK (phosphoglycerate kinase).

### Example I

The following are exemplary materials and methods for use with the inventions described herein in particular for human patients. In one preferred embodiment, methods for pre-treatment of hematopoietic stem and progenitor cells with shRNA for a GASP gene family member prior to transplant to enhance their ability to stably engraft and reconstitute an ablated hematopoietic system are provided herein.

### Exemplary Human Cell Populations.

Sources of human cell populations contemplated for use in human transplantation in include but are not limited to: bone marrow cells, umbilical cord blood-derived cells (HSCs), mobilized peripheral blood cells (mPB), etc. Exemplary bone marrow cells are obtained from bone marrow, e.g. collected via syringe from the pelvic bone. umbilical cord blood-derived cells as HSCs may be obtained from umbilical cord blood, e.g. collected via syringe from newborn umbilical cords and then frozen for storage until needed. Blood banking facilities may also be sources of cells for transplant, e.g. from blood or umbilical cord blood banking. Cord blood cells from siblings is contemplated for use as host cells for transplantation. Mobilized peripheral blood may be collected via apheresis from donors pre-treated for 4-6 days with Gm-CSF (Granulocyte-macrophage colony-stimulating factor). In one embodiment, these populations may not be enriched for specific populations prior to transplantation. In other embodiments, populations for use in transplantation may be enriched for selecting specific cell populations. For example, apheresis involves removal of whole blood from a patient or donor with an instrument that is designed as a centrifuge for separating components of whole blood. The components which are separated and withdrawn include: Plasma (plasmapheresis); Platelets (plateletpheresis); and Leukocytes (leukapheresis).

As used herein, treatment includes non-enriched populations (total cells from each of these sources), since this is the more common current therapy, in addition to treating enriched population of CD34+ cells prior to transplant. One example of obtaining CD34+ enriched populations includes staining hematopoetic cells with fluorescently labeled anti-CD34 antibodies and then collecting this population via fluorescence activated cell sorting using a flow cytometer. In other examples, CD34+ enriched populations may be obtaining by using a combination of monoclonal antibodies (negative selections) using the Stem Sep method or with positive selection based on collecting cells having surface CD34 antigens using the Mini Macs system, panning, bead separation, etc.

### Exemplary Procedure for Human Transformation.

Methods of using shRNAs targeting GASP family members for delivery to human hematopoietic stem and progenitor cells (i.e. human CD34+ cells) are briefly, as follows.

Human CD34+ cells will be isolated from a human cell population by flow cytometry and cultured in tissue culture medium such as X-vivo-10 (Lonza Group Ltd., Basel, Switzerland) in the presence of recombinant human cytokines such as SCF (Stem cell factor), TPO (thrombopoietin) and FLT3 (receptor-type tyrosine-protein kinase FLT3) for 24-48 hours. These cells will then be transduced with lentiviral vectors or integration defective lentiviral vectors carrying the appropriate shRNAs in tissue culture plates or flasks that are coated with retronectin. shRNAs may also be introduced into cells via electroporation. Lentiviral vectors will be used at a multiplicity of infection (MOI) of 25-150.

Sources of lentiviral vectors for expressing shRNA Gprasp1, Gprasp2, or Armcx1 (GASP7) and other GASP family genes, such as Bhlhb9 (Gprasp3), etc., for use in methods of the present inventions for reducing expression of human genes in human HSC cells, include but are not limited to: lentiviral expression vector constructs comprising predesigned shRNA inhibitory siRNA directed against mouse Gprasp1 and human Gprasp1; and against mouse Gprasp2 and human Gprasp2; and against mouse Armcx1 and human Armcx1, may be obtained commercially from several companies, including but not limited to Qiagen (27220 Turnberry Lane, Suite 200, Valencia, Calif. 91355: www.qiagen.com/us/), OriGene (9620 Medical Center Dr., Suite 200, Rockville, Md. 20850: www.origene.com) and Santa Cruz Biotechnology (10410 Finnell Street Dallas, Tex. 75220: www.scbt.com/). For at least one company, OriGene Technologies, Inc., (www.origene.com) predesigned shRNA inhibitory siRNA lentiviral particles for silencing Gprasp1, accessed 4-11-2016; Gprasp2 accessed 4-05-2016; and Armcx1 accessed 3-11-2016, have a guaranteed knockdown of >70%.

Another example of a shGASP-1 lentiviral vector for reducing expression of a human Gprasp1 shRNA in human cells that may find use in the present inventions includes a description in Kargl, et al., “The trafficking of GPR55 is regulated by the G protein-coupled receptor-associated sorting protein 1.” BMC Pharmacol. 10 (Suppl. 1): A1. Published online 2010. This reference describes knockdown of endogenous GASP-1 levels in Human Embryonic Kidney cells induced by infection with Lenti-shGASP-1 (shGASP-1).

An example for a Bhlhb9-shRNA may be obtained from Virigene Biosciences, See Table 12.

### Example II

Exemplary Human Gprasp1 and Gprasp2 shRNA Reduces Gprasp1 and Gprasp2 Expression in Human Hematopoetic Stem Cells, Respectively.

Silencing vectors for knocking down human Gprasp1 and Gprasp2 gene expression were constructed, including but were not limited to a promoter, a shRNA sequence and a lentiviral expression vector. Exemplary shRNA sequences are shown in Table 11. Exemplary FIG. 11 demonstrates knock down levels for each of the genes in human cell lines.

FIG. 15. Validation of shRNAs that efficiently knock-down human GPRASP1 or GPRASP2 RNA expression in human cell lines. Validation of shRNAs showing a robust knock-down of human Gprasp1 or Gprasp2 in human cell lines.

### Example III

Exemplary Procedure for Engineering Alleles that Lack the Coding Region of Gprasp Genes Using CRISPR/Cas9 Technology.

Methods of using CRISPR/Cas9 technology for reducing Gprasp gene expression in human hematopoietic stem and progenitor cells (i.e. human CD34+ cells) are briefly, as follows. Human stem cells may be engineered to contain an allele that lacks the coding region of one, or both Gprasp1 and Gprasp2 by CRISPR/Cas9 technology. Thus, one contemplated method for enhancing stem cell transplantation is to alter or remove one or more nucleotides from Gprasp1 and/or Gprasp2 coding sequences to reduce expression of one or more Gprasp genes prior to transplantation.

### Example IV

### Exemplary Procedure for Human Implantation.

Patients will be conditioned for transplant according to the standard recommendation of care for their disease and indication for transplant. Bone marrow, mPB, or umbilical cord blood, or umbilical cord blood-derived cells will then be infused into patients intravenously.

### Examples of how Engraftment Will be Evaluated as a Success.

Patients are considered engrafted when their absolute neutrophil count (ANC) exceeds 500 cells/4Lof peripheral blood. This typically occurs between 14-35 days and >35 days post infusion of cells for bone marrow/mPB and umbilical cord blood; umbilical cord blood-derived cells, respectively and depending on the disease indication and conditioning of patient prior to transplant. Any acceleration of engraftment will be considered a success, especially for umbilical cord blood-derived cells, where delayed engraftment is a particular problem in adult transplant recipients. Also, enhanced hematopoietic chimerism of the transplanted cells will also be considered a success, especially for umbilical cord blood-derived cells transplantation where hematopoietic chimerism can be poor.

In one contemplative embodiment, autologous human hematopoietic stem cells may be used in methods described herein for medical treatments requiring bone marrow transplantation. In another contemplated embodiment, human hematopoietic stem cells considered having a matching HLA haplotype may be used as described herein for bone marrow transplantation.

Treatment of cord blood stem cells with shRNA for reducing expression of a GASP gene is unexpected in part because although there was no mention of lowering expression of Gprasp1 or Gprasp2, Lanza, et al., U.S. Pat. No. 8,796,021. “Blastomere culture to produce mammalian embryonic stem cells.” Publication date Aug. 5, 2014, lists Gprasp1 and Gprasp2 as factors for adding to cell cultures of blastomeres for producing blastomere-derived human (h) ESCs in order to produce hematopoietic precursors for therapeutic use, including transplantation.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, molecular biology, cell biology, genetics, statistics or related fields are intended to be within the scope of the following claims.

1. A method for enhancing hematopoietic stem cell (HSC) engraftment, comprising,
a) providing,
i) a human hematopoietic stem cell (HSC) population, wherein said HSCs have a HLA haplotype and express a gene in the G-protein coupled receptor Associated Sorting Protein (GASP) gene family, and
ii) a human patient having an HLA haplotype,
b) treating said HSCs under conditions such that expression of said GASP gene in said HSC population is reduced, and
c) transplanting said treated HSCs into said patient.

2. The method of claim 1, wherein said treatment is shRNA-mediated knockdown of said GASP gene.

3. The method of claim 2, wherein said knockdown is up to but not including a 100% reduction in gene expression.

4. The method of claim 1, wherein after said transplantation said GASP gene expression increases in treated HSCs.

5. The method of claim 1, wherein after said transplantation said GASP gene expression increases in progeny cells of said treated HSCs.

6. The method of claim 1, wherein after said treatment said GASP gene is expressed in progeny cells of said treated HSCs.

7. The method of claim 1, wherein after said treatment said GASP gene is not knocked down in progeny cells of said treated HSCs.

8. The method of claim 1, wherein said GASP gene is selected from the group consisting of Gprasp2 and Armcx1.

9. The method of claim 1, wherein said GASP gene is the Gprasp1 gene.

10. The method of claim 1, wherein said GASP gene is a Basic Helix-Loop-Helix Domain Containing, Class B, 9.

11-12. (canceled)

13. The method of claim 11, wherein said HSCs of step a) express three GASP genes, wherein said three GASP genes are Gprasp1, Gprasp2 and Basic Helix-Loop-Helix Domain Containing, Class B, 9.

14. The method of claim 1, wherein said human hematopoietic stem population is obtained from a sample selected from the group consisting of bone marrow, mobilized peripheral blood and umbilical cord blood.

15. (canceled)

16. The method of claim 15, wherein said HSC HLA haplotype is a mismatch (allogeneic) between the stem cell population of said umbilical cord blood (UCB) and said HLA haplotype of said patient.

17. A method for enhancing hematopoietic stem cell (HSC) engraftment, comprising,
a) providing,
i) a human umbilical cord blood (UCB) stem cell population, wherein said UCBs have a HLA haplotype and express a gene in the G-protein coupled receptor Associated Sorting Protein (GASP) gene family, wherein said GASP gene is selected from the group consisting of Gprasp1, Gprasp2, Basic Helix-Loop-Helix Domain Containing, Class B, 9, and Armcx1, and
ii) a human patient, wherein said patient has a major Human Leukocyte Antigen (HLA) haplotype, and
b) treating said HSCs to reduce expression of said GASP gene, and
c) transplanting said treated HSCs into said patient.

18. The method of claim 17, wherein said HSC HLA haplotype is a mismatch (allogeneic) between said umbilical cord blood (UCB) stem cell population and said HLA haplotype of said patient.

19-29. (canceled)

30. A method of treating a hematopoietic stem cell (HSC) population, comprising,
1) providing a hematopoietic stem cell (HSC) population, wherein said HSCs express a gene in the G-protein coupled receptor Associated Sorting Protein (GASP) gene family, and
2) treating said HSCs ex vivo under conditions such that expression of said GASP gene in said HSC population is reduced.

31. The method of claim 30, wherein said treatment is shRNA-mediated knockdown of said GASP gene.

32. The method of claim 31, wherein said knockdown of said GASP gene is between 80% up to but not including 100% reduction in expression.

33-35. (canceled)

36. The method of claim 30, wherein said HSCs of step a) express two or more GASP genes.

37-39. (canceled)

40. The method of claim 30, wherein said hematopoietic stem population is obtained from umbilical cord blood (UCB).

41-44. (canceled)
